(19) United States
(12) Patent Application Publication
(10) Pub. No.: US 2018/0303952 A1
(43) Pub. Date: Oct. 25, 2018

(54) CD147 ANTIBODIES, ACTIVATABLE CD147 ANTIBODIES, AND METHODS OF MAKING AND USE THEREOF

(71) Applicant: CytomX Therapeutics, Inc., South San Francisco, CA (US)

(72) Inventors: Jason Gary SAGERT, San Mateo, CA (US); Shuoyen Jack Lin, Millbrae, CA (US); James William West, Bend, OR (US); Jonathan Alexander Terrett, Lexington, MA (US)

(21) Appl. No.: 15/917,404

(22) Filed: Mar. 9, 2018

Related U.S. Application Data
(60) Provisional application No. 62/469,429, filed on Mar. 9, 2017.

(51) Int. Cl. A61K 47/68; C07K 16/30; A61P 35/00; A61K 45/06

(52) U.S. Cl. CPC A61K 47/6849; C07K 16/30; G01N 33/56966; A61P 35/00; A61K 45/06; A61K 47/6803

(57) ABSTRACT

The invention relates generally to antibodies that bind CD147, activatable antibodies that bind to CD147 and methods of making and using these antibodies and activatable antibodies in a variety of therapeutic, prophylactic, and diagnostic contexts. In some embodiments, the CD147 antibodies and CD147 activatable antibodies bind human and cynomolgus monkey CD147.

### CROSS-REFERENCE TO RELATED APPLICATIONS

This application claims the benefit of U.S. provisional application No. 62/469,429, filed Mar. 9, 2017, the contents of which are incorporated by reference in their entirety.

### INCORPORATION OF SEQUENCE LISTING

The contents of the text file named “CYTM_050_001US_SeqList_ST25.txt”, which was created on Jul. 12, 2018 and is 580 KB in size, are hereby incorporated by reference in their entirety.

### FIELD OF THE INVENTION

The invention relates generally to antibodies that bind CD147, activatable antibodies that bind to CD147 and methods of making and using these antibodies and activatable antibodies in a variety of therapeutic, prophylactic, and diagnostic contexts.

### BACKGROUND OF THE INVENTION

Antibody-based therapies have proven effective treatments for several diseases but in some cases, toxicities due to broad target expression have limited their therapeutic effectiveness. In addition, antibody-based therapeutics have exhibited other limitations such as rapid clearance from the circulation following administration.

In the realm of small molecule therapeutics, strategies have been developed to provide prodrugs of an active chemical entity. Such prodrugs are administered in a relatively inactive (or significantly less active) form. Once administered, the prodrug is metabolized in vivo into the active compound. Such prodrug strategies can provide for increased selectivity of the drug for its intended target and for a reduction of adverse effects.

Accordingly, there is a continued need in the field of antibody-based therapeutics for antibodies that mimic the desirable characteristics of the small molecule prodrug.

### BRIEF SUMMARY OF THE INVENTION

Provided herein are antibodies that bind CD147, activatable antibodies that bind to CD147 and methods of making and using these antibodies and activatable antibodies in a variety of therapeutic, prophylactic, and diagnostic contexts. In some embodiments, the CD147 antibodies and CD147 activatable antibodies bind human and cynomolgus monkey CD147. In some embodiments, the CD147 antibodies and CD147 activatable antibodies bind both the glycosylated and deglycosylated forms of the CD147 antigen.

In one aspect of the invention, provided herein is an antibody or an antigen binding fragment thereof (AB) that specifically binds human CD147 and cynomolgus monkey CD147. In another aspect of the invention, provided herein is an antibody or an antigen binding fragment thereof (AB) that specifically binds human CD147 and/or cynomolgus monkey CD147. In some embodiments, the CD147 includes both deglycosylated CD147 and glycosylated CD147. In some embodiments, the AB specifically binds human CD147. In some embodiments, the AB only binds human CD147. In any of the antibodies or antibody binding fragments thereof, the AB can be selected from the group consisting of a Fab fragment, a F(ab′)2 fragment, a scFv, a scAb, a dAb, a single domain heavy chain antibody, and a single domain light chain antibody. In some embodiments, the antibody or antigen binding fragment thereof comprises an amino acid sequence comprising amino acid sequences selected from the group consisting of: (a) the VH CDR1 sequence GFTFSNYWMN (SEQ ID NO: 10) or GFTFSNYWMD (SEQ ID NO: 11); the VH CDR2 sequence EIRLKSYNYATH (SEQ ID NO: 12); the VH CDR3 sequence AGTDY (SEQ ID NO: 13); the VL CDR1 sequence KASQSVRTDVA (SEQ ID NO: 14) or RASQSVRTDVG (SEQ ID NO: 15); the VL CDR2 sequence YSSNRYT (SEQ ID NO: 16); and the VL CDR3 sequence QQDYSSPFT (SEQ ID NO: 17) or QQDYSSPYT (SEQ ID NO: 18), (b) the VH CDR1 sequence GFTFSNYWMD (SEQ ID NO: 11); the VH CDR2 sequence EIRLKSYNYATH (SEQ ID NO: 12); the VH CDR3 sequence AGTDY (SEQ ID NO: 13); the VL CDR1 sequence RASQSVRTDVG (SEQ ID NO: 15); the VL CDR2 sequence YSSNRYT (SEQ ID NO: 16); and the VL CDR3 sequence QQDYSSPYT (SEQ ID NO: 18), and (c) the VH CDR1 sequence GFTFSNYWMN (SEQ ID NO: 10); the VH CDR2 sequence EIRLKSYNYATH (SEQ ID NO: 12); the VH CDR3 sequence AGTDY (SEQ ID NO: 13); the VL CDR1 sequence KASQSVRTDVA (SEQ ID NO: 14); the VL CDR2 sequence YSSNRYT (SEQ ID NO: 16); and the VL CDR3 sequence QQDYSSPFT (SEQ ID NO: 17). In some embodiments, the antibody or antigen binding fragment thereof comprises a heavy chain variable region comprising an amino acid sequence selected from the group consisting of SEQ ID NOs: 1-4, and a light chain variable region comprising an amino acid sequence selected from the group consisting of SEQ ID NOs: 5-9.

In another aspect of the invention, provided herein is an activatable antibody that, in an activated state, binds CD147 comprising: (a) an antibody or an antigen binding fragment thereof (AB) that specifically binds to human CD147 and cynomolgus monkey CD147; (b) a masking moiety (MM) coupled to the AB, wherein the MM inhibits the binding of the AB to CD147 when the activatable antibody is in an uncleaved (unactivated state) state; and (c) a cleavable moiety (CM) coupled to the AB, wherein the CM is a polypeptide that functions as a substrate for a protease. In some embodiments, the CD147 includes both deglycosylated CD147 and glycosylated CD147. In any of the activatable antibodies provided herein, the AB can be selected from the group consisting of a Fab fragment, a F(ab′)2 fragment, a scFv, a scAb, a dAb, a single domain heavy chain antibody, and a single domain light chain antibody. In any of the activatable antibodies provided herein, the AB specifically binds human CD147. In any of the activatable antibodies provided herein, the AB only binds human CD147. In some embodiments, the AB of the activatable antibody comprises an amino acid sequence comprising amino acid sequences selected from the group consisting of: (a) the VH CDR1 sequence GFTFSNYWMN (SEQ ID NO: 10) or GFTFSNYWMD (SEQ ID NO: 11); the VH CDR2 sequence EIRLKSYNYATH (SEQ ID NO: 12); the VH CDR3 sequence AGTDY (SEQ ID NO: 13); the VL CDR1 sequence KASQSVRTDVA (SEQ ID NO: 14) or RASQSVRTDVG (SEQ ID NO: 15); the VL CDR2 sequence YSSNRYT (SEQ ID NO: 16); and the VL CDR3 sequence QQDYSSPFT (SEQ ID NO: 17) or QQDYSSPYT (SEQ ID NO: 18), (b) the VH CDR1 sequence GFTFSNYWMD (SEQ ID NO: 11); the VH CDR2 sequence EIRLKSYNYATH (SEQ ID NO: 12); the VH CDR3 sequence AGTDY (SEQ ID NO: 13); the VL CDR1 sequence RASQSVRTDVG (SEQ ID NO: 15); the VL CDR2 sequence YSSNRYT (SEQ ID NO: 16); and the VL CDR3 sequence QQDYSSPYT (SEQ ID NO: 18), and (c) the VH CDR1 sequence GFTFSNYWMN (SEQ ID NO: 10); the VH CDR2 sequence EIRLKSYNYATH (SEQ ID NO: 12); the VH CDR3 sequence AGTDY (SEQ ID NO: 13); the VL CDR1 sequence KASQSVRTDVA (SEQ ID NO: 14); the VL CDR2 sequence YSSNRYT (SEQ ID NO: 16); and the VL CDR3 sequence QQDYSSPFT (SEQ ID NO: 17). In some embodiments, the AB of the activatable antibody comprises a heavy chain variable region comprising an amino acid sequence selected from the group consisting of SEQ ID NOs: 1-4, and a light chain variable region comprising an amino acid sequence selected from the group consisting of SEQ ID NOs: 5-9. In some embodiments, the AB of the activatable antibody comprises a heavy chain variable region comprising an amino acid sequence selected from the group consisting of SEQ ID NOs: 1-3, and a light chain variable region comprising an amino acid sequence selected from the group consisting of SEQ ID NOs: 5-8. In some embodiments, the activatable antibody comprises the heavy chain sequence selected from the group consisting of SEQ ID NOs: 1-4 and 19-22 and a light chain comprising an amino acid sequence selected from the group consisting of SEQ ID NOs: 5-9, 23-27, 140-182, 185-227, 230-272, and 275-317. In some embodiments, the MM has a dissociation constant for binding to the AB that is greater than the dissociation constant of the AB to CD147. In some embodiments, the MM does not interfere or compete with the AB for binding to CD147 when the activatable antibody is in a cleaved state. In some embodiments, the MM is a polypeptide of no more than 40 amino acids in length. In some embodiments, the MM polypeptide sequence is different from that of human CD147. In some embodiments, the MM polypeptide sequence is no more than 50% identical to any natural binding partner of the AB. In some embodiments, the MM comprises an amino acid sequence selected from the group consisting of SEQ ID NOs: 30-100. In some embodiments, the MM comprises an amino acid sequence selected from the group consisting of SEQ ID NOs: 30, 31, 33, 44, 46, 75-80, 82, 83, and 86-100. In some embodiments, the CM is a substrate for a protease that is active in diseased tissue. In some embodiments, the CM comprises an amino acid sequence selected from the group consisting of SEQ ID NOs: 356-423, 680-698, 713, 714, and 789-808. In some embodiments, the CM comprises an amino acid sequence selected from the group consisting of SEQ ID NOs: 359, 370, 377, 382, 390, 397, 406-423, 680-698, 713, 714, and 807-808.

In another aspect provided herein, any one of the antibodies or activatable antibodies provided herein are conjugated to an agent, generating a conjugated antibody or conjugated activatable antibody. In some embodiments, the agent is a toxin or fragment thereof. In some embodiments, the agent is a microtubule inhibitor. In some embodiments, the agent is a nucleic acid damaging agent. In some embodiments, the agent is a detectable moiety. In some embodiments, the detectable moiety is a diagnostic agent.

In another aspect, provided herein is a pharmaceutical composition comprising any of the antibodies, activatable antibodies, conjugated antibodies or conjugated activatable antibodies provided herein; and a carrier. In some embodiments, the pharmaceutical composition of comprises an additional agent. In some embodiments, the additional agent is a therapeutic agent.

In another aspect, provided herein is an isolated nucleic acid molecule encoding any one of the antibodies or activatable antibodies described herein. In a related aspect, provided herein is a vector comprising the isolated nucleic acid molecule. In another related aspect provided herein is a method of producing an antibody or an activatable antibody by culturing a cell under conditions that lead to expression of the antibody or the activatable antibody, wherein the cell comprises the nucleic acid molecules or the vectors provided herein.

In another aspect, provided herein is a method of manufacturing an activatable antibody that, in an activated state, binds CD147, the method comprising: (a) culturing a cell comprising a nucleic acid construct that encodes the activatable antibody under conditions that lead to the expression of any one of the activatable antibodies described herein, and (b) recovering the activatable antibody.

In another aspect, provided herein is a method of treating, alleviating a symptom of, or delaying the progression of a disorder or disease in which diseased cells express CD147 comprising administering a therapeutically effective amount of any one of the antibodies, conjugated antibodies, activatable antibodies, conjugated activatable antibodies provided herein, or pharmaceutical compositions of the same to a subject in need thereof. In another aspect, provided herein is a method of treating, alleviating a symptom of, or delaying the progression of a disorder or disease associated with cells expressing CD147 comprising administering a therapeutically effective amount of any one of the antibodies, conjugated antibodies, activatable antibodies, conjugated activatable antibodies provided herein, or pharmaceutical compositions of the same to a subject in need thereof. In some embodiments the disorder or disease is cancer. In some embodiments, the cancer is an adenocarcinoma, a bile duct (biliary) cancer, a bladder cancer, a bone cancer, a breast cancer, a triple-negative breast cancer, a Her2-negative breast cancer, a carcinoid cancer, a cervical cancer, a cholangiocarcinoma, a colorectal cancer, a colon cancer, an endometrial cancer, an esophageal cancer, a glioma, a head and neck cancer, a head and neck squamous cell cancer, a leukemia, a liver cancer, a lung cancer, a non-small cell lung cancer, a small cell lung cancer, a lymphoma, a melanoma, an oropharyngeal cancer, an ovarian cancer, a pancreatic cancer, a prostate cancer, a metastatic castration-resistant prostate carcinoma, a renal cancer, a sarcoma, a skin cancer, a squamous cell cancer, a stomach cancer, a testis cancer, a thyroid cancer, a urogenital cancer, or a urothelial cancer. In another aspect, provided herein is a method of inhibiting or reducing the growth, proliferation, or metastasis of cells expressing CD147 comprising administering a therapeutically effective amount of any one of the antibodies, conjugated antibodies, activatable antibodies, conjugated activatable antibodies provided herein, or pharmaceutical compositions of the same to a subject in need thereof. In some embodiments, the expression and/or activity of the CD147 is aberrant. In another aspect, provided herein is a method of inhibiting, blocking, or preventing the binding of a natural ligand to CD147, comprising administering a therapeutically effective amount of any one of the antibodies, conjugated antibodies, activatable antibodies, conjugated activatable antibodies provided herein, or pharmaceutical compositions of the same to a subject in need thereof. In some embodiments, the expression and/or activity of the CD147 is aberrant. In any of the methods provided herein comprising administering any one of the antibodies, conjugated antibodies, activatable antibodies, conjugated activatable antibodies provided herein, or pharmaceutical compositions of the same to a subject in need thereof, the method can comprise administering an additional agent. In some embodiments, the additional agent is a therapeutic agent.

### BRIEF DESCRIPTION OF THE DRAWINGS

FIG. 1 is a graph depicting the ability of mouse and humanized anti-human CD147 antibodies of the disclosure to bind human CD147.

FIG. 2 is a graph depicting the ability of humanized anti-human CD147 antibodies of the disclosure to bind human CD147.

FIG. 3A-3H are graphs depicting the in vitro cytotoxicity of conjugated anti-human CD147 antibodies of the disclosure.

FIG. 4 is a schematic representation of the screening and sorting of masking peptides of the disclosure.

FIG. 5 is a graph depicting the affinity of bacterial surface-displayed masking peptides of the disclosure to an anti-human CD147 antibody of the disclosure.

FIGS. 6A and 6B are graphs depicting exemplary assays of the relative binding affinity of activatable anti-human CD147 antibodies of the disclosure compared to the unmasked parental anti-human CD147 antibody of the disclosure.

FIGS. 7A, 7B, and 7C are graphs depicting the relative binding affinity of activatable anti-human CD147 antibodies of the disclosure compared to the unmasked parental anti-human CD147 antibody of the disclosure.

FIGS. 8A to 8D show exemplary immunohistochemical assay results of anti-human CD147 antibodies of the disclosure to various cancer-derived tissues.

FIG. 9 is a graph depicting exemplary studies of the ability of anti-human CD147 antibodies of the disclosure to bind human CD147 on various human-derived cell lines and the cytotoxicity of anti-human CD147 antibody drug conjugates of the disclosure to the various human-derived cell lines.

FIG. 10 is a graph depicting an exemplary binding affinity study of anti-human CD147 antibodies of the disclosure to human and cynomolgus cell lines.

FIG. 11A is a graph depicting an exemplary binding affinity study of intact and protease-activated anti-human CD147 activatable antibody drug conjugates of the disclosure to human cell lines.

FIG. 11B is a graph depicting an exemplary in vitro cytotoxicity study of intact and protease-activated anti-human CD147 activatable antibody drug conjugates of the disclosure to a cancer-derived cell line.

FIGS. 12A and 12B are graphs depicting exemplary in vivo efficacy studies of anti-human CD147 activatable antibody drug conjugates of the disclosure in a xenograft model.

FIGS. 13A to 13D shows an exemplary toxicology study that cynomolgus monkeys that received a conjugated activatable anti-human CD147 antibody of the disclosure (anti-huCD147 3A11-440.1-2012 DM4) showed no or lower evidence of hematological toxicity (based on neutrophil, reticulocyte, and monocytes counts) or liver toxicity (based on levels of alanine transaminase (ALT)) as compared to a corresponding conjugated anti-human CD147 antibody of the disclosure (anti-huCD147 3A11 DM4).

FIG. 14 is a graph depicting an exemplary pharmacokinetics study of a conjugated activatable anti-human CD147 antibody of the disclosure and a conjugated parental anti-human CD147 antibody of the disclosure when administered to cynomolgus monkeys.

FIGS. 15A and 15B are graphs depicting an exemplary tolerability study of a conjugated activatable anti-human CD147 antibody of the disclosure and a conjugated parental anti-human CD147 antibody of the disclosure in cynomolgus monkeys by monitoring alanine transaminase levels and neutrophil counts in cynomolgus monkeys to which the conjugated antibodies were administered.

FIG. 16 is a graph depicting the ability of humanized anti-human CD147 antibodies of the disclosure to bind both glycosylated and deglycosylated human CD147 fusion protein.

### DETAILED DESCRIPTION OF THE INVENTION

CD147 (also known as Basigin, extracellular matrix metalloproteinase inducer (EMMPRIN), gp42, BSG, HT7, neurothelin, OX-47, M6, and 5A11) is a transmembrane glycoprotein that that belongs to the immunoglobulin superfamily and plays essential roles in intercellular communication. (The use of the term “CD147” is intended to cover any variation thereof, such as, by way of non-limiting example, CD-147 and/or CD 147, and all variations are used herein interchangeably.) CD147 is the receptor for cyclophilins A and B, S100A9 and platelet glycoprotein VI, whereas CD147 serves as the receptor for the rod-derived cone viability factor. CD147 associates with monocarboxylate transporters and is essential for their cell surface translocation and activities. CD147 also interacts with several integrins. In the same membrane plane, CD147 also associates with other proteins including GLUT1, CD44 and CD98. The carbohydrate portion of CD147 is recognized by lectins, such as galectin-3 and E-selectin. These molecular recognitions form the basis for the role of CD147 in the transport of nutrients, migration of inflammatory leukocytes and induction of matrix metalloproteinases (MMPs). CD147 plays roles in vision, spermatogenesis and other physiological phenomena, and also plays roles in the pathogenesis of numerous diseases, including cancer. CD147 is also the receptor for an invasive protein RH5, which is present in malaria parasites.

CD147 has a broad expression pattern on hematopoietic and non-hematopoietic cells such as monocytes, granulocytes, epithelial and endothelial cells. CD147 is upregulated on active T-lymphocytes. Some CD147 antibodies, to specific epitopes, inhibit proliferation induced by a CD3 mAb.

CD147 is desirable target because it is prevalent across multiple cancer indications.

Accordingly, the present disclosure provides antibodies, activatable antibodies, conjugated antibodies, and conjugated activatable antibodies that specifically bind mammalian CD147, methods of making and use thereof.

More specifically, the disclosure provides anti-mammalian CD147 antibodies and fragments thereof (interchangeably referred to herein as CD147 antibodies, or ABs), conjugated CD147 antibodies, activatable CD147 antibodies, and conjugated activatable CD147 antibodies that are useful in methods of treating, preventing, delaying the progression of, ameliorating and/or alleviating a symptom of a disease or disorder associated with cells expressing CD147. In some embodiments, the cells are associated with normal CD147 expression and/or activity. In some embodiments, the cells are associated with aberrant CD147 expression and/or activity. In some embodiments, the cells are associated with CD147 expression and/or activity in diseased cells. For example, any of the antibodies/activatable antibodies described herein can be used in methods of treating, preventing, delaying the progression of, ameliorating and/or alleviating a symptom of a cancer or other neoplastic condition. Any of the antibodies/activatable antibodies described herein can also be used for detection/diagnostic applications.

In some embodiments the antibodies and activatable antibodies specifically bind human CD147 and cynomolgus monkey CD147. In some embodiments, the antibodies and activatable antibodies bind human CD147. In some embodiments, the antibodies and activatable antibodies bind cynomolgus monkey CD147. In some embodiments, the antibodies and activatable antibodies are internalized by CD147-containing cells. In some embodiments, the antibodies and activatable antibodies bind both the glycosylated and deglycosylated forms of the CD147 antigen.

### Definitions

Unless otherwise defined, scientific and technical terms used in connection with the present disclosure shall have the meanings that are commonly understood by those of ordinary skill in the art. The term “a” entity or “an” entity refers to one or more of that entity. For example, a compound refers to one or more compounds. As such, the terms “a”, “an”, “one or more” and “at least one” can be used interchangeably. Further, unless otherwise required by context, singular terms shall include pluralities and plural terms shall include the singular. Generally, nomenclatures utilized in connection with, and techniques of, cell and tissue culture, molecular biology, and protein and oligo- or polynucleotide chemistry and hybridization described herein are those well-known and commonly used in the art. Standard techniques are used for recombinant DNA, oligonucleotide synthesis, and tissue culture and transformation (e.g., electroporation, lipofection). Enzymatic reactions and purification techniques are performed according to manufacturer's specifications or as commonly accomplished in the art or as described herein. The foregoing techniques and procedures are generally performed according to conventional methods well known in the art and as described in various general and more specific references that are cited and discussed throughout the present specification. See e.g., Sambrook et al. Molecular Cloning: A Laboratory Manual (2d ed., Cold Spring Harbor Laboratory Press, Cold Spring Harbor, N.Y. (1989)). The nomenclatures utilized in connection with, and the laboratory procedures and techniques of, analytical chemistry, synthetic organic chemistry, and medicinal and pharmaceutical chemistry described herein are those well-known and commonly used in the art. Standard techniques are used for chemical syntheses, chemical analyses, pharmaceutical preparation, formulation, and delivery, and treatment of patients.

As utilized in accordance with the present disclosure, the following terms, unless otherwise indicated, shall be understood to have the following meanings:

As used herein, the term “antibody” refers to immunoglobulin molecules and immunologically active portions of immunoglobulin (Ig) molecules, i.e., molecules that contain an antigen binding site that specifically binds (immunoreacts with) an antigen. By “specifically bind” or “immunoreacts with” or “immunospecifically bind” is meant that the antibody reacts with one or more antigenic determinants of the desired antigen and does not react with other polypeptides or binds at much lower affinity (K_{d}>10⁻⁶). Antibodies include, but are not limited to, polyclonal, monoclonal, chimeric, domain antibody, single chain, Fab, and F(ab′)2 fragments, scFvs, and an Fab expression library. The antibodies provided herein can be of any of the IgG, IgM, IgA, IgE and IgD classes (or subclasses thereof).

The term “monoclonal antibody” (mAb) or “monoclonal antibody composition”, as used herein, refers to a population of antibody molecules that contain only one molecular species of antibody molecule consisting of a unique light chain gene product and a unique heavy chain gene product. In particular, the complementarity determining regions (CDRs) of the monoclonal antibody are identical in all the molecules of the population. MAbs contain an antigen binding site capable of immunoreacting with a particular epitope of the antigen characterized by a unique binding affinity for it.

The term “antigen-binding site” or “binding portion” refers to the part of the immunoglobulin molecule that participates in antigen binding. The antigen binding site is formed by amino acid residues of the N-terminal variable (“V”) regions of the heavy (“H”) and light (“L”) chains. Three highly divergent stretches within the V regions of the heavy and light chains, referred to as “hypervariable regions,” are interposed between more conserved flanking stretches known as “framework regions,” or “FRs”. Thus, the term “FR” refers to amino acid sequences that are naturally found between, and adjacent to, hypervariable regions in immunoglobulins. In an antibody molecule, the three hypervariable regions of a light chain and the three hypervariable regions of a heavy chain are disposed relative to each other in three dimensional space to form an antigen-binding surface. The antigen-binding surface is complementary to the three-dimensional surface of a bound antigen, and the three hypervariable regions of each of the heavy and light chains are referred to as “complementarity-determining regions,” or “CDRs.” The assignment of amino acids to each domain is in accordance with the definitions of Kabat Sequences of Proteins of Immunological Interest (National Institutes of Health, Bethesda, Md. (1987 and 1991)), or Chothia & Lesk J. Mol. Biol. 196:901-917 (1987), Chothia et al. Nature 342:878-883 (1989).

As used herein, the term “epitope” includes any protein determinant capable of specific binding to an immunoglobulin, a scFv, or a T-cell receptor. The term “epitope” includes any protein determinant capable of specific binding to an immunoglobulin or T-cell receptor. Epitopic determinants usually consist of chemically active surface groupings of molecules such as amino acids or sugar side chains and usually have specific three dimensional structural characteristics, as well as specific charge characteristics. For example, antibodies can be raised against N-terminal or C-terminal peptides of a polypeptide. An antibody is said to specifically bind an antigen when the dissociation constant is ≤1 μM; in some embodiments, ≤100 nM and in some embodiments, ≤10 nM.

As used herein, the terms “specific binding,” “immunological binding,” and “immunological binding properties” refer to the non-covalent interactions of the type which occur between an immunoglobulin molecule and an antigen for which the immunoglobulin is specific. The strength, or affinity of immunological binding interactions can be expressed in terms of the dissociation constant (K_{d}) of the interaction, wherein a smaller K_{d} represents a greater affinity. Immunological binding properties of selected polypeptides can be quantified using methods well known in the art. One such method entails measuring the rates of antigen-binding site/antigen complex formation and dissociation, wherein those rates depend on the concentrations of the complex partners, the affinity of the interaction, and geometric parameters that equally influence the rate in both directions. Thus, both the “on rate constant” (Kₒₙ) and the “off rate constant” (K_{off}) can be determined by calculation of the concentrations and the actual rates of association and dissociation. (See Nature 361:185-87 (1993)). The ratio of K_{off}/Kₒₙ enables the cancellation of all parameters not related to affinity, and is equal to the dissociation constant K_{d}. (See, generally, Davies et al. (1990) Annual Rev Biochem 59:439-473). An antibody of the present disclosure is said to specifically bind to the target, when the binding constant (K_{d}) is ≤1 μM, in some embodiments ≤100 nM, in some embodiments ≤10 nM, and in some embodiments ≤100 pM to about 1 pM, as measured by assays such as radioligand binding assays or similar assays known to those skilled in the art.

The term “isolated polynucleotide” as used herein shall mean a polynucleotide of genomic, cDNA, or synthetic origin or some combination thereof, which by virtue of its origin the “isolated polynucleotide” (1) is not associated with all or a portion of a polynucleotide in which the “isolated polynucleotide” is found in nature, (2) is operably linked to a polynucleotide which it is not linked to in nature, or (3) does not occur in nature as part of a larger sequence. Polynucleotides in accordance with the disclosure include the nucleic acid molecules encoding the heavy chain immunoglobulin molecules shown herein, and nucleic acid molecules encoding the light chain immunoglobulin molecules shown herein.

The term “isolated protein” referred to herein means a protein of cDNA, recombinant RNA, or synthetic origin or some combination thereof, which by virtue of its origin, or source of derivation, the “isolated protein” (1) is not associated with proteins found in nature, (2) is free of other proteins from the same source, e.g., free of murine proteins, (3) is expressed by a cell from a different species, or (4) does not occur in nature.

The term “polypeptide” is used herein as a generic term to refer to native protein, fragments, or analogs of a polypeptide sequence. Hence, native protein fragments, and analogs are species of the polypeptide genus. Polypeptides in accordance with the disclosure comprise the heavy chain immunoglobulin molecules shown herein, and the light chain immunoglobulin molecules shown herein, as well as antibody molecules formed by combinations comprising the heavy chain immunoglobulin molecules with light chain immunoglobulin molecules, such as kappa light chain immunoglobulin molecules, and vice versa, as well as fragments and analogs thereof.

The term “naturally-occurring” as used herein as applied to an object refers to the fact that an object can be found in nature. For example, a polypeptide or polynucleotide sequence that is present in an organism (including viruses) that can be isolated from a source in nature and that has not been intentionally modified by man in the laboratory or otherwise is naturally-occurring.

The term “operably linked” as used herein refers to positions of components so described are in a relationship permitting them to function in their intended manner. A control sequence “operably linked” to a coding sequence is ligated in such a way that expression of the coding sequence is achieved under conditions compatible with the control sequences.

The term “control sequence” as used herein refers to polynucleotide sequences that are necessary to effect the expression and processing of coding sequences to which they are ligated. The nature of such control sequences differs depending upon the host organism in prokaryotes, such control sequences generally include promoter, ribosomal binding site, and transcription termination sequence in eukaryotes, generally, such control sequences include promoters and transcription termination sequence. The term “control sequences” is intended to include, at a minimum, all components whose presence is essential for expression and processing, and can also include additional components whose presence is advantageous, for example, leader sequences and fusion partner sequences. The term “polynucleotide” as referred to herein means nucleotides of at least 10 bases in length, either ribonucleotides or deoxynucleotides or a modified form of either type of nucleotide. The term includes single and double stranded forms of DNA.

The term oligonucleotide referred to herein includes naturally occurring, and modified nucleotides linked together by naturally occurring, and non-naturally occurring oligonucleotide linkages. Oligonucleotides are a polynucleotide subset generally comprising a length of 200 bases or fewer. In some embodiments, oligonucleotides are 10 to 60 bases in length and in some embodiments, 12, 13, 14, 15, 16, 17, 18, 19, or 20 to 40 bases in length. Oligonucleotides are usually single stranded, e.g., for probes, although oligonucleotides may be double stranded, e.g., for use in the construction of a gene mutant. Oligonucleotides of the disclosure are either sense or antisense oligonucleotides.

The term “naturally occurring nucleotides” referred to herein includes deoxyribonucleotides and ribonucleotides. The term “modified nucleotides” referred to herein includes nucleotides with modified or substituted sugar groups and the like. The term “oligonucleotide linkages” referred to herein includes oligonucleotide linkages such as phosphorothioate, phosphorodithioate, phosphoroselerloate, phosphorodiselenoate, phosphoroanilothioate, phoshoraniladate, phosphoronmidate, and the like. See e.g., LaPlanche et al. Nucl. Acids Res. 14:9081 (1986); Stec et al. J. Am. Chem. Soc. 106:6077 (1984), Stein et al. Nucl. Acids Res. 16:3209 (1988), Zon et al. Anti Cancer Drug Design 6:539 (1991); Zon et al. Oligonucleotides and Analogues: A Practical Approach, pp. 87-108 (F. Eckstein, Ed., Oxford University Press, Oxford England (1991)); Stec et al. U.S. Pat. No. 5,151,510; Uhlmann and Peyman Chemical Reviews 90:543 (1990). An oligonucleotide can include a label for detection, if desired.

As used herein, the twenty conventional amino acids and their abbreviations follow conventional usage. See Immunology—A Synthesis (2nd Edition, E. S. Golub and D. R. Green, Eds., Sinauer Associates, Sunderland, Mass. (1991)). Stereoisomers (e.g., D-amino acids) of the twenty conventional amino acids, unnatural amino acids such as α-, α-disubstituted amino acids, N-alkyl amino acids, lactic acid, and other unconventional amino acids may also be suitable components for polypeptides of the present disclosure. Examples of unconventional amino acids include: 4 hydroxyproline, γ-carboxyglutamate, ε-N,N,N-trimethyllysine, ε-N-acetyllysine, O-phosphoserine, N-acetylserine, N-formylmethionine, 3-methylhistidine, 5-hydroxylysine, σ-N-methylarginine, and other similar amino acids and imino acids (e.g., 4-hydroxyproline). In the polypeptide notation used herein, the left-hand direction is the amino terminal direction and the right-hand direction is the carboxy-terminal direction, in accordance with standard usage and convention.

Similarly, unless specified otherwise, the left-hand end of single-stranded polynucleotide sequences is the 5′ end the left-hand direction of double-stranded polynucleotide sequences is referred to as the 5′ direction. The direction of 5′ to 3′ addition of nascent RNA transcripts is referred to as the transcription direction sequence regions on the DNA strand having the same sequence as the RNA and that are 5′ to the 5′ end of the RNA transcript are referred to as “upstream sequences”, sequence regions on the DNA strand having the same sequence as the RNA and that are 3′ to the 3′ end of the RNA transcript are referred to as “downstream sequences”.

As applied to polypeptides, the term “substantial identity” means that two peptide sequences, when optimally aligned, such as by the programs GAP or BESTFIT using default gap weights, share at least 80 percent sequence identity, in some embodiments, at least 90 percent sequence identity, in some embodiments, at least 95 percent sequence identity, and in some embodiments, at least 99 percent sequence identity.

In some embodiments, residue positions that are not identical differ by conservative amino acid substitutions.

As discussed herein, minor variations in the amino acid sequences of antibodies or immunoglobulin molecules are contemplated as being encompassed by the present disclosure, providing that the variations in the amino acid sequence maintain at least 75%, in some embodiments, at least 80%, 90%, 95%, and in some embodiments, 99%. In particular, conservative amino acid replacements are contemplated. Conservative replacements are those that take place within a family of amino acids that are related in their side chains. Genetically encoded amino acids are generally divided into families: (1) acidic amino acids are aspartate, glutamate; (2) basic amino acids are lysine, arginine, histidine; (3) non-polar amino acids are alanine, valine, leucine, isoleucine, proline, phenylalanine, methionine, tryptophan, and (4) uncharged polar amino acids are glycine, asparagine, glutamine, cysteine, serine, threonine, tyrosine. The hydrophilic amino acids include arginine, asparagine, aspartate, glutamine, glutamate, histidine, lysine, serine, and threonine. The hydrophobic amino acids include alanine, cysteine, isoleucine, leucine, methionine, phenylalanine, proline, tryptophan, tyrosine and valine. Other families of amino acids include (i) serine and threonine, which are the aliphatic-hydroxy family; (ii) asparagine and glutamine, which are the amide containing family; (iii) alanine, valine, leucine and isoleucine, which are the aliphatic family; and (iv) phenylalanine, tryptophan, and tyrosine, which are the aromatic family. For example, it is reasonable to expect that an isolated replacement of a leucine with an isoleucine or valine, an aspartate with a glutamate, a threonine with a serine, or a similar replacement of an amino acid with a structurally related amino acid will not have a major effect on the binding or properties of the resulting molecule, especially if the replacement does not involve an amino acid within a framework site. Whether an amino acid change results in a functional peptide can readily be determined by assaying the specific activity of the polypeptide derivative. Assays are described in detail herein. Fragments or analogs of antibodies or immunoglobulin molecules can be readily prepared by those of ordinary skill in the art. Suitable amino- and carboxy-termini of fragments or analogs occur near boundaries of functional domains. Structural and functional domains can be identified by comparison of the nucleotide and/or amino acid sequence data to public or proprietary sequence databases. In some embodiments, computerized comparison methods are used to identify sequence motifs or predicted protein conformation domains that occur in other proteins of known structure and/or function. Methods to identify protein sequences that fold into a known three-dimensional structure are known. Bowie et al. Science 253:164 (1991). Thus, the foregoing examples demonstrate that those of skill in the art can recognize sequence motifs and structural conformations that can be used to define structural and functional domains in accordance with the disclosure.

Suitable amino acid substitutions are those that: (1) reduce susceptibility to proteolysis, (2) reduce susceptibility to oxidation, (3) alter binding affinity for forming protein complexes, (4) alter binding affinities, and (5) confer or modify other physicochemical or functional properties of such analogs. Analogs can include various muteins of a sequence other than the naturally-occurring peptide sequence. For example, single or multiple amino acid substitutions (for example, conservative amino acid substitutions) can be made in the naturally-occurring sequence (for example, in the portion of the polypeptide outside the domain(s) forming intermolecular contacts. A conservative amino acid substitution should not substantially change the structural characteristics of the parent sequence (e.g., a replacement amino acid should not tend to break a helix that occurs in the parent sequence, or disrupt other types of secondary structure that characterizes the parent sequence). Examples of art-recognized polypeptide secondary and tertiary structures are described in Proteins, Structures and Molecular Principles (Creighton, Ed., W. H. Freeman and Company, New York (1984)); Introduction to Protein Structure (C. Branden and J. Tooze, eds., Garland Publishing, New York, N.Y. (1991)); and Thornton et at. Nature 354:105 (1991).

The term “polypeptide fragment” as used herein refers to a polypeptide that has an amino terminal and/or carboxy-terminal deletion and/or one or more internal deletion(s), but where the remaining amino acid sequence is identical to the corresponding positions in the naturally-occurring sequence deduced, for example, from a full length cDNA sequence. Fragments typically are at least 5, 6, 8 or 10 amino acids long, in some embodiments, at least 14 amino acids long, in some embodiments, at least 20 amino acids long, usually at least 50 amino acids long, and in some embodiments, at least 70 amino acids long. The term “analog” as used herein refers to polypeptides that are comprised of a segment of at least 25 amino acids that has substantial identity to a portion of a deduced amino acid sequence and that has specific binding to the target, under suitable binding conditions. Typically, polypeptide analogs comprise a conservative amino acid substitution (or addition or deletion) with respect to the naturally-occurring sequence. Analogs typically are at least 20 amino acids long, in some embodiments, at least 50 amino acids long or longer, and can often be as long as a full-length naturally-occurring polypeptide.

The term “agent” is used herein to denote a chemical compound, a mixture of chemical compounds, a biological macromolecule, or an extract made from biological materials.

As used herein, the terms “label” or “labeled” refers to incorporation of a detectable marker, e.g., by incorporation of a radiolabeled amino acid or attachment to a polypeptide of biotinyl moieties that can be detected by marked avidin (e.g., streptavidin containing a fluorescent marker or enzymatic activity that can be detected by optical or calorimetric methods). In certain situations, the label or marker can also be therapeutic. Various methods of labeling polypeptides and glycoproteins are known in the art and can be used. Examples of labels for polypeptides include, but are not limited to, the following: radioisotopes or radionuclides (e.g., ³H, ¹⁴C, ¹⁵N, ³⁵S, ⁹⁰Y, ⁹⁹Tc, ¹¹¹In, ¹²⁵I, ¹³¹I), fluorescent labels (e.g., FITC, rhodamine, lanthanide phosphors), enzymatic labels (e.g., horseradish peroxidase, p-galactosidase, luciferase, alkaline phosphatase), chemiluminescent, biotinyl groups, predetermined polypeptide epitopes recognized by a secondary reporter (e.g., leucine zipper pair sequences, binding sites for secondary antibodies, metal binding domains, epitope tags). In some embodiments, labels are attached by spacer arms of various lengths to reduce potential steric hindrance. The term “pharmaceutical agent or drug” as used herein refers to a chemical compound or composition capable of inducing a desired therapeutic effect when properly administered to a patient.

Other chemistry terms herein are used according to conventional usage in the art, as exemplified by The McGraw-Hill Dictionary of Chemical Terms (Parker, S., Ed., McGraw-Hill, San Francisco (1985)).

As used herein, “substantially pure” means an object species is the predominant species present (i.e., on a molar basis it is more abundant than any other individual species in the composition), and in some embodiments, a substantially purified fraction is a composition wherein the object species comprises at least about 50 percent (on a molar basis) of all macromolecular species present.

Generally, a substantially pure composition will comprise more than about 80 percent of all macromolecular species present in the composition, in some embodiments, more than about 85%, 90%, 95%, and 99%. In some embodiments, the object species is purified to essential homogeneity (contaminant species cannot be detected in the composition by conventional detection methods) wherein the composition consists essentially of a single macromolecular species.

The term patient includes human and veterinary subjects.

CD147 Antibodies

Provided herein are antibodies and antigen binding fragments thereof (ABs) that specifically bind to mammalian CD147. In some embodiments, the AB specifically binds human CD147 and cynomolgus monkey CD147.

The ABs provided herein that bind CD147 includes a monoclonal antibody, a domain antibody, a single chain antibody, a Fab fragment, a F(ab′)2 fragment, a scFv, a scAb, a dAb, a single domain heavy chain antibody, or a single domain light chain antibody. In some embodiments, such an ABs that binds CD147 is a mouse, other rodent, chimeric, humanized or fully human monoclonal antibody.

Also provided herein are activatable CD147 antibodies that include an antibody or antigen-binding fragment thereof (AB) that specifically binds CD147 coupled to a masking moiety (MM), such that coupling of the MM reduces the ability of the antibody or antigen-binding fragment thereof to bind CD147. In some embodiments, the MM is coupled via a sequence that includes a substrate for a protease (cleavable moiety, CM), for example, a protease that is co-localized with CD147 at a treatment site in a subject. (The activatable CD147 antibodies of the disclosure are described in greater detail in a below section).

The CD147 antibodies of the disclosure specifically bind a mammalian CD147 target, such as, for example, human CD147. Also included in the disclosure are CD147 antibodies and ABs that bind to the same CD147 epitope as an antibody of the disclosure and/or an activated activatable antibody described herein. Also included in the disclosure are CD147 antibodies compete with a CD147 antibody described herein for binding to a CD147 target, e.g., human CD147. Also included in the disclosure are CD147 antibodies that cross-compete with (inhibit the binding of) a CD147 antibody and/or an activated CD147 activatable antibody described herein for binding to a CD147 target, e.g., human CD147.

Antibodies and/or activatable antibodies of the disclosure specifically bind a mammalian CD147, e.g. human CD147 and cynomologous CD147. Also included in the disclosure are antibodies and/or activatable antibodies that bind to the same epitope as any of the antibodies and/or activatable antibodies described herein. Also included in the disclosure are antibodies and/or antibodies activatable antibodies that compete with a CD147 antibody (inhibit the binding of) and/or a CD147 activatable antibody described herein for binding to CD147, e.g., human CD147. Also included in the disclosure are antibodies and/or antibodies activatable antibodies that cross-compete with a CD147 antibody and/or a CD147 activatable antibody described herein for binding to CD147 (inhibits the binding to CD147), e.g., human CD147.

In some embodiments, the mammalian CD147 is selected from the group consisting of a human CD147, a murine CD147, a rat CD147, and a cynomolgus monkey CD147. In some embodiments, the AB specifically binds to human CD147, murine CD147 or cynomolgus monkey CD147 with a dissociation constant of less than 1 nM. In some embodiments, the mammalian CD147 is a human CD147.

In some embodiments, the AB has one or more of the following characteristics: (a) the AB specifically binds to human CD147; and (b) the AB specifically binds to human CD147 and cynomolgus monkey CD147.

In some embodiments, the AB has one or more of the following characteristics: (a) the AB specifically binds human CD147 and cynomolgus monkey CD147; (b) the AB inhibits binding of one or more of the natural mammalian ligands of CD147 to mammalian CD147; (c) the AB inhibits binding of one or more of the natural human ligands of CD147 to human CD147; and (d) the AB inhibits binding of one or more of the natural cynomolgus monkey ligands of CD147 to cynomolgus monkey CD147.

In some embodiments the AB binds both glycosylated and deglycosylated forms of CD147.

In some embodiments, the AB blocks the ability of a natural ligand to bind to the mammalian CD147 with an EC₅₀ less than or equal to 5 nM, less than or equal to 10 nM, less than or equal to 50 nM, less than or equal to 100 nM, less than or equal to 500 nM, and/or less than or equal to 1000 nM. In some embodiments, the AB blocks the ability of a natural ligand to bind to the mammalian CD147 with an EC₅₀ less than or equal to 5 nM, less than or equal to 10 nM, less than or equal to 50 nM, less than or equal to 100 nM, less than or equal to 500 nM, and/or less than or equal to 1000 nM.

In some embodiments, the AB blocks the ability of a natural ligand to bind to the mammalian CD147 with an EC₅₀ of 5 nM to 1000 nM, 5 nM to 500 nM, 5 nM to 100 nM 5 nM to 50 nM, 5 nM to 10 nM, 10 nM to 1000 nM, 10 nM to 500 nM, 10 nM to 100 nM 10 nM to 50 nM, 50 nM to 1000 nM, 50 nM to 500 nM, 50 nM to 100 nM, 100 nM to 1000 nM, 100 nM to 500 nM, 500 nM to 1000 nM. In some embodiments, the AB blocks the ability of a natural ligand to bind to the mammalian CD147 with an EC₅₀ of 5 nM to 1000 nM, 5 nM to 500 nM, 5 nM to 100 nM 5 nM to 50 nM, 5 nM to 10 nM, 10 nM to 1000 nM, 10 nM to 500 nM, 10 nM to 100 nM 10 nM to 50 nM, 50 nM to 1000 nM, 50 nM to 500 nM, 50 nM to 100 nM, 100 nM to 1000 nM, 100 nM to 500 nM, 500 nM to 1000 nM.

In some embodiments, the AB of the present disclosure inhibits or reduces the growth, proliferation, and/or metastasis of cells expressing mammalian CD147. Without intending to be bound by any theory, the AB of the present disclosure may inhibit or reduce the growth, proliferation, and/or metastasis of cells expressing mammalian CD147 by specifically binding to CD147 and inhibiting, blocking, and/or preventing the binding of a natural ligand to mammalian CD147.

In some embodiments, the AB has a dissociation constant of about 100 nM or less for binding to mammalian CD147. In some embodiments, the AB has a dissociation constant of about 10 nM or less for binding to mammalian CD147. In some embodiments, the AB has a dissociation constant of about 5 nM or less for binding to CD147. In some embodiments, the AB has a dissociation constant of about 1 nM or less for binding to CD147. In some embodiments, the AB has a dissociation constant of about 0.5 nM or less for binding to CD147. In some embodiments, the AB has a dissociation constant of about 0.1 nM or less for binding to CD147. In some embodiments, the AB has a dissociation constant of 0.01 nM to 100 nM, 0.01 nM to 10 nM, 0.01 nM to 5 nM, 0.01 nM to 1 nM, 0.01 to 0.5 nM, 0.01 nm to 0.1 nM, 0.01 nm to 0.05 nM, 0.05 nM to 100 nM, 0.05 nM to 10 nM, 0.05 nM to 5 nM, 0.05 nM to 1 nM, 0.05 to 0.5 nM, 0.05 nm to 0.1 nM, 0.1 nM to 100 nM, 0.1 nM to 10 nM, 0.1 nM to 5 nM, 0.1 nM to 1 nM, 0.1 to 0.5 nM, 0.5 nM to 100 nM, 0.5 nM to 10 nM, 0.5 nM to 5 nM, 0.5 nM to 1 nM, 1 nM to 100 nM, 1 nM to 10 nM, 1 nM to 5 nM, 5 nM to 100 nM, 5 nM to 10 nM, or 10 nM to 100 nM, for binding to mammalian CD147.

Exemplary CD147 antibodies and activatable CD147 antibodies of the invention may include a heavy chain and a light chain that are, or are derived from, the heavy chain variable and light chain variable sequences shown below (CDR sequences are shown in bold and underline):

mu 3A11 VH:

(SEQ ID NO: 4)

EVKLEESGGGLVQPGGSMKLSCVASGFTFSNYWMNWVRQSPEKGLEWVGE
IRLKSYNyATHYVESVEGRFTISRDDSKSSVYLQMNNLRAEDTGIYYCTA
AGTDYWGQGTTLTVSS
mu 3A11 VL:

(SEQ ID NO: 9)

SIVMTQIPKILLVSAGDRVTITCKASQSVRTDVAWYQQKPGQSPKLLIYY
SSNRYTGVPDRFTGSGYGTDFTFTISTVQAEDLAVYFCQQDYSSPFTFGS
GTKLEIK
3A11 hu Hc1

(SEQ ID NO: 1)

EVQLVESGGGLVQPGGSLRLSCAASGFTFSNYWMDWVRQAPGKGLEWVGE
IRLKSYNYATHYAASVKGRFTISRDDSKNSVYLQMNSLKTEDTAVYYCTA
AGTDYWGQGTLVTVSS
3A11 hu Hc2

(SEQ ID NO: 2)

EVQLVESGGGLVQPGGSLRLSCAASGFTFSNYWMNWVRQAPGKGLEWVGE
IRLKSYNYATHYAASVKGRFTISRDDSKNSLYLQMNSLKTEDTAVYYCAR
AGTDYWGQGTLVTVSS
3A11 hu Hc3

(SEQ ID NO: 3)

EVQLVESGGGLVQPGGSLRLSCAASGFTFSNYWMNWVRQAPGKGLEWVGE
IRLKSYNYATHYVASVKGRFTISRDDSKNSVYLQMNSLKTEDTAVYYCTA
AGTDYWGQGTLLTVSS
3A11 hu Lc1

(SEQ ID NO: 5)

DIQMTQSPSSLSASVGDRVTITCRASQSVRTDVGWYQQKPGKAPKLLIYY
SSNRYTGVPSRFSGSGSGTDFTLTISSLQPEDFATYYCQQDYSSPYTFGQ
GTKLEIK
3A11 hu Lc2

(SEQ ID NO: 6)

DIQMTQSPSSLSASVGDRVTITCKASQSVRTDVAWYQQKPGKAPKLLIYY
SSNRYTGVPSRFSGSGSGTDFTLTISSLQPEDFATYYCQQDYSSPFTFGQ
GTKLEIK
3A11 hu Lc3

(SEQ ID NO: 7)

DIQMTQSPSSLSVSVGDRVTITCKASQSVRTDVAWYQQKPGKAPKLLIYY
SSNRYTGVPSRFSGSGSGTDFTFTISSVQPEDFATYYCQQDYSSPFTFGQ
GTKLEIK
3A11 hu Lc4

(SEQ ID NO: 8)

DIQMTQSPSSLSVSVGDRVTITCKASQSVRTDVAWYQQKPGKAPKLLIYY
SSNRYTGVPSRFSGSGYGTDFTFTISSVQPEDFATYYCQQDYSSPFTFGQ
GTKLEIK

In some embodiments, the antibody or antigen-binding fragment thereof of the CD147 antibody/activatable antibody comprises a heavy chain variable region amino acid sequence selected from the group consisting of SEQ ID NOs: 1-4. In some embodiments, the antibody or antigen-binding fragment thereof of the CD147 antibody/activatable antibody comprises a heavy chain variable region amino acid sequence selected from the group consisting of SEQ ID NOs: 1-3.

In some embodiments, the antibody or antigen-binding fragment thereof of the CD147 antibody/activatable antibody comprises a light chain variable region amino acid sequence selected from the group consisting of SEQ ID NOs: 5-9. In some embodiments, the antibody or antigen-binding fragment thereof of the CD147 antibody/activatable antibody comprises a light chain variable region amino acid sequence selected from the group consisting of SEQ ID NOs: 5-8.

In some embodiments, the antibody or antigen-binding fragment thereof of the CD147 antibody/activatable antibody comprises a heavy chain variable region amino acid sequence selected from the group consisting of SEQ ID NOs: 1-4, and a light chain variable region amino acid sequence selected from the group consisting of SEQ ID NOs: 5-9.

In some embodiments, the antibody or antigen-binding fragment thereof of the CD147 antibody/activatable antibody comprises a heavy chain variable region amino acid sequence selected from the group consisting of SEQ ID NOs: 1-3, and a light chain variable region amino acid sequence selected from the group consisting of SEQ ID NOs: 5-8.

In some embodiments, the antibody or antigen-binding fragment thereof of the CD147 antibody/activatable antibody comprises a heavy chain variable region amino acid sequence that is at least 90%, 91%, 92%, 93%, 94%, 95%, 96%, 97%, 98% or 99% identical to an amino acid sequence selected from the group consisting of SEQ ID NOs: 1-4. In some embodiments, the antibody or antigen-binding fragment thereof of the CD147 antibody/activatable antibody comprises a heavy chain variable region amino acid sequence that is at least 90%, 91%, 92%, 93%, 94%, 95%, 96%, 97%, 98% or 99% identical to an amino acid sequence selected from the group consisting of SEQ ID NOs: 1-3.

In some embodiments, the antibody or antigen-binding fragment thereof of the CD147 antibody/activatable antibody comprises a light chain variable region amino acid sequence that is at least 90%, 91%, 92%, 93%, 94%, 95%, 96%, 97%, 98% or 99% identical to an amino acid sequence selected from the group consisting of SEQ ID NOs: 5-9. In some embodiments, the antibody or antigen-binding fragment thereof of the CD147 antibody/activatable antibody comprises a light chain variable region amino acid sequence that is at least 90%, 91%, 92%, 93%, 94%, 95%, 96%, 97%, 98% or 99% identical to an amino acid sequence selected from the group consisting of SEQ ID NOs: 5-8.

In some embodiments, the antibody or antigen-binding fragment thereof of the CD147 antibody/activatable antibody comprises a heavy chain variable region amino acid sequence that is at least 90%, 91%, 92%, 93%, 94%, 95%, 96%, 97%, 98% or 99% identical to an amino acid sequence selected from the group consisting of SEQ ID NOs: 1-4, and a light chain variable region amino acid sequence that is at least 90%, 91%, 92%, 93%, 94%, 95%, 96%, 97%, 98% or 99% identical to an amino acid sequence selected from the group consisting of SEQ ID NOs: 5-9.

In some embodiments, the antibody or antigen-binding fragment thereof of the CD147 antibody/activatable antibody comprises a heavy chain variable region amino acid sequence that is at least 90%, 91%, 92%, 93%, 94%, 95%, 96%, 97%, 98% or 99% identical to an amino acid sequence selected from the group consisting of SEQ ID NOs: 1-3, and a light chain variable region amino acid sequence that is at least 90%, 91%, 92%, 93%, 94%, 95%, 96%, 97%, 98% or 99% identical to an amino acid sequence selected from the group consisting of SEQ ID NOs: 5-8.

Exemplary CD147 antibodies and activatable CD147 antibodies of the invention include a combination of a variable heavy chain complementarity determining region 1 (VH CDR1, also referred to herein as CDRH1) sequence, a variable heavy chain complementarity determining region 2 (VH CDR2, also referred to herein as CDRH2) sequence, a variable heavy chain complementarity determining region 3 (VH CDR3, also referred to herein as CDRH3) sequence, a variable light chain complementarity determining region 1 (VL CDR1, also referred to herein as CDRL1) sequence, a variable light chain complementarity determining region 2 (VL CDR2, also referred to herein as CDRL2) sequence, and a variable light chain complementarity determining region 3 (VL CDR3, also referred to herein as CDRL3) sequence, wherein at least one CDR sequence is selected from the group consisting of a VH CDR1 sequence comprising the amino acid sequence GFTFSNYWMN (SEQ ID NO: 10) or GFTFSNYWMD (SEQ ID NO: 11); a VH CDR2 sequence comprising the amino acid sequence EIRLKSYNYATH (SEQ ID NO: 12); a VH CDR3 sequence comprising the amino acid sequence AGTDY (SEQ ID NO: 13); a VL CDR1 sequence comprising the amino acid sequence KASQSVRTDVA (SEQ ID NO: 14) or RASQSVRTDVG (SEQ ID NO: 15); a VL CDR2 sequence comprising the amino acid sequence YSSNRYT (SEQ ID NO: 16); and a VL CDR3 sequence comprising the amino acid sequence QQDYSSPFT (SEQ ID NO: 17) or QQDYSSPYT (SEQ ID NO: 18).

In some embodiments, the CD147 antibody or antigen-binding fragment thereof comprises a combination of a variable heavy chain complementarity determining region 1 (VH CDR1, also referred to herein as CDRH1) sequence, a variable heavy chain complementarity determining region 2 (VH CDR2, also referred to herein as CDRH2) sequence, a variable heavy chain complementarity determining region 3 (VH CDR3, also referred to herein as CDRH3) sequence, a variable light chain complementarity determining region 1 (VL CDR1, also referred to herein as CDRL1) sequence, a variable light chain complementarity determining region 2 (VL CDR2, also referred to herein as CDRL2) sequence, and a variable light chain complementarity determining region 3 (VL CDR3, also referred to herein as CDRL3) sequence, wherein at least one complementarity determining region (CDR) sequence is selected from the group consisting of a VH CDR1 sequence comprising the amino acid sequence GFTFSNYWMN (SEQ ID NO: 10) or GFTFSNYWMD (SEQ ID NO: 11); a VH CDR2 sequence comprising the amino acid sequence EIRLKSYNYATH (SEQ ID NO: 12); a VH CDR3 sequence comprising the amino acid sequence AGTDY (SEQ ID NO: 13); a VL CDR1 sequence comprising the amino acid sequence KASQSVRTDVA (SEQ ID NO: 14) or RASQSVRTDVG (SEQ ID NO: 15); a VL CDR2 sequence comprising the amino acid sequence YSSNRYT (SEQ ID NO: 16); and a VL CDR3 sequence comprising the amino acid sequence QQDYSSPFT (SEQ ID NO: 17) or QQDYSSPYT (SEQ ID NO: 18).

In some embodiments, the CD147 antibody or antigen-binding fragment thereof comprises a combination of a VH CDR1 sequence, a VH CDR2 sequence, a VH CDR3 sequence, a VL CDR1 sequence, a VL CDR2 sequence, and a VL CDR3 sequence, wherein at least one CDR sequence is selected from the group consisting of a VH CDR1 sequence that includes a sequence that is at least 90%, 91%, 92%, 93%, 94%, 95%, 96%, 97%, 98%, 99% or more identical to a VH CDR1 sequence comprising the amino acid sequence GFTFSNYWMN (SEQ ID NO: 10) or GFTFSNYWMD (SEQ ID NO: 11); a VH CDR2 sequence that includes a sequence that is at least 90%, 91%, 92%, 93%, 94%, 95%, 96%, 97%, 98%, 99% or more identical to a VH CDR2 sequence comprising the amino acid sequence EIRLKSYNYATH (SEQ ID NO: 12); a VH CDR3 sequence that includes a sequence that is at least 90%, 91%, 92%, 93%, 94%, 95%, 96%, 97%, 98%, 99% or more identical to a VH CDR3 sequence comprising the amino acid sequence AGTDY (SEQ ID NO: 13); a VL CDR1 sequence that includes a sequence that is at least 90%, 91%, 92%, 93%, 94%, 95%, 96%, 97%, 98%, 99% or more identical to a VL CDR1 sequence comprising the amino acid sequence KASQSVRTDVA (SEQ ID NO: 14) or RASQSVRTDVG (SEQ ID NO: 15); a VL CDR2 sequence that includes a sequence that is at least 90%, 91%, 92%, 93%, 94%, 95%, 96%, 97%, 98%, 99% or more identical to a VL CDR2 sequence comprising the amino acid sequence YSSNRYT (SEQ ID NO: 16); and a VL CDR3 sequence that includes a sequence that is at least 90%, 91%, 92%, 93%, 94%, 95%, 96%, 97%, 98%, 99% or more identical to a VL CDR3 sequence comprising the amino acid sequence QQDYSSPFT (SEQ ID NO: 17) or QQDYSSPYT (SEQ ID NO: 18).

In some embodiments, the CD147 antibody or antigen-binding fragment thereof comprises a combination of a VH CDR1 sequence, a VH CDR2 sequence, a VH CDR3 sequence, a VL CDR1 sequence, a VL CDR2 sequence, and a VL CDR3 sequence, wherein the VH CDR1 sequence comprises the amino acid sequence GFTFSNYWMN (SEQ ID NO: 10) or GFTFSNYWMD (SEQ ID NO: 11); the VH CDR2 sequence comprises the amino acid sequence EIRLKSYNYATH (SEQ ID NO: 12); the VH CDR3 sequence comprises the amino acid sequence AGTDY (SEQ ID NO: 13); the VL CDR1 sequence comprises the amino acid sequence KASQSVRTDVA (SEQ ID NO: 14) or RASQSVRTDVG (SEQ ID NO: 15); the VL CDR2 sequence comprises the amino acid sequence YSSNRYT (SEQ ID NO: 16); and the VL CDR3 sequence comprises the amino acid sequence QQDYSSPFT (SEQ ID NO: 17) or QQDYSSPYT (SEQ ID NO: 18).

In some embodiments, the CD147 antibody or antigen-binding fragment thereof comprises an amino acid sequence comprising amino acid sequences selected from the group consisting of: (a) the VH CDR1 sequence GFTFSNYWMN (SEQ ID NO: 10) or GFTFSNYWMD (SEQ ID NO: 11); the VH CDR2 sequence EIRLKSYNYATH (SEQ ID NO: 12); the VH CDR3 sequence AGTDY (SEQ ID NO: 13); the VL CDR1 sequence KASQSVRTDVA (SEQ ID NO: 14) or RASQSVRTDVG (SEQ ID NO: 15); the VL CDR2 sequence YSSNRYT (SEQ ID NO: 16); and the VL CDR3 sequence QQDYSSPFT (SEQ ID NO: 17) or QQDYSSPYT (SEQ ID NO: 18); (b) the VH CDR1 sequence GFTFSNYWMD (SEQ ID NO: 11); the VH CDR2 sequence EIRLKSYNYATH (SEQ ID NO: 12); the VH CDR3 sequence AGTDY (SEQ ID NO: 13); the VL CDR1 sequence RASQSVRTDVG (SEQ ID NO: 15); the VL CDR2 sequence YSSNRYT (SEQ ID NO: 16); and the VL CDR3 sequence QQDYSSPYT (SEQ ID NO: 18); and (c) the VH CDR1 sequence GFTFSNYWMN (SEQ ID NO: 10); the VH CDR2 sequence EIRLKSYNYATH (SEQ ID NO: 12); the VH CDR3 sequence AGTDY (SEQ ID NO: 13); the VL CDR1 sequence KASQSVRTDVA (SEQ ID NO: 14); the VL CDR2 sequence YSSNRYT (SEQ ID NO: 16); and the VL CDR3 sequence QQDYSSPFT (SEQ ID NO: 17).

In some embodiments, the CD147 antibody or antigen-binding fragment thereof comprises a combination of a VH CDR1 sequence, a VH CDR2 sequence, a VH CDR3 sequence, a VL CDR1 sequence, a VL CDR2 sequence, and a VL CDR3 sequence, wherein the VH CDR1 sequence comprises a sequence that is at least 90^{%}, 91%, 92%, 93%, 94%, 95%, 96%, 97%, 98%, 99% or more identical to the amino acid sequence GFTFSNYWMN (SEQ ID NO: 10) or GFTFSNYWMD (SEQ ID NO: 11); the VH CDR2 sequence comprises a sequence that is at least 90%, 91%, 92%, 93%, 94%, 95%, 96%, 97%, 98%, 99% or more identical to the amino acid sequence EIRLKSYNYATH (SEQ ID NO: 12); the VH CDR3 sequence comprises a sequence that is at least 90%, 91%, 92%, 93%, 94%, 95%, 96%, 97%, 98%, 99% or more identical to the amino acid sequence AGTDY (SEQ ID NO: 13); the VL CDR1 sequence comprises a sequence that is at least 90%, 91%, 92%, 93%, 94%, 95%, 96%, 97%, 98%, 99% or more identical to the amino acid sequence KASQSVRTDVA (SEQ ID NO: 14) or RASQSVRTDVG (SEQ ID NO: 15); the VL CDR2 sequence comprises a sequence that is at least 90%, 91%, 92%, 93%, 94%, 95%, 96%, 97%, 98%, 99% or more identical to the amino acid sequence YSSNRYT (SEQ ID NO: 16); and the VL CDR3 sequence a sequence that is at least 90%, 91%, 92%, 93%, 94%, 95%, 96%, 97%, 98%, 99% or more identical to comprises the amino acid sequence QQDYSSPFT (SEQ ID NO: 17) or QQDYSSPYT (SEQ ID NO: 18).

Suitable CD147 antibodies of the disclosure also include an antibody or antigen binding fragment thereof that binds to the same epitope on human CD147 and/or cynomolgus monkey CD147 as a CD147 antibody comprising a heavy chain variable region amino acid sequence selected from the group consisting of SEQ ID NOs: 1-4, and a light chain variable region amino acid sequence selected from the group consisting of SEQ ID NOs: 5-9.

Suitable CD147 antibodies of the disclosure also include an antibody or antigen binding fragment thereof that binds to the same epitope on human CD147 and/or cynomolgus monkey CD147 as a CD147 antibody comprising a VH CDR1 sequence comprising the amino acid sequence GFTFSNYWMN (SEQ ID NO: 10) or GFTFSNYWMD (SEQ ID NO: 11); a VH CDR2 sequence comprising the amino acid sequence EIRLKSYNYATH (SEQ ID NO: 12); a VH CDR3 sequence comprising the amino acid sequence AGTDY (SEQ ID NO: 13); a VL CDR1 sequence comprising the amino acid sequence KASQSVRTDVA (SEQ ID NO: 14) or RASQSVRTDVG (SEQ ID NO: 15); a VL CDR2 sequence comprising the amino acid sequence YSSNRYT (SEQ ID NO: 16); and a VL CDR3 sequence comprising the amino acid sequence QQDYSSPFT (SEQ ID NO: 17) or QQDYSSPYT (SEQ ID NO: 18).

Suitable CD147 antibodies of the disclosure also include an antibody or antigen binding fragment thereof that cross-competes for binding to (inhibits the binding of) human CD147 and/or cynomolgus monkey CD147 to a CD147 antibody comprising a heavy chain variable region amino acid sequence selected from the group consisting of SEQ ID NOs: 1-4, and a light chain variable region amino acid sequence selected from the group consisting of SEQ ID NOs: 5-9.

Suitable CD147 antibodies of the disclosure also include an antibody or antigen binding fragment thereof that cross-competes for binding to (inhibits the binding of) human CD147 and/or cynomolgus monkey CD147 to a CD147 antibody comprising a VH CDR1 sequence comprising the amino acid sequence GFTFSNYWMN (SEQ ID NO: 10) or GFTFSNYWMD (SEQ ID NO: 11); a VH CDR2 sequence comprising the amino acid sequence EIRLKSYNYATH (SEQ ID NO: 12); a VH CDR3 sequence comprising the amino acid sequence AGTDY (SEQ ID NO: 13); a VL CDR1 sequence comprising the amino acid sequence KASQSVRTDVA (SEQ ID NO: 14) or RASQSVRTDVG (SEQ ID NO: 15); a VL CDR2 sequence comprising the amino acid sequence YSSNRYT (SEQ ID NO: 16); and a VL CDR3 sequence comprising the amino acid sequence QQDYSSPFT (SEQ ID NO: 17) or QQDYSSPYT (SEQ ID NO: 18).

In some embodiments, the CD147 antibody of the disclosure comprises an isolated antibody or an antigen binding fragment thereof (AB) that specifically binds to mammalian CD147, wherein the AB specifically binds human CD147 and cynomolgus monkey CD147. In some embodiments, the antibody or antigen binding fragment thereof comprises the VH CDR1 sequence GFTFSNYWMN (SEQ ID NO: 10) or GFTFSNYWMD (SEQ ID NO: 11); the VH CDR2 sequence EIRLKSYNYATH (SEQ ID NO: 12); the VH CDR3 sequence AGTDY (SEQ ID NO: 13); the VL CDR1 sequence KASQSVRTDVA (SEQ ID NO: 14) or RASQSVRTDVG (SEQ ID NO: 15); the VL CDR2 sequence YSSNRYT (SEQ ID NO: 16); and the VL CDR3 sequence QQDYSSPFT (SEQ ID NO: 17) or QQDYSSPYT (SEQ ID NO: 18). In some embodiments, the antibody or antigen binding fragment thereof comprises a heavy chain variable region comprising an amino acid sequence selected from the group consisting of SEQ ID NOs: 1-4, and a light chain variable region comprising an amino acid sequence selected from the group consisting of SEQ ID NOs: 5-9.

In some embodiments, the isolated antibody or antigen binding fragment thereof binds to the same epitope on human CD147 and/or cynomolgus monkey CD147 as an isolated antibody that comprises the VH CDR1 sequence GFTFSNYWMN (SEQ ID NO: 10) or GFTFSNYWMD (SEQ ID NO: 11); the VH CDR2 sequence EIRLKSYNYATH (SEQ ID NO: 12); the VH CDR3 sequence AGTDY (SEQ ID NO: 13); the VL CDR1 sequence KASQSVRTDVA (SEQ ID NO: 14) or RASQSVRTDVG (SEQ ID NO: 15); the VL CDR2 sequence YSSNRYT (SEQ ID NO: 16); and the VL CDR3 sequence QQDYSSPFT (SEQ ID NO: 17) or QQDYSSPYT (SEQ ID NO: 18). In some embodiments, the isolated antibody or antigen binding fragment thereof binds to the same epitope on human CD147 and/or cynomolgus monkey CD147 as an isolated antibody that comprises a heavy chain variable region comprising an amino acid sequence selected from the group consisting of SEQ ID NOs: 1-4, and a light chain variable region comprising an amino acid sequence selected from the group consisting of SEQ ID NOs: 5-9.

In some embodiments, the isolated antibody or antigen binding fragment thereof cross-competes with (inhibits the binding of) an isolated antibody that comprises the VH CDR1 sequence GFTFSNYWMN (SEQ ID NO: 10) or GFTFSNYWMD (SEQ ID NO: 11); the VH CDR2 sequence EIRLKSYNYATH (SEQ ID NO: 12); the VH CDR3 sequence AGTDY (SEQ ID NO: 13); the VL CDR1 sequence KASQSVRTDVA (SEQ ID NO: 14) or RASQSVRTDVG (SEQ ID NO: 15); the VL CDR2 sequence YSSNRYT (SEQ ID NO: 16); and the VL CDR3 sequence QQDYSSPFT (SEQ ID NO: 17) or QQDYSSPYT (SEQ ID NO: 18). In some embodiments, the isolated antibody or antigen binding fragment thereof cross-competes with (inhibits the binding of) an isolated antibody that comprises a heavy chain variable region comprising an amino acid sequence selected from the group consisting of SEQ ID NOs: 1-4, and a light chain variable region comprising an amino acid sequence selected from the group consisting of SEQ ID NOs: 5-9.

In some embodiments, the isolated antibody or antigen binding fragment thereof cross-competes with (inhibits the binding of) an isolated antibody that comprises the VH CDR1 sequence GFTFSNYWMN (SEQ ID NO: 10) or GFTFSNYWMD (SEQ ID NO: 11); the VH CDR2 sequence EIRLKSYNYATH (SEQ ID NO: 12); the VH CDR3 sequence AGTDY (SEQ ID NO: 13); the VL CDR1 sequence KASQSVRTDVA (SEQ ID NO: 14) or RASQSVRTDVG (SEQ ID NO: 15); the VL CDR2 sequence YSSNRYT (SEQ ID NO: 16); and the VL CDR3 sequence QQDYSSPFT (SEQ ID NO: 17) or QQDYSSPYT (SEQ ID NO: 18). In some embodiments, the isolated antibody or antigen binding fragment thereof cross-competes with (inhibits the binding of) an isolated antibody that comprises a heavy chain variable region comprising an amino acid sequence selected from the group consisting of SEQ ID NOs: 1-4, and a light chain variable region comprising an amino acid sequence selected from the group consisting of SEQ ID NOs: 5-9.

In some embodiments, the CD147 antibody/activatable CD147 antibody includes a heavy chain that comprises or is derived from a heavy chain amino acid sequence shown in Table 1. In some embodiments, the CD147 antibody/activatable CD147 antibody includes a light chain that comprises or is derived from a heavy chain amino acid sequence shown in Table 1. In some embodiments, the CD147 antibody/activatable CD147 antibody includes a heavy chain that comprises or is derived from a heavy chain amino acid sequence shown in Table 1, and a light chain that comprises or is derived from a light chain amino acid sequence shown in Table 1. In some embodiments, the CD147 antibody/activatable CD147 antibody includes a combination of heavy chain variable region and light chain variable region sequences from the combinations shown in Group A in Table 1. In some embodiments, the CD147 antibody/activatable CD147 antibody includes a combination of heavy chain variable region and light chain variable region sequences from the sequences shown in Group B in Table 1. In some embodiments, the CD147 antibody/activatable CD147 antibody includes a combination of heavy chain variable region and light chain variable region sequences from the sequences shown in Group C in Table 1. In some embodiments, the CD147 antibody/activatable CD147 antibody includes a combination of heavy chain variable region and light chain variable region sequences from the sequences shown in Group D in Table 1. In some embodiments, the CD147 antibody/activatable CD147 antibody includes the combination of heavy chain variable region and light chain variable region sequences shown in Group E in Table 1. In some embodiments, the CD147 antibody/activatable CD147 antibody includes the combination of heavy chain variable region and light chain variable region sequences shown in Group F in Table 1. In some embodiments, the CD147 antibody/activatable CD147 antibody includes a combination of heavy chain variable region and light chain variable region sequences from the sequences shown in Group G in Table 1. In some embodiments, the CD147 antibody/activatable CD147 antibody includes a combination of heavy chain variable region and light chain variable region sequences from the sequences shown in Group H in Table 1. In some embodiments, the CD147 antibody/activatable CD147 antibody includes the combination of heavy chain variable region and light chain variable region sequences shown in Group I in Table 1. In some embodiments, the CD147 antibody/activatable CD147 antibody includes the heavy chain variable region sequence shown in Group J in Table 1. In some embodiments, the CD147 antibody/activatable CD147 antibody includes the heavy chain variable region sequence shown in Group J in Table 1, or the combination of heavy chain variable region and light chain variable region sequences shown in Group K in Table 1. In some embodiments, the CD147 antibody/activatable CD147 antibody includes a combination of heavy chain variable region and light chain variable region sequences from the sequences shown in Group L in Table 1.

In some embodiments, the CD147 antibody/activatable CD147 antibody includes a combination of the complementarity determining region (CDR) sequences of a heavy chain sequence from the heavy chain sequences shown in Group A Table 1. In some embodiments, the CD147 antibody/activatable CD147 antibody includes a combination of the CDRs of a light chain sequence from the light chain sequences shown in Group A Table 1. In some embodiments, the CD147 antibody/activatable CD147 antibody includes a combination of the CDRs of a heavy chain sequence from the heavy chain sequences shown in Group A Table 1 and the CDRs of a light chain sequence from the heavy chain sequences shown in Group A Table 1.

In some embodiments, the CD147 antibody/activatable CD147 antibody includes a combination of CDRs of a heavy chain sequence from the heavy chain sequences shown in Group B Table 1. In some embodiments, the CD147 antibody/activatable CD147 antibody includes a combination of the CDRs of a light chain sequence from the light chain sequences shown in Group B Table 1. In some embodiments, the CD147 antibody/activatable CD147 antibody includes a combination of the CDRs of a heavy chain sequence from the heavy chain sequences shown in Group B Table 1 and the CDRs of a light chain sequence from the heavy chain sequences shown in Group B Table 1.

In some embodiments, the CD147 antibody/activatable CD147 antibody includes a combination of the CDRs of a heavy chain sequence from the heavy chain sequences shown in Group C Table 1. In some embodiments, the CD147 antibody/activatable CD147 antibody includes a combination of the CDRs of a light chain sequence from the light chain sequences shown in Group C Table 1. In some embodiments, the CD147 antibody/activatable CD147 antibody includes a combination of the CDRs of a heavy chain sequence from the heavy chain sequences shown in Group C Table 1 and the CDRs of a light chain sequence from the heavy chain sequences shown in Group C Table 1.

In some embodiments, the CD147 antibody/activatable CD147 antibody includes a combination of the CDRs of a heavy chain sequence from the heavy chain sequences shown in Group D Table 1. In some embodiments, the CD147 antibody/activatable CD147 antibody includes a combination of the CDRs of a light chain sequence from the light chain sequences shown in Group D Table 1. In some embodiments, the CD147 antibody/activatable CD147 antibody includes a combination of the CDRs of a heavy chain sequence from the heavy chain sequences shown in Group D Table 1 and the CDRs of a light chain sequence from the heavy chain sequences shown in Group D Table 1.

In some embodiments, the CD147 antibody/activatable CD147 antibody includes a combination of the CDRs of a heavy chain sequence from the heavy chain sequences shown in Group E Table 1. In some embodiments, the CD147 antibody/activatable CD147 antibody includes a combination of the CDRs of a light chain sequence from the light chain sequences shown in Group E Table 1. In some embodiments, the CD147 antibody/activatable CD147 antibody includes a combination of the CDRs of a heavy chain sequence from the heavy chain sequences shown in Group E Table 1 and the CDRs of a light chain sequence from the heavy chain sequences shown in Group E Table 1.

In some embodiments, the CD147 antibody/activatable CD147 antibody includes a combination of the CDRs of a heavy chain sequence from the heavy chain sequences shown in Group F Table 1. In some embodiments, the CD147 antibody/activatable CD147 antibody includes a combination of the CDRs of a light chain sequence from the light chain sequences shown in Group F Table 1. In some embodiments, the CD147 antibody/activatable CD147 antibody includes a combination of the CDRs of a heavy chain sequence from the heavy chain sequences shown in Group F Table 1 and the CDRs of a light chain sequence from the heavy chain sequences shown in Group F Table 1.

In some embodiments, the CD147 antibody/activatable CD147 antibody includes a combination of the CDRs of a heavy chain sequence from the heavy chain sequences shown in Group G Table 1. In some embodiments, the CD147 antibody/activatable CD147 antibody includes a combination of the CDRs of a light chain sequence from the light chain sequences shown in Group G Table 1. In some embodiments, the CD147 antibody/activatable CD147 antibody includes a combination of the CDRs of a heavy chain sequence from the heavy chain sequences shown in Group G Table 1 and the CDRs of a light chain sequence from the heavy chain sequences shown in Group G Table 1.

In some embodiments, the CD147 antibody/activatable CD147 antibody includes a combination of the CDRs of a heavy chain sequence from the heavy chain sequences shown in Group H Table 1. In some embodiments, the CD147 antibody/activatable CD147 antibody includes a combination of the CDRs of a light chain sequence from the light chain sequences shown in Group H Table 1. In some embodiments, the CD147 antibody/activatable CD147 antibody includes a combination of the CDRs of a heavy chain sequence from the heavy chain sequences shown in Group H Table 1 and the CDRs of a light chain sequence from the heavy chain sequences shown in Group H Table 1.

In some embodiments, the CD147 antibody/activatable CD147 antibody includes a combination of the CDRs of a heavy chain sequence from the heavy chain sequences shown in Group I Table 1. In some embodiments, the CD147 antibody/activatable CD147 antibody includes a combination of the CDRs of a light chain sequence from the light chain sequences shown in Group I Table 1. In some embodiments, the CD147 antibody/activatable CD147 antibody includes a combination of the CDRs of a heavy chain sequence from the heavy chain sequences shown in Group I Table 1 and the CDRs of a light chain sequence from the heavy chain sequences shown in Group I Table 1.

In some embodiments, the CD147 antibody/activatable CD147 antibody includes a combination of the CDRs of a heavy chain sequence from the heavy chain sequences shown in Group J Table 1. In some embodiments, the CD147 antibody/activatable CD147 antibody includes a combination of the CDRs of a light chain sequence from the light chain sequences shown in Group J Table 1. In some embodiments, the CD147 antibody/activatable CD147 antibody includes a combination of the CDRs of a heavy chain sequence from the heavy chain sequences shown in Group J Table 1 and the CDRs of a light chain sequence from the heavy chain sequences shown in Group J Table 1.

In some embodiments, the CD147 antibody/activatable CD147 antibody includes a combination of the CDRs of a heavy chain sequence from the heavy chain sequences shown in Group K Table 1. In some embodiments, the CD147 antibody/activatable CD147 antibody includes a combination of the CDRs of a light chain sequence from the light chain sequences shown in Group K Table 1. In some embodiments, the CD147 antibody/activatable CD147 antibody includes a combination of the CDRs of a heavy chain sequence from the heavy chain sequences shown in Group K Table 1 and the CDRs of a light chain sequence from the heavy chain sequences shown in Group K Table 1.

In some embodiments, the CD147 antibody/activatable CD147 antibody includes a combination of the CDRs of a heavy chain sequence from the heavy chain sequences shown in Group L Table 1. In some embodiments, the CD147 antibody/activatable CD147 antibody includes a combination of the CDRs of a light chain sequence from the light chain sequences shown in Group L Table 1. In some embodiments, the CD147 antibody/activatable CD147 antibody includes a combination of the CDRs of a heavy chain sequence from the heavy chain sequences shown in Group L Table 1 and the CDRs of a light chain sequence from the heavy chain sequences shown in Group L Table 1.

TABLE 1
Variable Heavy Chain Region (VH) and Variable Light Chain Region (VL)
Sequences for Antibodies and Activatable Antibodies that Bind CD147
Group A

| | |
|---|---|
| VH | EVQLQQSGPELVKPGASVKISCKASGYTFTDYYMNWVKQSHGKSLEWIGGINPNNGGTSYNQKFKGKATLTV |
| | DKSSSTAYMELRSLTSEDSAVYYCARNDGYRGYAMDYWGQGTSVTVSS (SEQ ID NO: 425) |
| VH | QVQLQQSGAELAKPGASVKLSCKASGYTFTSYTFLMHWVKQRPGQGLEWIGYINPGSGYTKYNQTFKDKATLTA |
| | DKSSSTAYMQLSSLTYEDSAVYYCARVEGYRTARYFDVWGTGTTVTVSS (SEQ ID NO: 426) |
| VH | EMKLEESGGGLVQPGGSMKLSCVASGFTFSNYWMNWVRQSPEKGLELAFVAQIRLKSYNYATHYAESVKGRFTI |
| | SRDDSKSSVYLQMNNLRAEDTGIYYCTPDGSDYWGQGTTLTVSS (SEQ ID NO: 427) |
| VH | EMKLEESGGGLVQPGGSMKLSCVASGFTFSNYRMNWVRQSPEKGLEWVAQIRLKSYNYATHYAESVKGRFTT |
| | SRDDSKSSVYLQMNNLRAEDTGIYYCTPDGSDYWGQGTTLTVSS (SEQ ID NO: 428) |
| VL | DIVMSQSPSSLAVSVGEKVTMSCKSSQSLLYSSNQKNYLAWYQQKPGQSPKLLIYWASTRESGVPDRFTGSG |
| | SGTDFTLTISSVKAEDLAVYYCQQYYSYPTFGAGTKLELK (SEQ ID NO: 429) |
| VL | DIVMSQSPSSLAVSVGEKVTMSCKSSQSLLYNNNQKNYLAWYQQKPGQSPKLLIYWASTRESGVPDRFTGSG |
| | SGTDFTLTISSVKAEDLAVYYCQQYYSYPFTFGSGTKLEIK (SEQ ID NO: 430) |
| VL | SIVMTQTPKFLLVSAGDRVTITCKASQSVSNDVAWYQQKPGQSPKLLIYYASNRYTGVPDRFTGSGYGTDFT |
| | FTISTVQAEDLAVYFCQQDYSSPYTFGGGTKLEIK (SEQ ID NO: 431) |
| VL | DILMTQSPSSMSVSLGDTVSITCHASQGISSSIGWLQQKPGKSFKGLIYHGTNLEDGVPSRFTGSGSGADYS |
| | LTISSLESEDFADYYCVQYAQFPYTFGGGTKLEIK (SEQ ID NO: 432) |

Group B

| | |
|---|---|
| VH | GLLKPSETLSLTCAVYGGSFSGYYWSWIRQPPGKGLEWIGEINHSGSTNYNPSLKSRVTISVDTSKMQFSLK |
| | LSSVTAADTAVYYCARGTTEYYYYYYGMDVWGQGTTVTVSSGSASAPTLFPLVSCENSPSDTSSVAVGCLAQ |
| | DFLPDXITFSWKYKNNSDISSTRGFPSVLRGGKYAATSQVLLPSKDVMQGTDEHWTGSKE (SEQ ID |
| | NO: 433) |
| VH | LVKPSETLSLTCTVSGGSISSYYWNWIRQPPGKGLEWIGYIYYSGSTNYNPSLKSRVTISVDTSKNQFSLKL |
| | SSVTAADTAVYYCARDRGVGATGFDYWGQGTLVTVSSGSASAPTLFPLVSCENSPSDTSSVAVGCLAQDFLP |
| | DSITFSWKYKNNSDISSTRGFPSVLRGGKYAATSQVLLPSKDVMQGTDEHKVC (SEQ ID NO: 434) |
| VH | KKPGASVKVSCKASGYTFTSYDINWVRQATGQGLEWMGWMNPNSGNTGYAQKFQGRVTMNRNTSISTAYMEL |
| | SSLRSEDTAVYYCARGGHGGSYFYSYYGMDVWGQGTTVTVSSGSASAPTLFPLVSCENSPSDTSSVAVGCLA |
| | QDFLPDSITFSWKYKNNSDISSTRGFPSVLRGGKYAATSQVLLPSKDVMQGTDEHWCK (SEQ ID |
| | NO: 435) |
| VH | EVKKPGASVKVSCPCASGYTFTSYDINWVRQATGQGLEWMGWMNPNSGNTGYAQKFQGRVTMTRNTSISTAYM |
| | ELSSLRSEDTAVYYCAREEWLVRYYGMDVWGQGTTVTVSSGSASAPTLFPLVSCENSPSDTSSVAVGCLAQD |
| | FLPDSITFSWKYKNNSDISSTRGFPSVLRGGKYAATSQVLLPSKDVMQGTDEHKV (SEQ ID NO: 436) |
| VH | KLPETLSLTCAVYGGSFSGYYWSWIRQPPGKGLEWIGEINHSGSTNYNPSLKSRVTISVDTSKNQFSLKLSS |
| | VTAADTAVYYCARGAAEYYYYYYGMDVWGQGTTVTVSSGSASAPTLFPLVSCENSPSDTSSVAVGCLAQDFL |
| | PDXITFXWKYKNNSDISSTRGFPSVLRGGKYAATSQVLLPSKDVMQGTDEHWTGSKE (SEQ ID |
| | NO: 437) |
| VH | SETLSLTCAVYGGSFSGYYWSWIRQPPGKGLEWIGEINHSGSTNYNPSLKSRVTISVDTSKNQFSLKLSSVT |
| | AADTAVYYCARGGTTVTFDAFDIWGQGTMVTVSSGSASAPTLFPLVSCENSPSDTSSVAVGCLAQDFLPDSI |
| | TFSWKYKNNSDISSTRGFPSVLRGGKYAATSQVLLPSKDVMQGTDE (SEQ ID NO: 438) |
| VH | NPQTTLTLTCTFSGFSLITRGVGVDWIRQPPGKALQWLALIYWNDDKRYSPSLKSRLTITKDTSKNQWLTM |
| | TNMDPVDTATYYCAHHFFDSSGYYPFDSWGQGTLVSVSSASTKGPSVFPLAPCSRSTSESTAALGCLVKDYF |
| | PEPVT (SEQ ID NO: 439) |
| VH | GEGLVKPGGSLRLSCAASGFTFSSYSMNWVRQAPGKGLEWVSSISSSSSYIYYADSVKGRFTISRDNAKNSL |
| | YLQMNSLRAEDTAVYYCARDSSGWYEDYFDYWGQGTLVTVSSASTKGPSVFPLAPCSRSTSESTAALGCLVK |
| | DYFPEPVTVSWNSGALTSGVHTFPAVLQS (SEQ ID NO: 440) |
| VH | LTCTFSGFSLITRGVGVDWIRQPPGKALQWLALIYWNDDKRYSPSLKSRLTITKDTSKNQWLTMTNMDPVD |
| | TATYYCAHHFFDSSGYYPFDSWGQGTLVSVSSASTKGPSVFPLAPCSRSTSESTAALGCLVKDYFPEPVTVS |
| | WNSGALTSGVHTFQL (SEQ ID NO: 441) |
| VH | GGGLVQPGGSLRLSCAASGFTFSSYAMSWVRQAPGKGLEWVSTISVSGITTYYVDSVKGRFTISRDNSKNIL |
| | YLQMNSLRAEDTAVYYCAKRIFGVVWGQGTLVTVSSASTKGPSVFPLAPCSRSTSESTAALGCLVKDYFPEP |
| | VTVSWNLGALTSGVHTFPAVLQS (SEQ ID NO: 442) |
| VL | LSLPVTPGEPASISCRSSQSLLHSNGYNYLDWYLQKPGQSPQLLIYLGSNRASGVPDRFSGSGSGTDFTLKI |
| | SRVEAEDVGIYYCMQTRQTPRTFGQGTKVEIKRTVAAPSVFIFPPSDEQLKSGTASWCLLNNFYPREAKEH |
| | QKSP (SEQ ID NO: 443) |
| VL | SQSPSSLSASVGERVTITCRASQGIRDELGWYQQKPGKAPKRLIYVASSLQSGVPSRFSGSGSGTEFTLTIS |
| | SLQPEDFATYYCLQHNGYPRTFGQGTKVEIKRTVAAPSVFIFPPSDEQLKSGTASWCLLNNFYPREAKEHQ |
| | KSP (SEQ ID NO: 444) |
| VL | HSLAVSLGERATINCKSSQSVLYSFNNKNYLAWYQQKPGQPPKLLIYWASTRESGVPDRFGGSGSGTDFTLT |
| | ISSLQAEDVAVYYCQQYYSTPRTFGQGTKVEIKRTVAAPSVFIFPPSDEQLKSGTASWCLLNNFYPREAKE |
| | HQKSP (SEQ ID NO: 445) |
| VL | GQSPSSLSASVGDRVTITCRASQDIRDNLGWYQQKPGKAPKRLIYAASNLQSGVPSRFSGSGSGTEFTLTIS |
| | SLQPEDFATYYCLQYKTYPWTFGQGTKVEIKRTVAAPSVFIFPPSDEQLKSGTASWCLLNNFYPREXKEHQ |
| | KSP (SEQ ID NO: 446) |
| VL | MPVTPGEPASISCRSSQSLLHSNGYNYLDWYLQKPGQSPQLLIYLGSNRASGVPDRFSGSGSGTDFTLKISR |
| | VEAEDVGIYYCMQSLQIPRLFGPGTKVDIKRTVAAPSWIFPPSDEQLKSGTASWCLLSNFYPREAKVQW |
| | (SEQ ID NO: 447) |
| VL | LAVSLGERATINCKSSQSVLYSFNNKNYLAWYQQKPGQPPKLLIYWASTRESGVPDRFSGSGSGTDFTLTIS |
| | SLQAEDVAVYYCQQYYSTPRTFGQGTKVEIKRTVAAPSVFIFPPSDEQLKSGTASWCLLNNFYPREAKVQW |
| | KVI (SEQ ID NO: 448) |
| VL | VTQSPLSLSVTPGQPASISCKSSQSLLHSDGKTYLYWYLQKPGQPPQLLIYEAFNRFSGVPDRFSGSGSGTD |
| | FTLKISRVEAEDVGLYYCMQSIELPFTFGGGTKVEIKRTVAAPSVFIFPPSDEQLKSGTASWCLLNNFYPR |
| | KERV (SEQ ID NO: 449) |
| VL | LDIQLTQSPSSLSASVGDRVTITCRASQDISIYIAWFQQRPGKAPKSLIYAASSLQSGVPSKFSGSGSGTDF |
| | TLTISSLQPEDFATYYCQQYNSYPFTFGP (SEQ ID NO: 450) |
| VL | GIRLDIQLTQSPSSLSASVGDRVTITCRASQGISIYLAWFQQRPGKAPKSLIYAASSLQSGVPSKFSGSGSG |
| | TDFTLTISSLQPEDFATYYCQQYNSYPFTFGPGTKVDIKRTVAAPSVFIFPPSDEQLKSGTASWCLLNNFY |
| | PREAKVQWKVDNALQSGKPN (SEQ ID NO: 451) |

Group C

| | |
|---|---|
| VH | GLLKPSETLSLTCAVYGGSFSGYYWSWIRQPPGKGLEWIGEINHSGSTNYNPSLKSRVTISVDTSKNQFSLK |
| | LSSVTAADTAVYYCARGTTEYYYYYYGMDVWGQGTTVTVSSGSASAPTLFPLVSCENSPSDTSSVAVGCLAQ |
| | DFLPDXITFSWKYKNNSDISSTRGFPSVLRGGKYAATSQVLLPSKDVMQGTDEHWTGSKE (SEQ ID |
| | NO: 433) |
| VH | LVKPSETLSLTCTVSGGSISSYYWNWIRQPPGKGLEWIGYIYYSGSTNYNPSLKSRVTISVDTSKNQFSLKL |
| | SSVTAADTAVYYCARDRGVGATGFDYWGQGLVTVSSGSASAPTLFPLVSCENSPSDTSSVAVGCLAQDFLPD |
| | SITFSWKYKNNSDISSTRGFPSVLRGGKYAATSQVLLPSKDVMQGTDEHKVC (SEQ ID NO: 452) |
| VH | KKPGASVKVSCKASGYTFTSYDINWVRQATGQGLEWMGWMNPNSGNTGYAQKFQGRVTMNRNTSISTAYMEL |
| | SSLRSEDTAVYYCARGGHGGSYFYSYYGMDVWGQGTTVTVSSGSASAPTLFPLVSCENSPSDTSSVAVGCLA |
| | QDFLPDSITFSWKYKNNSDISSTRGFPSVLRGGKYAATSQVLLPSKDVMQGTDEHWCK (SEQ ID |
| | NO: 435) |
| VH | EVKKPGASVKVSCKASGYTFTSYDINWVRQATGQGLEWMGWMNPNSGNTGYAQKFQGRVTMTRNTSISTAYM |
| | ELSSLRSEDTAVYYCAREEWLVRYYGMDVWGQGTTVTVSSGSASAPTLFPLVSCENSPSDTSSVAVGCLAQD |
| | FLPDSITHSWKYKNNSDISSTRGFPSVLRGGKYAATSQVLLPSKDVMQGTDEHKV (SEQ ID NO: 436) |
| VH | KLPETLSLTCAVYGGSFSGYYWSWIRQPPGKGLEWIGEINHSGSTNYNPSLKSRVTISVDTSKNQFSLKLSS |
| | VTAADTAVYYCARGAAEYYYYYYGMDVWGQGTTVTVSSGSASAPTLFPLVSCENSPSDTSSVAVGCLAQDFL |
| | PDXITFXWKYKNNSDISSTRGFPSVLRGGKYAATSQVLLPSKDVMQGTDEHVVTGSKE (SEQ ID |
| | NO: 437) |
| VH | GEGLVKPGGSLRLSCAASGFTFSSYSMNWVRQAPGKGLEWVSSISSSSSYIYYADSVKGRFTISRDNAKNSL |
| | YLQMNSLRAEDTAVYYCARDSSGWYEDYFDYWGQGTLVTVSSASTKGPSVFPLAPCSRSTSESTAALGCLVK |
| | DYFPEPVTVSWNSGALTSGVHTFPAVLQS (SEQ ID NO: 440) |
| VH | SETLSLTCAVYGGSFSGYYWSWIRQPPGKGLEWIGEINHSGSTNYNPSLKSRVTISVDTSKHQFSOIOSSVT |
| | AADTAWCARGGTTWFDAFDIWGQGTMVTVSSGSASAPTLFPLVSCENSPSDTSSVAVGCLAQDFLPDSIT |
| | FSWKYKNNSDISSTRGFPSVLRGGKYAATSQVLLPSKDVMQGTDE (SEQ ID NO: 453) |
| VH | LTCTFSGFSLITRGVGVDWIRQPPGKALQWLALIYWNDDKRYSPSLKSRLTITKDTSKNQWLTMTNMDPVD |
| | TATYYCAHHFFDSSGYYPFDSWGQGTLVSVSSASTKGPSVFPLAPCSRSTSESTAALGCLVKDYFPEPVTVS |
| | WNSGALTSGVHTFQL (SEQ ID NO: 441) |
| VH | GGGLVQPGGSLRLSCAASGFTFSSYAMSWVRQAPGKGLEWVSTISVSGITTYYVDSVKGRFTISRDNSKNIL |
| | YLQMNSLRAEDTAVYYCAKRIFGVVWGQGTLVTVSSASTKGPSVFPLAPCSRSTSESTAALGCLVKDYFPEP |
| | VTVSWNLGALTSGVHTFPAVLQS (SEQ ID NO: 442) |
| VH | GIRLDIQLTQSPSSLSASVGDRVTITCRASQGISIYLAWFQQRPGKAPKSLIYAASSLQSGVPSKFSGSGSG |
| | TDFTLTISSLQPBDFATYYCQQYNSYPFTFGPGTKVDIKRTVAAPSVFIFPPSDEQLKSGTASWCLLNNFY |
| | PREAKVQWKVDNALQSGKPN (SEQ ID NO: 451) |
| VH | ERATINCKSSQSVLYSSNNKNYLAWYQQKPGQPPKLLIYWASTRESGVPDRFSGSGSGTDFTLTISSLQAED |
| | VAVYYCQQYYSTP (SEQ ID NO: 454) |
| VH | ERATINCKSSQSVLYSFNNKNYLAWYQQKPGQPPKLLIYWASTRESGVPDRFSGSGSGTDFTLTISSLQAED |
| | VAVYYCQQYYSTRT (SEQ ID NO: 455) |
| VH | ERATINCKSSQSVLYSFNNKNYLAWYQQKPGQPPKLLIYWASTRESGVPDRFGGSGSGTDFTLTISSLQAED |
| | VAVYYCQQYYSTRT (SEQ ID NO: 456) |
| VL | LSLPVTPGEPASISCRSSQSLLHSNGYNYLDWYLQKPGQSPQLLIYLGSNRASGVPDRFSGSGSGTDFTLKI |
| | SRVEAEDVGIYYCMQTRQTPRTFGQGTKVEIKRTVAAPSVFIFPPSDEQLKSGTASWCLLNNFYPREAKEH |
| | QKSP (SEQ ID NO: 443) |
| VL | SQSPSSLSASVGERVTITCRASQGIRDELGWYQQKPGKAPKRLIYVASSLQSGVPSRFSGSGSGTEFTLTIS |
| | SLQPEDFATYYCLQHNGYPRTFGQGTKVEIKRTVAAPSVFIFPPSDEQLKSGTASWCLLNNFYPREAKEHQ |
| | KSP (SEQ ID NO: 444) |
| VL | HSLAVSLGERATINCKSSQSVLYSFNNKNYLAWYQQKPGQPPKLLIYWASTRESGVPDRFGGSGSGTDFTLT |
| | ISSLQAEDVAVYYCQQYYSTPRTFGQGTKVEIKRTVAAPSVFIFPPSDEQLKSGTASWCLLNNFYPREAKE |
| | HQKSP (SEQ ID NO: 445) |
| VL | GQSPSSLSASVGDRVTITCRASQDIRDNLGWYQQKPGKAPKRLIYAASNLQSGVPSRFSGSGSGTEFTLTIS |
| | SLQPEDFATYYCLQYKTYPWTFGQGTKVEIKRTVAAPSVFIFPPSDEQLKSGTASWCLLNNFYPREXKEHQ |
| | KSP (SEQ ID NO: 446) |
| VL | MPVTPGEPASISCRSSQSLLHSNGYNYLDWYLQKPGQSPQLLIYLGSNRASGVPDRFSGSGSGTDFTLKISR |
| | VEAEDVGIYYCMQSLQIPRLFGPGTKVDIKRTVAAPSVFIFPPSDEQLKSGTASWCLLSNFPREAKVQW |
| VL | LAVSLGERATINCKSSQSVLYSFNNKNYLAWYQQKPGQPPKLLIYWASTRESGVPDRFSGSGSGTDFTLTIS |
| | SLQAEDVAVYYCQQYYSTPRTFGQGTKVEIKRTVAAPSVFIFPPSDEQLKSGTASWCLLNNFYPREAKVQW |
| | KVI (SEQ ID NO: 448) |
| VL | NPQTTLTLTCTFSGFSLITRGVGVDWIRQPPGKALQWLALIYWNDDKRYSPSLKSRLTITKDTSKNQVVLTM |
| | TNMDPVDTATYYCAHHFFDSSGYYPFDSWGQGTLVSVSSASTKGPSVFPLAPCSRSTSESTAALGCLVKDYF |
| | PEPVT (SEQ ID NO: 439) |
| VL | VTQSPLSLSVTPGQPASISCKSSQSLLHSDGKTYLYWYLQKPGQPPQLLIYEAFNRFSGVPDRFSGSGSGTD |
| | FTLKISRVEAEDVGLYYCMQSIELPFTFGGGTKVEIKRTVAAPSVFIFPPSDEQLKSGTASWCLLNNFYPR |
| | KERV (SEQ ID NO: 449) |
| VL | LDIQLTQSPSSLSASVGDRVTITCRASQDISIYLAWFQQRPGKAPKSLIYAASSLQSGVPSKFSGSGSGTDF |
| | TLTISSLQPEDFATYYCQQYNSYPFTFGP (SEQ ID NO: 450) |
| VL | HSLAVSLGERATINCKSSQSVLYSFNNKNYLAWYQQKPGQPPKLLIYWASTRESGVPDRFGGSGSGTDFTLT |
| | ISSLQAEDVAVYYCQQYYSTPRTFGQGTKVEIKRTVAAPSVFIFPPSDEQLKSGTASVVCLLNNFYPREAKE |
| | HQKSP (SEQ ID NO: 445) |
| VL | GQSPSSLSASVGDRVTITCRASQDIRDNLGWYQQKPGKAPKRLIYAASNLQSGVPSRFSGSGSGTEFTLTIS |
| | SLQPEDFATYYCLQYKTYPWTFGQGTKVEIKRTVAAPSVFIFPPSDEQLKSGTASWCLLNNFYPREXKEHQ |
| | KSP (SEQ ID NO: 446) |
| VL | LAVSLGERATINCKSSQSVLYSFNNKNYLAWYQQKPGQPPKLLIYWASTRESGVPDRFSGSGSGTDFTLTIS |
| | SLQAEDVAVYYCQQYYSTPRTFGQGTKVEIKRTVAAPSVFIFPPSDEQLKSGTASVVCLLNNFYPREAKVQW |
| | KVI (SEQ ID NO: 448) |
| VL | DRVTITCRASQDIRDNLGWYQQKPGKAPKRLIYAASNLQSGVPSRFSGSGSGTEFTLTISSLQPEDFATYYC |
| | LQYKTYP (SEQ ID NO: 457) |
| VL | DRVTITCRASQGIRNDLGWYQQKPGKAPKRLIYAASSLQSGVPSRFSGSGSGTEFTLTISSLQPEDFATYYC |
| | LQHNSYP (SEQ ID NO: 458) |

Group D

| | |
|---|---|
| VH | EVKLEESGGGLVQPGGSMKLSCVASGFRRFSNFWMDVVVRQSPFKGLEWIAGIRLKSYNYARRHYAESVKGRFTI |
| | SRDDSKSSWLQMNNLRAEDTGIYYCTDWDGAYWGQGTLVTVSA (SEQ ID NO: 459) |
| VH | EVQLVESGGGLVQPGGSLKLSCAASGFTFSNFWMDWVRQASGKGLETWGGIRLKSYNYATHYAESVKGRFTI |
| | SRDDSKNTAYLQMNSLKTEDTAVYYCTRWDGAYWGQGTLVTVSS (SEQ ID NO: 460) |
| VH | EVQLVESGGGLVQPGGSLRLSCAASGFTFSNFWMDWVRQAPGKGLETWGGIRLKSYNYATHYAESVKGRFTI |
| | SRDDSKNTAYLQMNSLRAEDTAVYYCTRWDGAYWGQGTLVTVSS (SEQ ID NO: 461) |
| VH | EVQLVESGGGLVQPGGSLRLSCAASGFTFSNFWMDWVRQAPGKGLEWIAGIRLKSYNYATHYAESVKGRFTI |
| | SRDDSKSTVYLQMNSLRAEDTAVYYCTDWDGAYWGQGTLVTVSS (SEQ ID NO: 462) |
| VH | EVQLVESGGGLVQPGGSLKLSCAASGFTFSNFWMDVIFVRQASGKGLEWVGEIRLKSTNYATHYAESVKGRFTI |
| | SRDDSKNTAYLQMNSLKTEDTAVYYCTRTSTGYWGQGTTVTVSS (SEQ ID NO: 463) |
| VH | EVQLVESGGGLVQPGGSLKLSCAASGFTFSNFWMDWVRQASGKGLEWVAEIRLKSTNYATHYAESVKGRFTI |
| | SRDDSKSTVYLQMNSLKTEDTAVYYCTATSTGYWGQGTTVTVSS (SEQ ID NO: 464) |
| VH | EVQLVESGGGLVQPGGSLKLSCAASGFTFSNEWMDWVRQASGKGLEWVGEIRLKSTNYATHYAESVKGRFTI |
| | SRDDSKNTAYLQMNSLKTEDTAVYYCTATSTGYWGQGTTVTVSS (SEQ ID NO: 465) |
| VH | EVKLEESGGGLVQPGGSMKLSCVASGFTFSNFWMDWVRQSPFKGLEWVAFIRLKSTNYATHYAESVKGRFTI |
| | SRDDSKSSVYLQMNNLRAEDTGIYYCTATSTGYWGQGTTLTVSS (SEQ ID NO: 466) |
| VH | QVQLQQPGAEIVRPGASVKLSCKASGYTFTDYWMNWVKLRPGQGLEWIGIIDPSDSYASYNQKFKGKATLTV |
| | DESSSTAYMQLSSLTSEDSAVYYCARKSYYGGNYYYAMDYWGQGTSVTVSS (SEQ ID NO: 467) |
| VL | DIVMTQSHKFMSTSVGDRVSITCKASQDVSTDVAWYQQKPGQSPKLLIYSASYRYTGVPDRFTGSGSGTDFT |
| | FTISSVQAEDLAVYYCQQHYSTPFTFGSGTKLEIK (SEQ ID NO: 468) |
| VL | DIQMTQSPSSLSASVGDRVTITCKASQDVSTDVAWYQQKPGKAPKLLIYSASYRYTGVPSRFSGSGSGTDFT |
| | FTISSLQPEDIATYYCQQHYSTPFTFGQGTKLEIK (SEQ ID NO: 469) |
| VL | DIQMTQSPSSLSASVGDRVTITCKASQDVSTDVAWYQQKPGKAPKLLIYSASYRYTGVPSRFSGSGSGTDFT |
| | LTISSLQPEDIATYYCQQHYSTPFTFGQGTKLEIK (SEQ ID NO: 470) |
| VL | DIQMTQSPSSLSASVGDRVTITCKASQDVSTDVAWYQQKPGKSPKLLIYSASYRYTGVPSRFSGSGSGTDFT |
| | LTISSLQPEDIATYYCQQHYSTPFTFGQGTKLEIK (SEQ ID NO: 471) |
| VL | DIVMTQSPSSLSASVGDRVTITCKASQDVSTDVAWYQQKPGKSPKLLIYSASYRYTGVPSRFSGSGSGTDFT |
| | LTISSLQPEDIATYYCQQHYSTPFTFGQGTKLEIK (SEQ ID NO: 472) |
| VL | DIQMTQSPSSLSASVGDRVTITCKASQSVSNDVAWYQQKPGKAPKLLIYYASNRYTGVPSRFSGSGSGTDFT |
| | FTISSLQPEDIATYYCQQDYSSPYTFGGGTKVEIK (SEQ ID NO: 473) |
| VL | DIQMTQSPSSLSASVGDRVTITCKASQSVSNDVAWYQQKPGKSPKLLIYYASNRYTGVPSRFSGSGSGTDFT |
| | FTISSLQPEDIATYFCQQDYSSPYTFGGGTKVEIK (SEQ ID NO: 474) |
| VL | EIVMTQSPATLSVSPGERATLSCKASQSVSNDVAWYQQKPGQAPRLLIYYASNRYTGIPARFSGSGSGTEFT |
| | LTISSLQSEDFAVYYCQQDYSSPYTFGGGTKVEIK (SEQ ID NO: 475) |
| VL | EIVMTQSPATLSVSPGERATLSCKASQSVSNDVAWYQQKPGQSPRLLIYYASNRYTGVPARFSGSGSGTEFT |
| | LTISSLQSEDFAVYFCQQDYSSPYTFGGGTKVEIK (SEQ ID NO: 476) |
| VL | SIVMTQSPKILLVSAGDRVTITCKASQSVSNDVAWYQQKPGQSPKLLIYYASNRYTGVPDRFTGSGYGTDFT |
| | FTISTVQAEDLAVYFCQQDYSSPYTFGGGTKLEIK (SEQ ID NO: 477) |
| VL | EIVLTQSPALMAASPGEKVTITCSVSSSINSINLHWYRQKSETSPKPWIYGTSNLASGVPVRFSGSGSGTSY |
| | SLTISSMEAEDAATYYCQQWSSYPLTFGAGTKLELK (SEQ ID NO: 478) |

Group E

| | |
|---|---|
| VH | VQLVQSGAEVKKPGASVKVSCKASGYTFTGYYMHWVRQAPGQGLEWMGWINPNSGGTNYAQKFQGRVTMTRD |
| | TSASTAYMELSGLRSEDTAVYYCARGNLFIDYWGQGTLVTVSS (SEQ ID NO: 479) |
| VH | EVQLVESGAEVKKPGASVRVSCRASGYTFTNYAISWVRQAPGQGLEWMGWISTYNGNTLYAQKLQGRVTMTT |
| | DTSTSTAYMELRSLRSDDTAVYYCARDTDTYYFDYWGQGTLVTVSS (SEQ ID NO: 480) |
| VH | EVQLLESGAEVKKPGASVKVSCKASGYTFTSHYMHWVRQAPGQGLEWMGVINPSGGSTSYAQKFQGRVTMTR |
| | DTSTSTVYMDLSSLRSEDTAVYYCARRSEAYYHGMDVWGQGTTVTVSS (SEQ ID NO: 481) |
| VH | EVQLVESGAEVKKPGASVKVSCKASGYTFTGYYMHWVRQAPGQGLEWMGWINPNSGGTNYAQKFQGRVTMTR |
| | DTSISTAYMELSSLRSDDTAVYYCARGATGGYGMDVWGQGTTVTVSS (SEQ ID NO: 482) |
| VH | QVQLVQSGAEVKKPGASVKVSCKASGYGFTSYAIHWLRQAPGQRLEWMGWINPGNGNTKYSQKFQGRVTITR |
| | DTSATTAYMELTSLRSEDSDTAVYYCARDLDGGSFDHWGQGTLVTVSS (SEQ ID NO: 483) |
| VH | QVQLVQSGAEVKKPGASVKISCKASGYTFTTYWIHWVRQAPGQGPEWMGLIKPSSGSTTYPQKFQGRVTMTR |
| | DTSTSTVYMELSSLRSEDTAVYYCARLEGIGAASNDWGQGTLVTVSS (SEQ ID NO: 484) |
| VH | QVQLVQSGSELKKPGASVKVSCKASGYSFRSYDINWVRQAPGQGLEWMGFLNPSDGGTTYAQKFQGRVTVTS |
| | DTSTSTVYMELSSLRSENTAVYYCARVGITSTETRAEYFQHWGQGTLVTVSS (SEQ ID NO: 485) |
| VH | QVQLVQSGAEVKKPGASWLSCKASGYTFTRYWVHWVRQAPGQGPEWMGLIKPRDGATTYAQKFQGRVTLTR |
| | DTSTTTVYMELTSLRSEDTGIYYCGLLEGDDAFDVWGQGTMVTVSS (SEQ ID NO: 486) |
| VH | QVQLVESGAEVKKPGASVKVSCKASGYTFTSYYMHWVRQAPGQGLEWMGIINPSGGSTSYAQKFQGRVTMTR |
| | DTSTSTVYMELSSLRSEDTAVYYCARESYGSGSLDYWGQGTLVTVSS (SEQ ID NO: 487) |
| VH | EVQLLESGAEVKKPGASVKVSCKASGYTFTGYYMHWVRQAPGQGLEWMGWINPNSGGTNYAQKFQGRVTMTR |
| | DTSISTAYMELSRLRSDDTAVYYCARPKGHSGGWYAFDIWGQGTMVTVSS (SEQ ID NO: 488) |
| VH | QVQLQQSGPGLVKPAQTLSLTCAISGDSVSSSRAAWNWIRQSPSRGLEWLGRTFYRSRWNNEYAETVKSRIT |
| | INPDTSTNHFSLQLTSVSPEDTAIYYCARGGGNFDSWGQGTLVTVSS (SEQ ID NO: 489) |
| VH | QVQLVQSGAEVKKPGASVKVSCKASGYTFTSYYMHWVRQAPGQGLEWMGIINPSGGSTSYAQKFQGRVTMTR |
| | DTSISTVYMELSSLRSEDTAVYYCARESEDSIAFDIWGQGTMVTVSS (SEQ ID NO: 490) |
| VH | QVQLVQSGAEVRKPGASVMVSCKASGYPFTSYAIHWLRQAPGQSLEWMGWIKPANGDITYSQKFQGRVTITG |
| | DISATTAYMELSSLRSEDTAMYYCTKGGGGYFDYWGQGTLVTVSS (SEQ ID NO: 491) |
| VH | QVQLVQSGAEVKKPGESLRISCQGSGYSFINHWISWVRQMPGKGLEWLGRIDPSDSYTNYSPSVQGHVTISV |
| | DKSISTAYLQWSSLKASDTAIYYCARHDRNVYFDPWGQGTLVTVSS (SEQ ID NO: 492) |
| VH | QVQLVQSGAEVRKPGASVMVSCKASGYPFTSYAIHWLRQAPGQSLEWMGWIKPANGDITYSQKFQGRVTITG |
| | DISATTAYMELSSLRSEDTAMYYCAKGGGGYFDYWGQGTLVTVSS (SEQ ID NO: 493) |
| VH | EVQLVQSGAEVKKPGASVKVSCKASGYTFTGYYMHWVRQAPGQGLEWMGWINPNSGGTNYAQKFQGWVTMTR |
| | DTSISTAYMELSRLRSDDTAVYYCARDQDFDYWGQGTLVTVSS (SEQ ID NO: 494) |
| VH | QVQLVESGGGWQPGRSLRLSCAASGFTFSTYAMHWVRQAPGRGLEWVAGISYDGSNKYHADPVKGRFTISR |
| | DNSKNTLYLQMNNLRVEDSAVYYCAGDRSGGLDVWGQGTTVTVSS (SEQ ID NO: 495) |
| VH | EVQLVESGAEVKKPGASVKVSCKASGYTFTGYYMHWVRQAPGQGLEWMGWINPNSGGTNYAQKFQGRVTMTR |
| | DTSISTAYMELRSLRSDDTAVYYCARGGGAFDIWGQGTMVTVSS (SEQ ID NO: 496) |
| VH | QVQLVQSGAEVKKPGASVKVSCKASGYTFTGYYMHWVRQAPGQGLEWMGWINPNSGGTNYAQKFQGWVTMTR |
| | DTSISTAYMELSRLRSDDTAVYYCARDQDFDYWGQGTLVTVSS (SEQ ID NO: 497) |
| VH | QMQLVQSGAEVKKPGASVKVSCKASGYTFTGYYIHWVRQAPGQGLEWMGWINPNSGGTIYAQKFQGRVTMTR |
| | DTSISTAYMELSRLRSDDTAVYYCARGSTNFDSWGQGTLVTVSS (SEQ ID NO: 498) |
| VH | QVQLVESGAEVKKPGASVKVSCKASGYTFTSHYMHWVRQAPGQGLEWMGVINPSGGSTSYAQKFQGRVTMTR |
| | DTSTSTVYMDLSSLRSEDTAVYYCARRSEAYYHGMDVWGQGTTVTVSS (SEQ ID NO: 499) |
| VH | EVQLLESGAEVKKPGSSVKVSCKASGYPFTSYAIHWLRQAPGQSLEWMGWIKPANGDITYSQKFQGRVTITG |
| | DISATTAYMELSSLRSEDTAMYYCAKGGGGYFDYWGQGTLVTVSS (SEQ ID NO: 500) |
| VH | QVQLVQSGGGVVQPGRSLRLSCAASGFTFSSYAMHWVRQAPGKGLEWVAVISYDGTNKYYADSVKGRFTISR |
| | DSSKNALYLQMNSLRTEDTALYYCARGGGWWHAMDWGQGTTVTVSS (SEQ ID NO: 501) |
| VL | LPVLTQPASVSGSPGQSITISCTGTSSDVGSYNLVSWYQQHPGKAPKLMIYDVSKRPSGVSNRFSGSKSGNT |
| | ASLTISGLQAEDEADYYCSSYTSSSTFVFGGGTKLTVL (SEQ ID NO: 502) |
| VL | QPVLTQPASVSGSPGQSITISCTGTSSDVGGYNYVSWYQQHPDKPPKLIIYYVSNRPSGVSNRFSGSKSGNT |
| | ASLTISGLQAEDEADYYCASYRSNTNYVFGTGTKVTVL (SEQ ID NO: 503) |
| VL | QSVLTQPPSASGTPGQRVTISCSGSSSNIGSNYVYWYQQFPGTTPKLLIYRNNQRPSGVPDRFSGSKSATSA |
| | SLAISGLRSEDEADYYCAAWDDSLSGWVFGGGTKLTVL (SEQ ID NO: 504) |
| VL | QAGLTQPASVSGSPGQSITISCTGTSSDVGGYNYVSWYQQHPGKAPKLMIYNVTKRPSGVSNRFSGSKSGNT |
| | ASLTISGLQAEDEADYYCSSYTSGTTLSVFGTGTKLTVL (SEQ ID NO: 505) |
| VL | QSALTQPRSVSGSPGQSVTISCTGTSNDVGAYNYVSWYQQHPAKAPKLMIYGVTKRPSGVPDRFSGANSGNT |
| | ATLTITRVEAGDEADYFCQWERSSGQYVFGTGTKLTVL (SEQ ID NO: 506) |
| VL | QSWTQPASVSGSPGQSITISCTGTSSDVGSYNLVSWYQQHPGKAPKLMIYDVTNRPSGVSNRFSGSKSGNT |
| | ASLTISGLQAEDEADYYCSSYTRSSTYVFGTGTKVTVL (SEQ ID NO: 507) |
| VL | QAVVTQEPSLTVSPGGTVTLTCGSSTGAVTSGHYPYWFQQKPGQAPRTLIYDTSNKHSWTPARFSGSLLGGK |
| | AALTLSGAQPEDEAEYYCLLSYSGARVFGTGTKVTVL (SEQ ID NO: 508) |
| VL | QTVVTQEPSLTVSPGGTVTLTCGSSTGDVTSGHYPYWFQQKPGQAPRTLIYDTSNKHSWTPARFSASLLGGK |
| | AALTLSGAQPEDEADYYCLLAYSEVRVFGGGTQLTVL (SEQ ID NO: 509) |
| VL | QSVLTQPPSASGSPGQSVTISCTGSASDIGHSFYVSWYRQYPGKAPDLLIFQVNQRPSGVPNRFSASKSGNT |
| | ASLTVSGLQIEDEADYYCSSYAGGTSIVFGSGTKLTVL (SEQ ID NO: 510) |
| VL | QTVVTQEPSLTVSPGGTVTLTCGSSTGAVTSGHYPYWFQQKPGQPPRTLIYDTSNKHSWTPARFSGSLLGGK |
| | AALTLSGAQPEDEAEYYCLLSYSGARVFGGGTKLTVL (SEQ ID NO: 511) |
| VL | ETTLTQSPATLSVSPGERATLSCRASQSVSSYLAWYQQKPGQAPRLLIYDASNRATGIPARFSGSGSGTDFT |
| | LTISSLEPEDFAVYYCQQRSNVIPQITFGQGTRLEIK (SEQ ID NO: 512) |
| VL | QTVVTQEPSLTVSPGGTVTLTCGSDTGAWSGHYPYWFQQKPGQAPRALIYDTGNKHSWTPARFSGSLLGGK |
| | AALTLSGAQPEDEAEYYCLLSYSGTRIFGGGTKLTVL (SEQ ID NO: 513) |
| VL | QSVLTQPPSASGTPGQRVTISCSGSSSNIGRRAVNWYQQLPGTAPKLLIYDNDRRPSGIPDRFSGSKSGTSA |
| | TLGITELQTGDEADYYCGTWDTNLSAGLFGGGTKLTVL (SEQ ID NO: 514) |
| VL | SGSSSNIGNNYVSWYQQLPGTAPKLLIYDNNKRPSGIPDRFSGSKSGTSATLGITGLQTGDEADYYCGTWDS |
| | SLSAGDWFGGGTKLTVL (SEQ ID NO: 515) |
| VL | AIQLTQSPGTLSLAPGERATLSCRASQSVSSSYIAWYQQRPGQAPRLLIYGASNRATDIPARFIGSGSGTDF |
| | TLTISSLEPEDFAVYYCQQRSNWPRNTFGQGTRLEIK (SEQ ID NO: 516) |
| VL | QTVVTQEPSLTVSPGGTVTLTCGSNTGAVTSGHYPYWFQQKPGQAPRTLIYDATNKQSWTPARFSGSLLGDK |
| | AALTLSGAQPEDEAEYYCLLSYSGVRVFGGGTKLTVL (SEQ ID NO: 517) |
| VL | QTVVTQEPSLTVSPGGTVTLTCGSSTGAVTSGHYPYWFQQKPGQAPRTLIYDTSNKHSWTPARFSGSLLGGK |
| | AALTLSGAQPEDEADYFCLLSSSGARVFGGGTKLTVL (SEQ ID NO: 518) |
| VL | QSVLTQPRSVSGSPGQSVTISCTGTSSDVGGYNLVSWYQQHPGKAPKLMIYDVHKRPSGTSTRFSGSKSGNT |
| | ASLTISGLQAEDEADYYCSSYRSGSTYVFGTGTKVTVL (SEQ ID NO: 519) |
| VL | SYELTQPRSVSGSPGQSVTISCTGTSSDVGGYKYVSWYQQHPGKAPKLMIYDVSKRPSGVSNRFSGSKSGNT |
| | ASLTISGLQAEDEADYYCSSYTSSSTYVFGTGTKVTVL (SEQ ID NO: 520) |
| VL | SYVLTQPASVSGSPGQSITISCTGTSSDVGNYNLVSWYQQHPGKAPKLLVYDVSNRPSGVSNRFSGSKSGNT |
| | ASLTISGLQAEDEADYYCSSYTTSSTYVFGIGTKVTVL (SEQ ID NO: 521) |
| VL | QSVLTQPASMSGSPGQSITISCTGTSSDVGTYDLVSWYQQYPGKAPKLLIYDVANRPSGVSNRFSGSKSGNT |
| | ASLTVSGLQAEDEADYYCSSYAGTKVYVFGTGTKVTVL (SEQ ID NO: 522) |
| VL | DVVMTQSPSSLSASVGDRVTITCFASQGIATNLAWFQQKPGKAPKSLIYAASSLQSGVPSKFSGSGSGTAFT |
| | LTISSLQAEDFGTYYCQQYNNYPYTFGQGTKVEIK (SEQ ID NO: 523) |
| VL | DIVMTQSPSSLSASVGDRVTITCPASQGISNSLAWYQQKSGKAPKLLLYAASGLESGVPSRFSGSGSGTDYT |
| | LTISSLQPBDFATYYCQQSYSMPLTFGGGTKVEIK (SEQ ID NO: 524) |
| VL | QPVLTQPASVSGSPGQSITISCTGTSSDVGGYNLVAWYQQHPGKAPKLMIYDVSKRPSGVSNRFSGSKSGNT |
| | ASLTISGLQAEDEADYYCSSYTSSSTSHYVFGTGTKVTVL (SEQ ID NO: 525) |
| VL | QAVLTQPASVSGSPGQSITISCTGTSSDVGSYNLVSWYQQHPGKAPKLMIYEVRKRPSGVSNRFSASKSGNT |
| | ASLTISGLQAEDEADYYCSSYTSSSTFVFFGAGTKLTVL (SEQ ID NO: 526) |
| VL | SYVLTQPASVSGSPGQSITISCTGTSSDVGGTNYVSWYQQHPGKAPKLMIFDVSNRPSGVSNRFSGSKSGNT |
| | ASLTISGLQAEDEADYYCSSYTSMRTLVFGGGTKLTVL (SEQ ID NO: 527) |
| VL | QAGLTQPPSASGSPGQSVTISCTGTSSDVGGYNSVSWYQQHPGKAPKLMIYDVSNRPSGVSNRFSGSKSGNT |
| | ASLTISGLQAEDEADYYCSSYINSGTLVFGGGTKLTVL (SEQ ID NO: 528) |
| VL | QSVLTQPASVSGSPGQSITISCTGTSADVGHYNLVSWYQQHPGKAPKLMIYDVTKRPSGVSTRFSGSKSGNT |
| | ASLTISGLQAEDESDYYCSSYTSSSTYVFGTGTKLTVL (SEQ ID NO: 529) |
| VL | EIVMTQSPSSLSASVGDRWITCRASRNIKTALAWFQQRPGQAPKSLIYAASSLHSGVTSRFSGSGFGTDFT |
| | LTINSLQPEDVATYYCQQYDSYPITFGQGTRLEIK (SEQ ID NO: 530) |
| VL | QAGLTQPASVSGSPGQWITISCTGTSSDVGAYNLVSWYQQYPGKAPKLMIYDVTKRPSGVSDRFSGSKSGNT |
| | ASLTISGLQAEDEADYYCSSYTSSTTYVFGTGTQLTVL (SEQ ID NO: 531) |
| VL | QPVLTQPRSVSGSPGQSVTISCTGTSSDVGGYNLVSWYQQHPGRAPKLMIYDVSDRPSGVSDRFSGSKSGNT |
| | ASLTISGLQAEDEADYYCSSFTSRTTPAYVFGTGTKLTVL (SEQ ID NO: 532) |
| VL | QSVVTQPPSVSAAPGQKVTISCSGSSSNIGNNYVSWYQQLPGTAPKLLIYDNNKRPSGIPDRFSGSKSGNTA |
| | SLTISGLQAEDEADYYCCSYAGSNTLIFGGGTKVTVL (SEQ ID NO: 533) |
| VL | QSVLTQPASVSGSPGQSITISCTGTSSDVGSYNLVSWYQQHPGKAPKLMIYDVSERPSGVPDRFSGSKSGNT |
| | ASLTISGLQAEDEADYYCSSYTSSSTLYVFGTGTKLTVL (SEQ ID NO: 534) |

Group F

| | |
|---|---|
| VH | MGWSCILFLVATATGSIVMTQTPTFLVVSAGDRVTITCPCASQSVINDVAWYQQKPGQSPKLLIFYASNRNTG |
| | VPDRFRGSGYGTDFRFTISTVQAEDLAVYFCQQDYSPPRTRGSGTK (SEQ ID NO: 535) |
| VH | MGWSCIILFLVATATGEVKLEESGGGLVQPGGSMKLSCVASGFTFSDAWMDWVRQSPEKGLEWVAEIRSKAN |
| | NHAPYYTESVKGRFTISRDDSKSIIYLQMNNLRAEDTGIYYCTRDSTATHWGQGT (SEQ ID NO: 535) |
| VL | MGWSCIILFLVATATGSIVMTQTPTFLWSAGDRVTITCKASQSVINDVAWYQQKPGQSPKLLIFYASNRNT |
| | GVPDRFTGSGYGTDFTFTISTVQAEDLAVYFCQQDYSPPFTFGSGTKLEIKRTVAAPSVFIFPPSDEQLKSG |
| | TASWCLLNNFYPREAKVQWKVDNALQSGNSQESVTEQDSKDSTYSLLSSTLTLSKADYEKHKWACEVTHQ |
| | GLSSPVTKSFNRGEC (SEQ ID NO: 536) |
| * | MGWSCIILFLVATATGEVKLEESGGGLVQPGGSMKLSCVASGFTFSDAWMDWVRQSPEKGLEWAEIRSKAN |
| HC | NHAPYYTESVKGRFTISRDDSKSIIYLQMNNLRAEDTFIYYCTRDSTATHWGQGTLVTVSAASTKGPSVFPL |
| | APSSKSTSGGTAALGCLVKDYFPEPVTVSWNSGALTSGVHTFPAVLQSSGLYSLSSVVTVPSSSLGTQTYIC |
| | NVNHKPSNTKVDKRVEPKSCDKTHTCPPCPAPELLGGPSVTXFPPKPKDTLMLSRTPEVTCVVVDVSHEDPE |
| | VKFNWYVDGVEVHNAKTKPREEQYNSTYRWSVLTVLTVLHQDWLNGKEYKCKVSNKALPAPIEKTISKAKG |
| | QPREPQVYTLPPSRDELTKNQVSLTCLVKGFYPSDIAVEWESNGQPENNYKTTPPVLDSDGSFFLYSKLTVD |
| | KSRWQQGNVFSCSVMHEALHNHYTQKSLSLSPGK (SEQ ID NO: 537) |

Group H

| | |
|---|---|
| VH | EVQLVESGGGLVQPGGSLRLSCAASGFTFSNFWMNWVRQAPGKGLEWVSEIRLKSNNYATHYAESVKGRFTI |
| | SRDDSKNTLYLQMNSLKTEDTAVYYCTSYDYEYWGQGTLVTVSA (SEQ ID NO: 538) |
| VH | EVQLVESGGGLVQPGGSLKLSCAASGFTFSNFWMNWVRQAPGKGLEWVAEIRLKSNNYATHYAESVKGRFTI |
| | SRDDSKNTLYLQMNSLRTEDTAVYYCTSYDYEYWGQGTLVTVSA (SEQ ID NO: 539) |
| VH | EVQLVESGGGLVQPGGSLKLSCAASGFTFSNFWMNWVRQAPGKGLEWVAEIRLKSNNYATHYAESVKGRFTI |
| | SRDDSKNTLYLQMNSLKTEDTAVYYCTSYDYEYWGQGTLVTVSA (SEQ ID NO: 539) |
| VL | DIQMTQSPSTLSASVGDRVTLSCKASENVGTYVSWYQQKPGKAPKLLIYGASNRYTGVPSRFTGSGSGTDFT |
| | LTISSLQPDDFATYYCGQSYSYPFTFGSGTKLEIK (SEQ ID NO: 540) |
| VL | DIQMTQSPASLSASVGDRVTISCKASENVGTYVSWYQQKPGQTPKLLIYGASNRYTGVPSRFSGSGSGTDFT |
| | LTISSLQPDDFATYYCGQSYSYPFTFGSGTKLEIK (SEQ ID NO: 541) |
| VL | DIQMTQSPSSLSASVGDRVTLTCKASENVGTYVSWYQQKPGQAPKLLIYGASNRYTGVPSRFTGSGSGTDFT |
| | LTISSLQPDDFATYYCGQSYSYPFTFGSGTKLEIK (SEQ ID NO: 542) |

Group I

| | |
|---|---|
| VH | EVQLVESGGGLVQPGGSLRLSCAASGFTFSDAWMDWVRQAPGKGLEWVGEIRSKANNHAPYYTESVKGRFTI |
| | SRDDSKMSLYLQMNSLKTEDTAVTYCARDSTATHWGRGTLVTVSS (SEQ ID NO: 543) |
| VL | AIQMTQSPSSLSASVGDRVTITCKASQSVINDVAWYQQKPGKAPKLLIYYASNRNTGVPSRFSGSGSGTDFT |
| | LTISSLQPEDFATYYCQQDYSPPFTFGPGTKVDIKR (SEQ ID NO: 544) |
| VL | EIVLTQSPATLSLSPGERATLSCKASQSVINDVAWYQQKPGQAPRLLIYYASNRNTGIPARFSGSGSGTDFT |
| | LTISSLEPEDFAVYYCQQDYSPPFTFGPGTKVDIKR (SEQ ID NO: 545) |

Group J

| | |
|---|---|
| VH | DIQMTQSPXXLSXSVGDRVTXXCKASENVGTYVSWYQQKPGXXPKLLIYGASNRYTGVPXRFTGXGSGTDFT |
| | LTISSLQXXDXATYYCGQSYSYPFTFGSGTKLEIK (SEQ ID NO: 580) |
| VL | EVQLXESGGGLVQPGGSLRLSCXASGFTFSNFWMNWVRQAPGKGLEWVXEIRLKSNNYATHYAESVKGRFTI |
| | SRDDSKXXLYLQMNSLXTEDTXVYYCTSYDYEYWGQGTLVTVSA (SEQ ID NO: 581) |

Group K

| | |
|---|---|
| VL | QVQLQESGGGSVQAGGSLRLSCAASGYTFSSSNCMGWFRQAPGKEREDVAIISVRGGITYYADSVKGRFTIS |
| | RDSAKNTLSLQMNSLKPEDTAVYYCAADNPKYRALSDAHCNWSYRYWGQGTQVTVS (SEQ ID |
| | NO: 582) |

Group L

| | |
|---|---|
| VH | QVQLVQSGAEVKEPGASVKVSCKASGYTFTSYGISWVRQAPGQGLEWMGWINPNSGGTNYAQKFQGRVTMTR |
| | DMSISTAYMELSRLRSDDTAVYYCARGRGSYYAFDIWGQGTMVTVSS (SEQ ID NO: 609) |
| VH | EVQLVESGGDLVKPGGSLKLSCAASGFTFSSYGMSWVRQTPDKRLEWVATISSGGSYTYYQDSIKGRFTISR |
| | DNAKNTLYLQMSSLKSEDTAMYYCSIGDWADYWGQGTTLTVSS (SEQ ID NO: 610) |
| VH | QVQLQQSGAELMKPGASVKISCKTSGYTFSTYWIEWVKQRPGHGPEWIGEFLPGSGSTNFNEKFKGPVATFTA |
| | DKSSDTAYMQLSSLTSEDSAVYYCTRSGGNFGARFASWGQGTLVTVSA (SEQ ID NO: 611) |
| LC* | DSVLTQPPSVSGAPRQTVTISCSGSSSNIGQNSVTWYQRLPGEAPKLLIYYDDLLHSGVSDRFSGSKSGTSA |
| | SLAISGLQSEDEAEYYCASWDDSLKGPVFGGGTKLTVLGQPKAAPSVTLFPPSSEELQANKATLVCLISDFY |
| | PGAVTVAWKADSSPVKAGVETTTPSKQSNNKYAASSYLSLTPEQWKSHKSYSCQVTHEGSTVEKTVAPTECS |
| | (SEQ ID NO: 612) |
| VL | DIQMTQSPDSLAVSLGERATINCKSSQSVLYSSNNKNYLAWYQQKPGQPPKLLIYWASTRESGVPDRFSGSG |
| | SGTDFTLTISSLQAEDVAVYYCQQYYSTPTFGQGTKVEIK (SEQ ID NO: 613) |
| VL | DIKMTQSPSSMYASLGERVTITCKASQDINSYLSWFQQKPGKSPKTLIHRANRLVAGVPSRFSGSGSGQVYS |
| | LTISSLEYEDMGIYFCLQYDEFPLTFGAGTKLELN (SEQ ID NO: 614) |
| VL | DIVMTQAAPSVPVTPGESVSISCRSSKSLLSNNGNTYLYWFLQRPGQSPHLLIYRMSSIASGVPDRFSGSGS |
| | GTAFTLRISRVEAEDVGVYYCMQHLEYPFTFGGGTKLEIK (SEQ ID NO: 615) |

*the indicated sequence provided in Group G designated as “HC” is a heavy chain amino acid sequence, and the indicated sequence provided in Group L designated as “LC” is a light chain amino acid sequence; all other sequences presented in Table 1 are variable heavy chain and variable light chain sequences.

In some embodiments, the CD147 antibody/activatable CD147 antibody includes a CDR sequence shown in Table 2, a combination of VL CDR sequences (VL CDR1, VL CDR2, VL CDR3) selected from the group consisting of those combinations shown in a single row Table 2, a combination of VH CDR sequences (VH CDR1, VH CDR2, VH CDR3) selected from the group consisting of those combinations shown in Table 2, or a combination of VL CDR and VH CDR sequences (VL CDR1, VL CDR2, VL CDR3, VH CDR1, VH CDR2, VH CDR3) selected the group consisting of those combinations shown in Table 2.

In some embodiments, the CD147 antibody/activatable CD147 antibody includes a combination of heavy chain CDR sequences selected from the group consisting of the combinations shown in Group A in Table 2. In some embodiments, the CD147 antibody/activatable CD147 antibody includes a combination of light chain CDR sequences selected from the group consisting of the combinations shown in Group A in Table 2. In some embodiments, the CD147 antibody/activatable CD147 antibody includes a combination of heavy chain CDR sequences selected from the group consisting of the combinations shown in Group A in Table 2, and a combination of light chain CDR sequences selected from the group consisting of the combinations shown in Group A in Table 2.

In some embodiments, the CD147 antibody/activatable CD147 antibody includes a combination of heavy chain CDR sequences selected from the group consisting of the combinations shown in Group B in Table 2. In some embodiments, the CD147 antibody/activatable CD147 antibody includes a combination of light chain CDR sequences selected from the group consisting of the combinations shown in Group B in Table 2. In some embodiments, the CD147 antibody/activatable CD147 antibody includes a combination of heavy chain CDR sequences selected from the group consisting of the combinations shown in Group B in Table 2, and a combination of light chain CDR sequences selected from the group consisting of the combinations shown in Group B in Table 2.

In some embodiments, the CD147 antibody/activatable CD147 antibody includes a combination of heavy chain CDR sequences selected from the group consisting of the combinations shown in Group C in Table 2. In some embodiments, the CD147 antibody/activatable CD147 antibody includes a combination of light chain CDR sequences selected from the group consisting of the combinations shown in Group C in Table 2. In some embodiments, the CD147 antibody/activatable CD147 antibody includes a combination of heavy chain CDR sequences selected from the group consisting of the combinations shown in Group C in Table 2, and a combination of light chain CDR sequences selected from the group consisting of the combinations shown in Group C in Table 2.

In some embodiments, the CD147 antibody/activatable CD147 antibody includes a combination of heavy chain CDR sequences selected from the Group E consisting of the combinations shown in Group E in Table 2. In some embodiments, the CD147 antibody/activatable CD147 antibody includes a combination of light chain CDR sequences selected from the Group E consisting of the combinations shown in Group E in Table 2. In some embodiments, the CD147 antibody/activatable CD147 antibody includes a combination of heavy chain CDR sequences selected from the Group E consisting of the combinations shown in Group E in Table 2, and a combination of light chain CDR sequences selected from the Group E consisting of the combinations shown in Group E in Table 2.

TABLE 2
CDR Sequences for Antibodies and Activatable Antibodies
that Bind CD147

| VH | | | VL | | |
|---|---|---|---|---|---|
| CDR1 | | CDR3 | | CDR2 | CDR3 |
| (SEQ ID | CDR2 | (SEQ ID | CDR1 | (SEQ ID | (SEQ ID |
| NO:) | (SEQ ID NO:) | NO:) | (SEQ ID NO:) | NO:) | NO:) |

Group A

| | | | | | |
|---|---|---|---|---|---|
| NFWMD | (G/E)IRLKS(Y/T)NY | (W/T)(D/S) | KASQ(D/S)VS(T/ | (S/Y)AS | QQ(H/D)YS |
| (546) | ATHYAESVKG (547) | (G/T)(A/G)Y | N)DVA (549) | (Y/N)RY | (TS)P(F/Y)T |
| | | (548) | | T (550) | (551) |
| NFWMD | GIRLKSYNYATHYAESV | WDGAY (553) | KASQDVSTDVA | SASYRYT | QQHYSTPFT |
| (546) | KG (552) | | (554) | (555) | (556) |
| NFWMD | EIRLKSTNIATHYAESV | TSTGY (558) | KASQSVSNDVA | YASNRYT | QQDYSSPYT |
| (546) | KG (557) | | (559) | (560) | (561) |
| DYWMN | IIDPSDSYASYNQKFKG | KSYYGGNYYYA | SVSSSINSINLH | GTSNLAS | QQWSSYPLT |
| (562) | (563) | MDY (564) | (565) | (566) | (567) |

Group B

| | | | | | |
|---|---|---|---|---|---|
| GFTFSNFW | EIRLKSNNYATHYAES | YDYEY (570) | KASENVGTYVS | GASNRYT | GQSYSYPFT |
| MN (568) | VKG (569) | | (571) | (572) | (573) |

Group C

| | | | | | |
|---|---|---|---|---|---|
| DAWMD | EIRSKANNHAPYYTES | DSTATH | KASQSVINDVA | YASNRNT | QQDYSPPFT |
| (574) | VKG (575) | (576) | (577) | (578) | (579) |

Group D

| | | |
|---|---|---|
| GYTFSSSNCM | SVRGGIT | AADNPKRALS |
| (583) | (584) | DAHCNWSYRY |
| | | (585) |

Group E

| | | | | | |
|---|---|---|---|---|---|
| SYGIS | WINPNSGGTNYAQKFQ | GRGSYYAFDI | KSSQSVLYSSNNKN | WASTRES | QQYYSTPT |
| (586) | G (587) | (588) | YLA (589) | (590) | (591) |
| SGGYYWS | YIYYSGSTYYNPSLKS | DRGTGDAFDI | SGSSSNIGQNSVT | YDDLLHS | ASWDDSLKGP |
| (592) | (593) | (593) | (594) | (595) | V (596) |
| SYGMS | TISSGGSYTYYQDSIK | GDWADY | KASQDINSYLS | RANRLVA | LQYDEFPLT |
| (597) | G (598) | (599) | (600) | (601) | (602) |
| TYWIE | EFLPGSGSTNFNEKFK | SGGNFGARFAS | RSSKSLLSNNGNTY | RMSSLAS | MQHLEYPFT |
| (603) | G (604) | (605) | LY (606) | (607) | (608) |

In some embodiments, the CD147 antibody/activatable CD147 antibody comprises or is derived from an antibody that is manufactured, secreted or otherwise produced by a hybridoma, such as, for example, the hybridoma(s) disclosed in U.S. Pat. No. 5,330,896 and deposited at ATCC under deposit number HB 8214.

In some embodiments, the CD147 antibody/activatable CD147 antibody comprises or is derived from an antibody that is manufactured, secreted or otherwise produced by a hybridoma, such as, for example, the hybridoma(s) designated BA120 as disclosed in U.S. Pat. No. 7,736,647 and deposited at the Collection Nationale de Cultures de Microorganismes (CNCM) (Institut Pasteur, Paris, France, 25, Rue du Docteur Roux, F-75724, Paris, Cedex 15) on Jun. 14, 2005, under number CNCM I-3449; the hybridoma(s) disclosed in U.S. Pat. No. 7,572,895 and deposited at the ATCC under PTA-6055; the hybridoma(s) disclosed in PCT Publication No. WO 2014/020140 and WO 2005/111082 and deposited with CNCM on May 10 2001, under number 1-2665; the hybridoma(s) disclosed in U.S. Pat. No. 4,434,156 and deposited at the ATCC under HB-8094; the hybridoma(s) disclosed in U.S. Pat. No. 5,648,469 and deposited at the ATCC under HB-11011 and HB-11010.

In some embodiments, the CD147 antibody/activatable CD147 antibody includes a heavy chain that comprises or is derived from a heavy chain amino acid sequence shown in PCT Publication Nos. WO 2014/144060, WO 2014/189973, WO 2014/020140, in U.S. Pat. Nos. 8,663,598; 8,129,503; 7,736,647; 7,572,895; 4,434,156; in US Patent Application Publication Nos. US2014114054, US20140212423, US2013177579, US2013045206, US20130216476, US20120282176, and/or in Chinese Patent No. CN101245107B, the contents of each of which are hereby incorporated by reference in their entirety.

The disclosure also provides methods for producing a CD147 AB of the disclosure by culturing a cell under conditions that lead to expression of the antibody or fragment thereof, wherein the cell comprises a nucleic acid molecule of the disclosure or a vector of the disclosure.

In some embodiments, the CD147 antibody or antigen-binding fragment thereof is encoded by a nucleic acid sequence that comprises a nucleic acid sequence encoding a heavy chain amino acid sequence comprising an amino acid sequence selected from the group consisting of SEQ ID NOs: 1-4. In some embodiments, the antibody or antigen-binding fragment thereof is encoded by a nucleic acid sequence that comprises a nucleic acid sequence encoding a heavy chain amino acid sequence comprising an amino acid sequence selected from the group consisting of SEQ ID NOs: 1-3.

In some embodiments, the CD147 antibody or antigen-binding fragment thereof is encoded by a nucleic acid sequence that comprises a nucleic acid sequence encoding a light chain amino acid sequence comprising an amino acid sequence selected from the group consisting of SEQ ID NOs: 5-9. In some embodiments, the antibody or antigen-binding fragment thereof is encoded by a nucleic acid sequence that comprises a nucleic acid sequence encoding a light chain amino acid sequence comprising an amino acid sequence selected from the group consisting of SEQ ID NOs: 5-8.

In some embodiments, the CD147 antibody or antigen-binding fragment thereof is encoded by a nucleic acid sequence that comprises a nucleic acid sequence encoding a heavy chain amino acid sequence comprising an amino acid sequence selected from the group consisting of SEQ ID NOs: 1-4, and a nucleic acid sequence encoding a light chain amino acid sequence comprising an amino acid sequence selected from the group consisting of SEQ ID NOs: 5-9.

In some embodiments, the CD147 antibody or antigen-binding fragment thereof is encoded by a nucleic acid sequence that comprises a nucleic acid sequence encoding a heavy chain amino acid sequence comprising an amino acid sequence selected from the group consisting of SEQ ID NOs: 1-3, and a nucleic acid sequence encoding a light chain amino acid sequence comprising an amino acid sequence selected from the group consisting of SEQ ID NOs: 5-8.

In some embodiments, the CD147 antibody or antigen-binding fragment thereof is encoded by a nucleic acid sequence that comprises a nucleic acid sequence that is at least 90%, 91%, 92%, 93%, 94%, 95%, 96%, 97%, 98% or 99% identical to a nucleic acid sequence encoding a heavy chain amino acid sequence comprising an amino acid sequence selected from the group consisting of SEQ ID NOs: 1-4. In some embodiments, the antibody or antigen-binding fragment thereof is encoded by a nucleic acid sequence that comprises a nucleic acid sequence that is at least 90%, 91%, 92%, 93%, 94%, 95%, 96%, 97%, 98% or 99% identical to a nucleic acid sequence encoding a heavy chain amino acid sequence comprising an amino acid sequence selected from the group consisting of SEQ ID NOs: 1-3.

In some embodiments, the CD147 antibody or antigen-binding fragment thereof is encoded by a nucleic acid sequence that comprises a nucleic acid sequence that is at least 90%, 91%, 92%, 93%, 94%, 95%, 96%, 97%, 98% or 99% identical to a nucleic acid sequence encoding a light chain amino acid sequence comprising an amino acid sequence selected from the group consisting of SEQ ID NOs: 5-9. In some embodiments, the antibody or antigen-binding fragment thereof is encoded by a nucleic acid sequence that comprises a nucleic acid sequence that is at least 90%, 91%, 92%, 93%, 94%, 95%, 96%, 97%, 98% or 99% identical to a nucleic acid sequence encoding a light chain amino acid sequence comprising an amino acid sequence selected from the group consisting of SEQ ID NOs: 5-8.

In some embodiments, the CD147 antibody or antigen-binding fragment thereof is encoded by a nucleic acid sequence that comprises a nucleic acid sequence that is at least 90%, 91%, 92%, 93%, 94%, 95%, 96%, 97%, 98% or 99% identical to a nucleic acid sequence encoding a heavy chain amino acid sequence comprising an amino acid sequence selected from the group consisting of SEQ ID NOs: 1-4, and a nucleic acid sequence that is at least 90%, 91%, 92%, 93%, 94%, 95%, 96%, 97%, 98% or 99% identical to a nucleic acid sequence encoding a light chain amino acid sequence comprising an amino acid sequence selected from the group consisting of SEQ ID NOs: 5-9.

In some embodiments, the CD147 antibody or antigen-binding fragment thereof is encoded by a nucleic acid sequence that comprises a nucleic acid sequence that is at least 90%, 91%, 92%, 93%, 94%, 95%, 96%, 97%, 98% or 99% identical to a nucleic acid sequence encoding a heavy chain amino acid sequence comprising an amino acid sequence selected from the group consisting of SEQ ID NOs: 1-3, and a nucleic acid sequence that is at least 90%, 91%, 92%, 93%, 94%, 95%, 96%, 97%, 98% or 99% identical to a nucleic acid sequence encoding a light chain amino acid sequence comprising an amino acid sequence selected from the group consisting of SEQ ID NOs: 5-8.

Activatable CD147 Antibodies

As described above, the disclosure also provides activatable antibodies that include an antibody or antigen-binding fragment thereof that specifically binds CD147 coupled to a masking moiety (MM), such that coupling of the MM reduces the ability of the antibody or antigen-binding fragment thereof to bind CD147. In some embodiments, the MM is coupled via a sequence that includes a substrate for a protease (CM, cleavable moiety), for example, a protease that is active in diseased tissue and/or a protease that is co-localized with CD147 at a treatment site in a subject. The activatable CD147 antibodies provided herein are stable in circulation, activated at intended sites of therapy and/or diagnosis but not in normal, e.g., healthy tissue or other tissue not targeted for treatment and/or diagnosis, and, when activated, exhibit binding to CD147 that is at least comparable to the corresponding, unmodified antibody, also referred to herein as the parental antibody.

The activatable CD147 antibodies described herein overcome a limitation of antibody therapeutics, particularly antibody therapeutics that are known to be toxic to at least some degree in vivo. Target-mediated toxicity constitutes a major limitation for the development of therapeutic antibodies. The activatable CD147 antibodies provided herein are designed to address the toxicity associated with the inhibition of the target in normal tissues by traditional therapeutic antibodies. These activatable CD147 antibodies remain masked until proteolytically activated at the site of disease. Starting with a CD147 antibody as a parental therapeutic antibody, the activatable CD147 antibodies of the invention were engineered by coupling the antibody to an inhibitory mask through a linker that incorporates a protease substrate.

As used herein, the term cleaved state of the activatable antibody refers to the condition of the activatable antibodies following modification of the CM by at least one protease. The term uncleaved state, as used herein, refers to the condition of the activatable antibodies in the absence of cleavage of the CM by a protease. As discussed above, the term “activatable antibodies” is used herein to refer to an activatable antibody in both its uncleaved (native) state, as well as in its cleaved state. It will be apparent to the ordinarily skilled artisan that in some embodiments a cleaved activatable antibody may lack an MM due to cleavage of the CM by protease, resulting in release of at least the MM (e.g., where the MM is not joined to the activatable antibodies by a covalent bond (e.g., a disulfide bond between cysteine residues).

By activatable or switchable is meant that the activatable antibody exhibits a first level of binding to a target when the activatable antibody is in a inhibited, masked or uncleaved state (i.e., a first conformation), and a second level of binding to the target in the uninhibited, unmasked and/or cleaved state (i.e., a second conformation), where the second level of target binding is greater than the first level of binding. In general, the access of target to the AB of the activatable antibody is greater in the presence of a cleaving agent capable of cleaving the CM, i.e., a protease, than in the absence of such a cleaving agent. Thus, when the activatable antibody is in the uncleaved state, the AB is inhibited from target binding and can be masked from target binding (i.e., the first conformation is such the AB cannot bind the target), and in the cleaved state the AB is not inhibited or is unmasked to target binding.

The CM and AB of the activatable antibodies are selected so that the AB represents a binding moiety for a given target, and the CM represents a substrate for a protease. In some embodiments, the protease is co-localized with the target at a treatment site or diagnostic site in a subject. As used herein, co-localized refers to being at the same site or relatively close nearby. In some embodiments, a protease cleaves a CM yielding an activated antibody that binds to a target located nearby the cleavage site. The activatable antibodies disclosed herein find particular use where, for example, a protease capable of cleaving a site in the CM, i.e., a protease, is present at relatively higher levels in target-containing tissue of a treatment site or diagnostic site than in tissue of non-treatment sites (for example in healthy tissue). In some embodiments, a CM of the disclosure is also cleaved by one or more other proteases. In some embodiments, it is the one or more other proteases that is co-localized with the target and that is responsible for cleavage of the CM in vivo.

In some embodiments activatable antibodies provide for reduced toxicity and/or adverse side effects that could otherwise result from binding of the AB at non-treatment sites if the AB were not masked or otherwise inhibited from binding to the target.

In general, an activatable antibody can be designed by selecting an AB of interest (such as any CD147 antibody or fragment thereof described herein) and constructing the remainder of the activatable antibody so that, when conformationally constrained, the MM provides for masking of the AB or reduction of binding of the AB to its target. Structural design criteria can be to be taken into account to provide for this functional feature.

Activatable antibodies exhibiting a switchable phenotype of a desired dynamic range for target binding in an inhibited versus an uninhibited conformation are provided. Dynamic range generally refers to a ratio of (a) a maximum detected level of a parameter under a first set of conditions to (b) a minimum detected value of that parameter under a second set of conditions. For example, in the context of an activatable antibody, the dynamic range refers to the ratio of (a) a maximum detected level of target protein binding to an activatable antibody in the presence of at least one protease capable of cleaving the CM of the activatable antibodies to (b) a minimum detected level of target protein binding to an activatable antibody in the absence of the protease. The dynamic range of an activatable antibody can be calculated as the ratio of the dissociation constant of an activatable antibody cleaving agent (e.g., enzyme) treatment to the dissociation constant of the activatable antibodies cleaving agent treatment. The greater the dynamic range of an activatable antibody, the better the switchable phenotype of the activatable antibody. Activatable antibodies having relatively higher dynamic range values (e.g., greater than 1) exhibit more desirable switching phenotypes such that target protein binding by the activatable antibodies occurs to a greater extent (e.g., predominantly occurs) in the presence of a cleaving agent (e.g., enzyme) capable of cleaving the CM of the activatable antibodies than in the absence of a cleaving agent.

As described above, the activatable CD147 antibodies provided herein include a masking moiety (MM). In some embodiments, the masking moiety is an amino acid sequence that is coupled or otherwise attached to the CD147 antibody and is positioned within the activatable CD147 antibody construct such that the masking moiety reduces the ability of the CD147 antibody to specifically bind CD147. Suitable masking moieties are identified using any of a variety of known techniques. For example, peptide masking moieties are identified using the methods described in PCT Publication No. WO 2009/025846 by Daugherty et al., the contents of which are hereby incorporated by reference in their entirety.

As described above, the activatable CD147 antibodies provided herein include a cleavable moiety (CM). In some embodiments, the cleavable moiety includes an amino acid sequence that is a substrate for a protease, usually an extracellular protease. Suitable substrates are identified using any of a variety of known techniques. For example, peptide substrates are identified using the methods described in U.S. Pat. No. 7,666,817 by Daugherty et al.; in U.S. Pat. No. 8,563,269 by Stagliano et al.; and in PCT Publication No. WO 2014/026136 by La Porte et al., the contents of each of which are hereby incorporated by reference in their entirety. (See also Boulware et al. “Evolutionary optimization of peptide substrates for proteases that exhibit rapid hydrolysis kinetics.” Biotechnol Bioeng. 106.3 (2010): 339-46).

Exemplary substrates include but are not limited to substrates cleavable by one or more of the following enzymes or proteases listed in Table 3.

TABLE 3
Exemplary Proteases and/or Enzymes

| | | |
|---|---|---|
| ADAMS, ADAMTS, e.g. | Cysteine proteinases, e.g., | Serine proteases, e.g, |
| ADAM8 | Cruzipain | activated protein C |
| ADAM9 | Legumain | Cathepsin A |
| ADAM10 | Otubain-2 | Cathepsin G |
| ADAM12 | | Chymase |
| ADAM15 | KLKs, e.g., | coagulation factor proteases |
| ADAM17/TACE | KLK4 | (e.g., FVIIa, FIXa, FXa, FXIa, |
| ADAMDEC1 | KLK5 | FXIIa) |
| ADAMTS1 | KLK6 | Elastase |
| ADAMTS4 | KLK7 | Granzyme B |
| ADAMTS5 | KLK8 | Guanidinobenzoatse |
| | KLK10 | HtrA1 |
| Aspartate proteases, e.g., | KLK11 | Human Neutrophil Elastase |
| BACE | KLK13 | Lactoferrin |
| Renin | KLK14 | Marapsin |
| | | NS3/4A |
| Aspartic cathepsins, e.g., | Metallo proteinases, e.g., | PACE4 |
| Cathepsin D | Meprin | Plasmin |
| Cathepsin E | Neprilysin | PSA |
| | PSMA | tPA |
| Caspases, e.g., | BMP-1 | Thrombin |
| Caspase 1 | | Tryptase |
| Caspase 2 | MMPs, e.g., | uPA |
| Caspase 3 | MMP1 | |
| Caspase 4 | MMP2 | Type II Transmembrane |
| Caspase 5 | MMP3 | Serine Proteases (TTSPs), e.g., |
| Caspase 6 | MMP7 | DESC1 |
| Caspase 7 | MMP8 | DPP-4 |
| Caspase 8 | MMP9 | FAP |
| Caspase 9 | MMP10 | Hepsin |
| Caspase 10 | MMP11 | Matriptase-2 |
| Caspase 14 | MMP12 | MT-SP1/Matriptase |
| | MMP13 | TMPRSS2 |
| Cysteine cathepsins, e.g., | MMP14 | TMPRSS3 |
| Cathepsin B | MMP15 | TMPRSS4 |
| Cathepsin C | MMP16 | |
| Cathepsin K | MMP17 | |
| Cathepsin L | MMP19 | |
| Cathepsin S | MMP20 | |
| Cathepsin V12 | MMP23 | |
| Cathepsin X/Z/p | MMP24 | |
| | MMP26 | |
| | MMP27 | |

The activatable antibodies in an activated state bind CD147 and include (i) an antibody or an antigen binding fragment thereof (AB) that specifically binds to CD147; (ii) a masking moiety (MM) that, when the activatable antibody is in an uncleaved state, inhibits the binding of the AB to CD147; and (c) a cleavable moiety (CM) coupled to the AB, wherein the CM is a polypeptide that functions as a substrate for a protease.

In some embodiments, the activatable antibody in the uncleaved state has the structural arrangement from N-terminus to C-terminus as follows: MM-CM-AB or AB-CM-MM.

In some embodiments, the activatable antibody comprises a linking peptide between the MM and the CM.

In some embodiments, the activatable antibody comprises a linking peptide between the CM and the AB.

In some embodiments, the activatable antibody comprises a first linking peptide (LP1) and a second linking peptide (LP2), and wherein the activatable antibody in the uncleaved state has the structural arrangement from N-terminus to C-terminus as follows: MM-LP1-CM-LP2-AB or AB-LP2-CM-LP1-MM. In some embodiments, the two linking peptides need not be identical to each other.

In some embodiments, at least one of LP1 or LP2 comprises an amino acid sequence selected from the group consisting of (GS)ₙ, (GGS)ₙ, (GSGGS)ₙ (SEQ ID NO: 339) and (GGGS)ₙ (SEQ ID NO: 340), where n is an integer of at least one.

In some embodiments, at least one of LP1 or LP2 comprises an amino acid sequence selected from the group consisting of GGSG (SEQ ID NO: 341), GGSGG (SEQ ID NO: 342), GSGSG (SEQ ID NO: 343), GSGGG (SEQ ID NO: 344), GGGSG (SEQ ID NO: 345), and GSSSG (SEQ ID NO: 346).

In some embodiments, LP1 comprises the amino acid sequence GSSGGSGGSGGSG (SEQ ID NO: 347), GSSGGSGGSGG (SEQ ID NO: 348), GSSGGSGGSGGS (SEQ ID NO: 349), GSSGGSGGSGGSGGGS (SEQ ID NO: 350), GSSGGSGGSG (SEQ ID NO: 351), or GSSGGSGGSGS (SEQ ID NO: 352).

In some embodiments, LP2 comprises the amino acid sequence GSS, GGS, GGGS (SEQ ID NO: 353), GSSGT (SEQ ID NO: 354) or GSSG (SEQ ID NO: 355).

In some embodiments, the antibody or antigen-binding fragment thereof that binds CD147 is a monoclonal antibody, domain antibody, single chain, Fab fragment, a F(ab′)₂ fragment, a scFv, a scAb, a dAb, a single domain heavy chain antibody, or a single domain light chain antibody. In some embodiments, such an antibody or antigen-binding fragment thereof that binds CD147 is a mouse, other rodent, chimeric, humanized or fully human monoclonal antibody.

In some embodiments, the activatable antibody in an uncleaved state specifically binds to the mammalian CD147 with a dissociation constant less than or equal to 1 nM, less than or equal to 5 nM, less than or equal to 10 nM, less than or equal to 15 nM, less than or equal to 20 nM, less than or equal to 25 nM, less than or equal to 50 nM, less than or equal to 100 nM, less than or equal to 150 nM, less than or equal to 250 nM, less than or equal to 500 nM, less than or equal to 750 nM, less than or equal to 1000 nM, and/or less than or equal to 2000 nM.

In some embodiments, the activatable antibody in an uncleaved state specifically binds to the mammalian CD147 with a dissociation constant in the range of 1 nM to 2000 nM, 1 nM to 1000 nM, 1 nM to 750 nM, 1 nM to 500 nM, 1 nM to 250 nM, 1 nM to 150 nM, 1 nM to 100 nM, 1 nM to 50 nM, 1 nM to 25 nM, 1 nM to 15 nM, 1 nM to 10 nM, 1 nM to 5 nM, 5 nM to 2000 nM, 5 nM to 1000 nM, 5 nM to 750 nM, 5 nM to 500 nM, 5 nM to 250 nM, 5 nM to 150 nM, 5 nM to 100 nM, 5 nM to 50 nM, 5 nM to 25 nM, 5 nM to 15 nM, 5 nM to 10 nM, 10 nM to 2000 nM, 10 nM to 1000 nM, 10 nM to 750 nM, 10 nM to 500 nM, 10 nM to 250 nM, 10 nM to 150 nM, 10 nM to 100 nM, 10 nM to 50 nM, 10 nM to 25 nM, 10 nM to 15 nM, 15 nM to 2000 nM, 15 nM to 1000 nM, 15 nM to 750 nM, 15 nM to 500 nM, 15 nM to 250 nM, 15 nM to 150 nM, 15 nM to 100 nM, 15 nM to 50 nM, 15 nM to 25 nM, 25 nM to 2000 nM, 25 nM to 1000 nM, 25 nM to 750 nM, 25 nM to 500 nM, 25 nM to 250 nM, 25 nM to 150 nM, 25 nM to 100 nM, 25 nM to 50 nM, 50 nM to 2000 nM, 50 nM to 1000 nM, 50 nM to 750 nM, 50 nM to 500 nM, 50 nM to 250 nM, 50 nM to 150 nM, 50 nM to 100 nM, 100 nM to 2000 nM, 100 nM to 1000 nM, 100 nM to 750 nM, 100 nM to 500 nM, 100 nM to 250 nM, 100 nM to 150 nM, 150 nM to 2000 nM, 150 nM to 1000 nM, 150 nM to 750 nM, 150 nM to 500 nM, 150 nM to 250 nM, 250 nM to 2000 nM, 250 nM to 1000 nM, 250 nM to 750 nM, 250 nM to 500 nM, 500 nM to 2000 nM, 500 nM to 1000 nM, 500 nM to 750 nM, 500 nM to 500 nM, 500 nM to 250 nM, 500 nM to 150 nM, 500 nM to 100 nM, 500 nM to 50 nM, 750 nM to 2000 nM, 750 nM to 1000 nM, or 1000 nM to 2000 nM.

In some embodiments, the activatable antibody in an activated state specifically binds to the mammalian CD147 with a dissociation constant is less than or equal to 0.01 nM, 0.05 nM, 0.1 nM, 0.5 nM, 1 nM, 5 nM, or 10 nM.

In some embodiments, the activatable antibody in an activated state specifically binds to the mammalian CD147 with a dissociation constant in the range of 0.01 nM to 100 nM, 0.01 nM to 10 nM, 0.01 nM to 5 nM, 0.01 nM to 1 nM, 0.01 to 0.5 nM, 0.01 nm to 0.1 nM, 0.01 nm to 0.05 nM, 0.05 nM to 100 nM, 0.05 nM to 10 nM, 0.05 nM to 5 nM, 0.05 nM to 1 nM, 0.05 to 0.5 nM, 0.05 nm to 0.1 nM, 0.1 nM to 100 nM, 0.1 nM to 10 nM, 0.1 nM to 5 nM, 0.1 nM to 1 nM, 0.1 to 0.5 nM, 0.5 nM to 100 nM, 0.5 nM to 10 nM, 0.5 nM to 5 nM, 0.5 nM to 1 nM, 1 nM to 100 nM, 1 nM to 10 nM, 1 nM to 5 nM, 5 nM to 100 nM, 5 nM to 10 nM, or 10 nM to 100 nM.

When the AB is modified with a MM and is in the presence of the target, specific binding of the AB to its target is reduced or inhibited, as compared to the specific binding of the AB not modified with an MM or the specific binding of the parental AB to the target.

The K_{d} of the AB modified with a MM towards the CD147 target is at least 5, 10, 25, 50, 100, 250, 500, 1,000, 2,500, 5,000, 10,000, 50,000, 100,000, 500,000, 1,000,000, 5,000,000, 10,000,000, 50,000,000 or greater, or between 5-10, 10-100, 10-1,000, 10-10,000, 10-100,000, 10-1,000,000, 10-10,000,000, 100-1,000, 100-10,000, 100-100,000, 100-1,000,000, 100-10,000,000, 1,000-10,000, 1,000-100,000, 1,000-1,000,000, 1000-10,000,000, 10,000-100,000, 10,000-1,000,000, 10,000-10,000,000, 100,000-1,000,000, or 100,000-10,000,000 times greater than the K_{d} of the AB not modified with an MM or of the parental AB towards the CD147 target. Conversely, the binding affinity of the AB modified with a MM towards the CD147 target is at least 2, 3, 4, 5, 10, 25, 50, 100, 250, 500, 1,000, 2,500, 5,000, 10,000, 50,000, 100,000, 500,000, 1,000,000, 5,000,000, 10,000,000, 50,000,000 or greater, or between 5-10, 10-100, 10-1,000, 10-10,000, 10-100,000, 10-1,000,000, 10-10,000,000, 100-1,000, 100-10,000, 100-100,000, 100-1,000,000, 100-10,000,000, 1,000-10,000, 1,000-100,000, 1,000-1,000,000, 1000-10,000,000, 10,000-100,000, 10,000-1,000,000, 10,000-10,000,000, 100,000-1,000,000, or 100,000-10,000,000 times lower than the binding affinity of the AB not modified with an MM or of the parental AB towards the CD147 target.

The dissociation constant (K_{d}) of the MM towards the AB is generally greater than the K_{d} of the AB towards the CD147 target. The K_{d} of the MM towards the AB can be at least 5, 10, 25, 50, 100, 250, 500, 1,000, 2,500, 5,000, 10,000, 100,000, 1,000,000 or even 10,000,000 times greater than the K_{d} of the AB towards the CD147 target. Conversely, the binding affinity of the MM towards the AB is generally lower than the binding affinity of the AB towards the CD147 target. The binding affinity of MM towards the AB can be at least 5, 10, 25, 50, 100, 250, 500, 1,000, 2,500, 5,000, 10,000, 100,000, 1,000,000 or even 10,000,000 times lower than the binding affinity of the AB towards the CD147 target.

In some embodiments, the dissociation constant (K_{d}) of the MM towards the AB is approximately equal to the K_{d} of the AB towards the CD147 target. In some embodiments, the dissociation constant (K_{d}) of the MM towards the AB is no more than the dissociation constant of the AB towards the CD147 target. In some embodiments, the dissociation constant (K_{d}) of the MM towards the AB is equivalent to the dissociation constant of the AB towards the CD147 target.

In some embodiments, the dissociation constant (K_{d}) of the MM towards the AB is less than the dissociation constant of the AB towards the CD147 target.

In some embodiments, the dissociation constant (K_{d}) of the MM towards the AB is greater than the dissociation constant of the AB towards the CD147 target.

In some embodiments, the MM has a K_{d} for binding to the AB that is no more than the K_{d} for binding of the AB to the target.

In some embodiments, the MM has a K_{d} for binding to the AB that is no less than the K_{d} for binding of the AB to the target.

In some embodiments, the MM has a K_{d} for binding to the AB that is approximately equal to the K_{d} for binding of the AB to the target.

In some embodiments, the MM has a K_{d} for binding to the AB that is less than the K_{d} for binding of the AB to the target.

In some embodiments, the MM has a K_{d} for binding to the AB that is greater than the K_{d} for binding of the AB to the target.

In some embodiments, the MM has a K_{d} for binding to the AB that is no more than 2, 3, 4, 5, 10, 25, 50, 100, 250, 500, or 1,000 fold greater than the K_{d} for binding of the AB to the target. In some embodiments, the MM has a K_{d} for binding to the AB that is between 1-5, 2-5, 2-10, 5-10, 5-20, 5-50, 5-100, 10-100, 10-1,000, 20-100, 20-1000, or 100-1,000 fold greater than the K_{d} for binding of the AB to the target.

In some embodiments, the MM has an affinity for binding to the AB that is less than the affinity of binding of the AB to the target.

In some embodiments, the MM has an affinity for binding to the AB that is no more than the affinity of binding of the AB to the target.

In some embodiments, the MM has an affinity for binding to the AB that is approximately equal of the affinity of binding of the AB to the target.

In some embodiments, the MM has an affinity for binding to the AB that is no less than the affinity of binding of the AB to the target.

In some embodiments, the MM has an affinity for binding to the AB that is greater than the affinity of binding of the AB to the target.

In some embodiments, the MM has an affinity for binding to the AB that is 2, 3, 4, 5, 10, 25, 50, 100, 250, 500, or 1,000 less than the affinity of binding of the AB to the target. I In some embodiments, the MM has an affinity for binding to the AB that is between 1-5, 2-5, 2-10, 5-10, 5-20, 5-50, 5-100, 10-100, 10-1,000, 20-100, 20-1000, or 100-1,000 fold less than the affinity of binding of the AB to the target. In some embodiments, the MM has an affinity for binding to the AB that is 2 to 20 fold less than the affinity of binding of the AB to the target. In some embodiments, a MM not covalently linked to the AB and at equimolar concentration to the AB does not inhibit the binding of the AB to the target.

When the AB is modified with a MM and is in the presence of the target specific binding of the AB to its target is reduced or inhibited, as compared to the specific binding of the AB not modified with an MM or the specific binding of the parental AB to the target. When compared to the binding of the AB not modified with an MM or the binding of the parental AB to the target the AB's ability to bind the target when modified with an MM can be reduced by at least 50%, 60%, 70%, 80%, 90%, 92%, 93%, 94%, 95%, 96%, 97%, 98%, 99% and even 100% for at least 2, 4, 6, 8, 12, 28, 24, 30, 36, 48, 60, 72, 84, or 96 hours, or 5, 10, 15, 30, 45, 60, 90, 120, 150, or 180 days, or 1, 2, 3, 4, 5, 6, 7, 8, 9, 10, 11, or 12 months or more when measured in vivo or in an in vitro assay.

The MM inhibits the binding of the AB to the target. The MM binds the antigen binding domain of the AB and inhibits binding of the AB to the target. The MM can sterically inhibit the binding of the AB to the target. The MM can allosterically inhibit the binding of the AB to its target. In these embodiments when the AB is modified or coupled to a MM and in the presence of target there is no binding or substantially no binding of the AB to the target, or no more than 0.001%, 0.01%, 0.1%, 1%, 2° %, 3%, 4%, 5%, 6%, 7%, 8%, 9%, 10%, 15%, 20%, 25%, 30%, 35%, 40%, or 50% binding of the AB to the target, as compared to the binding of the AB not modified with an MM, the parental AB, or the AB not coupled to an MM to the target, for at least 2, 4, 6, 8, 12, 28, 24, 30, 36, 48, 60, 72, 84, or 96 hours, or 5, 10, 15, 30, 45, 60, 90, 120, 150, or 180 days, or 1, 2, 3, 4, 5, 6, 7, 8, 9, 10, 11, or 12 months or longer when measured in vivo or in an in vitro assay.

When an AB is coupled to or modified by a MM, the MM ‘masks’ or reduces or otherwise inhibits the specific binding of the AB to the target. When an AB is coupled to or modified by a MM, such coupling or modification can effect a structural change that reduces or inhibits the ability of the AB to specifically bind its target.

An AB coupled to or modified with an MM can be represented by the following formulae (in order from an amino (N) terminal region to carboxyl (C) terminal region:
(MM)-(AB)
(AB)-(MM)
(MM)-L-(AB)
(AB)-L-(MM)
   where MM is a masking moiety, the AB is an antibody or antibody fragment thereof, and the L is a linker. In many embodiments, it can be desirable to insert one or more linkers, e.g., flexible linkers, into the composition so as to provide for flexibility.

In certain embodiments, the MM is not a natural binding partner of the AB. In some embodiments, the MM contains no or substantially no homology to any natural binding partner of the AB. In some embodiments, the MM is no more than 5%, 10%, 15%, 20%, 25%, 30%, 35%, 40%, 45%, 50%, 55%, 60%, 65%, 70%, 75%, or 80 similar to any natural binding partner of the AB. In some embodiments, the MM is no more than 5%, 10%, 15%, 20%, 250%, 30%, 35%, 40%, 45%, 50%, 55%, 60%, 65%, 70%, 75%, or 80% identical to any natural binding partner of the AB. In some embodiments, the MM is no more than 25% identical to any natural binding partner of the AB. In some embodiments, the MM is no more than 50% identical to any natural binding partner of the AB. In some embodiments, the MM is no more than 20% identical to any natural binding partner of the AB. In some embodiments, the MM is no more than 10% identical to any natural binding partner of the AB.

In some embodiments, the activatable antibodies include an AB that is modified by an MM and also includes one or more cleavable moieties (CM). Such activatable antibodies exhibit activatable/switchable binding, to the AB's target. Activatable antibodies generally include an antibody or antibody fragment (AB), modified by or coupled to a masking moiety (MM) and a modifiable or cleavable moiety (CM). In some embodiments, the CM contains an amino acid sequence that serves as a substrate for at least one protease.

The elements of the activatable antibodies are arranged so that the MM and CM are positioned such that in a cleaved (or relatively active) state and in the presence of a target, the AB binds a target while the activatable antibody is in an uncleaved (or relatively inactive) state in the presence of the target, specific binding of the AB to its target is reduced or inhibited. The specific binding of the AB to its target can be reduced due to the inhibition or masking of the AB's ability to specifically bind its target by the MM.

The K_{d} of the AB modified with a MM and a CM towards the CD147 target is at least 5, 10, 25, 50, 100, 250, 500, 1,000, 2,500, 5,000, 10,000, 50,000, 100,000, 500,000, 1,000,000, 5,000,000, 10,000,000, 50,000,000 or greater, or between 5-10, 10-100, 10-1,000, 10-10,000, 10-100,000, 10-1,000,000, 10-10,000,000, 100-1,000, 100-10,000, 100-100,000, 100-1,000,000, 100-10,000,000, 1,000-10,000, 1,000-100,000, 1,000-1,000,000, 1000-10,000,000, 10,000-100,000, 10,000-1,000,000, 10,000-10,000,000, 100,000-1,000,000, or 100,000-10,000,000 times greater than the K_{d} of the AB not modified with an MM and a CM or of the parental AB towards the CD147 target. Conversely, the binding affinity of the AB modified with a MM and a CM towards the CD147 target is at least 5, 10, 25, 50, 100, 250, 500, 1,000, 2,500, 5,000, 10,000, 50,000, 100,000, 500,000, 1,000,000, 5,000,000, 10,000,000, 50,000,000 or greater, or between 5-10, 10-100, 10-1,000, 10-10,000, 10-100,000, 10-1,000,000, 10-10,000,000, 100-1,000, 100-10,000, 100-100,000, 100-1,000,000, 100-10,000,000, 1,000-10,000, 1,000-100,000, 1,000-1,000,000, 1000-10,000,000, 10,000-100,000, 10,000-1,000,000, 10,000-10,000,000, 100,000-1,000,000, or 100,000-10,000,000 times lower than the binding affinity of the AB not modified with an MM and a CM or of the parental AB towards the CD147 target.

When the AB is modified with a MM and a CM and is in the presence of the target but not in the presence of a modifying agent (for example at least one protease), specific binding of the AB to its target is reduced or inhibited, as compared to the specific binding of the AB not modified with an MM and a CM or of the parental AB to the target. When compared to the binding of the parental AB or the binding of an AB not modified with an MM and a CM to its target, the AB's ability to bind the target when modified with an MM and a CM can be reduced by at least 50%, 60%, 70%, 8%, 90%, 92%, 93%, 94%, 95%, 960%, 97%, 98%, 99% and even 100% for at least 2, 4, 6, 8, 12, 28, 24, 30, 36, 48, 60, 72, 84, or 96 hours or 5, 10, 15, 30, 45, 60, 90, 120, 150, or 180 days, or 1, 2, 3, 4, 5, 6, 7, 8, 9, 10, 11, or 12 months or longer when measured in vivo or in an in vitro assay.

Activatable antibodies can be provided in a variety of structural configurations. Exemplary formulae for activatable antibodies are provided below. It is specifically contemplated that the N- to C-terminal order of the AB, MM and CM can be reversed within an activatable antibody. It is also specifically contemplated that the CM and MM may overlap in amino acid sequence, e.g., such that the CM is contained within the MM.

For example, activatable antibodies can be represented by the following formula (in order from an amino (N) terminal region to carboxyl (C) terminal region:
(MM)-(CM)-(AB)
(AB)-(CM)-(MM)
   where MM is a masking moiety, CM is a cleavable moiety, and AB is an antibody or fragment thereof. It should be noted that although MM and CM are indicated as distinct components in the formulae above, in all exemplary embodiments (including formulae) disclosed herein it is contemplated that the amino acid sequences of the MM and the CM could overlap, e.g., such that the CM is completely or partially contained within the MM. In addition, the formulae above provide for additional amino acid sequences that can be positioned N-terminal or C-terminal to the activatable antibodies elements.

In certain embodiments, the MM is not a natural binding partner of the AB. In some embodiments, the MM contains no or substantially no homology to any natural binding partner of the AB. In some embodiments, the MM is no more than 5%, 10%, 15%, 20%, 250%, 30%, 35%, 40%, 45%, 50%, 55%, 60%, 65%, 70%, 75%, or 80% similar to any natural binding partner of the AB. In some embodiments, the MM is no more than 5%, 10%, 15%, 20%, 25%, 30%, 35%, 40%, 45%, 50%, 55%, 60%, 65%, 70%⁰, 75%, or 80% identical to any natural binding partner of the AB. In some embodiments, the MM is no more than 50% identical to any natural binding partner of the AB. In some embodiments, the MM is no more than 25% identical to any natural binding partner of the AB. In some embodiments, the MM is no more than 20% identical to any natural binding partner of the AB. In some embodiments, the MM is no more than 10% identical to any natural binding partner of the AB.

In many embodiments it may be desirable to insert one or more linkers, e.g., flexible linkers, into the activatable antibody construct so as to provide for flexibility at one or more of the MM-CM junction, the CM-AB junction, or both. For example, the AB, MM, and/or CM may not contain a sufficient number of residues (e.g., Gly, Ser, Asp, Asn, especially Gly and Ser, particularly Gly) to provide the desired flexibility. As such, the switchable phenotype of such activatable antibody constructs may benefit from introduction of one or more amino acids to provide for a flexible linker. In addition, as described below, where the activatable antibody is provided as a conformationally constrained construct, a flexible linker can be operably inserted to facilitate formation and maintenance of a cyclic structure in the uncleaved activatable antibody.

For example, in certain embodiments an activatable antibody comprises one of the following formulae (where the formula below represent an amino acid sequence in either N- to C-terminal direction or C- to N-terminal direction):
(MM)-L1-(CM)-(AB)
(MM)-(CM)-L2-(AB)
(MM)-L1-(CM)-L2-(AB)
   wherein MM, CM, and AB are as defined above; wherein L1 and L2 are each independently and optionally present or absent, are the same or different flexible linkers that include at least 1 flexible amino acid (e.g., Gly). In addition, the formulae above provide for additional amino acid sequences that can be positioned N-terminal or C-terminal to the activatable antibodies elements. Examples include, but are not limited to, targeting moieties (e.g., a ligand for a receptor of a cell present in a target tissue) and serum half-life extending moieties (e.g., polypeptides that bind serum proteins, such as immunoglobulin (e.g., IgG) or serum albumin (e.g., human serum albumin (HAS)).

The CM is specifically cleaved by at least one protease at a rate of about 0.001-1500×10⁴ M⁻¹S⁻¹ or at least 0.001, 0.005, 0.01, 0.05, 0.1, 0.5, 1, 2.5, 5, 7.5, 10, 15, 20, 25, 50, 75, 100, 125, 150, 200, 250, 500, 750, 1000, 1250, or 1500×10⁴ M⁻¹S⁻¹. In some embodiments, the CM is specifically cleaved at a rate of about 100,000 M⁻¹S⁻¹. In some embodiments, the CM is specifically cleaved at a rate from about 1×10E2 to about 1×10E6 M⁻¹S⁻¹ (i.e., from about 1×10² to about 1×10⁶ M⁻¹S⁻¹).

For specific cleavage by an enzyme, contact between the enzyme and CM is made. When the activatable antibody comprising an AB coupled to a MM and a CM is in the presence of target and sufficient enzyme activity, the CM can be cleaved. Sufficient enzyme activity can refer to the ability of the enzyme to make contact with the CM and effect cleavage. It can readily be envisioned that an enzyme may be in the vicinity of the CM but unable to cleave because of other cellular factors or protein modification of the enzyme.

Linkers suitable for use in compositions described herein are generally ones that provide flexibility of the modified AB or the activatable antibodies to facilitate the inhibition of the binding of the AB to the target. Such linkers are generally referred to as flexible linkers. Suitable linkers can be readily selected and can be of any of a suitable of different lengths, such as from 1 amino acid (e.g., Gly) to 20 amino acids, from 2 amino acids to 15 amino acids, from 3 amino acids to 12 amino acids, including 4 amino acids to 10 amino acids, 5 amino acids to 9 amino acids, 6 amino acids to 8 amino acids, or 7 amino acids to 8 amino acids, and can be 1, 2, 3, 4, 5, 6, 7, 8, 9, 10, 11, 12, 13, 14, 15, 16, 17, 18, 19, or 20 amino acids in length.

Exemplary flexible linkers include glycine polymers (G)n, glycine-serine polymers (including, for example, (GS)n, (GSGGS)n (SEQ ID NO: 339) and (GGGS)n (SEQ ID NO: 340), where n is an integer of at least one), glycine-alanine polymers, alanine-serine polymers, and other flexible linkers known in the art. Glycine and glycine-serine polymers are relatively unstructured, and therefore may be able to serve as a neutral tether between components. Glycine accesses significantly more phi-psi space than even alanine, and is much less restricted than residues with longer side chains (see Scheraga, Rev. Computational Chem. 11173-142 (1992)). Exemplary flexible linkers include, but are not limited to Gly-Gly-Ser-Gly (SEQ ID NO: 341), Gly-Gly-Ser-Gly-Gly (SEQ ID NO: 342), Gly-Ser-Gly-Ser-Gly (SEQ ID NO: 343), Gly-Ser-Gly-Gly-Gly (SEQ ID NO: 344), Gly-Gly-Gly-Ser-Gly (SEQ ID NO: 345), Gly-Ser-Ser-Ser-Gly (SEQ ID NO: 346), and the like. The ordinarily skilled artisan will recognize that design of an activatable antibodies can include linkers that are all or partially flexible, such that the linker can include a flexible linker as well as one or more portions that confer less flexible structure to provide for a desired activatable antibodies structure.

The disclosure also provides compositions and methods that include an activatable CD147 antibody that includes an antibody or antibody fragment (AB) that specifically binds CD147, where the AB is coupled to a masking moiety (MM) that decreases the ability of the AB to bind its target. In some embodiments, the activatable CD147 antibody further includes a cleavable moiety (CM) that is a substrate for a protease. The compositions and methods provided herein enable the attachment of one or more agents to one or more cysteine residues in the AB without compromising the activity (e.g., the masking, activating or binding activity) of the activatable CD147 antibody. In some embodiments, the compositions and methods provided herein enable the attachment of one or more agents to one or more cysteine residues in the AB without reducing or otherwise disturbing one or more disulfide bonds within the MM. The compositions and methods provided herein produce an activatable CD147 antibody that is conjugated to one or more agents, e.g., any of a variety of therapeutic, diagnostic and/or prophylactic agents, for example, in some embodiments, without any of the agent(s) being conjugated to the MM of the activatable CD147 antibody. The compositions and methods provided herein produce conjugated activatable CD147 antibodies in which the MM retains the ability to effectively and efficiently mask the AB of the activatable antibody in an uncleaved state. The compositions and methods provided herein produce conjugated activatable CD147 antibodies in which the activatable antibody is still activated, i.e., cleaved, in the presence of a protease that can cleave the CM.

In some embodiments, the activatable antibody comprises a heavy chain variable region amino acid sequence selected from the group consisting of SEQ ID NOs: 1-4. In some embodiments, the activatable antibody comprises a heavy chain variable region amino acid sequence selected from the group consisting of SEQ ID NOs: 1-3.

In some embodiments, the activatable antibody comprises a light chain variable region amino acid sequence selected from the group consisting of SEQ ID NOs: 5-9. In some embodiments, the activatable antibody comprises a light chain variable region amino acid sequence selected from the group consisting of SEQ ID NOs: 5-8.

In some embodiments, the activatable antibody comprises a heavy chain variable region amino acid sequence selected from the group consisting of SEQ ID NOs: 1-4, and a light chain variable region amino acid sequence selected from the group consisting of SEQ ID NOs: 5-9.

In some embodiments, the activatable antibody comprises a heavy chain variable region amino acid sequence selected from the group consisting of SEQ ID NOs: 1-3, and a light chain variable region amino acid sequence selected from the group consisting of SEQ ID NOs: 5-8.

In some embodiments, the activatable antibody comprises a heavy chain variable region amino acid sequence that is at least 90%, 91%, 92%, 93%, 94%, 95%, 96%, 97%, 98% or 99% identical to an amino acid sequence selected from the group consisting of SEQ ID NOs: 1-4. In some embodiments, the activatable antibody comprises a heavy chain variable region amino acid sequence that is at least 90%, 91%, 92%, 93%, 94%, 95%, 96%, 97%, 98% or 99% identical to an amino acid sequence selected from the group consisting of SEQ ID NOs: 1-3.

In some embodiments, the activatable antibody comprises a light chain variable region amino acid sequence that is at least 90%, 91%, 92%, 93%, 94%, 95%, 96%, 97%, 98% or 99% identical to an amino acid sequence selected from the group consisting of SEQ ID NOs: 5-9. In some embodiments, the activatable antibody comprises a light chain variable region amino acid sequence that is at least 90%, 91%, 92%, 93%, 94%, 95%, 96%, 97%, 98% or 99% identical to an amino acid sequence selected from the group consisting of SEQ ID NOs: 5-8.

In some embodiments, the activatable antibody comprises a heavy chain variable region amino acid sequence that is at least 90%, 91%, 92%, 93%, 94%, 95%, 96%, 97%, 98% or 99% identical to an amino acid sequence selected from the group consisting of SEQ ID NOs: 1-4, and a light chain variable region amino acid sequence that is at least 90%, 91%, 92%, 93%, 94%, 95%, 96%, 97%, 98% or 99% identical to an amino acid sequence selected from the group consisting of SEQ ID NOs: 5-9.

In some embodiments, the activatable antibody comprises a heavy chain variable region amino acid sequence that is at least 90%, 91%, 92%, 93%, 94%, 95%, 96%, 97%, 98% or 99% identical to an amino acid sequence selected from the group consisting of SEQ ID NOs: 1-3, and a light chain variable region amino acid sequence that is at least 90%, 91%, 92%, 93%, 94%, 95%, 96%, 97%, 98% or 99% identical to an amino acid sequence selected from the group consisting of SEQ ID NOs: 5-8.

In some embodiments, the activatable antibody comprises a combination of a variable heavy chain complementarity determining region 1 (VH CDR1, also referred to herein as CDRH1) sequence, a variable heavy chain complementarity determining region 2 (VH CDR2, also referred to herein as CDRH2) sequence, a variable heavy chain complementarity determining region 3 (VH CDR3, also referred to herein as CDRH3) sequence, a variable light chain complementarity determining region 1 (VL CDR1, also referred to herein as CDRL1) sequence, a variable light chain complementarity determining region 2 (VL CDR2, also referred to herein as CDRL2) sequence, and a variable light chain complementarity determining region 3 (VL CDR3, also referred to herein as CDRL3) sequence, wherein at least one CDR sequence is selected from the group consisting of a VH CDR1 sequence comprising the amino acid sequence GFTFSNYWMN (SEQ ID NO: 10) or GFTFSNYWMD (SEQ ID NO: 11); a VH CDR2 sequence comprising the amino acid sequence EIRLKSYNYATH (SEQ ID NO: 12); a VH CDR3 sequence comprising the amino acid sequence AGTDY (SEQ ID NO: 13); a VL CDR1 sequence comprising the amino acid sequence KASQSVRTDVA (SEQ ID NO: 14) or RASQSVRTDVG (SEQ ID NO: 15); a VL CDR2 sequence comprising the amino acid sequence YSSNRYT (SEQ ID NO: 16); and a VL CDR3 sequence comprising the amino acid sequence QQDYSSPFT (SEQ ID NO: 17) or QQDYSSPYT (SEQ ID NO: 18).

In some embodiments, the activatable antibody comprises a combination of a VH CDR1 sequence, a VH CDR2 sequence, a VH CDR3 sequence, a VL CDR1 sequence, a VL CDR2 sequence, and a VL CDR3 sequence, wherein at least one CDR sequence is selected from the group consisting of a VH CDR1 sequence that includes a sequence that is at least 90%, 91%, 92%, 93%, 94%, 95%, 96%, 97%, 98%, 99% or more identical to a VH CDR1 sequence comprising the amino acid sequence GFTFSNYWMN (SEQ ID NO: 10) or GFTFSNYWMD (SEQ ID NO: 11); a VH CDR2 sequence that includes a sequence that is at least 90%, 91%, 92%, 93%, 94%, 95%, 96%, 97%, 98%, 99% or more identical to a VH CDR2 sequence comprising the amino acid sequence EIRLKSYNYATH (SEQ ID NO: 12); a VH CDR3 sequence that includes a sequence that is at least 90%, 91%, 92%, 93%, 94%, 95%, 96%, 97%, 98%, 99% or more identical to a VH CDR3 sequence comprising the amino acid sequence AGTDY (SEQ ID NO: 13); a VL CDR1 sequence that includes a sequence that is at least 90%6, 91%, 92%, 93%, 94%, 95%, 96%, 97%, 98%, 99% or more identical to a VL CDR1 sequence comprising the amino acid sequence KASQSVRTDVA (SEQ ID NO: 14) or RASQSVRTDVG (SEQ ID NO: 15); a VL CDR2 sequence that includes a sequence that is at least 90%, 91%, 92%, 93%, 94%, 95%, 96%, 97%, 98%, 99% or more identical to a VL CDR2 sequence comprising the amino acid sequence YSSNRYT (SEQ ID NO: 16); and a VL CDR3 sequence that includes a sequence that is at least 90%, 91%, 92%, 93%, 94%, 95%, 96%, 97%, 98%, 99% or more identical to a VL CDR3 sequence comprising the amino acid sequence QQDYSSPFT (SEQ ID NO: 17) or QQDYSSPYT (SEQ ID NO: 18).

In some embodiments, the activatable antibody comprises a combination of a VH CDR1 sequence, a VH CDR2 sequence, a VH CDR3 sequence, a VL CDR1 sequence, a VL CDR2 sequence, and a VL CDR3 sequence, wherein the VH CDR1 sequence comprises the amino acid sequence GFTFSNYWMN (SEQ ID NO: 10) or GFTFSNYWMD (SEQ ID NO: 11); the VH CDR2 sequence comprises the amino acid sequence EIRLKSYNYATH (SEQ ID NO: 12); the VH CDR3 sequence comprises the amino acid sequence AGTDY (SEQ ID NO: 13); the VL CDR1 sequence comprises the amino acid sequence KASQSVRTDVA (SEQ ID NO: 14) or RASQSVRTDVG (SEQ ID NO: 15); the VL CDR2 sequence comprises the amino acid sequence YSSNRYT (SEQ ID NO: 16); and the VL CDR3 sequence comprises the amino acid sequence QQDYSSPFT (SEQ ID NO: 17) or QQDYSSPYT (SEQ ID NO: 18).

In some embodiments, the activatable antibody comprises a combination of a VH CDR1 sequence, a VH CDR2 sequence, a VH CDR3 sequence, a VL CDR1 sequence, a VL CDR2 sequence, and a VL CDR3 sequence, wherein the VH CDR1 sequence comprises a sequence that is at least 90%, 91%, 92%, 93%, 94%, 95%, 96%, 97%, 98%, 99% or more identical to the amino acid sequence GFTFSNYWMN (SEQ ID NO: 10) or GFTFSNYWMD (SEQ ID NO: 11); the VH CDR2 sequence comprises a sequence that is at least 90%, 91%, 92%, 93%, 94%, 95%, 96%, 97%, 98%, 99%, or more identical to the amino acid sequence EIRLKSYNYATH (SEQ ID NO: 12); the VH CDR3 sequence comprises a sequence that is at least 90%, 91%, 92%, 93%, 94%, 95%, 96%, 97%, 98%, 99% or more identical to the amino acid sequence AGTDY (SEQ ID NO: 13); the VL CDR1 sequence comprises a sequence that is at least 90%, 91%, 92%, 93%, 94%, 95%, 96%, 97%, 98%, 99% or more identical to the amino acid sequence KASQSVRTDVA (SEQ ID NO: 14) or RASQSVRTDVG (SEQ ID NO: 15); the VL CDR2 sequence comprises a sequence that is at least 90%, 91%, 92%, 93%, 94%, 95%, 96%, 97%, 98%, 99% or more identical to the amino acid sequence YSSNRYT (SEQ ID NO: 16); and the VL CDR3 sequence comprises a sequence that is at least 90%, 91%, 92%, 93%, 94%, 95%, 96%, 97%, 98%, 99% or more identical to the amino acid sequence QQDYSSPFT (SEQ ID NO: 17) or QQDYSSPYT (SEQ ID NO: 18).

In some embodiments, the AB of the activatable CD147 antibody comprises a heavy chain variable region amino acid sequence selected from the group consisting of the heavy chain variable region sequences shown in Table 1. In some embodiments, the AB of the activatable CD147 antibody comprises a light chain variable region amino acid sequence selected from the group consisting of the light chain variable region sequences shown in Table 1. In some embodiments, the AB of the activatable CD147 antibody comprises a heavy chain variable region amino acid sequence selected from the group consisting of the heavy chain variable region sequences shown in Table 1 and a light chain variable region amino acid sequence selected from the group consisting of the light chain variable region sequences shown in Table 1.

In some embodiments, the AB of the activatable CD147 antibody comprises a heavy chain variable region amino acid sequence that is at least 90%, 91%, 92%, 93%, 94%, 95° %, 96%, 97%, 98% or 99% identical to an amino acid sequence selected from the group consisting of the heavy chain variable region sequences shown in Table 1. In some embodiments, the AB of the activatable CD147 antibody comprises a light chain variable region amino acid sequence that is at least 90%, 91%, 92%, 93%, 94%, 95%, 96%, 97%, 98% or 99% identical to an amino acid sequence selected from the group consisting of the light chain variable region sequences shown in Table 1. In some embodiments, the AB of the activatable CD147 antibody comprises a heavy chain variable region amino acid sequence that is at least 90%, 91%, 92%, 93%, 94%, 95%, 96%, 97%, 98% or 99% identical to an amino acid sequence selected from the group consisting of the heavy chain variable region sequences shown in Table 1 and a light chain variable region amino acid sequence that is at least 90%, 91%, 92%, 93%, 94%, 95%, 96%, 97%, 98% or 99% identical to an amino acid sequence selected from the group consisting of the light chain variable region sequences shown in Table 1.

In some embodiments, the activatable antibody comprises a combination of a variable heavy chain complementarity determining region 1 (VH CDR1, also referred to herein as CDRH1) sequence, a variable heavy chain complementarity determining region 2 (VH CDR2, also referred to herein as CDRH2) sequence, a variable heavy chain complementarity determining region 3 (VH CDR3, also referred to herein as CDRH3) sequence, a variable light chain complementarity determining region 1 (VL CDR1, also referred to herein as CDRL1) sequence, a variable light chain complementarity determining region 2 (VL CDR2, also referred to herein as CDRL2) sequence, and a variable light chain complementarity determining region 3 (VL CDR3, also referred to herein as CDRL3) sequence, wherein at least one CDR sequence is selected from the group consisting of a VH CDR1 sequence shown in Table 2; a VH CDR2 sequence shown in Table 2; a VH CDR3 sequence shown in Table 2; a VL CDR1 sequence shown in Table 2; a VL CDR2 sequence shown in Table 2; and a VL CDR3 sequence shown in Table 2.

In some embodiments, the activatable antibody comprises a combination of a VH CDR1 sequence, a VH CDR2 sequence, a VH CDR3 sequence, a VL CDR1 sequence, a VL CDR2 sequence, and a VL CDR3 sequence, wherein at least one CDR sequence is selected from the group consisting of a VH CDR1 sequence that includes a sequence that is at least 90%, 91%, 92%, 93%, 94%, 95%, 96%, 97%, 98%, 99% or more identical to a VH CDR1 sequence shown in Table 2; a VH CDR2 sequence that includes a sequence that is at least 90%, 91%, 92%, 93%, 94%, 95%, 96%, 97%, 98%, 99% or more identical to a VH CDR2 sequence shown in Table 2; a VH CDR3 sequence that includes a sequence that is at least 90%, 91%, 92%, 93%, 94%, 95%, 96%, 97%, 98%, 99% or more identical to a VH CDR3 sequence shown in Table 2; a VL CDR1 sequence that includes a sequence that is at least 90%, 91%, 92%, 93%, 94%, 95%, 96%, 97%, 98%, 99% or more identical to a VL CDR1 sequence shown in Table 2; a VL CDR2 sequence that includes a sequence that is at least 90%, 91%, 92%, 93%, 94%, 95%, 96%, 97%, 98%, 99% or more identical to a VL CDR2 sequence shown in Table 2; and a VL CDR3 sequence that includes a sequence that is at least 90%, 91%, 92%, 93%, 94/O, 95%, 96%, 97%, 98%, 99% or more identical to a VL CDR3 sequence shown in Table 2.

In some embodiments, the activatable antibody comprises a combination of a VH CDR1 sequence, a VH CDR2 sequence, a VH CDR3 sequence, a VL CDR1 sequence, a VL CDR2 sequence, and a VL CDR3 sequence, wherein the combination is a combination of the six CDR sequences (VH CDR1, VH CDR2, VH CDR3, VL CDR1, VL CDR2, and VL CDR3) shown in a single row in Table 2.

In some embodiments, the activatable antibody comprises a heavy chain that comprises a combination of a VH CDR1 sequence, a VH CDR2 sequence, and a VH CDR3 sequence, wherein the combination is a combination of the three heavy chain CDR sequences (VH CDR1, VH CDR2, VH CDR3) shown in a single row in Table 2.

In some embodiments, the activatable antibody comprises a light chain that comprises a combination of a VL CDR sequence, a VL CDR2 sequence, and a VL CDR3 sequence, wherein the combination is a combination of the three light chain CDR sequences (VL CDR1, VL CDR2, VL CDR3) shown in a single row in Table 2.

In some embodiments, the activatable antibody comprises a combination of a VH CDR1 sequence, a VH CDR2 sequence, a VH CDR3 sequence, a VL CDR1 sequence, a VL CDR2 sequence, and a VL CDR3 sequence, wherein each CDR sequence in the combination comprises a sequence that is at least 90%, 91%, 92%, 93%, 94%, 95%, 96%, 97%, 98%, 99% or more identical to the corresponding CDR sequence in a combination of the six CDR sequences (VH CDR1, VH CDR2, VH CDR3, VL CDR1, VL CDR2, and VL CDR3) shown in a single row in Table 2.

In some embodiments, the activatable antibody comprises a heavy chain variable region that comprises a combination of a VH CDR1 sequence, a VH CDR2 sequence, and a VH CDR3 sequence, wherein each CDR sequence in the combination comprises a sequence that is at least 90%, 91%, 92%, 93%, 94%, 95%, 96%, 97%, 98%, 99% or more identical to the corresponding CDR sequence in a combination of three heavy chain CDR sequences (VH CDR1, VH CDR2, VH CDR3) shown in a single row in Table 2.

In some embodiments, the activatable antibody comprises a light chain variable region that comprises a combination of a VL CDR1 sequence, a VL CDR2 sequence, and a VL CDR3 sequence, wherein each CDR sequence in the combination comprises a sequence that is at least 90%, 91%, 92%, 93%, 94%, 95%, 96%, 97%, 98%, 99% or more identical to the corresponding CDR sequence in a combination of three light chain CDR sequences (VL CDR1, VL CDR2, VL CDR3) shown in a single row in Table 2.

In some embodiments, the MM has a dissociation constant for binding to the AB which is greater than the dissociation constant of the AB to CD147.

In some embodiments, the MM has a dissociation constant for binding to the AB which is no more than the dissociation constant of the AB to CD147.

In some embodiments, the MM has a dissociation constant for binding to the AB is equivalent to the dissociation constant of the AB to CD147.

In some embodiments, the MM has a dissociation constant for binding to the AB which is less than the dissociation constant of the AB to CD147.

In some embodiments, the dissociation constant (K_{d}) of the MM towards the AB is no more than 2, 3, 4, 5, 10, 25, 50, 100, 250, 500, 1,000, 2,500, 5,000, 10,000, 50,000, 100,000, 500,000, 1,000,000, 5,000,000, 10,000,000, 50,000,000 times or greater, or between 1-5, 5-10, 10-100, 10-1,000, 10-10,000, 10-100,000, 10-1,000,000, 10-10,000,000, 100-1,000, 100-10,000, 100-100,000, 100-1,000,000, 100-10,000,000, 1,000-10,000, 1,000-100,000, 1,000-1,000,000, 1000-10,000,000, 10,000-100,000, 10,000-1,000,000, 10,000-10,000,000, 100,000-1,000,000, or 100,000-10,000,000 times or greater than the dissociation constant of the AB towards the CD147 target.

In some embodiments, the MM does not interfere or compete with the AB for binding to CD147 when the activatable antibody is in a cleaved state.

In some embodiments, the MM is a polypeptide of about 2 to 40 amino acids in length. In some embodiments, the MM is a polypeptide of up to about 40 amino acids in length.

In some embodiments, the MM polypeptide sequence is different from that of CD147. In some embodiments, the MM polypeptide sequence is no more than 50% identical to any natural binding partner of the AB. In some embodiments, the MM polypeptide sequence is different from that of CD147 and is no more than 40%, 30%, 25%, 20%, 15%, or 10% identical to any natural binding partner of the AB.

In some embodiments, the coupling of the MM to the AB reduces the ability of the AB to bind CD147 such that the dissociation constant (K_{d}) of the AB when coupled to the MM towards CD147 is at least two times greater than the K_{d} of the AB when not coupled to the MM towards CD147.

In some embodiments, the coupling of the MM to the AB reduces the ability of the AB to bind CD147 such that the dissociation constant (K_{d}) of the AB when coupled to the MM towards CD147 is at least five times greater than the K_{d} of the AB when not coupled to the MM towards CD147.

In some embodiments, the coupling of the MM to the AB reduces the ability of the AB to bind CD147 such that the dissociation constant (K_{d}) of the AB when coupled to the MM towards CD147 is at least 10 times greater than the K_{d} of the AB when not coupled to the MM towards CD147.

In some embodiments, the coupling of the MM to the AB reduces the ability of the AB to bind CD147 such that the dissociation constant (K_{d}) of the AB when coupled to the MM towards CD147 is at least 20 times greater than the K_{d} of the AB when not coupled to the MM towards CD147.

In some embodiments, the coupling of the MM to the AB reduces the ability of the AB to bind CD147 such that the dissociation constant (K_{d}) of the AB when coupled to the MM towards CD147 is at least 40 times greater than the K_{d} of the AB when not coupled to the MM towards CD147.

In some embodiments, the coupling of the MM to the AB reduces the ability of the AB to bind CD147 such that the dissociation constant (K_{d}) of the AB when coupled to the MM towards CD147 is at least 100 times greater than the K_{d} of the AB when not coupled to the MM towards CD147.

In some embodiments, the coupling of the MM to the AB reduces the ability of the AB to bind CD147 such that the dissociation constant (K_{d}) of the AB when coupled to the MM towards CD147 is at least 1000 times greater than the K_{d} of the AB when not coupled to the MM towards CD147.

In some embodiments, the coupling of the MM to the AB reduces the ability of the AB to bind CD147 such that the dissociation constant (K_{d}) of the AB when coupled to the MM towards CD147 is at least 10,000 times greater than the K_{d} of the AB when not coupled to the MM towards CD147.

In some embodiments, in the presence of CD147, the MM reduces the ability of the AB to bind CD147 by at least 90% when the CM is uncleaved, as compared to when the CM is cleaved when assayed in vitro using a target displacement assay such as, for example, the assay described in PCT Publication No. WO 2010/081173, the contents of which are hereby incorporated by reference in their entirety.

In some embodiments, MM comprises an amino acid sequence selected from the group consisting of SEQ ID NO: 30-100. In some embodiments, the MM comprises an amino acid sequence selected from the group consisting of SEQ ID NOs: 30, 31, 33, 44, 46, 75-80, 82, 83, 86-89, and 90-100. In some embodiments, the MM comprises an amino acid sequence selected from the group consisting of SEQ ID NOs: 30, 31, 33, 44, 46, 75, 76, 77, 78, 79, 80, 82, 83, 86, 87, 88, 89, 90, 91, 92, 93, 94, 96, 97, 98, 99, and 100,

In some embodiments, the protease that cleaves the CM is active, e.g., up-regulated or otherwise unregulated, in diseased tissue, and the protease cleaves the CM in the activatable antibody when the activatable antibody is exposed to the protease.

In some embodiments, the protease is co-localized with CD147 in a tissue, and the protease cleaves the CM in the activatable antibody when the activatable antibody is exposed to the protease.

In some embodiments, the CM is positioned in the activatable antibody such that when the activatable antibody is in the uncleaved state, binding of the activatable antibody to CD147 is reduced to occur with a dissociation constant that is at least twofold greater than the dissociation constant of an unmodified AB binding to CD147, whereas in the cleaved state (i.e., when the activatable antibody is in the cleaved state), the AB binds CD147.

In some embodiments, the CM is positioned in the activatable antibody such that when the activatable antibody is in the uncleaved state, binding of the activatable antibody to CD147 is reduced to occur with a dissociation constant that is at least fivefold greater than the dissociation constant of an unmodified AB binding to CD147, whereas in the cleaved state (i.e., when the activatable antibody is in the cleaved state), the AB binds CD147.

In some embodiments, the CM is positioned in the activatable antibody such that when the activatable antibody is in the uncleaved state, binding of the activatable antibody to CD147 is reduced to occur with a dissociation constant that is at least 10-fold greater than the dissociation constant of an unmodified AB binding to CD147, whereas in the cleaved state (i.e., when the activatable antibody is in the cleaved state), the AB binds CD147.

In some embodiments, the CM is positioned in the activatable antibody such that when the activatable antibody is in the uncleaved state, binding of the activatable antibody to CD147 is reduced to occur with a dissociation constant that is at least 20-fold greater than the dissociation constant of an unmodified AB binding to CD147, whereas in the cleaved state (i.e., when the activatable antibody is in the cleaved state), the AB binds CD147.

In some embodiments, the CM is positioned in the activatable antibody such that when the activatable antibody is in the uncleaved state, binding of the activatable antibody to CD147 is reduced to occur with a dissociation constant that is at least 40-fold greater than the dissociation constant of an unmodified AB binding to CD147, whereas in the cleaved state, the AB binds CD147.

In some embodiments, the CM is positioned in the activatable antibody such that when the activatable antibody is in the uncleaved state, binding of the activatable antibody to CD147 is reduced to occur with a dissociation constant that is at least 50-fold greater than the dissociation constant of an unmodified AB binding to CD147, whereas in the cleaved state, the AB binds CD147.

In some embodiments, the CM is positioned in the activatable antibody such that when the activatable antibody is in the uncleaved state, binding of the activatable antibody to CD147 is reduced to occur with a dissociation constant that is at least 100-fold greater than the dissociation constant of an unmodified AB binding to CD147, whereas in the cleaved state, the AB binds CD147.

In some embodiments, the CM is positioned in the activatable antibody such that when the activatable antibody is in the uncleaved state, binding of the activatable antibody to CD147 is reduced to occur with a dissociation constant that is at least 200-fold greater than the dissociation constant of an unmodified AB binding to CD147, whereas in the cleaved state, the AB binds CD147.

In some embodiments, the CM is a polypeptide of up to 15 amino acids in length.

In some embodiments, the CM is a polypeptide that includes a first cleavable moiety (CM1) that is a substrate for at least one matrix metalloprotease (MMP) and a second cleavable moiety (CM2) that is a substrate for at least one serine protease (SP). In some embodiments, each of the CM1 substrate sequence and the CM2 substrate sequence of the CM1-CM2 substrate is independently a polypeptide of up to 15 amino acids in length.

In some embodiments, the CM is a substrate for at least one protease that is or is believed to be up-regulated or otherwise unregulated in cancer.

In some embodiments, the CM is a substrate for at least one protease selected from the group consisting of a matrix metalloprotease (MMP), thrombin, a neutrophil elastase, a cysteine protease, legumain, and a serine protease, such as matriptase (MT-SP1), and urokinase (uPA). Without being bound by theory, it is believed that these proteases are up-regulated or otherwise unregulated in at least one of cancer.

Exemplary substrates include but are not limited to substrates cleavable by one or more of the following enzymes or proteases listed in Table 3.

In some embodiments, the CM is selected for use with a specific protease, for example a protease that is known to be co-localized with the target of the activatable antibody.

In some embodiments, the CM is a substrate for at least one MMP. Examples of MMPs include the MMPs listed in the Table 3. In some embodiments, the CM is a substrate for a protease selected from the group consisting of MMP 9, MMP14, MMP1, MMP3, MMP13, MMP17, MMP11, and MMP19. In some embodiments the CM is a substrate for MMP9. In some embodiments, the CM is a substrate for MMP14.

In some embodiments, the CM is a substrate that includes the sequence TGRGPSWV (SEQ ID NO: 356); SARGPSRW (SEQ ID NO: 357); TARGPSFK (SEQ ID NO: 358); LSGRSDNH (SEQ ID NO: 359); GGWHTGRN (SEQ ID NO: 360); HTGRSGAL (SEQ ID NO: 361); PLTGRSGG (SEQ ID NO: 362); AARGPAIH (SEQ ID NO: 363); RGPAFNPM (SEQ ID NO: 364); SSRGPAYL (SEQ ID NO: 365); RGPATPIM (SEQ ID NO: 366); RGPA (SEQ ID NO: 367); GGQPSGMWGW (SEQ ID NO: 368); FPRPLGITGL (SEQ ID NO: 369); VHMPLGFLGP (SEQ ID NO: 370); SPLTGRSG (SEQ ID NO: 371); SAGFSLPA (SEQ ID NO: 372); LAPLGLQRR (SEQ ID NO: 373); SGGPLGVR (SEQ ID NO: 374); PLGL (SEQ ID NO: 375); LSGRSGNH (SEQ ID NO: 789), SGRSANPRG (SEQ ID NO: 790); LSGRSDDH (SEQ ID NO: 791); LSGRSDIH (SEQ ID NO: 792); LSGRSDQH (SEQ ID NO: 793); LSGRSDTH (SEQ ID NO: 794); LSGRSDYH (SEQ ID NO: 795); LSGRSDNP (SEQ ID NO: 796); LSGRSANP (SEQ ID NO: 797); LSGRSANI (SEQ ID NO: 798); LSGRSDNI (SEQ ID NO: 799); MIAPVAYR (SEQ ID NO: 800); RPSPMWAY (SEQ ID NO: 801); WATPRPMR (SEQ ID NO: 802); FRLLDWQW (SEQ ID NO: 803); ISSGL (SEQ ID NO: 804); ISSGLLS (SEQ ID NO: 805); and/or ISSGLL (SEQ ID NO: 806).

In some embodiments, the CM comprises the amino acid sequence LSGRSDNH (SEQ ID NO: 359). In some embodiments, the CM comprises the amino acid sequence TGRGPSWV (SEQ ID NO: 356). In some embodiments, the CM comprises the amino acid sequence PLTGRSGG (SEQ ID NO: 362). In some embodiments, the CM comprises the amino acid sequence GGQPSGMWGW (SEQ ID NO: 368). In some embodiments, the CM comprises the amino acid sequence FPRPLGITGL (SEQ ID NO: 369). In some embodiments, the CM comprises the amino acid sequence VHMPLGFLGP (SEQ ID NO: 370). In some embodiments, the CM comprises the amino acid sequence PLGL (SEQ ID NO: 375). In some embodiments, the CM comprises the amino acid sequence SARGPSRW (SEQ ID NO: 357). In some embodiments, the CM comprises the amino acid sequence TARGPSFK (SEQ ID NO: 358). In some embodiments, the CM comprises the amino acid sequence GGWHTGRN (SEQ ID NO: 360). In some embodiments, the CM comprises the amino acid sequence HTGRSGAL (SEQ ID NO: 361). In some embodiments, the CM comprises the amino acid sequence AARGPAIH (SEQ ID NO: 363). In some embodiments, the CM comprises the amino acid sequence RGPAFNPM (SEQ ID NO: 364). In some embodiments, the CM comprises the amino acid sequence SSRGPAYL (SEQ ID NO: 365). In some embodiments, the CM comprises the amino acid sequence RGPATPIM (SEQ ID NO: 366). In some embodiments, the CM comprises the amino acid sequence RGPA (SEQ ID NO: 367). In some embodiments, the CM comprises the amino acid sequence LSGRSGNH (SEQ ID NO: 789). In some embodiments, the CM comprises the amino acid sequence SGRSANPRG (SEQ ID NO: 790). In some embodiments, the CM comprises the amino acid sequence LSGRSDDH (SEQ ID NO: 791). In some embodiments, the CM comprises the amino acid sequence LSGRSDIH (SEQ ID NO: 792). In some embodiments, the CM comprises the amino acid sequence LSGRSDQH (SEQ ID NO: 793). In some embodiments, the CM comprises the amino acid sequence LSGRSDTH (SEQ ID NO: 794). In some embodiments, the CM comprises the amino acid sequence LSGRSDYH (SEQ ID NO: 795). In some embodiments, the CM comprises the amino acid sequence LSGRSDNP (SEQ ID NO: 796). In some embodiments, the CM comprises the amino acid sequence LSGRSANP (SEQ ID NO: 797). In some embodiments, the CM comprises the amino acid sequence LSGRSANI (SEQ ID NO: 798). In some embodiments, the CM comprises the amino acid sequence LSGRSDNI (SEQ ID NO: 799). In some embodiments, the CM comprises the amino acid sequence MIAPVAYR (SEQ ID NO: 800). In some embodiments, the CM comprises the amino acid sequence RPSPMWAY (SEQ ID NO: 801). In some embodiments, the CM comprises the amino acid sequence WATPRPMR (SEQ ID NO: 802). In some embodiments, the CM comprises the amino acid sequence FRLLDWQW (SEQ ID NO: 803). In some embodiments, the CM comprises the amino acid sequence ISSGL (SEQ ID NO: 804). In some embodiments, the CM comprises the amino acid sequence ISSGLLS (SEQ ID NO: 805). In some embodiments, the CM comprises the amino acid sequence and/or ISSGLL (SEQ ID NO: 806).

In some embodiments, the CM is a substrate for an MMP and includes the sequence ISSGLSS (SEQ ID NO: 376); QNQALRMA (SEQ ID NO: 377); AQNLLGMV (SEQ ID NO: 378); STFPFGMF (SEQ ID NO: 379); PVGYTSSL (SEQ ID NO: 380); DWLYWPGI (SEQ ID NO: 381), ISSGLLSS (SEQ ID NO: 382), LKAAPRWA (SEQ ID NO: 383); GPSHLVLT (SEQ ID NO: 384); LPGGLSPW (SEQ ID NO: 385); MGLFSEAG (SEQ ID NO: 386); SPLPLRVP (SEQ ID NO: 387); RMHLRSLG (SEQ ID NO: 388); LAAPLGLL (SEQ ID NO: 389); AVGLLAPP (SEQ ID NO: 390); LLAPSHRA (SEQ ID NO: 391); and/or PAGLWLDP (SEQ ID NO: 392).

In some embodiments, the CM comprises the amino acid sequence ISSGLSS (SEQ ID NO: 376). In some embodiments, the CM comprises the amino acid sequence QNQALRMA (SEQ ID NO: 377). In some embodiments, the CM comprises the amino acid sequence AQNLLGMV (SEQ ID NO: 378). In some embodiments, the CM comprises the amino acid sequence STFPFGMF (SEQ ID NO: 379). In some embodiments, the CM comprises the amino acid sequence PVGYTSSL (SEQ ID NO: 380). In some embodiments, the CM comprises the amino acid sequence DWLYWPGI (SEQ ID NO: 381). In some embodiments, the CM comprises the amino acid sequence ISSGLLSS (SEQ ID NO: 382). In some embodiments, the CM comprises the amino acid sequence LKAAPRWA (SEQ ID NO: 383). In some embodiments, the CM comprises the amino acid sequence GPSHLVLT (SEQ ID NO: 384). In some embodiments, the CM comprises the amino acid sequence LPGGLSPW (SEQ ID NO: 385). In some embodiments, the CM comprises the amino acid sequence MGLFSEAG (SEQ ID NO: 386). In some embodiments, the CM comprises the amino acid sequence SPLPLRVP (SEQ ID NO: 387). In some embodiments, the CM comprises the amino acid sequence RMHLRSLG (SEQ ID NO: 388). In some embodiments, the CM comprises the amino acid sequence LAAPLGLL (SEQ ID NO: 389). In some embodiments, the CM comprises the amino acid sequence AVGLLAPP (SEQ ID NO: 390). In some embodiments, the CM comprises the amino acid sequence LLAPSHRA (SEQ ID NO: 391). In some embodiments, the CM comprises the amino acid sequence PAGLWLDP (SEQ ID NO: 392).

In some embodiments, the CM is a substrate for thrombin. In some embodiments, the CM is a substrate for thrombin and includes the sequence GPRSFGL (SEQ ID NO: 393) or GPRSFG (SEQ ID NO: 394). In some embodiments, the CM comprises the amino acid sequence GPRSFGL (SEQ ID NO: 393). In some embodiments, the CM comprises the amino acid sequence GPRSFG (SEQ ID NO: 394).

In some embodiments, the CM comprises an amino acid sequence selected from the group consisting of NTLSGRSENHSG (SEQ ID NO: 395); NTLSGRSGNHGS (SEQ ID NO: 396); TSTSGRSANPRG (SEQ ID NO: 397); TSGRSANP (SEQ ID NO: 398); VAGRSMRP (SEQ ID NO: 399); VVPEGRRS (SEQ ID NO: 400); ILPRSPAF (SEQ ID NO: 401); MVLGRSLL (SEQ ID NO: 402); QGRAITFI (SEQ ID NO: 403); SPRSIMLA (SEQ ID NO: 404); and SMLRSMPL (SEQ ID NO: 405).

In some embodiments, the CM comprises the amino acid sequence NTLSGRSENHSG (SEQ ID NO: 395). In some embodiments, the CM comprises the amino acid sequence NTLSGRSGNHGS (SEQ ID NO: 396). In some embodiments, the CM comprises the amino acid sequence TSTSGRSANPRG (SEQ ID NO: 397). In some embodiments, the CM comprises the amino acid sequence TSGRSANP (SEQ ID NO: 398). In some embodiments, the CM comprises the amino acid sequence VAGRSMRP (SEQ ID NO: 399). In some embodiments, the CM comprises the amino acid sequence VVPEGRRS (SEQ ID NO: 400). In some embodiments, the CM comprises the amino acid sequence ILPRSPAF (SEQ ID NO: 401). In some embodiments, the CM comprises the amino acid sequence MVLGRSLL (SEQ ID NO: 402). In some embodiments, the CM comprises the amino acid sequence QGRAITFI (SEQ ID NO: 403). In some embodiments, the CM comprises the amino acid sequence SPRSIMLA (SEQ ID NO: 404). In some embodiments, the CM comprises the amino acid sequence SMLRSMPL (SEQ ID NO: 405).

In some embodiments, the CM is a substrate for a neutrophil elastase. In some embodiments, the CM is a substrate for a serine protease. In some embodiments, the CM is a substrate for uPA. In some embodiments, the CM is a substrate for legumain. In some embodiments, the CM is a substrate for matriptase. In some embodiments, the CM is a substrate for a cysteine protease. In some embodiments, the CM is a substrate for a cysteine protease, such as a cathepsin.

In some embodiments, the CM is a CM1-CM2 substrate and includes the sequence ISSGLLSGRSDNH (SEQ ID NO: 406); ISSGLLSSGGSGGSLSGRSDNH (SEQ ID NO: 407); AVGLLAPPGGTSTSGRSANPRG (SEQ ID NO: 408); TSTSGRSANPRGGGAVGLLAPP (SEQ ID NO: 409); VHMPLGFLGPGGTSTSGRSANPRG (SEQ ID NO: 410); TSTSGRSANPRGGGVHMPLGFLGP (SEQ ID NO: 411); AVGLLAPPGGLSGRSDNH (SEQ ID NO: 412); LSGRSDNHGGAVGLLAPP (SEQ ID NO: 413); VHMPLGFLGPGGLSGRSDNH (SEQ ID NO: 414); LSGRSDNHGGVHMPLGFLGP (SEQ ID NO: 415); LSGRSDNHGGSGGSISSGLLSS (SEQ ID NO: 416); LSGRSGNHGGSGGSISSGLLSS (SEQ ID NO: 417); ISSGLLSSGGSGGSLSGRSGNH (SEQ ID NO: 418); LSGRSDNHGGSGGSQNQALRMA (SEQ ID NO: 419); QNQALRMAGGSGGSLSGRSDNH (SEQ ID NO: 420); LSGRSGNHGGSGGSQNQALRMA (SEQ ID NO: 421); QNQALRMAGGSGGSLSGRSGNH (SEQ ID NO: 422); ISSGLLSGRSGNH (SEQ ID NO: 423); ISSGLLSGRSANPRG (SEQ ID NO: 680); AVGLLAPPTSGRSANPRG (SEQ ID NO: 681); AVGLLAPPSGRSANPRG (SEQ ID NO: 682); ISSGLLSGRSDDH (SEQ ID NO: 683); ISSGLLSGRSDIH (SEQ ID NO: 684); ISSGLLSGRSDQH (SEQ ID NO: 685); ISSGLLSGRSDTH (SEQ ID NO: 686); ISSGLLSGRSDYH (SEQ ID NO: 687); ISSGLLSGRSDNP (SEQ ID NO: 688); ISSGLLSGRSANP (SEQ ID NO: 689); ISSGLLSGRSANI (SEQ ID NO: 690); AVGLLAPPGGLSGRSDDH (SEQ ID NO: 691); AVGLLAPPGGLSGRSDIH (SEQ ID NO: 692); AVGLLAPPGGLSGRSDQH (SEQ ID NO: 693); AVGLLAPPGGLSGRSDTH (SEQ ID NO: 694); AVGLLAPPGGLSGRSDYH (SEQ ID NO: 695); AVGLLAPPGGLSGRSDNP (SEQ ID NO: 696); AVGLLAPPGGLSGRSANP (SEQ ID NO: 697); AVGLLAPPGGLSGRSANI (SEQ ID NO: 698), ISSGLLSGRSDNI (SEQ ID NO: 713); AVGLLAPPGGLSGRSDNI (SEQ ID NO: 714); GLSGRSDNHGGAVGLLAPP (SEQ ID NO: 807); and/or GLSGRSDNHGGVHMPLGFLGP (SEQ ID NO: 808).

In some embodiments, the CM1-CM2 substrate includes the sequence ISSGLLSGRSDNH (SEQ ID NO: 406), which is also referred to herein as substrate 2001. In some embodiments, the CM1-CM2 substrate includes the sequence ISSGLLSSGGSGGSLSGRSDNH (SEQ ID NO: 407), which is also referred to herein as substrate 1001/LP′/0001, where LP′ as used in this CM1-CM2 substrate is the amino acid sequence GGSGGS (SEQ ID NO: 1037). In some embodiments, the CM1-CM2 substrate includes the sequence AVGLLAPPGGTSTSGRSANPRG (SEQ ID NO: 408), which is also referred to herein as substrate 2015 and/or substrate 1004/LP′/0003, where LP′ as used in this CM1-CM2 substrate is the amino acid sequence GG. In some embodiments, the CM1-CM2 substrate includes the sequence TSTSGRSANPRGGGAVGLLAPP (SEQ ID NO: 409), which is also referred to herein as substrate 0003/LP′/1004, where LP′ as used in this CM1-CM2 substrate is the amino acid sequence GG. In some embodiments, the CM1-CM2 substrate includes the sequence VHMPLGFLGPGGTSTSGRSANPRG (SEQ ID NO: 410), which is also referred to herein as substrate 1003/LP′/0003, where LP′ as used in this CM1-CM2 substrate is the amino acid sequence GG. In some embodiments, the CM1-CM2 substrate includes the sequence TSTSGRSANPRGGGVHMPLGFLGP (SEQ ID NO: 411), which is also referred to herein as substrate 0003/LP′/1003, where LP′ as used in this CM1-CM2 substrate is the amino acid sequence GG. In some embodiments, the CM1-CM2 substrate includes the sequence AVGLLAPPGGLSGRSDNH (SEQ ID NO: 412), which is also referred to herein as substrate 3001 and/or substrate 1004/LP′/0001, where LP′ as used in this CM1-CM2 substrate is the amino acid sequence GG. In some embodiments, the CM1-CM2 substrate includes the sequence LSGRSDNHGGAVGLLAPP (SEQ ID NO: 413), which is also referred to herein as substrate 0001/LP′/1004, where LP′ as used in this CM1-CM2 substrate is the amino acid sequence GG. In some embodiments, the CM1-CM2 substrate includes the sequence VHMPLGFLGPGGLSGRSDNH (SEQ ID NO: 414), which is also referred to herein as substrate 1003/LP′/0001, wherein LP′ as used in this CM1-CM2 substrate is the amino acid sequence GG. In some embodiments, the CM1-CM2 substrate includes the sequence LSGRSDNHGGVHMPLGFLGP (SEQ ID NO: 415), which is also referred to herein as substrate 0001/LP′/1003, where LP′ as used in this CM1-CM2 substrate is the amino acid sequence GG. In some embodiments, the CM1-CM2 substrate includes the sequence LSGRSDNHGGSGGSISSGLLSS (SEQ ID NO: 416), which is also referred to herein as substrate 0001/LP′/1001, where LP′ as used in this CM1-CM2 substrate is the amino acid sequence GGSGGS (SEQ ID NO: 1037). In some embodiments, the CM1-CM2 substrate includes the sequence LSGRSGNHGGSGGSISSGLLSS (SEQ ID NO: 417), which is also referred to herein as substrate 0002/LP′/1001, where LP′ as used in this CM1-CM2 substrate is the amino acid sequence GGSGGS (SEQ ID NO: 1037). In some embodiments, the CM1-CM2 substrate includes the sequence ISSGLLSSGGSGGSLSGRSGNH (SEQ ID NO: 418), which is also referred to herein as substrate 1001/LP′/0002, where LP′ as used in this CM1-CM2 substrate is the amino acid sequence GGSGGS (SEQ ID NO: 1037). In some embodiments, the CM1-CM2 substrate includes the sequence LSGRSDNHGGSGGSQNQALRMA (SEQ ID NO: 419), which is also referred to herein as substrate 0001/LP′/1002, where LP′ as used in this CM1-CM2 substrate is the amino acid sequence GGSGGS (SEQ ID NO: 1037). In some embodiments, the CM1-CM2 substrate includes the sequence QNQALRMAGGSGGSLSGRSDNH (SEQ ID NO: 420), which is also referred to herein as substrate 1002/LP′/0001, where LP′ as used in this CM1-CM2 substrate is the amino acid sequence GGSGGS (SEQ ID NO: 1037). In some embodiments, the CM1-CM2 substrate includes the sequence LSGRSGNHGGSGGSQNQALRMA (SEQ ID NO: 421), which is also referred to herein as substrate 0002/LP′/1002, where LP′ as used in this CM1-CM2 substrate is the amino acid sequence GGSGGS (SEQ ID NO: 1037). In some embodiments, the CM1-CM2 substrate includes the sequence QNQALRMAGGSGGSLSGRSGNH (SEQ ID NO: 422), which is also referred to herein as substrate 1002/LP′/0002, where LP′ as used in this CM1-CM2 substrate is the amino acid sequence GGSGGS (SEQ ID NO: 1037). In some embodiments, the CM1-CM2 substrate includes the sequence ISSGLLSGRSGNH (SEQ ID NO: 423), which is also referred to herein as substrate 2002. In some embodiments, the CM1-CM2 substrate includes the sequence ISSGLLSGRSANPRG (SEQ ID NO: 680), which is also referred to herein as substrate 2003. In some embodiments, the CM1-CM2 substrate includes the sequence AVGLLAPPTSGRSANPRG (SEQ ID NO: 681), which is also referred to herein as substrate 2004. In some embodiments, the CM1-CM2 substrate includes the sequence AVGLLAPPSGRSANPRG (SEQ ID NO: 682), which is also referred to herein as substrate 2005. In some embodiments, the CM1-CM2 substrate includes the sequence ISSGLLSGRSDDH (SEQ ID NO: 683), which is also referred to herein as substrate 2006. In some embodiments, the CM1-CM2 substrate includes the sequence ISSGLLSGRSDIH (SEQ ID NO: 684), which is also referred to herein as substrate 2007. In some embodiments, the CM1-CM2 substrate includes the sequence ISSGLLSGRSDQH (SEQ ID NO: 685), which is also referred to herein as substrate 2008. In some embodiments, the CM1-CM2 substrate includes the sequence ISSGLLSGRSDTH (SEQ ID NO: 686), which is also referred to herein as substrate 2009. In some embodiments, the CM1-CM2 substrate includes the sequence ISSGLLSGRSDYH (SEQ ID NO: 687), which is also referred to herein as substrate 2010. In some embodiments, the CM1-CM2 substrate includes the sequence ISSGLLSGRSDNP (SEQ ID NO: 688), which is also referred to herein as substrate 2011. In some embodiments, the CM1-CM2 substrate includes the sequence ISSGLLSGRSANP (SEQ ID NO: 689), which is also referred to herein as substrate 2012. In some embodiments, the CM1-CM2 substrate includes the sequence ISSGLLSGRSANI (SEQ ID NO: 690), which is also referred to herein as substrate 2013. In some embodiments, the CM1-CM2 substrate includes the sequence AVGLLAPPGGLSGRSDDH (SEQ ID NO: 691), which is also referred to herein as substrate 3006. In some embodiments, the CM1-CM2 substrate includes the sequence AVGLLAPPGGLSGRSDIH (SEQ ID NO: 692), which is also referred to herein as substrate 3007. In some embodiments, the CM1-CM2 substrate includes the sequence AVGLLAPPGGLSGRSDQH (SEQ ID NO: 693), which is also referred to herein as substrate 3008. In some embodiments, the CM1-CM2 substrate includes the sequence AVGLLAPPGGLSGRSDTH (SEQ ID NO: 694), which is also referred to herein as substrate 3009. In some embodiments, the CM1-CM2 substrate includes the sequence AVGLLAPPGGLSGRSDYH (SEQ ID NO: 695), which is also referred to herein as substrate 3010. In some embodiments, the CM1-CM2 substrate includes the sequence AVGLLAPPGGLSGRSDNP (SEQ ID NO: 696), which is also referred to herein as substrate 3011. In some embodiments, the CM1-CM2 substrate includes the sequence AVGLLAPPGGLSGRSANP (SEQ ID NO: 697), which is also referred to herein as substrate 3012. In some embodiments, the CM1-CM2 substrate includes the sequence AVGLLAPPGGLSGRSANI (SEQ ID NO: 698), which is also referred to herein as substrate 3013. In some embodiments, the CM1-CM2 substrate includes the sequence ISSGLLSGRSDNI (SEQ ID NO: 713), which is also referred to herein as substrate 2014. In some embodiments, the CM1-CM2 substrate includes the sequence AVGLLAPPGGLSGRSDNI (SEQ ID NO: 714), which is also referred to herein as substrate 3014. In some embodiments, the CM1-CM2 substrate includes the sequence GLSGRSDNHGGAVGLLAPP (SEQ ID NO: 807), which is also referred to herein as substrate 0001/LP′/1004, where LP′ as used in this CM1-CM2 substrate is the amino acid sequence GG. In some embodiments, the CM1-CM2 substrate includes the sequence GLSGRSDNHGGVHMPLGFLGP (SEQ ID NO: 808), which is also referred to herein as substrate 0001/LP′/1003, where LP′ as used in this CM1-CM2 substrate is the amino acid sequence GG.

In some embodiments, the CM is a substrate for at least two proteases. In some embodiments, each protease is selected from the group consisting of those shown in Table 3. In some embodiments, the CM is a substrate for at least two proteases, wherein one of the proteases is selected from the group consisting of a MMP, thrombin, a neutrophil elastase, a cysteine protease, uPA, legumain and matriptase and the other protease is selected from the group consisting of those shown in Table 3. In some embodiments, the CM is a substrate for at least two proteases selected from the group consisting of a MMP, thrombin, a neutrophil elastase, a cysteine protease, uPA, legumain and matriptase.

In some embodiments, the activatable antibody includes at least a first CM and a second CM. In some embodiments, the first CM and the second CM are each polypeptides of no more than 15 amino acids long. In some embodiments, the first CM and the second CM in the activatable antibody in the uncleaved state have the structural arrangement from N-terminus to C-terminus as follows: MM-CM1-CM2-AB or AB-CM2-CM1-MM. In some embodiments, at least one of the first CM and the second CM is a polypeptide that functions as a substrate for a protease selected from the group consisting of a MMP, thrombin, a neutrophil elastase, a cysteine protease, uPA, legumain, and matriptase. In some embodiments, the first CM is cleaved by a first cleaving agent selected from the group consisting of a MMP, thrombin, a neutrophil elastase, a cysteine protease, uPA, legumain, and matriptase in a target tissue and the second CM is cleaved by a second cleaving agent in a target tissue. In some embodiments, the other protease is selected from the group consisting of those shown in Table 3. In some embodiments, the first cleaving agent and the second cleaving agent are the same protease selected from the group consisting of a MMP, thrombin, a neutrophil elastase, a cysteine protease, uPA, legumain, and matriptase, and the first CM and the second CM are different substrates for the enzyme. In some embodiments, the first cleaving agent and the second cleaving agent are the same protease selected from the group consisting of those shown in Table 3. In some embodiments, the first cleaving agent and the second cleaving agent are different proteases. In some embodiments, the first cleaving agent and the second cleaving agent are co-localized in the target tissue. In some embodiments, the first CM and the second CM are cleaved by at least one cleaving agent in the target tissue.

In some embodiments, the activatable antibody is exposed to and cleaved by a protease such that, in the activated or cleaved state, the activated antibody includes a light chain amino acid sequence that includes at least a portion of LP2 and/or CM sequence after the protease has cleaved the CM.

Suitable activatable CD147 antibodies of the disclosure also include an antibody or antigen binding fragment thereof that binds to the same epitope on human CD147 and/or cynomolgus monkey CD147 as a CD147 antibody comprising a heavy chain variable region amino acid sequence selected from the group consisting of SEQ ID NOs: 1-4, and a light chain variable region amino acid sequence selected from the group consisting of SEQ ID NOs: 5-9.

Suitable activatable CD147 antibodies of the disclosure also include an antibody or antigen binding fragment thereof that binds to the same epitope on human CD147 and/or cynomolgus monkey CD147 as a CD147 antibody comprising a VH CDR1 sequence comprising the amino acid sequence GFTFSNYWMN (SEQ ID NO: 10) or GFTFSNYWMD (SEQ ID NO: 11); a VH CDR2 sequence comprising the amino acid sequence EIRLKSYNYATH (SEQ ID NO: 12); a VH CDR3 sequence comprising the amino acid sequence AGTDY (SEQ ID NO: 13); a VL CDR1 sequence comprising the amino acid sequence KASQSVRTDVA (SEQ ID NO: 14) or RASQSVRTDVG (SEQ ID NO: 15); a VL CDR2 sequence comprising the amino acid sequence YSSNRYT (SEQ ID NO: 16); and a VL CDR3 sequence comprising the amino acid sequence QQDYSSPFT (SEQ ID NO: 17) or QQDYSSPYT (SEQ ID NO: 18).

Suitable activatable CD147 antibodies of the disclosure also include an antibody or antigen-binding fragment thereof that binds to the same epitope on human CD147 and/or cynomolgus monkey CD147 as a CD147 antibody comprising a heavy chain variable region amino acid sequence selected from the group consisting of the heavy chain variable region sequences shown in Table 1 and a light chain variable region amino acid sequence selected from the group consisting of the light chain variable region sequences shown in Table 1.

Suitable activatable CD147 antibodies of the disclosure also include an antibody or antigen-binding fragment thereof that binds to the same epitope on human CD147 and/or cynomolgus monkey CD147 as a CD147 antibody comprising a combination of a VH CDR1 sequence, a VH CDR2 sequence, a VH CDR3 sequence, a VL CDR1 sequence, a VL CDR2 sequence, and a VL CDR3 sequence, wherein the combination is a combination of the six CDR sequences (VH CDR1, VH CDR2, VH CDR3, VL CDR1, VL CDR2, and VL CDR3) shown in a single row in Table 2.

Suitable activatable CD147 antibodies of the disclosure also include an antibody or antigen binding fragment thereof that cross-competes for binding to (inhibits the binding of) human CD147 and/or cynomolgus monkey CD147 to a CD147 antibody comprising a heavy chain variable region amino acid sequence selected from the group consisting of SEQ ID NOs: 1-4, and a light chain variable region amino acid sequence selected from the group consisting of SEQ ID NOs: 5-9.

Suitable activatable CD147 antibodies of the disclosure also include an antibody or antigen binding fragment thereof that cross-competes for binding to (inhibits the binding of) human CD147 and/or cynomolgus monkey CD147 to a CD147 antibody comprising a VH CDR1 sequence comprising the amino acid sequence GFTFSNYWMN (SEQ ID NO: 10) or GFTFSNYWMD (SEQ ID NO: 11); a VH CDR2 sequence comprising the amino acid sequence EIRLKSYNYATH (SEQ ID NO: 12); a VH CDR3 sequence comprising the amino acid sequence AGTDY (SEQ ID NO: 13); a VL CDR1 sequence comprising the amino acid sequence KASQSVRTDVA (SEQ ID NO: 14) or RASQSVRTDVG (SEQ ID NO: 15); a VL CDR2 sequence comprising the amino acid sequence YSSNRYT (SEQ ID NO: 16); and a VL CDR3 sequence comprising the amino acid sequence QQDYSSPFT (SEQ ID NO: 17) or QQDYSSPYT (SEQ ID NO: 18).

Suitable activatable CD147 antibodies of the disclosure also include an antibody or antigen-binding fragment thereof that cross-competes for binding to (inhibits the binding of) human CD147 and/or cynomolgus monkey CD147 as a CD147 antibody comprising a heavy chain variable region amino acid sequence selected from the group consisting of the heavy chain variable region sequences shown in Table 1 and a light chain variable region amino acid sequence selected from the group consisting of the light chain variable region sequences shown in Table 1.

Suitable activatable CD147 antibodies of the disclosure also include an antibody or antigen-binding fragment thereof that cross-competes for binding to (inhibits the binding of) human CD147 and/or cynomolgus monkey CD147 as a CD147 antibody comprising a combination of a VH CDR1 sequence, a VH CDR2 sequence, a VH CDR3 sequence, a VL CDR1 sequence, a VL CDR2 sequence, and a VL CDR3 sequence, wherein the combination is a combination of the six CDR sequences (VH CDR1, VH CDR2, VH CDR3, VL CDR1, VL CDR2, and VL CDR3) shown in a single row in Table 2.

In some embodiments, the activatable CD147 antibody is an activatable antibody that, in an activated state, binds CD147 comprising: an antibody or an antigen binding fragment thereof (AB) that specifically binds to mammalian CD147, wherein the AB specifically binds human CD147 and cynomolgus monkey CD147; a masking moiety (MM) that inhibits the binding of the AB to CD147 when the activatable antibody is in an uncleaved state; and a cleavable moiety (CM) coupled to the AB, wherein the CM is a polypeptide that functions as a substrate for a protease.

In some embodiments, the MM has a dissociation constant for binding to the AB that is greater than the dissociation constant of the AB to CD147. In some embodiments, the MM does not interfere or compete with the AB for binding to CD147 when the activatable antibody is in a cleaved state. In some embodiments, the MM is a polypeptide of no more than 40 amino acids in length. In some embodiments, the MM polypeptide sequence is different from that of human CD147. In some embodiments, the MM polypeptide sequence is no more than 50% identical to any natural binding partner of the AB. In some embodiments, the MM comprises an amino acid sequence selected from the group consisting of SEQ ID NOs: 30-100. In some embodiments, the MM comprises an amino acid sequence selected from the group consisting of SEQ ID NOs: 30, 31, 33, 44, 46, 75-80, 82, 83, 86-89, and 90-100.

In some embodiments, the CM is a substrate for a protease that is active in diseased tissue. In some embodiments, the CM comprises an amino acid sequence selected from the group consisting of SEQ ID NOs: 356-423, 680-698, 713, 714, and 789-808. In some embodiments, the CM comprises an amino acid sequence selected from the group consisting of SEQ ID NOs: 359, 370, 377, 382, 390, 397, 406-423, 680-698, 713, 714, and 807-808.

In some embodiments, the activatable antibody comprises an antigen binding fragment thereof is selected from the group consisting of a Fab fragment, a F(ab′)2 fragment, a scFv, a scAb, a dAb, a single domain heavy chain antibody, and a single domain light chain antibody. In some embodiments, the AB of the activatable antibody specifically binds human CD147. In some embodiments, the AB comprises the VH CDR1 sequence GFTFSNYWMN (SEQ ID NO: 10) or GFTFSNYWMD (SEQ ID NO: 11); the VH CDR2 sequence EIRLKSYNYATH (SEQ ID NO: 12); the VH CDR3 sequence AGTDY (SEQ ID NO: 13); the VL CDR1 sequence KASQSVRTDVA (SEQ ID NO: 14) or RASQSVRTDVG (SEQ ID NO: 15); the VL CDR2 sequence YSSNRYT (SEQ ID NO: 16); and the VL CDR3 sequence QQDYSSPFT (SEQ ID NO: 17) or QQDYSSPYT (SEQ ID NO: 18). In some embodiments, the AB comprises a heavy chain variable region comprising an amino acid sequence selected from the group consisting of SEQ ID NOs: 1-4, and a light chain variable region comprising an amino acid sequence selected from the group consisting of SEQ ID NOs: 5-9.

In some embodiments, the AB is linked to the CM. In some embodiments, the AB is linked directly to the CM. In some embodiments, the AB is linked to the CM via a linking peptide. In some embodiments, the MM is linked to the CM such that the activatable antibody in an uncleaved state comprises the structural arrangement from N-terminus to C-terminus as follows: MM-CM-AB or AB-CM-MM. In some embodiments, the activatable antibody comprises a linking peptide between the MM and the CM. In some embodiments, the activatable antibody comprises a linking peptide between the CM and the AB. In some embodiments, the activatable antibody comprises a first linking peptide (LP1) and a second linking peptide (LP2), and wherein the activatable antibody in the uncleaved state has the structural arrangement from N-terminus to C-terminus as follows: MM-LP1-CM-LP2-AB or AB-LP2-CM-LP1-MM. In some embodiments, the two linking peptides need not be identical to each other. In some embodiments, each of LP1 and LP2 is a peptide of about 1 to 20 amino acids in length.

In some embodiments, the activatable antibody comprises the heavy chain sequence selected from the group consisting of SEQ ID NOs: 1-4 and 19-22 and a light chain comprising an amino acid sequence selected from the group consisting of SEQ ID NOs: 5-9, 23-27, 140-182, 185-227, 230-272, and 275-317.

In some embodiments, the activatable antibody comprises a combination of amino acid sequences, wherein the combination of amino acid sequences is selected from a single row in Table 4, wherein for a given combination, (a) the heavy chain of the AB comprises the amino acid sequences of the VH CDR sequences corresponding to the given combination in the single row listed in Table 4, (b) the light chain of the AB comprises the amino acid sequences of the VL CDR sequences corresponding to the given combination in the single row listed in Table 4, (c) the MM comprises the amino acid sequence of the mask sequence (MM) corresponding to the given combination in the single row listed in Table 4, and (d) the CM comprises the amino acid sequence of the substrate sequence (CM) corresponding to the given combination in the single row listed in Table 4.

In some embodiments, the activatable antibody comprises a combination of amino acid sequences, wherein for a given combination of amino acid sequences, (a) the heavy chain of the AB comprises the amino acid sequences of the VH sequence or VH CDR sequences selected from the group consisting of: the VH sequence or VH CDR sequences listed in the corresponding column of Table 5, (b) the light chain of the AB comprises the amino acid sequences of the VL sequence or VL CDR sequences selected from the group consisting of: the VL sequence or VL CDR sequences listed in the corresponding column of Table 5, (c) the MM comprises the amino acid sequence of the mask sequence (MM) selected from the group consisting of: the MM sequences listed in the corresponding column of Table 5, and (d) the CM comprises the amino acid sequence of the substrate sequence (CM) selected from the group consisting of: the CM sequences listed in the corresponding column of Table 5.

In some embodiments, the activatable CD147 antibody comprises an antibody or an antigen binding fragment thereof (AB) that specifically binds to mammalian CD147, a MM, and a CM, wherein the activatable antibody comprises: a heavy chain sequence of SEQ ID NOs: 19-22; and a light chain comprising an amino acid sequence selected from the group consisting of SEQ ID NOs: 5-9 and 23-27. In some embodiments, the MM comprises an amino acid sequence selected from the group consisting of SEQ ID NOs: 30-100, and the CM comprises an amino acid sequence selected from the group consisting of SEQ ID NOs: 359, 370, 377, 382, 390, 397, 406-423, 680-698, 713, 714, and 789-808. In some embodiments, the AB comprises the VH CDR1 sequence GFTFSNYWMN (SEQ ID NO: 10) or GFTFSNYWMD (SEQ ID NO: 11); the VH CDR2 sequence EIRLKSYNYATH (SEQ ID NO: 12); the VH CDR3 sequence AGTDY (SEQ ID NO: 13); the VL CDR1 sequence KASQSVRTDVA (SEQ ID NO: 14) or RASQSVRTDVG (SEQ ID NO: 15); the VL CDR2 sequence YSSNRYT (SEQ ID NO: 16), and the VL CDR3 sequence QQDYSSPFT (SEQ ID NO: 17) or QQDYSSPYT (SEQ ID NO: 18). In some embodiments, the AB comprises a heavy chain variable region comprising an amino acid sequence selected from the group consisting of SEQ ID NOs: 1-3, and a light chain variable region comprising an amino acid sequence selected from the group consisting of SEQ ID NOs: 5-9.

In some embodiments, the activatable CD147 antibody comprises an antibody or an antigen binding fragment thereof (AB) that specifically binds to mammalian CD147, a MM comprising an amino acid sequence selected from the group consisting of SEQ ID NOs: 30-100, and a CM comprising an amino acid sequence selected from the group consisting of SEQ ID NOs: 356-423, 680-698, 713, 714, and 789-808. In some embodiments, the MM comprises an amino acid sequence selected from the group consisting of SEQ ID NOs: 30-100, and the CM comprises an amino acid sequence selected from the group consisting of SEQ ID NOs: 359, 370, 377, 382, 390, 397, 406-423, 680-698, 713, 714, and 807-808. In some embodiments, the AB comprises the VH CDR1 sequence GFTFSNYWMN (SEQ ID NO: 10) or GFTFSNYWMD (SEQ ID NO: 11); the VH CDR2 sequence EIRLKSYNYATH (SEQ ID NO: 12); the VH CDR3 sequence AGTDY (SEQ ID NO: 13); the VL CDR1 sequence KASQSVRTDVA (SEQ ID NO: 14) or RASQSVRTDVG (SEQ ID NO: 15); the VL CDR2 sequence YSSNRYT (SEQ ID NO: 16); and the VL CDR3 sequence QQDYSSPFT (SEQ ID NO: 17) or QQDYSSPYT (SEQ ID NO: 18). In some embodiments, the AB comprises a heavy chain variable region comprising an amino acid sequence selected from the group consisting of SEQ ID NOs: 1-3, and a light chain variable region comprising an amino acid sequence selected from the group consisting of SEQ ID NOs: 5-9.

In some embodiments, the activatable CD147 antibody comprises an antibody or an antigen binding fragment thereof (AB) that specifically binds to mammalian CD147, wherein the AB specifically binds to the same epitope on human CD147 and/or cynomolgus monkey CD147 as an isolated antibody of the disclosure; a masking moiety (MM) that inhibits the binding of the AB to CD147 when the activatable antibody is in an uncleaved state; and a cleavable moiety (CM) coupled to the AB, wherein the CM is a polypeptide that functions as a substrate for a protease.

In some embodiments, the CD147 activatable antibody of the disclosure comprises an isolated antibody or an antigen binding fragment thereof (AB) that specifically binds to mammalian CD147, wherein the AB specifically binds human CD147 and cynomolgus monkey CD147. In some embodiments, the antibody or antigen binding fragment thereof comprises the VH CDR1 sequence GFTFSNYWMN (SEQ ID NO: 10) or GFTFSNYWMD (SEQ ID NO: 11); the VH CDR2 sequence EIRLKSYNYATH (SEQ ID NO: 12); the VH CDR3 sequence AGTDY (SEQ ID NO: 13); the VL CDR1 sequence KASQSVRTDVA (SEQ ID NO: 14) or RASQSVRTDVG (SEQ ID NO: 15); the VL CDR2 sequence YSSNRYT (SEQ ID NO: 16); and the VL CDR3 sequence QQDYSSPFT (SEQ ID NO: 17) or QQDYSSPYT (SEQ ID NO: 18). In some embodiments, the activatable antibody or antigen binding fragment thereof comprises a heavy chain variable region comprising an amino acid sequence selected from the group consisting of SEQ ID NOs: 1-4, and a light chain variable region comprising an amino acid sequence selected from the group consisting of SEQ ID NOs: 5-9.

In some embodiments, the activatable CD147 antibody comprises an antibody or an antigen binding fragment thereof (AB) that specifically binds to mammalian CD147, wherein the AB specifically cross-competes with (inhibits the binding of) an isolated antibody of the disclosure for binding to human CD147 and/or cynomolgus monkey CD147; a masking moiety (MM) that inhibits the binding of the AB to CD147 when the activatable antibody is in an uncleaved state; and a cleavable moiety (CM) coupled to the AB, wherein the CM is a polypeptide that functions as a substrate for a protease.

In some embodiments, the CD147 activatable antibody of the disclosure comprises an isolated antibody or an antigen binding fragment thereof (AB) that specifically binds to mammalian CD147, wherein the AB specifically binds human CD147 and cynomolgus monkey CD147. In some embodiments, the antibody or antigen binding fragment thereof comprises the VH CDR1 sequence GFTFSNYWMN (SEQ ID NO: 10) or GFTFSNYWMD (SEQ ID NO: 11); the VH CDR2 sequence EIRLKSYNYATH (SEQ ID NO: 12); the VH CDR3 sequence AGTDY (SEQ ID NO: 13); the VL CDR1 sequence KASQSVRTDVA (SEQ ID NO: 14) or RASQSVRTDVG (SEQ ID NO: 15); the VL CDR2 sequence YSSNRYT (SEQ ID NO: 16); and the VL CDR3 sequence QQDYSSPFT (SEQ ID NO: 17) or QQDYSSPYT (SEQ ID NO: 18). In some embodiments, the activatable antibody or antigen binding fragment thereof comprises a heavy chain variable region comprising an amino acid sequence selected from the group consisting of SEQ ID NOs: 1-4 and a light chain variable region comprising an amino acid sequence selected from the group consisting of SEQ ID NOs: 5-9.

In some embodiments, the activatable antibody also includes an agent conjugated to the AB. In some embodiments, the agent conjugated to the AB or the AB of an activatable antibody is a therapeutic agent. In some embodiments, the agent is an antineoplastic agent. In some embodiments, the agent is a toxin or fragment thereof. As used herein, a fragment of a toxin is a fragment that retains toxic activity. In some embodiments, the agent is conjugated to the AB via a cleavable linker. In some embodiments, the agent is conjugated to the AB via a linker that includes at least one CM1-CM2 substrate sequence. In some embodiments, the agent is conjugated to the AB via a noncleavable linker. In some embodiments, the agent is conjugated to the AB via a linker that is cleavable in an intracellular or lysosomal environment. In some embodiments, the agent is a microtubule inhibitor. In some embodiments, the agent is a nucleic acid damaging agent, such as a DNA alkylator, a DNA cleaving agent, a DNA cross-linker, a DNA intercalator, or other DNA damaging agent. In some embodiments, the agent is an agent selected from the group listed in Table 5. In some embodiments, the agent is a dolastatin. In some embodiments, the agent is an auristatin or derivative thereof. In some embodiments, the agent is auristatin E or a derivative thereof. In some embodiments, the agent is monomethyl auristatin E (MMAE). In some embodiments, the agent is monomethyl auristatin D (MMAD). In some embodiments, the agent is a maytansinoid or maytansinoid derivative. In some embodiments, the agent is DM1 or DM4. In some embodiments, the agent is a duocarmycin or derivative thereof. In some embodiments, the agent is a calicheamicin or derivative thereof. In some embodiments, the agent is a pyrrolobenzodiazepine. In some embodiments, the agent is a pyrrolobenzodiazepine dimer.

In some embodiments, the activatable antibody is conjugated to one or more equivalents of an agent. In some embodiments, the activatable antibody is conjugated to one equivalent of the agent. In some embodiments, the activatable antibody is conjugated to two, three, four, five, six, seven, eight, nine, ten, or greater than ten equivalents of the agent. In some embodiments, the activatable antibody is part of a mixture of activatable antibodies having a homogeneous number of equivalents of conjugated agents. In some embodiments, the activatable antibody is part of a mixture of activatable antibodies having a heterogeneous number of equivalents of conjugated agents. In some embodiments, the mixture of activatable antibodies is such that the average number of agents conjugated to each activatable antibody is between zero to one, between one to two, between two and three, between three and four, between four and five, between five and six, between six and seven, between seven and eight, between eight and nine, between nine and ten, and ten and greater. In some embodiments, the mixture of activatable antibodies is such that the average number of agents conjugated to each activatable antibody is one, two, three, four, five, six, seven, eight, nine, ten, or greater.

In some embodiments, the activatable antibody comprises one or more site-specific amino acid sequence modifications such that the number of lysine and/or cysteine residues is increased or decreased with respect to the original amino acid sequence of the activatable antibody, thus in some embodiments correspondingly increasing or decreasing the number of agents that can be conjugated to the activatable antibody, or in some embodiments limiting the conjugation of the agents to the activatable antibody in a site-specific manner. In some embodiments, the modified activatable antibody is modified with one or more non-natural amino acids in a site-specific manner, thus in some embodiments limiting the conjugation of the agents to only the sites of the non-natural amino acids.

In some embodiments, the agent is an anti-inflammatory agent.

In some embodiments, the activatable antibody also includes a detectable moiety. In some embodiments, the detectable moiety is a diagnostic agent.

In some embodiments, the activatable antibody also includes a signal peptide. In some embodiments, the signal peptide is conjugated to the activatable antibody via a spacer. In some embodiments, the spacer is conjugated to the activatable antibody in the absence of a signal peptide. In some embodiments, the spacer is joined directly to the MM of the activatable antibody. In some embodiments, the spacer is joined directly to the MM of the activatable antibody in the structural arrangement from N-terminus to C-terminus of spacer-MM-CM-AB. An example of a spacer joined directly to the N-terminus of MM of the activatable antibody is QGQSGQ (SEQ ID NO: 424). Other examples of a spacer joined directly to the N-terminus of MM of the activatable antibody include QGQSGQG (SEQ ID NO: 645), QGQSG (SEQ ID NO: 646), QGQS (SEQ ID NO: 647), QGQ (SEQ ID NO: 648), QG (SEQ ID NO: 649), and Q. Other examples of a spacer joined directly to the N-terminus of MM of the activatable antibody include GQSGQG (SEQ ID NO: 666), QSGQG (SEQ ID NO: 667), SGQG (SEQ ID NO: 668), GQG (SEQ ID NO: 669), and G. In some embodiments, no spacer is joined to the N-terminus of the MM. In some embodiments, the spacer includes at least the amino acid sequence QGQSGQ (SEQ ID NO: 424). In some embodiments, the spacer includes at least the amino acid sequence QGQSGQG (SEQ ID NO: 645). In some embodiments, the spacer includes at least the amino acid sequence QGQSG (SEQ ID NO: 646). In some embodiments, the spacer includes at least the amino acid sequence QGQS (SEQ ID NO: 647). In some embodiments, the spacer includes at least the amino acid sequence QGQ (SEQ ID NO: 648). In some embodiments, the spacer includes at least the amino acid sequence QG (SEQ ID NO: 649). In some embodiments, the spacer includes at least the amino acid residue Q. In some embodiments, the spacer includes at least the amino acid sequence GQSGQG (SEQ ID NO: 666). In some embodiments, the spacer includes at least the amino acid sequence QSGQG (SEQ ID NO: 667). In some embodiments, the spacer includes at least the amino acid sequence SGQG (SEQ ID NO: 668). In some embodiments, the spacer includes at least the amino acid sequence GQG (SEQ ID NO: 669). In some embodiments, the spacer includes at least the amino acid sequence G. In some embodiments, the spacer is absent.

In some embodiments, the AB of the activatable antibody naturally contains one or more disulfide bonds. In some embodiments, the AB can be engineered to include one or more disulfide bonds.

In some embodiments, activatable antibody or antigen binding fragment thereof is conjugated to an agent. In some embodiments, the activatable antibody comprises an antibody or antigen binding fragment thereof cross-competes with (inhibits the binding of) an isolated antibody that comprises the VH CDR1 sequence GFTFSNYWMN (SEQ ID NO: 10) or GFTFSNYWMD (SEQ ID NO: 11); the VH CDR2 sequence EIRLKSYNYATH (SEQ ID NO: 12); the VH CDR3 sequence AGTDY (SEQ ID NO: 13); the VL CDR1 sequence KASQSVRTDVA (SEQ ID NO: 14) or RASQSVRTDVG (SEQ ID NO: 15); the VL CDR2 sequence YSSNRYT (SEQ ID NO: 16); and the VL CDR3 sequence QQDYSSPFT (SEQ ID NO: 17) or QQDYSSPYT (SEQ ID NO: 18). In some embodiments, the activatable antibody comprises an antibody or antigen binding fragment thereof cross-competes with (inhibits the binding of) an isolated antibody that comprises a heavy chain variable region comprising an amino acid sequence selected from the group consisting of SEQ ID NOs: 1-3, and a light chain variable region comprising an amino acid sequence selected from the group consisting of SEQ ID NOs: 5-9. In some embodiments, the agent is a toxin or fragment thereof. In some embodiments, the agent is a microtubule inhibitor. In some embodiments, the agent is a nucleic acid damaging agent. In some embodiments, the agent is selected from the group consisting of a dolastatin or a derivative thereof, an auristatin or a derivative thereof, a maytansinoid or a derivative thereof, a duocarmycin or a derivative thereof, a calicheamicin or a derivative thereof, and a pyrrolobenzodiazepine or a derivative thereof. In some embodiments, the agent is auristatin E or a derivative thereof. In some embodiments, the agent is monomethyl auristatin E (MMAE). In some embodiments, the agent is monomethyl auristatin D (MMAD). In some embodiments, the agent is a maytansinoid selected from the group consisting of DM1 and DM4. In some embodiments, the agent is maytansinoid DM4. In some embodiments, the agent is duocarmycin. In some embodiments, the agent is conjugated to the AB via a linker. In some embodiments, the linker with which the agent is conjugated to the AB comprises an SPDB moiety, a vc moiety, or a PEG2-vc moiety. In some embodiments, the linker and toxin conjugated to the AB comprises an SPDB-DM4 moiety, a vc-MMAD moiety, a vc-MMAE moiety, vc-duocarmycin, or a PEG2-vc-MMAD moiety. In some embodiments, the linker is a cleavable linker. In some embodiments, the linker is a non-cleavable linker. In some embodiments, the agent is a detectable moiety. In some embodiments, the detectable moiety is a diagnostic agent.

In some embodiments, the conjugated activatable antibody comprises a conjugated activatable antibody that, in an activated state, binds CD147 comprising: an antibody or an antigen binding fragment thereof (AB) that specifically binds to mammalian CD147, wherein the AB specifically binds human CD147 and cynomolgus monkey CD147; a masking moiety (MM) that inhibits the binding of the AB to CD147 when the activatable antibody is in an uncleaved state; a cleavable moiety (CM) coupled to the AB, wherein the CM is a polypeptide that functions as a substrate for a protease; and an agent conjugated to the AB. In some embodiments, the agent is selected from the group consisting of a dolastatin or a derivative thereof, an auristatin or a derivative thereof, a maytansinoid or a derivative thereof, a duocarmycin or a derivative thereof, a calicheamicin or a derivative thereof, and a pyrrolobenzodiazepine or a derivative thereof. In some embodiments, the agent is selected from the group consisting of auristatin E, monomethyl auristatin F (MMAF), monomethyl auristatin E (MMAE), monomethyl auristatin D (MMAD), maytansinoid DM4, maytansinoid DM1, a duocarmycin, a pyrrolobenzodiazepine, and a pyrrolobenzodiazepine dimer. In some embodiments, the agent is conjugated to the AB via a linker. In some embodiments, the linker with which the agent is conjugated to the AB comprises an SPDB moiety, a vc moiety, or a PEG2-vc moiety. In some embodiments, the linker and toxin conjugated to the AB comprises an SPDB-DM4 moiety, a vc-MMAD moiety, a vc-MMAE moiety, vc-duocarmycin, or a PEG2-vc-MMAD moiety. In some embodiments, the AB of the conjugated activatable antibody or antigen binding fragment thereof comprises the VH CDR1 sequence GFTFSNYWMN (SEQ ID NO: 10) or GFTFSNYWMD (SEQ ID NO: 11); the VH CDR2 sequence EIRLKSYNYATH (SEQ ID NO: 12); the VH CDR3 sequence AGTDY (SEQ ID NO: 13); the VL CDR1 sequence KASQSVRTDVA (SEQ ID NO: 14) or RASQSVRTDVG (SEQ ID NO: 15); the VL CDR2 sequence YSSNRYT (SEQ ID NO: 16); and the VL CDR3 sequence QQDYSSPFT (SEQ ID NO: 17) or QQDYSSPYT (SEQ ID NO: 18). In some embodiments, the AB of the conjugated activatable antibody or antigen binding fragment thereof comprises a heavy chain variable region comprising an amino acid sequence selected from the group consisting of SEQ ID NOs: 1-3, and a light chain variable region comprising an amino acid sequence selected from the group consisting of SEQ ID NOs: 5-9. In some embodiments, the MM comprises an amino acid sequence selected from the group consisting of SEQ ID NOs: 30-100. In some embodiments, the MM comprises an amino acid sequence selected from the group consisting of SEQ ID NOs: 30-100. In some embodiments, the CM comprises an amino acid sequence selected from the group consisting of SEQ ID NOs: 356-423, 680-698, 713, 714, and 789-808. In some embodiments, the CM comprises an amino acid sequence selected from the group consisting of SEQ ID NOs: 359, 370, 377, 382, 390, 397, 406-423, 680-698, 713, 714, and 807-808. In some embodiments, the activatable antibody comprises a combination of amino acid sequences, wherein the combination of amino acid sequences is selected from a single row in Table 4, wherein for a given combination, (a) the heavy chain of the AB comprises the amino acid sequences of the VH CDR sequences corresponding to the given combination in the single row listed in Table 4, (b) the light chain of the AB comprises the amino acid sequences of the VL CDR sequences corresponding to the given combination in the single row listed in Table 4, (c) the MM comprises the amino acid sequence of the mask sequence (MM) corresponding to the given combination in the single row listed in Table 4, and (d) the CM comprises the amino acid sequence of the substrate sequence (CM) corresponding to the given combination in the single row listed in Table 4. In some embodiments, the activatable antibody comprises a combination of amino acid sequences, wherein for a given combination of amino acid sequences. (a) the heavy chain of the AB comprises the amino acid sequences of the VH sequence or VH CDR sequences selected from the group consisting of: the VH sequence or VH CDR sequences listed in the corresponding column of Table 5, (b) the light chain of the AB comprises the amino acid sequences of the VL sequence or VL CDR sequences selected from the group consisting of: the VL sequence or VL CDR sequences listed in the corresponding column of Table 5, (c) the MM comprises the amino acid sequence of the mask sequence (MM) selected from the group consisting of: the MM sequences listed in the corresponding column of Table 5, and (d) the CM comprises the amino acid sequence of the substrate sequence (CM) selected from the group consisting of: the CM sequences listed in the corresponding column of Table 5. In some embodiments, the activatable antibody comprises: a heavy chain comprising an amino acid sequence selected from the group consisting of SEQ ID NOs: 1-4 or 19-22, and a light chain comprising an amino acid sequence selected from the group consisting of SEQ ID NOs: 5-9, 23-27, 140-182, 185-227, 230-272, and 275-317.

In some embodiments, the conjugated activatable antibody comprises a conjugated activatable antibody that, in an activated state, binds to CD147, comprising: an antibody or an antigen binding fragment thereof (AB) that specifically binds to mammalian CD147, wherein the AB specifically binds human CD147 and cynomolgus monkey CD147; a masking moiety (MM) that inhibits the binding of the AB to CD147 when the activatable antibody is in an uncleaved state; a cleavable moiety (CM) coupled to the AB, wherein the CM is a polypeptide that functions as a substrate for a protease; and an agent conjugated to the AB, wherein the AB comprises: (i) the VH CDR1 sequence GFTFSNYWMN (SEQ ID NO: 10) or GFTFSNYWMD (SEQ ID NO: 11); the VH CDR2 sequence EIRLKSYNYATH (SEQ ID NO: 12); the VH CDR3 sequence AGTDY (SEQ ID NO: 13); the VL CDR1 sequence KASQSVRTDVA (SEQ ID NO: 14) or RASQSVRTDVG (SEQ ID NO: 15); the VL CDR2 sequence YSSNRYT (SEQ ID NO: 16); and the VL CDR3 sequence QQDYSSPFT (SEQ ID NO: 17) or QQDYSSPYT (SEQ ID NO: 18), or (ii) a heavy chain variable region comprising an amino acid sequence selected from the group consisting of SEQ ID NOs: 1-3, and a light chain variable region comprising an amino acid sequence selected from the group consisting of SEQ ID NOs: 5-9, or (iii) a heavy chain comprising an amino acid sequence selected from the group consisting of SEQ ID NOs: 1-4 or 19-22, and a light chain comprising an amino acid sequence selected from the group consisting of SEQ ID NOs: 5-9, 23-27, 140-182, 185-227, 230-272, and 275-317; and wherein the agent is selected from the group consisting of auristatin E, monomethyl auristatin F (MMAF), monomethyl auristatin E (MMAE), monomethyl auristatin D (MMAD), maytansinoid DM4, maytansinoid DM1, a pyrrolobenzodiazepine, a pyrrolobenzodiazepine dimer, and a duocarmycin. In some embodiments, the MM comprises an amino acid sequence selected from the group consisting of SEQ ID NOs: 30-100. In some embodiments, the MM comprises an amino acid sequence selected from the group consisting of SEQ ID NOs: 30, 31, 33, 44, 46, 75-80, 82, 83, and 86-100. In some embodiments, the CM comprises an amino acid sequence selected from the group consisting of SEQ ID NOs: 356-423, 680-698, 713, 714, and 789-808. In some embodiments, the CM comprises an amino acid sequence selected from the group consisting of SEQ ID NOs: 359, 370, 377, 382, 390, 397, 406-423, 680-698, 713, 714, and 807-808. In some embodiments, the agent is conjugated to the AB via a linker, and wherein the linker to which the agent is conjugated to the AB comprises an SPDB moiety, a vc moiety, or a PEG2-vc moiety. In some embodiments, the linker and toxin conjugated to the AB comprises an SPDB-DM4 moiety, a vc-MMAD moiety, a vc-MMAE moiety, vc-duocarmycin, or a PEG2-vc-MMAD moiety.

In some embodiments, the conjugated activatable antibody comprises a conjugated activatable antibody or conjugated antibody comprising: an antibody or antigen binding fragment thereof (AB) that, in an activated state, binds CD147; and a toxin conjugated to the AB via a linker, wherein the conjugated activatable antibody or the conjugated antibody comprises amino acid sequences, a linker, and a toxin selected from a single row in Table 9, wherein for the given combination: (a) the AB comprises a heavy chain comprising the amino acid sequence of the heavy chain sequence or heavy chain variable domain sequence corresponding to the given combination in the single row listed in Table 9, (b) the AB comprises a light chain comprising the amino acid sequence of the light chain sequence or light chain variable domain sequence corresponding to the given combination in the single row listed in Table 9, and (c) the linker and the toxin comprise the linker and the toxin corresponding to the given combination in the single row listed in Table 9.

In some embodiments, the activatable antibody is encoded by a nucleic acid sequence that comprises a nucleic acid sequence encoding a heavy chain variable region amino acid sequence selected from the group consisting of SEQ ID NOs: 1-4. In some embodiments, the activatable antibody is encoded by a nucleic acid sequence that comprises a nucleic acid sequence encoding a heavy chain variable region amino acid sequence selected from the group consisting of SEQ ID NOs: 1-3.

In some embodiments, the activatable antibody is encoded by a nucleic acid sequence that comprises a nucleic acid sequence encoding a light chain variable region amino acid sequence selected from the group consisting of SEQ ID NOs: 5-9. In some embodiments, the activatable antibody is encoded by a nucleic acid sequence that comprises a nucleic acid sequence encoding a light chain variable region amino acid sequence selected from the group consisting of SEQ ID NOs: 5-8.

In some embodiments, the activatable antibody is encoded by a nucleic acid sequence that comprises a nucleic acid sequence encoding a heavy chain variable region amino acid sequence selected from the group consisting of SEQ ID NOs: 1-4, and a nucleic acid sequence encoding a light chain variable region amino acid sequence selected from the group consisting of SEQ ID NOs: 5-9.

In some embodiments, the activatable antibody is encoded by a nucleic acid sequence that comprises a nucleic acid sequence encoding a heavy chain variable region amino acid sequence selected from the group consisting of SEQ ID NOs: 1-3, and a nucleic acid sequence encoding a light chain variable region amino acid sequence selected from the group consisting of SEQ ID NOs: 5-8.

In some embodiments, the activatable antibody is encoded by a nucleic acid sequence that comprises a nucleic acid sequence that is at least 90%, 91%, 92%, 93%, 94%, 95%, 96%, 97%, 98% or 99% identical to a nucleic acid sequence encoding a heavy chain variable region amino acid sequence selected from the group consisting of SEQ ID NOs: 1-4. In some embodiments, the activatable antibody is encoded by a nucleic acid sequence that comprises a nucleic acid sequence that is at least 90%, 91%, 92%, 93%, 94%, 95%, 96%, 97%, 98% or 99% identical to a nucleic acid sequence encoding a heavy chain variable region amino acid sequence comprising selected from the group consisting of SEQ ID NOs: 1-3.

In some embodiments, the activatable antibody is encoded by a nucleic acid sequence that comprises a nucleic acid sequence that is at least 90%, 91%, 92%, 93%, 94%, 95%, 96%, 97%, 98% or 99% identical to a nucleic acid sequence encoding a light variable region chain amino acid sequence selected from the group consisting of SEQ ID NOs: 5-9. In some embodiments, the activatable antibody is encoded by a nucleic acid sequence that comprises a nucleic acid sequence that is at least 90%, 91%, 92%, 93%, 94%, 95%, 96%, 97%, 98% or 99% identical to a nucleic acid sequence encoding a light chain variable region amino acid sequence selected from the group consisting of SEQ ID NOs: 5-8.

In some embodiments, the activatable antibody is encoded by a nucleic acid sequence that comprises a nucleic acid sequence that is at least 90%, 91%, 92%, 93%, 94%, 95%, 96%, 97%, 98% or 99% identical to a nucleic acid sequence encoding a heavy chain variable region amino acid sequence selected from the group consisting of SEQ ID NOs: 1-4, and a nucleic acid sequence that is at least 90%, 91%, 92%, 93%, 94%, 95%, 96%, 97%, 98% or 99% identical to a nucleic acid sequence encoding a light chain variable region amino acid sequence selected from the group consisting of SEQ ID NOs: 5-9.

In some embodiments, the activatable antibody is encoded by a nucleic acid sequence that comprises a nucleic acid sequence that is at least 90%, 91%, 92%, 93%, 94%, 95%, 96%, 97%, 98% or 99%, identical to a nucleic acid sequence encoding a heavy chain variable region amino acid sequence selected from the group consisting of SEQ ID NOs: 1-3, and a nucleic acid sequence that is at least 90%, 91%, 92%, 93%, 94%, 95%, 96%, 97%, 98% or 99% identical to a nucleic acid sequence encoding a light chain variable region amino acid sequence selected from the group consisting of SEQ ID NOs: 5-8.

In some embodiments, the activatable antibody is encoded by a nucleic acid sequence that comprises a nucleic acid sequence encoding a heavy chain variable region amino acid sequence selected from the group consisting of the heavy chain variable region sequences shown in Table 1. In some embodiments, the activatable antibody is encoded by a nucleic acid sequence that comprises a nucleic acid sequence encoding a light chain variable region amino acid sequence selected from the group consisting of the light chain variable region sequences shown in Table 1. In some embodiments, the activatable antibody is encoded by a nucleic acid sequence that comprises a nucleic acid sequence encoding a heavy chain variable region amino acid sequence selected from the group consisting of the heavy chain variable region sequences shown in Table 1 and a nucleic acid sequence encoding a light chain variable region amino acid sequence selected from the group consisting of the light chain variable region sequences shown in Table 1.

In some embodiments, the activatable antibody is encoded by a nucleic acid sequence that comprises a nucleic acid sequence encoding a heavy chain variable region amino acid sequence that is at least 90%, 91%, 92%, 93%, 94%, 95%, 96%, 97%, 98% or 99% identical to an amino acid sequence selected from the group consisting of the heavy chain variable region sequences shown in Table 1. In some embodiments, the activatable antibody is encoded by a nucleic acid sequence that comprises a nucleic acid sequence encoding a light chain variable region amino acid sequence that is at least 90%, 91%, 92%, 93%, 94%, 95%, 96%, 97%, 98% or 99% identical to an amino acid sequence selected from the group consisting of the light chain variable region sequences shown in Table 1. In some embodiments, the activatable antibody is encoded by a nucleic acid sequence that comprises a nucleic acid sequence encoding a heavy chain variable region amino acid sequence that is at least 90%, 91%, 92%, 93%, 94%, 95%, 96%, 97%, 98% or 99% identical to an amino acid sequence selected from the group consisting of the heavy chain variable region sequences shown in Table 1 and a nucleic acid sequence that comprises a nucleic acid sequence encoding a light chain variable region amino acid sequence that is at least 90%, 91%, 92%, 93%, 94%, 95%, 96%, 97%, 98% or 99% identical to an amino acid sequence selected from the group consisting of the light chain variable region sequences shown in Table 1.

In some embodiments, the activatable antibody is encoded by a nucleic acid sequence that comprises a nucleic acid sequence encoding a combination of a variable heavy chain complementarity determining region 1 (VH CDR1, also referred to herein as CDRH1) sequence, a variable heavy chain complementarity determining region 2 (VH CDR2, also referred to herein as CDRH2) sequence, a variable heavy chain complementarity determining region 3 (VH CDR3, also referred to herein as CDRH3) sequence, a variable light chain complementarity determining region 1 (VL CDR1, also referred to herein as CDRL1) sequence, a variable light chain complementarity determining region 2 (VL CDR2, also referred to herein as CDRL2) sequence, and a variable light chain complementarity determining region 3 (VL CDR3, also referred to herein as CDRL3) sequence, wherein at least one CDR sequence is selected from the group consisting of a VH CDR1 sequence shown in Table 2; a VH CDR2 sequence shown in Table 2; a VH CDR3 sequence shown in Table 2; a VL CDR1 sequence shown in Table 2; a VL CDR2 sequence shown in Table 2; and a VL CDR3 sequence shown in Table 2.

In some embodiments, the activatable antibody is encoded by a nucleic acid sequence that comprises a nucleic acid sequence encoding a combination of a VH CDR1 sequence, a VH CDR2 sequence, a VH CDR3 sequence, a VL CDR1 sequence, a VL CDR2 sequence, and a VL CDR3 sequence, wherein at least one CDR sequence is selected from the group consisting of a VH CDR1 sequence that includes a sequence that is at least 90%, 91%, 92%, 93%, 94%, 95%, 96%, 97%, 98%, 99% or more identical to a VH CDR1 sequence shown in Table 2; a VH CD2 sequence that includes a sequence that is at least 90%, 91%, 92%, 93%, 94%, 95%, 96%, 97%, 98%, 99% or more identical to a VH CDR2 sequence shown in Table 2; a VH CDR3 sequence that includes a sequence that is at least 90%, 91%, 92%, 93%, 94%, 95%, 96%, 97%, 98%, 99% or more identical to a VH CDR3 sequence shown in Table 2; a VL CDR1 sequence that includes a sequence that is at least 90%, 91%, 92%, 93%, 94%, 95%, 96%, 97%, 98%, 99% or more identical to a VL CDR1 sequence shown in Table 2; a VL CDR2 sequence that includes a sequence that is at least 90%, 91%, 92%, 93%, 94%, 95%, 96%, 97%, 98%, 99% or more identical to a VL CDR2 sequence shown in Table 2; and a VL CDR3 sequence that includes a sequence that is at least 90%, 91%, 92%, 93%, 94%, 95%, 96%, 97%, 98%, 99% or more identical to a VL CDR3 sequence shown in Table 2.

In some embodiments, the activatable antibody is encoded by a nucleic acid sequence that comprises a nucleic acid sequence encoding a combination of a VH CDR1 sequence, a VH CDR2 sequence, a VH CDR3 sequence, a VL CDR1 sequence, a VL CDR2 sequence, and a VL CDR3 sequence, wherein the combination is a combination of the six CDR sequences (VH CDR1, VH CDR2, VH CDR3, VL CDR1, VL CDR2, and VL CDR3) shown in a single row in Table 2.

In some embodiments, the activatable antibody is encoded by a nucleic acid sequence that comprises a nucleic acid sequence encoding a light chain variable region that comprise a combination of a VL CDR1 sequence, a VL CDR2 sequence, and a VL CDR3 sequence, wherein the combination is a combination of the three light chain CDR sequences (VL CDR1. VL CDR2. VL CDR3) shown in a single row in Table 2.

In some embodiments, the activatable antibody is encoded by a nucleic acid sequence that comprises a nucleic acid sequence encoding a heavy chain variable region that comprise a combination of a VH CDR1 sequence, a VH CDR2 sequence, and a VH CDR3 sequence, wherein the combination is a combination of the three heavy chain CDR sequences (VH CDR1, VH CDR2, VH CDR3) shown in a single row in Table 2.

In some embodiments, the activatable antibody is encoded by a nucleic acid sequence that comprises a nucleic acid sequence encoding a combination of a VH CDR1 sequence, a VH CDR2 sequence, a VH CDR3 sequence, a VL CDR1 sequence, a VL CDR2 sequence, and a VL CDR3 sequence, wherein each CDR sequence in the combination comprises a sequence that is at least 90%, 91%, 92%, 93%, 94%, 95%, 96%, 97%, 98%, 99% or more identical to the corresponding CDR sequence in a combination of the six CDR sequences (VH CDR1, VH CDR2, VH CDR3, VL CDR1, VL CDR2, and VL CDR3) shown in a single row in Table 2.

In some embodiments, the activatable antibody is encoded by a nucleic acid sequence that comprises a nucleic acid sequence encoding a heavy chain variable region that comprise a combination of a VH CDR1 sequence, a VH CDR2 sequence, and a VH CDR3 sequence, wherein each CDR sequence in the combination comprises a sequence that is at least 90%, 91%, 92%, 93%, 94%, 95%, 96%, 97%, 98%, 99% or more identical to the corresponding CDR sequence in a combination of three heavy chain CDR sequences (VH CDR1, VH CDR2, VH CDR3) shown in a single row in Table 2.

In some embodiments, the activatable antibody is encoded by a nucleic acid sequence that comprises a nucleic acid sequence encoding a light chain variable region that comprise a combination of a VL CDR1 sequence, a VL CDR2 sequence, and a VL CDR3 sequence, wherein each CDR sequence in the combination comprises a sequence that is at least 90%, 91%, 92%, 93%, 94%, 95%, 96%, 97%, 98%, 99% or more identical to the corresponding CDR sequence in a combination of three light chain CDR sequences (VL CDR1, VL CDR2, VL CDR3) shown in a single row in Table 2.

The disclosure also provides methods for producing an activatable antibody of the disclosure by culturing a cell under conditions that lead to expression of the activatable antibody, wherein the cell comprises a nucleic acid molecule of the disclosure or a vector of the disclosure.

The disclosure also provides methods of manufacturing an activatable antibody that, in an activated state, binds CD147, the method comprising: (a) culturing a cell comprising a nucleic acid construct that encodes the activatable antibody under conditions that lead to expression of the activatable antibody, wherein the activatable antibody comprises an activatable antibody of the disclosure; and (b) recovering the activatable antibody.

In some embodiments, the activatable antibody includes one or more polypeptides that include the combination of sequences in a given row of Table 4 or any combination of a mask sequence (MM), a substrate sequence (CM), a light chain variable domain sequence or light chain variable domain CDR sequences, and a heavy chain variable domain sequence or heavy chain variable domain CDR sequences of Table 5.

TABLE 4
CD147 Activatable Antibody Combinations

| | | | VL CDRs | VH CDRs |
|---|---|---|---|---|
| Comb. | Mask Sequence | Substrate Sequence | SEQ ID | SEQ ID |
| No. | (MM) | (CM) | NOs | NOs |
| 1 | THGPCHFKPNCSYPT | LSGRSDNH | 15, 16, | 11, 12, |
| | (SEQ ID NO: 75) | (SEQ ID NO: 359) | 18 | 13 |
| 2 | THGPCHFKPNCSYPT | ISSGLLSS | 15, 16, | 11, 12, |
| | (SEQ ID NO: 75) | (SEQ ID NO: 382) | 18 | 13 |
| 3 | THGPCHFKPNCSYPT | LSGRSGNH | 15, 16, | 11, 12, |
| | (SEQ ID NO: 75) | (SEQ ID NO: 789) | 18 | 13 |
| 4 | THGPCHFKPNCSYPT | AVGLLAPP | 15, 16, | 11, 12, |
| | (SEQ ID NO: 75) | (SEQ ID NO: 390) | 18 | 13 |
| 5 | THGPCHFKPNCSYPT | VHMPLGFLGP | 15, 16, | 11, 12, |
| | (SEQ ID NO: 75) | (SEQ ID NO: 370) | 18 | 13 |
| 6 | THGPCHFKPNCSYPT | TSTSGRSANTRG | 15, 16, | 11, 12, |
| | (SEQ ID NO: 75) | (SEQ ID NO: 397) | 18 | 13 |
| 7 | THGPCHFKPNCSYPT | QNQALRMA | 15, 16, | 11, 12, |
| | (SEQ ID NO: 75) | (SEQ ID NO: 377) | 18 | 13 |
| 8 | THGPCHFKPNCSYPT | ISSGLLSGRSDNH | 15, 16, | 11, 12, |
| | (SEQ ID NO: 75) | (SEQ ID NO: 406) | 18 | 13 |
| 9 | THGPCHFKPNCSYPT | ISSGLLSGRSGNH | 15, 16, | 11, 12, |
| | (SEQ ID NO: 75) | (SEQ ID NO: 423) | 18 | 13 |
| 10 | THGPCHFKPNCSYPT | ISSGLLSGRSANPRG | 15, 16, | 11, 12, |
| | (SEQ ID NO: 75) | (SEQ ID NO: 680) | 18 | 13 |
| 11 | THGPCHFKPNCSYPT | AVGLLAPPTSGRSANPRG | 15, 16, | 11, 12, |
| | (SEQ ID NO: 75) | (SEQ ID NO: 681) | 18 | 13 |
| 12 | THGPCHFKPNCSYPT | AVGLLAPPSGRSANPRG | 15, 16, | 11, 12, |
| | (SEQ ID NO: 75) | (SEQ ID NO: 682) | 18 | 13 |
| 13 | THGPCHFKPNCSYPT | ISSGLLSGRSDDH | 15, 16, | 11, 12, |
| | (SEQ ID NO: 75) | (SEQ ID NO: 683) | 18 | 13 |
| 14 | THGPCHFKPNCSYPT | ISSGLLSGRSDIH | 15, 16, | 11, 12, |
| | (SEQ ID NO: 75) | (SEQ ID NO: 684) | 18 | 13 |
| 15 | THGPCHFKPNCSYPT | ISSGLLSGRSDQH | 15, 16, | 11, 12, |
| | (SEQ ID NO: 75) | (SEQ ID NO: 685) | 18 | 13 |
| 16 | THGPCHFKPNCSYPT | ISSGLLSGRSDTH | 15, 16, | 11, 12, |
| | (SEQ ID NO: 75) | (SEQ ID NO: 686) | 18 | 13 |
| 17 | THGPCHFKPNCSYPT | ISSGLLSGRSDYH | 15, 16, | 11, 12, |
| | (SEQ ID NO: 75) | (SEQ ID NO: 687) | 18 | 13 |
| 18 | THGPCHFKPNCSYPT | ISSGLLSGRSDNP | 15, 16, | 11, 12, |
| | (SEQ ID NO: 75) | (SEQ ID NO: 688) | 18 | 13 |
| 19 | THGPCHFKPNCSYPT | ISSGLLSGRSANP | 15, 16, | 11, 12, |
| | (SEQ ID NO: 75) | (SEQ ID NO: 689) | 18 | 13 |
| 20 | THGPCHFKPNCSYPT | ISSGLLSGRSANI | 15, 16, | 11, 12, |
| | (SEQ ID NO: 75) | (SEQ ID NO: 690) | 18 | 13 |
| 21 | THGPCHEKTNCSYPT | ISSGLLSGRSDNI | 15, 16, | 11, 12, |
| | (SEQ ID NO: 75) | (SEQ ID NO: 713) | 18 | 13 |
| 22 | THGPCHFKPNCSYPT | AVGLLAPPGGLSGRSDNH | 15, 16, | 11, 12, |
| | (SEQ ID NO: 75) | (SEQ ID NO: 412) | 18 | 13 |
| 23 | THGPCHFKPNCSYPT | AVGLLAPPGGLSGRSDDH | 15, 16, | 11, 12, |
| | (SEQ ID NO: 75) | (SEQ ID NO: 691) | 18 | 13 |
| 24 | THGPCHFKPNCSYPT | AVGLLAPPGGLSGRSDIH | 15, 16, | 11, 12, |
| | (SEQ ID NO: 75) | (SEQ ID NO: 692) | 18 | 13 |
| 25 | THGPCHFKPNCSYPT | AVGLLAPPGGLSGRSDQH | 15, 16, | 11, 12, |
| | (SEQ ID NO: 75) | (SEQ ID NO: 693) | 18 | 13 |
| 26 | THGPCHFKPNCSYPT | AVGLLAPPGGLSGRSDTH | 15, 16, | 11, 12, |
| | (SEQ ID NO: 75) | (SEQ ID NO: 694) | 18 | 13 |
| 27 | THGPCHFKPNCSYPT | AVGLLAPPGGLSGRSDYH | 15, 16, | 11, 12, |
| | (SEQ ID NO: 75) | (SEQ ID NO: 695) | 18 | 13 |
| 28 | THGPCHFKPNCSYPT | AVGLLAPPGGLSGRSDNP | 15, 16, | 11, 12, |
| | (SEQ ID NO: 75) | (SEQ ID NO: 696) | 18 | 13 |
| 29 | THGPCHFKPNCSYPT | AVGLLAPPGGLSGRSANP | 15, 16, | 11, 12, |
| | (SEQ ID NO: 75) | (SEQ ID NO: 697) | 18 | 13 |
| 30 | THGPCHFKPNCSYPT | AVGLLAPPGGLSGRSANI | 15, 16, | 11, 12, |
| | (SEQ ID NO: 75) | (SEQ ID NO: 698) | 18 | 13 |
| 31 | THGPCHFKPNCSYPT | AVGLLAPPGGLSGRSDNI | 15, 16, | 11, 12, |
| | (SEQ ID NO: 75) | (SEQ ID NO: 714) | 18 | 13 |
| 32 | THGPCHFKPNCSYPT | ISSGLLSSGGSGGSLSGRSDNH | 15, 16, | 11, 12, |
| | (SEQ ID NO: 75) | (SEQ ID NO: 407) | 18 | 13 |
| 33 | AHGPCHYNTECNSNK | LSGRDNH | 15, 16, | 11, 12, |
| | (SEQ ID NO: 78) | (SEQ ID NO: 359) | 18 | 13 |
| 34 | AHGPCHYNTECNSNK | ISSGLLSS | 15, 16, | 11, 12, |
| | (SEQ ID NO: 78) | (SEQ ID NO: 382) | 18 | 13 |
| 35 | AHGPCHYNTECNSNK | LSGRSGNH | 15, 16, | 11, 12, |
| | (SEQ ID NO: 78) | (SEQ ID NO: 789) | 18 | 13 |
| 36 | AHGPCHYNTECNSNK | AVGLLAPP | 15, 16, | 11, 12, |
| | (SEQ ID NO: 78) | (SEQ ID NO: 390) | 18 | 13 |
| 37 | AHGPCHYNTECNSNK | VHMPLGFLGP | 15, 16, | 11, 12, |
| | (SEQ ID NO: 78) | (SEQ ID NO: 370) | 18 | 13 |
| 38 | AHGPCHYNTECNSNK | TSTSGRSANPRG | 15, 16, | 11, 12, |
| | (SEQ ID NO: 78) | (SEQ ID NO: 397) | 18 | 13 |
| 39 | AHGPCHYNTECNSNK | QNQALRMA | 15, 16, | 11, 12, |
| | (SEQ ID NO: 78) | (SEQ ID NO: 377) | 18 | 13 |
| 40 | AHGPCHYNTECNSNK | ISSGLLSGRSDNH | 15, 16, | 11, 12, |
| | (SEQ ID NO: 78) | (SEQ ID NO: 406) | 18 | 13 |
| 41 | AHGPCHYNTECNSNK | ISSGLLSGRSGNH | 15, 16, | 11, 12, |
| | (SEQ ID NO: 78) | (SEQ ID NO: 423) | 18 | 13 |
| 42 | AHGPCHYNTECNSNK | ISSGLLSGRSANPRG | 15, 16, | 11, 12, |
| | (SEQ ID NO: 78) | (SEQ ID NO: 680) | 18 | 13 |
| 43 | AHGPCHYNTECNSNK | AVGLLAPPTSGRSANPRG | 15, 16, | 11, 12, |
| | (SEQ ID NO: 78) | (SEQ ID NO: 681) | 18 | 13 |
| 44 | AHGPCHYNTECNSNK | AVGLLAPPSGRSANPRG | 15, 16, | 11, 12, |
| | (SEQ ID NO: 78) | (SEQ ID NO: 682) | 18 | 13 |
| 45 | AHGPCHYNTECNSNK | ISSGLLSGRSDDH | 15, 16, | 11, 12, |
| | (SEQ ID NO: 78) | (SEQ ID NO: 683) | 18 | 13 |
| 46 | AHGPCHYNTECNSNK | ISSGLLSGRSDIH | 15, 16, | 11, 12, |
| | (SEQ ID NO: 78) | (SEQ ID NO: 684) | 18 | 13 |
| 47 | AHGPCHYNTECNSNK | ISSGLLSGRSDQH | 15, 16, | 11, 12, |
| | (SEQ ID NO: 78) | (SEQ ID NO: 685) | 18 | 13 |
| 48 | AHGPCHYNTECNSNK | ISSGLLSGRSDTH | 15, 16, | 11, 12, |
| | (SEQ ID NO: 78) | (SEQ ID NO: 686) | 18 | 13 |
| 49 | AHGPCHYNTECNSNK | ISSGLLSGRSDYH | 15, 16, | 11, 12, |
| | (SEQ ID NO: 78) | (SEQ ID NO: 687) | 18 | 13 |
| 50 | AHGPCHYNTECNSNK | ISSGLLSGRSDNP | 15, 16, | 11, 12, |
| | (SEQ ID NO: 78) | (SEQ ID NO: 688) | 18 | 13 |
| 51 | AHGPCHYNTECNSNK | ISSGLLSGRSANP | 15, 16, | 11, 12, |
| | (SEQ ID NO: 78) | (SEQ ID NO: 689) | 18 | 13 |
| 52 | AHGPCHYNTECNSNK | ISSGLLSGRSANI | 15, 16, | 11, 12, |
| | (SEQ ID NO: 78) | (SEQ ID NO: 690) | 18 | 13 |
| 53 | AHGPCHYNTECNSNK | ISSGLLSGRSDNI | 15, 16, | 11, 12, |
| | (SEQ ID NO: 78) | (SEQ ID NO: 713) | 18 | 13 |
| 54 | AHGPCHYNTECNSNK | AVGLLAPPGGLSGRSDNH | 15, 16, | 11, 12, |
| | (SEQ ID NO: 78) | (SEQ ID NO: 412) | 18 | 13 |
| 55 | AHGPCHYNTECNSNK | AVGLLAPPGGLSGRSDDH | 15, 16, | 11, 12, |
| | (SEQ ID NO: 78) | (SEQ ID NO: 691) | 18 | 13 |
| 56 | AHGPCHYNTECNSNK | AVGLLAPPGGLSGRSDIH | 15, 16, | 11, 12, |
| | (SEQ ID NO: 78) | (SEQ ID NO: 692) | 18 | 13 |
| 57 | AHGPCHYNTECNSNK | AVGLLAPPGGLSGRSDQH | 15, 16, | 11, 12, |
| | (SEQ ID NO: 78) | (SEQ ID NO: 693) | 18 | 13 |
| 58 | AHGPCHYNTECNSNK | AVGLLAPPGGLSGRSDTH | 15, 16, | 11, 12, |
| | (SEQ ID NO: 78) | (SEQ ID NO: 694) | 18 | 13 |
| 59 | AHGPCHYNTECNSNK | AVGLLAPPGGLSGRSDYH | 15, 16, | 11, 12, |
| | (SEQ ID NO: 78) | (SEQ ID NO: 695) | 18 | 13 |
| 60 | AHGPCHYNTECNSNK | AVGLLAPPGGLSGRSDNP | 15, 16, | 11, 12, |
| | (SEQ ID NO: 78) | (SEQ ID NO: 696) | 18 | 13 |
| 61 | AHGPCHYNTECNSNK | AVGLLAPPGGLSGRSANP | 15, 16, | 11, 12, |
| | (SEQ ID NO: 78) | (SEQ ID NO: 697) | 18 | 13 |
| 62 | AHGPCHYNTECNSNK | AVGLLAPPGGLSGRSANI | 15, 16, | 11, 12, |
| | (SEQ ID NO: 78) | (SEQ ID NO: 698) | 18 | 13 |
| 63 | AHGPCHYNTECNSNK | AVGLLAPPGGLSGRSDNI | 15, 16, | 11, 12, |
| | (SEQ ID NO: 78) | (SEQ ID NO: 714) | 18 | 13 |
| 64 | AHGPCHYNTECNSNK | ISSGLLSSGGSGGSLSGRSDNH | 15, 16, | 11, 12, |
| | (SEQ ID NO: 78) | (SEQ ID NO: 407) | 18 | 13 |
| 65 | TCLHLTRFNYLSCNK | LSGRSDNH | 15, 16, | 11, 12, |
| | (SEQ ID NO: 88) | (SEQ ID NO: 359) | 18 | 13 |
| 66 | TCLHLTRFNYLSCNK | ISSGLLSS | 15, 16, | 11, 12, |
| | (SEQ ID NO: 88) | (SEQ ID NO: 382) | 18 | 13 |
| 67 | TCLHLTRFNYLSCNK | LSGRSGNH | 15, 16, | 11, 12, |
| | (SEQ ID NO: 88) | (SEQ ID NO: 789) | 18 | 13 |
| 68 | TCLHLTRFNYLSCNK | AVGLLAPP | 15, 16, | 11, 12, |
| | (SEQ ID NO: 88) | (SEQ ID NO: 390) | 18 | 13 |
| 69 | TCLHLTRFNYLSCNK | VHMPLGFLGP | 15, 16, | 11, 12, |
| | (SEQ ID NO: 88) | (SEQ ID NO: 370) | 18 | 13 |
| 70 | TCLHLTRFNYLSCNK | TSTSGRSANPRG | 15, 16, | 11, 12, |
| | (SEQ ID NO: 88) | (SEQ ID NO: 397) | 18 | 13 |
| 71 | TCLHLTRFNYLSCNK | QNQALRMA | 15, 16, | 11, 12, |
| | (SEQ ID NO: 88) | (SEQ ID NO: 377) | 18 | 13 |
| 72 | TCLHLTRFNYLSCNK | ISSGLLSGRSDNH | 15, 16, | 11, 12, |
| | (SEQ ID NO: 88) | (SEQ ID NO: 406) | 18 | 13 |
| 73 | TCLHLTRENYLSCNK | ISSGLLSGRSGNH | 15, 16, | 11, 12, |
| | (SEQ ID NO: 88) | (SEQ ID NO: 423) | 18 | 13 |
| 74 | TCLHLTRENYLSCNK | ISSGLLSGRSANPRG | 15, 16, | 11, 12, |
| | (SEQ ID NO: 88) | (SEQ ID NO: 680) | 18 | 13 |
| 75 | TCLHLTRENYLSCNK | AVGLLAPPTSGRSANPRG | 15, 16, | 11, 12, |
| | (SEQ ID NO: 88) | (SEQ ID NO: 681) | 18 | 13 |
| 76 | TCLHLTRENYLSCNK | AVGLLAPPSGRSANPRG | 15, 16, | 11, 12, |
| | (SEQ ID NO: 88) | (SEQ ID NO: 682) | 18 | 13 |
| 77 | TCLHLTRENYLSCNK | ISSGLLSGRSDDH | 15, 16, | 11, 12, |
| | (SEQ ID NO: 88) | (SEQ ID NO: 683) | 18 | 13 |
| 78 | TCLHLTRENYLSCNK | ISSGLLSGRSDIH | 15, 16, | 11, 12, |
| | (SEQ ID NO: 88) | (SEQ ID NO: 684) | 18 | 13 |
| 79 | TCLHLTRENYLSCNK | ISSGLLSGRSDQH | 15, 16, | 11, 12, |
| | (SEQ ID NO: 88) | (SEQ ID NO: 685) | 18 | 13 |
| 80 | TCLHLTRENYLSCNK | ISSGLLSGRSDTH | 15, 16, | 11, 12, |
| | (SEQ ID NO: 88) | (SEQ ID NO: 686) | 18 | 13 |
| 81 | TCLHLTRENYLSCNK | ISSGLLSGRSDYH | 15, 16, | 11, 12, |
| | (SEQ ID NO: 88) | (SEQ ID NO: 687) | 18 | 13 |
| 82 | TCLHLTRENYLSCNK | ISSGLLSGRSDNP | 15, 16, | 11, 12, |
| | (SEQ ID NO: 88) | (SEQ ID NO: 688) | 18 | 13 |
| 83 | TCLHLTRENYLSCNK | ISSGLLSGRSANP | 15, 16, | 11, 12, |
| | (SEQ ID NO: 88) | (SEQ ID NO: 689) | 18 | 13 |
| 84 | TCLHLTRENYLSCNK | ISSGLLSGRSANI | 15, 16, | 11, 12, |
| | (SEQ ID NO: 88) | (SEQ ID NO: 690) | 18 | 13 |
| 85 | TCLHLTRENYLSCNK | ISSGLLSGRSDNI | 15, 16, | 11, 12, |
| | (SEQ ID NO: 88) | (SEQ ID NO: 713) | 18 | 13 |
| 86 | TCLHLTRENYLSCNK | AVGLLAPPGGLSGRSDNH | 15, 16, | 11, 12, |
| | (SEQ ID NO: 88) | (SEQ ID NO: 412) | 18 | 13 |
| 87 | TCLHLTRENYLSCNK | AVGLLAPPGGLSGRSDDH | 15, 16, | 11, 12, |
| | (SEQ ID NO: 88) | (SEQ ID NO: 691) | 18 | 13 |
| 88 | TCLHLTRENYLSCNK | AVGLLAPPGGLSGRSDIH | 15, 16, | 11, 12, |
| | (SEQ ID NO: 88) | (SEQ ID NO: 692) | 18 | 13 |
| 89 | TCLHLTRENYLSCNK | AVGLLAPPGGLSGRSDQH | 15, 16, | 11, 12, |
| | (SEQ ID NO: 88) | (SEQ ID NO: 693) | 18 | 13 |
| 90 | TCLHLTRENYLSCNK | AVGLLAPPGGLSGRSDTH | 15, 16, | 11, 12, |
| | (SEQ ID NO: 88) | (SEQ ID NO: 694) | 18 | 13 |
| 91 | TCLHLTRENYLSCNK | AVGLLAPPGGLSGRSDYH | 15, 16, | 11, 12, |
| | (SEQ ID NO: 88) | (SEQ ID NO: 695) | 18 | 13 |
| 92 | TCLHLTRENYLSCNK | AVGLLAPPGGLSGRSDNP | 15, 16, | 11, 12, |
| | (SEQ ID NO: 88) | (SEQ ID NO: 696) | 18 | 13 |
| 93 | TCLHLTRENYLSCNK | AVGLLAPPGGLSGRSANP | 15, 16, | 11, 12, |
| | (SEQ ID NO: 88) | (SEQ ID NO: 697) | 18 | 13 |
| 94 | TCLHLTRENYLSCNK | AVGLLAPPGGLSGRSANI | 15, 16, | 11, 12, |
| | (SEQ ID NO: 88) | (SEQ ID NO: 698) | 18 | 13 |
| 95 | TCLHLTRENYLSCNK | AVGLLAPPGGLSGRSDNI | 15, 16, | 11, 12, |
| | (SEQ ID NO: 88) | (SEQ ID NO: 714) | 18 | 13 |
| 96 | TCLHLTRFNYLSCNK | ISSGLLSSGGSGGSLSGRSDNH | 15, 16, | 11, 12, |
| | (SEQ ID NO: 88) | (SEQ ID NO: 407) | 18 | 13 |
| 97 | LHGPCHYDLKCK | LSGRSDNH | 15, 16, | 11, 12, |
| | (SEQ ID NO: 96) | (SEQ ID NO: 359) | 18 | 13 |
| 98 | LHGPCHYDLKCK | ISSGLLSS | 15, 16, | 11, 12, |
| | (SEQ ID NO: 96) | (SEQ ID NO: 382) | 18 | 13 |
| 99 | LHGPCHYDLKCK | LSGRSGNH | 15, 16, | 11, 12, |
| | (SEQ ID NO: 96) | (SEQ ID NO: 789) | 18 | 13 |
| 100 | LHGPCHYDLKCK | AVGLLAPP | 15, 16, | 11, 12, |
| | (SEQ ID NO: 96) | (SEQ ID NO: 390) | 18 | 13 |
| 101 | LHGPCHYDLKCK | VHMPLGFLGP | 15, 16, | 11, 12, |
| | (SEQ ID NO: 96) | (SEQ ID NO: 370) | 18 | 13 |
| 102 | LHGPCHYDLKCK | TSTSGRSANPRG | 15, 16, | 11, 12, |
| | (SEQ ID NO: 96) | (SEQ ID NO: 397) | 18 | 13 |
| 103 | LHGPCHYDLKCK | QNQALRMA | 15, 16, | 11, 12, |
| | (SEQ ID NO: 96) | (SEQ ID NO: 377) | 18 | 13 |
| 104 | LHGPCHYDLKCK | ISSGLLSGRSDNH | 15, 16, | 11, 12, |
| | (SEQ ID NO: 96) | (SEQ ID NO: 406) | 18 | 13 |
| 105 | LHGPCHYDLKCK | ISSGLLSGRSGNH | 15, 16, | 11, 12, |
| | (SEQ ID NO: 96) | (SEQ ID NO: 423) | 18 | 13 |
| 106 | LHGPCHYDLKCK | ISSGLLSGRSANPRG | 15, 16, | 11, 12, |
| | (SEQ ID NO: 96) | (SEQ ID NO: 680) | 18 | 13 |
| 107 | LHGPCHYDLKCK | AVGLLAPPTSGRSANPRG | 15, 16, | 11, 12, |
| | (SEQ ID NO: 96) | (SEQ ID NO: 681) | 18 | 13 |
| 108 | LHGPCHYDLKCK | AVGLLAPPSGRSANPRG | 15, 16, | 11, 12, |
| | (SEQ ID NO: 96) | (SEQ ID NO: 682) | 18 | 13 |
| 109 | LHGPCHYDLKCK | ISSGLLSGRSDDH | 15, 16, | 11, 12, |
| | (SEQ ID NO: 96) | (SEQ ID NO: 683) | 18 | 13 |
| 110 | LHGPCHYDLKCK | ISSGLLSGRSDIH | 15, 16, | 11, 12, |
| | (SEQ ID NO: 96) | (SEQ ID NO: 684) | 18 | 13 |
| 111 | LHGPCHYDLKCK | ISSGLLGSRSDQH | 15, 16, | 11, 12, |
| | (SEQ ID NO: 96) | (SEQ ID NO: 685) | 18 | 13 |
| 112 | LHGPCHYDLKCK | ISSGLLSGRSDTH | 15, 16, | 11, 12, |
| | (SEQ ID NO: 96) | (SEQ ID NO: 686) | 18 | 13 |
| 113 | LHGPCHYDLKCK | ISSGLLSGRSDYH | 15, 16, | 11, 12, |
| | (SEQ ID NO: 96) | (SEQ ID NO: 687) | 18 | 13 |
| 114 | LHGPCHYDLKCK | ISSGLLSGRSDNP | 15, 16, | 11, 12, |
| | (SEQ ID NO: 96) | (SEQ ID NO: 688) | 18 | 13 |
| 115 | LHGPCHYDLKCK | ISSGLLSGRSANP | 15, 16, | 11, 12, |
| | (SEQ ID NO: 96) | (SEQ ID NO: 689) | 18 | 13 |
| 116 | LHGPCHYDLKCK | ISSGLLSGRSANI | 15, 16, | 11, 12, |
| | (SEQ ID NO: 96) | (SEQ ID NO: 690) | 18 | 13 |
| 117 | LHGPCHYDLKCK | ISSGLLSGRSDNI | 15, 16, | 11, 12, |
| | (SEQ ID NO: 96) | (SEQ ID NO: 713) | 18 | 13 |
| 118 | LHGPCHYDLKCK | AVGLLAPPGGLSGRSDNH | 15, 16, | 11, 12, |
| | (SEQ ID NO: 96) | (SEQ ID NO: 412) | 18 | 13 |
| 119 | LHGPCHYDLKCK | AVGLLAPPGGLSGRSDDH | 15, 16, | 11, 12, |
| | (SEQ ID NO: 96) | (SEQ ID NO: 691) | 18 | 13 |
| 120 | LHGPCHYDLKCK | AVGLLAPPGGLSGRSDIH | 15, 16, | 11, 12, |
| | (SEQ ID NO: 96) | (SEQ ID NO: 692) | 18 | 13 |
| 121 | LHGPCHYDLKCK | AVGLLAPPGGLSGRSDQH | 15, 16, | 11, 12, |
| | (SEQ ID NO: 96) | (SEQ ID NO: 693) | 18 | 13 |
| 122 | LHGPCHYDLKCK | AVGLLAPPGGLSGRSDTH | 15, 16, | 11, 12, |
| | (SEQ ID NO: 96) | (SEQ ID NO: 694) | 18 | 13 |
| 123 | LHGPCHYDLKCK | AVGLLAPPGGLSGRSDYH | 15, 16, | 11, 12, |
| | (SEQ ID NO: 96) | (SEQ ID NO: 695) | 18 | 13 |
| 124 | LHGPCHYDLKCK | AVGLLAPPGGLSGRSDNP | 15, 16, | 11, 12, |
| | (SEQ ID NO: 96) | (SEQ ID NO: 696) | 18 | 13 |
| 125 | LHGPCHYDLKCK | AVGLLAPPGGLSGRSANP | 15, 16, | 11, 12, |
| | (SEQ ID NO: 96) | (SEQ ID NO: 697) | 18 | 13 |
| 126 | LHGPCHYDLKCK | AVGLLAPPGGLSGRSANI | 15, 16, | 11, 12, |
| | (SEQ ID NO: 96) | (SEQ ID NO: 698) | 18 | 13 |
| 127 | LHGPCHYDLKCK | AVGLLAPPGGLSGRSDNI | 15, 16, | 11, 12, |
| | (SEQ ID NO: 96) | (SEQ ID NO: 714) | 18 | 13 |
| 128 | LHGPCHYDLKCK | ISSGLLSSGGSGGSLSGRSDNH | 15, 16, | 11, 12, |
| | (SEQ ID NO: 96) | (SEQ ID NO: 407) | 18 | 13 |
| 129 | TCLHLTRFNYLSC | LSGRSDNH | 15, 16, | 11, 12, |
| | (SEQ ID NO: 100) | (SEQ ID NO: 359) | 18 | 13 |
| 130 | TCLHLTRFNYLSC | ISSGLLSS | 15, 16, | 11, 12, |
| | (SEQ ID NO: 100) | (SEQ ID NO: 382) | 18 | 13 |
| 131 | TCLHLTRFNYLSC | LSGRSGNH | 15, 16, | 11, 12, |
| | (SEQ ID NO: 100) | (SEQ ID NO: 789) | 18 | 13 |
| 132 | TCLHLTRFNYLSC | AVGLLAPP | 15, 16, | 11, 12, |
| | (SEQ ID NO: 100) | (SEQ ID NO: 390) | 18 | 13 |
| 133 | TCLHLTRFNYLSC | VHMPLGFLGP | 15, 16, | 11, 12, |
| | (SEQ ID NO: 100) | (SEQ ID NO: 370) | 18 | 13 |
| 134 | TCLHLTRFNYLSC | TSTSGRSANPRG | 15, 16, | 11, 12, |
| | (SEQ ID NO: 100) | (SEQ ID NO: 397) | 18 | 13 |
| 135 | TCLHLTRFNYLSC | QNQALRMA | 15, 16, | 11, 12, |
| | (SEQ ID NO: 100) | (SEQ ID NO: 377) | 18 | 13 |
| 136 | TCLHLTRFNYLSC | ISSGLLSGRSDNH | 15, 16, | 11, 12, |
| | (SEQ ID NO: 100) | (SEQ ID NO: 406) | 18 | 13 |
| 137 | TCLHLTRFNYLSC | ISSGLLSGRSGNH | 15, 16, | 11, 12, |
| | (SEQ ID NO: 100) | (SEQ ID NO: 423) | 18 | 13 |
| 138 | TCLHLTRFNYLSC | ISSGLLSGRSANPRG | 15, 16, | 11, 12, |
| | (SEQ ID NO: 100) | (SEQ ID NO: 680) | 18 | 13 |
| 139 | TCLHLTRFNYLSC | AVGLLAPPTSGRSANPRG | 15, 16, | 11, 12, |
| | (SEQ ID NO: 100) | (SEQ ID NO: 681) | 18 | 13 |
| 140 | TCLHLTRFNYLSC | AVGLLAPPSGRSANPRG | 15, 16, | 11, 12, |
| | (SEQ ID NO: 100) | (SEQ ID NO: 682) | 18 | 13 |
| 141 | TCLHLTRFNYLSC | ISSGLLSGRSDDH | 15, 16, | 11, 12, |
| | (SEQ ID NO: 100) | (SEQ ID NO: 683) | 18 | 13 |
| 142 | TCLHLTRFNYLSC | ISSGLLSGRSDIH | 15, 16, | 11, 12, |
| | (SEQ ID NO: 100) | (SEQ ID NO: 684) | 18 | 13 |
| 143 | TCLHLTRFNYLSC | ISSGLLSGRSDQH | 15, 16, | 11, 12, |
| | (SEQ ID NO: 100) | (SEQ ID NO: 685) | 18 | 13 |
| 144 | TCLHLTRFNYLSC | ISSGLLSGRSDTH | 15, 16, | 11, 12, |
| | (SEQ ID NO: 100) | (SEQ ID NO: 686) | 18 | 13 |
| 145 | TCLHLTRFNYLSC | ISSGLLSGRSDYH | 15, 16, | 11, 12, |
| | (SEQ ID NO: 100) | (SEQ ID NO: 687) | 18 | 13 |
| 146 | TCLHLTRFNYLSC | ISSGLLSGRSDNP | 15, 16, | 11, 12, |
| | (SEQ ID NO: 100) | (SEQ ID NO: 688) | 18 | 13 |
| 147 | TCLHLTRFNYLSC | ISSGLLSGRSANP | 15, 16, | 11, 12, |
| | (SEQ ID NO: 100) | (SEQ ID NO: 689) | 18 | 13 |
| 148 | TCLHLTRFNYLSC | ISSGLLSGRSANI | 15, 16, | 11, 12, |
| | (SEQ ID NO: 100) | (SEQ ID NO: 690) | 18 | 13 |
| 149 | TCLHLTRFNYLSC | ISSGLLSGRSDNI | 15, 16, | 11, 12, |
| | (SEQ ID NO: 100) | (SEQ ID NO: 713) | 18 | 13 |
| 150 | TCLHLTRFNYLSC | AVGLLAPPGGLSGRSDNH | 15, 16, | 11, 12, |
| | (SEQ ID NO: 100) | (SEQ ID NO: 412) | 18 | 13 |
| 151 | TCLHLTRFNYLSC | AVGLLAPPGGLSGRSDDH | 15, 16, | 11, 12, |
| | (SEQ ID NO: 100) | (SEQ ID NO: 691) | 18 | 13 |
| 152 | TCLHLTRFNYLSC | AVGLLAPPGGLSGRSDIH | 15, 16, | 11, 12, |
| | (SEQ ID NO: 100) | (SEQ ID NO: 692) | 18 | 13 |
| 153 | TCLHLTRFNYLSC | AVGLLAPPGGLSGRSDQH | 15, 16, | 11, 12, |
| | (SEQ ID NO: 100) | (SEQ ID NO: 698) | 18 | 13 |
| 154 | TCLHLTRFNYLSC | AVGLLAPPGGLSGRSDTH | 15, 16, | 11, 12, |
| | (SEQ ID NO: 100) | (SEQ ID NO: 694) | 18 | 13 |
| 155 | TCLHLTRFNYLSC | AVGLLAPPGGLSGRSDYH | 15, 16, | 11, 12, |
| | (SEQ ID NO: 100) | (SEQ ID NO: 695) | 18 | 13 |
| 156 | TCLHLTRFNYLSC | AVGLLAPPGGLSGRSDNP | 15, 16, | 11, 12, |
| | (SEQ ID NO: 100) | (SEQ ID NO: 696) | 18 | 13 |
| 157 | TCLHLTRFNYLSC | AVGLLAPPGGLSGRSANP | 15, 16, | 11, 12, |
| | (SEQ ID NO: 100) | (SEQ ID NO: 697) | 18 | 13 |
| 158 | TCLHLTRFNYLSC | AVGLLAPPGGLSGRSANI | 15, 16, | 11, 12, |
| | (SEQ ID NO: 100) | (SEQ ID NO: 698) | 18 | 13 |
| 159 | TCLHLTRFNYLSC | AVGLLAPPGGLSGRSDNI | 15, 16, | 11, 12, |
| | (SEQ ID NO: 100) | (SEQ ID NO: 714) | 18 | 13 |
| 160 | TCLHLTRFNYLSC | ISSGLLSSGGSGGSLSGRSDNH | 15, 16, | 11, 12, |
| | (SEQ ID NO: 100) | (SEQ ID NO: 407) | 18 | 13 |

TABLE 5
CD147 Activatable Antibody Components

| Mask Sequence | Substrate Sequence | VL or | VH or |
|---|---|---|---|
| (MM) | (CM) | VL CDRs | VH CDRs |
| RYQSCHSYWCTEGNH | LSGRSDNH | SEQ ID | SEQ ID |
| (SEQ ID NO: 30) | (SEQ ID NO: 359) | NOS: 15, 16, | NOS: 11, 12, |
| | | 18 | 13 |
| QSLFCSGFRCDQYAS | TGRGPSWV | SEQ ID | SEQ ID |
| (SEQ ID NO: 31) | (SEQ ID NO: 356) | NOS: 14, 16, | NOS: 10, 12, |
| | | 17 | 13 |
| DHGPCHYVSCTTINH | PLTGRSGG | SEQ ID NO: 23 | SEQ ID |
| (SEQ ID NO: 33) | (SEQ ID NO: 362) | | NO: 19 |
| VHGPCHWSVECLSNV | TARGPSFK | SEQ ID NO: 24 | SEQ ID |
| (SEQ ID NO: 44) | (SEQ ID NO: 358) | | NO: 20 |
| HGPCHYNFNSGCAQF | NTLSGRSENHSG | SEQ ID NO: 25 | SEQ ID |
| (SEQ ID NO: 46) | (SEQ ID NO: 395) | | NO: 21 |
| THGPCHFKPNCSYPT | NTLSGRSGNHGS | SEQ ID NO: 26 | |
| (SEQ ID NO: 75) | (SEQ ID NO: 396) | | |
| LHGPCHYDLKCKNNT | TSTSGRSANPRG | | |
| (SEQ ID NO: 76) | (SEQ ID NO: 397) | | |
| SHGPCHFDYQCINNT | TSGRSANP | | |
| (SEQ ID NO: 77) | (SEQ ID NO: 398) | | |
| AHGPCHYNTECNSNK | VHMPLGFLGP | | |
| (SEQ ID NO: 78) | (SEQ ID NO: 370) | | |
| LHGPCHYMNTCHNVK | AVGLLAPP | | |
| (SEQ ID NO: 79) | (SEQ ID NO: 390) | | |
| LHGPCHFNNCNTLKL | AQNLLGMV | | |
| (SEQ ID NO: 80) | (SEQ ID NO: 378) | | |
| WHGPCHYTKCDDHTM | QNQALRMA | | |
| (SEQ ID NO: 82) | (SEQ ID NO: 377) | | |
| THGPCHYKECDWMTI | LAAPLGLL | | |
| (SEQ ID NO: 83) | (SEQ ID NO: 389) | | |
| KHGPCHFRLCPQNTS | STFPFGMF | | |
| (SEQ ID NO: 86) | (SEQ ID NO: 379) | | |
| WQRECSOKNTCQYYI | ISSGLLSS | | |
| (SEQ ID NO: 87) | (SEQ ID NO: 382) | | |
| TCLHLTRFNYLSCNK | PAGLWLDP | | |
| (SEQ ID NO: 88) | (SEQ ID NO: 392) | | |
| FSCGFRGGYMRLCGG | VAGRSMRP | | |
| (SEQ ID NO: 89) | (SEQ ID NO: 399) | | |
| THGPCHFKPNC | VVPEGRRS | | |
| (SEQ ID NO: 90) | (SEQ ID NO: 400) | | |
| THGPCHFKPQCSYPT | ILPRSPAF | | |
| (SEQ ID NO: 91) | (SEQ ID NO: 401) | | |
| THGPCHFKPNCAYPT | MVLGRSLL | | |
| (SEQ ID NO: 92) | (SEQ ID NO: 402) | | |
| THGPCHFRPNCAYPT | QGRAITFI | | |
| (SEQ ID NO: 93) | (SEQ ID NO: 403) | | |
| LHGPCHYDLKCKQNT | SPRSIMLA | | |
| (SEQ ID NO: 94) | (SEQ ID NO: 404) | | |
| LHGPCHYDLKCKNN | SMLRSMPL | | |
| (SEQ ID NO: 95) | (SEQ ID NO: 405) | | |
| LHGPCHYDLKCK | ISSGLLSGRSDNH | | |
| (SEQ ID NO: 96) | (SEQ ID NO: 406) | | |
| LHGPCHYDLKC | AVGLLAPPGGLSGRSDNH | | |
| (SEQ ID NO: 97) | (SEQ ID NO: 412) | | |
| LHGPCHYDLRC | ISSGLLSSGGSGGSLSGRSDNH | | |
| (SEQ ID NO: 98) | (SEQ ID NO: 407) | | |
| LHGPCHFNNCNTL | LSGRSGNH | | |
| (SEQ ID NO: 99) | (SEQ ID NO: 789) | | |
| TCLHLTRFNYLSC | SGRSANPRG | | |
| (SEQ ID NO: 100) | (SEQ ID NO: 790) | | |
| | LSGRSDDH | | |
| | (SEQ ID NO: 791) | | |
| | LSGRSDIH | | |
| | (SEQ ID NO: 792) | | |
| | LSGRSDQH | | |
| | (SEQ ID NO: 793) | | |
| | LSGRSDTH | | |
| | (SEQ ID NO: 794) | | |
| | LSGRSDYH | | |
| | (SEQ ID NO: 795) | | |
| | LSGRSDNP | | |
| | (SEQ ID NO: 796) | | |
| | LSGRSANP | | |
| | (SEQ ID NO: 797) | | |
| | LSGRSANI | | |
| | (SEQ ID NO: 798) | | |
| | LSGRSDNI | | |
| | (SEQ ID NO: 799) | | |
| | MIAPVAYR | | |
| | (SEQ ID NO: 800) | | |
| | RPSPMWAY | | |
| | (SEQ ID NO: 801) | | |
| | WATPRPMR | | |
| | (SEQ ID NO: 802) | | |
| | FRLLDWQW | | |
| | (SEQ ID NO: 803) | | |
| | ISSGL | | |
| | (SEQ ID NO: 804) | | |
| | ISSGLLS | | |
| | (SEQ ID NO: 805) | | |
| | ISSGLL | | |
| | (SEQ ID NO: 806) | | |
| | ISSGLLSGRSANPRG | | |
| | (SEQ ID NO: 680) | | |
| | AVGLLAPPTSGRSANPRG | | |
| | (SEQ ID NO: 681) | | |
| | AVGLLAPPSGRSANPRG | | |
| | (SEQ ID NO: 682) | | |
| | ISSGLLSGRSDDH | | |
| | (SEQ ID NO: 683) | | |
| | ISSGLLSGRSDIH | | |
| | (SEQ ID NO: 684) | | |
| | ISSGLLSGRSDQH | | |
| | (SEQ ID NO: 685) | | |
| | ISSGLLSGRSDTH | | |
| | (SEQ ID NO: 686) | | |
| | ISSGLLSGRSDYH | | |
| | (SEQ ID NO: 687) | | |
| | ISSGLLSGRSDNP | | |
| | (SEQ ID NO: 688) | | |
| | ISSGLLSGRSANP | | |
| | (SEQ ID NO: 689) | | |
| | ISSGLLSGRSANI | | |
| | (SEQ ID NO: 690) | | |
| | AVGLLAPPGGLSGRSDDH | | |
| | (SEQ ID NO: 691) | | |
| | AVGLLAPPGGLSGRSDIH | | |
| | (SEQ ID NO: 692) | | |
| | AVGLLAPPGGLSGRSDQH | | |
| | (SEQ ID NO: 693) | | |
| | AVGLLAPPGGLSGRSDTH | | |
| | (SEQ ID NO: 694) | | |
| | AVGLLAPPGGLSGRSDYH | | |
| | (SEQ ID NO: 695) | | |
| | AVGLLAPPGGLSGRSDNP | | |
| | (SEQ ID NO: 696) | | |
| | AVGLLAPPGGLSGRSANP | | |
| | (SEQ ID NO: 697) | | |
| | AVGLLAPPGGLSGRSANI | | |
| | (SEQ ID NO: 698) | | |
| | ISSGLLSGRSDNI | | |
| | (SEQ ID NO: 713) | | |
| | AVGLLAPPGGLSGRSDNI | | |
| | (SEQ ID NO: 714) | | |
| | GLSGRSDNHGGAVGLLAPP | | |
| | (SEQ ID NO: 807) | | |
| | GLSGRSDNHGGVHMPLGFLGP | | |
| | (SEQ ID NO: 808) | | |

In some embodiments, an activatable antibody of the present disclosure includes one or more polypeptides that include the combination of sequences selected from Table 4 or Table 5, where the polypeptide includes a combination of a masking sequence selected from the column titled “Mask Sequence (MM)” of Table 4 or Table 5, a substrate sequence from the column titled “Substrate Sequence (CM)” of Table 4 or Table 5, a light chain variable domain or light chain CDRs from the column titled “VL or VL CDRs” or “VL CDRs SEQ ID NOs” of Table 4 or Table 5, and a heavy chain variable domain or heavy chain CDRs from the column titled “VH or VH CDRs” or “VH CDRs SEQ ID Nos” of Table 4 or Table 5. For example, an activatable antibody of the present disclosure may include the amino acid sequences of combination no. 147, which includes the masking sequence of SEQ ID NO: 17, the substrate sequence of SEQ ID NO: 412, a light chain variable domain that includes the VL CDR sequences of SEQ ID NOs: 15, 16, and 18, and a heavy chain variable domain that includes the VH CDR sequences of 11, 12, and 13. Therefore, an activatable antibody that includes at least the combination of sequences in any given row of Table 4 is described herein. Similarly, any combination of a mask sequence (MM), a substrate sequence (CM), a light chain variable domain sequence or light chain variable domain CDR sequences, and a heavy chain variable domain sequence or heavy chain variable domain CDR sequences of Table 5 is described herein. An activatable antibody that includes at least any combination of a masking sequence, a substrate sequence, a variable heavy chain or variable heavy chain CDRs, and a variable light chain or variable light chain CDRs selected from the corresponding columns Table 4 or Table 5 is also described herein. In some exemplary embodiments, an activatable antibody that includes at least the combination of sequences in any given row of Table 4 or any combination of a mask sequence (MM), a substrate sequence (CM), a light chain variable domain sequence or light chain variable domain CDR sequences, and a heavy chain variable domain sequence or heavy chain variable domain CDR sequences of Table 5 can be combined with one or more toxins, including a dolastatin or a derivative thereof, an auristatin or a derivative thereof, a maytansinoid or a derivative thereof, a duocarmycin or a derivative thereof, a calicheamicin or a derivative thereof, or a pyrrolobenzodiazepine or a derivative thereof. In some exemplary embodiments, an activatable antibody that includes at least the combination of sequences in any given row of Table 4 or any combination of a mask sequence (MM), a substrate sequence (CM), a light chain variable domain sequence or light chain variable domain CDR sequences, and a heavy chain variable domain sequence or heavy chain variable domain CDR sequences of Table 5 can be combined with one or more toxins, including auristatin E, monomethyl auristatin F (MMAF), monomethyl auristatin E (MMAE), monomethyl auristatin D (MMAD), maytansinoid DM4, maytansinoid DM1, a pyrrolobenzodiazepine, a pyrrolobenzodiazepine dimer, and/or a duocarmycin.

Any of the combinations in Table 4 or Table 5 as described above can be combined with human immunoglobulin constant regions to result in fully human IgGs including IgG1, IgG2, IgG4 or mutated constant regions to result in human IgGs with altered functions such as IgG N297A, IgG1 N297Q, or IgG4 S228P. The combinations described in Table 4 or Table 5 are not limited by the particular combinations shown in any given row, and thus may include any mask sequence from column 2 of Table 4 (or column 1 of Table 5) combined with any substrate sequence from column 3 of Table 4 (or column 2 of Table 5) combined with any VL sequence or set of VL CDR sequences from column 4 of Table 4 (or column 3 or Table 5) combined with any VH sequence or set of VH CDR sequences from column 5 of Table 4 (or column 4 of Table 5). In addition to the mask sequences disclosed in column 2 of Table 4 or column 1 of Table 5, any mask sequence disclosed herein can be used in a combination. In addition to the substrate sequences disclosed in column 3 of Table 4 or column 2 of Table 5, any CM disclosed herein can be used in a combination. In addition to the light chain variable region sequence or light chain CDR sequences disclosed in column 4 of Table 4 or column 3 of Table 5, any light chain variable region sequence or light chain CDR sequences disclosed herein can be used in a combination. In addition to the heavy chain variable region sequence or heavy chain CDR sequences disclosed in column 5 of Table 4 or column 4 of Table 5, any heavy chain variable region sequence or heavy chain CDR sequences disclosed herein can be used in a combination.

In some embodiments, the serum half-life of the activatable antibody is longer than that of the corresponding antibody; e.g., the pK of the activatable antibody is longer than that of the corresponding antibody. In some embodiments, the serum half-life of the activatable antibody is similar to that of the corresponding antibody. In some embodiments, the serum half-life of the activatable antibody is at least 15 days when administered to an organism. In some embodiments, the serum half-life of the activatable antibody is at least 12 days when administered to an organism. In some embodiments, the serum half-life of the activatable antibody is at least 11 days when administered to an organism. In some embodiments, the serum half-life of the activatable antibody is at least 10 days when administered to an organism. In some embodiments, the serum half-life of the activatable antibody is at least 9 days when administered to an organism. In some embodiments, the serum half-life of the activatable antibody is at least 8 days when administered to an organism. In some embodiments, the serum half-life of the activatable antibody is at least 7 days when administered to an organism. In some embodiments, the serum half-life of the activatable antibody is at least 6 days when administered to an organism. In some embodiments, the serum half-life of the activatable antibody is at least 5 days when administered to an organism. In some embodiments, the serum half-life of the activatable antibody is at least 4 days when administered to an organism. In some embodiments, the serum half-life of the activatable antibody is at least 3 days when administered to an organism. In some embodiments, the serum half-life of the activatable antibody is at least 2 days when administered to an organism. In some embodiments, the serum half-life of the activatable antibody is at least 24 hours when administered to an organism. In some embodiments, the serum half-life of the activatable antibody is at least 20 hours when administered to an organism. In some embodiments, the serum half-life of the activatable antibody is at least 18 hours when administered to an organism. In some embodiments, the serum half-life of the activatable antibody is at least 16 hours when administered to an organism. In some embodiments, the serum half-life of the activatable antibody is at least 14 hours when administered to an organism. In some embodiments, the serum half-life of the activatable antibody is at least 12 hours when administered to an organism. In some embodiments, the serum half-life of the activatable antibody is at least 10 hours when administered to an organism. In some embodiments, the serum half-life of the activatable antibody is at least 8 hours when administered to an organism. In some embodiments, the serum half-life of the activatable antibody is at least 6 hours when administered to an organism. In some embodiments, the serum half-life of the activatable antibody is at least 4 hours when administered to an organism. In some embodiments, the serum half-life of the activatable antibody is at least 3 hours when administered to an organism.

The disclosure also provides methods of producing an activatable CD147 antibody polypeptide by culturing a cell under conditions that lead to expression of the polypeptide, wherein the cell comprises an isolated nucleic acid molecule encoding an antibody and/or an activatable antibody described herein, and/or vectors that include these isolated nucleic acid sequences. The disclosure provides methods of producing an antibody and/or activatable antibody by culturing a cell under conditions that lead to expression of the antibody and/or activatable antibody, wherein the cell comprises an isolated nucleic acid molecule encoding an antibody and/or an activatable antibody described herein, and/or vectors that include these isolated nucleic acid sequences.

The invention also provides a method of manufacturing activatable antibodies that in an activated state binds CD147 by (a) culturing a cell comprising a nucleic acid construct that encodes the activatable antibody under conditions that lead to expression of the activatable antibody, wherein the activatable antibody comprises a masking moiety (MM), a cleavable moiety (CM), and an antibody or an antigen binding fragment thereof (AB) that specifically binds CD147, (i) wherein the CM is a polypeptide that functions as a substrate for a protease; and (ii) wherein the CM is positioned in the activatable antibody such that, when the activatable antibody is in an uncleaved state, the MM interferes with specific binding of the AB to CD147 and in a cleaved state the MM does not interfere or compete with specific binding of the AB to CD147; and (b) recovering the activatable antibody. Suitable AB, MM, and/or CM include any of the AB, MM, and/or CM disclosed herein.

In some embodiments, the activatable antibody in the uncleaved state has the structural arrangement from N-terminus to C-terminus as follows: MM-CM-AB or AB-CM-MM. In some embodiments, the activatable antibody comprises a linking peptide between the MM and the CM. In some embodiments, the activatable antibody comprises a linking peptide between the CM and the AB. In some embodiments, the activatable antibody comprises a first linking peptide (LP1) and a second linking peptide (LP2), and wherein the activatable antibody in the uncleaved state has the structural arrangement from N-terminus to C-terminus as follows: MM-LP1-CM-LP2-AB or AB-LP2-CM-LP1-MM. In some embodiments, the two linking peptides need not be identical to each other. In some embodiments, the activatable antibody in the uncleaved state has the structural arrangement from N-terminus to C-terminus as follows: spacer-MM-LP1-CM-LP2-AB or AB-LP2-CM-LP1-MM-spacer.

In some embodiments, at least one of LP1 or LP2 comprises an amino acid sequence selected from the group consisting of (GS)ₙ, (GGS)ₙ, (GSGGS)ₙ (SEQ ID NO: 339) and (GGGS)ₙ (SEQ ID NO: 340), where n is an integer of at least one.

In some embodiments, at least one of LP1 or LP2 comprises an amino acid sequence selected from the group consisting of GGSG (SEQ ID NO: 341), GGSGG (SEQ ID NO: 342), GSGSG (SEQ ID NO: 343), GSGGG (SEQ ID NO: 344), GGGSG (SEQ ID NO: 345), and GSSSG (SEQ ID NO: 346).

In some embodiments, LP1 comprises the amino acid sequence GSSGGSGGSGGSG (SEQ ID NO: 347), GSSGGSGGSGG (SEQ ID NO: 348), GSSGGSGGSGGS (SEQ ID NO: 349), GSSGGSGGSGGSGGGS (SEQ ID NO: 350), GSSGGSGGSG (SEQ ID NO: 351), or GSSGGSGGSGS (SEQ ID NO: 352).

In some embodiments, LP2 comprises the amino acid sequence GSS, GGS, GGGS (SEQ ID NO: 353), GSSGT (SEQ ID NO: 354) or GSSG (SEQ ID NO: 356).

Conjugated CD147 Antibodies and Activatable Antibodies

In some embodiments, the CD147 antibodies and activatable antibodies described herein also include an agent conjugated to the antibody/activatable antibody. In some embodiments, the conjugated agent is a therapeutic agent, such as an anti-inflammatory and/or an antineoplastic agent. In such embodiments, the agent is conjugated to a carbohydrate moiety of the antibody/activatable antibody, for example, in some embodiments, where the carbohydrate moiety is located outside the antigen-binding region of the antibody or antigen-binding fragment in the activatable antibody. In some embodiments, the agent is conjugated to a sulfhydryl group of the antibody or antigen-binding fragment in the antibody/activatable antibody.

In some embodiments, the agent is a cytotoxic agent such as a toxin (e.g., an enzymatically active toxin of bacterial, fungal, plant, or animal origin, or fragments thereof), or a radioactive isotope (i.e., a radioconjugate).

In some embodiments, the agent is a detectable moiety such as, for example, a label or other marker. For example, the agent is or includes a radiolabeled amino acid, one or more biotinyl moieties that can be detected by marked avidin (e.g., streptavidin containing a fluorescent marker or enzymatic activity that can be detected by optical or calorimetric methods), one or more radioisotopes or radionuclides, one or more fluorescent labels, one or more enzymatic labels, and/or one or more chemiluminescent agents. In some embodiments, detectable moieties are attached by spacer molecules.

The disclosure also pertains to immunoconjugates comprising an antibody conjugated to a cytotoxic agent such as a toxin (e.g., an enzymatically active toxin of bacterial, fungal, plant, or animal origin, or fragments thereof), or a radioactive isotope (i.e., a radioconjugate). Suitable cytotoxic agents include, for example, dolastatins and derivatives thereof (e.g. auristatin E, AFP, MMAF, MMAE, MMAD, DMAF, DMAE). For example, the agent is monomethyl auristatin E (MMAE) or monomethyl auristatin D (MMAD). In some embodiments, the agent is an agent selected from the group listed in Table 5. In some embodiments, the agent is a dolastatin. In some embodiments, the agent is an auristatin or derivative thereof. In some embodiments, the agent is auristatin E or a derivative thereof. In some embodiments, the agent is monomethyl auristatin E (MMAE). In some embodiments, the agent is monomethyl auristatin D (MMAD). In some embodiments, the agent is a maytansinoid or maytansinoid derivative. In some embodiments, the agent is DM1 or DM4. In some embodiments, the agent is a duocarmycin or derivative thereof. In some embodiments, the agent is a calicheamicin or derivative thereof. In some embodiments, the agent is a pyrrolobenzodiazepine. In some embodiments, the agent is a pyrrolobenzodiazepine dimer.

Enzymatically active toxins and fragments thereof that can be used include diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), momordica charantia inhibitor, curcin, crotin, sapaonaria officinalis inhibitor, gelonin, mitogellin, restrictocin, phenomycin, enomycin, and the tricothecenes. A variety of radionuclides are available for the production of radioconjugated antibodies. Examples include ²¹²Bi, ¹³¹I, ¹³¹In, ⁹⁰Y, and ¹⁸⁶Re.

In some embodiments, the agent is a toxin or fragment thereof. In some embodiments, the agent is a microtubule inhibitor. In some embodiments, the agent is a nucleic acid damaging agent. In some embodiments, the agent is selected from the group consisting of a dolastatin or a derivative thereof, an auristatin or a derivative thereof, a maytansinoid or a derivative thereof, a duocarmycin or a derivative thereof, a calicheamicin or a derivative thereof, and a pyrrolobenzodiazepine or a derivative thereof. In some embodiments, the agent is auristatin E or a derivative thereof. In some embodiments, the agent is monomethyl auristatin E (MMAE). In some embodiments, the agent is monomethyl auristatin D (MMAD). In some embodiments, the agent is a maytansinoid selected from the group consisting of DM1 and DM4. In some embodiments, the agent is maytansinoid DM4. In some embodiments, the agent is duocarmycin. In some embodiments, the agent is conjugated to the AB via a linker. In some embodiments, the linker with which the agent is conjugated to the AB comprises an SPDB moiety, a vc moiety, or a PEG2-vc moiety. In some embodiments, the linker and toxin conjugated to the AB comprises an SPDB-DM4 moiety, a vc-MMAD moiety, a vc-MMAE moiety, vc-duocarmycin, or a PEG2-vc-MMAD moiety. In some embodiments, the linker is a cleavable linker. In some embodiments, the linker is a non-cleavable linker. In some embodiments, the agent is a detectable moiety. In some embodiments, the detectable moiety is a diagnostic agent.

In some embodiments, the conjugated antibody comprises a conjugated antibody comprising: (a) an antibody or an antigen binding fragment thereof (AB) that specifically binds to mammalian CD147, wherein the AB comprises: (i) the VH CDR1 sequence GFTFSNYWMN (SEQ ID NO: 10) or GFTFSNYWMD (SEQ ID NO: 11); the VH CDR2 sequence EIRLKSYNYATH (SEQ ID NO: 12); the VH CDR3 sequence AGTDY (SEQ ID NO: 13); the VL CDR1 sequence KASQSVRTDVA (SEQ ID NO: 14) or RASQSVRTDVG (SEQ ID NO: 15); the VL CDR2 sequence YSSNRYT (SEQ ID NO: 16); and the VL CDR3 sequence QQDYSSPFT (SEQ ID NO: 17) or QQDYSSPYT (SEQ ID NO: 18), or (ii) a heavy chain variable region comprising an amino acid sequence selected from the group consisting of SEQ ID NOs: 1-4, and a light chain variable region comprising an amino acid sequence selected from the group consisting of SEQ ID NOs: 5-9; (b) an agent conjugated to the AB, wherein the agent is selected from the group consisting of auristatin E, monomethyl auristatin F (MMAF), monomethyl auristatin E (MMAE), monomethyl auristatin D (MMAD), maytansinoid DM4, maytansinoid DM1, a pyrrolobenzodiazepine, a pyrrolobenzodiazepine dimer, and a duocarmycin.

The CD147 antibodies and activatable antibodies of the disclosure have at least one point of conjugation for an agent, but in the methods and compositions provided herein less than all possible points of conjugation are available for conjugation to an agent. In some embodiments, the one or more points of conjugation are sulfur atoms involved in disulfide bonds. In some embodiments, the one or more points of conjugation are sulfur atoms involved in interchain disulfide bonds. In some embodiments, the one or more points of conjugation are sulfur atoms involved in interchain sulfide bonds, but not sulfur atoms involved in intrachain disulfide bonds. In some embodiments, the one or more points of conjugation are sulfur atoms of cysteine or other amino acid residues containing a sulfur atom. Such residues may occur naturally in the antibody structure or can be incorporated into the antibody by site-directed mutagenesis, chemical conversion, or mis-incorporation of non-natural amino acids.

Also provided are methods of preparing a conjugate of a CD147 antibody/CD147 activatable antibody having one or more interchain disulfide bonds in the AB and one or more intrachain disulfide bonds in the MM, and a drug reactive with free thiols is provided. The method generally includes partially reducing interchain disulfide bonds in the activatable antibody with a reducing agent, such as, for example, TCEP; and conjugating the drug reactive with free thiols to the partially reduced antibody/activatable antibody. As used herein, the term partial reduction refers to situations where an act antibody/activatable antibody is contacted with a reducing agent and less than all disulfide bonds, e.g., less than all possible sites of conjugation are reduced. In some embodiments, less than 99%, 98%, 97%, 96%, 95%, 90%, 85%, 80%, 75%, 70%, 65%, 60%, 55%, 50%, 45%, 40%, 35%, 30%, 25%, 20%, 15%, 10% or less than 5% of all possible sites of conjugation are reduced.

In yet other embodiments, a method of reducing and conjugating an agent, e.g., a drug, to a CD147 antibody/CD147 activatable antibody resulting in selectivity in the placement of the agent is provided. The method generally includes partially reducing the CD147 antibody/CD147 activatable antibody with a reducing agent such that any conjugation sites in the masking moiety or other non-AB portion of the CD147 antibody/CD147 activatable antibody are not reduced, and conjugating the agent to interchain thiols in the AB. The conjugation site(s) are selected so as to allow desired placement of an agent to allow conjugation to occur at a desired site. The reducing agent is, for example, TCEP. The reduction reaction conditions such as, for example, the ratio of reducing agent to antibody/activatable antibody, the length of incubation, the temperature during the incubation, the pH of the reducing reaction solution, etc., are determined by identifying the conditions that produce a conjugated antibody/activatable antibody e.g. under conditions that produce a conjugated activatable antibody in which the MM retains the ability to effectively and efficiently mask the AB of the activatable antibody in an uncleaved state. The ratio of reduction agent to antibody/activatable antibody will vary depending on the antibody/activatable antibody. In some embodiments, the ratio of reducing agent to antibody/activatable antibody will be in a range from about 20:1 to 1:1, from about 10:1 to 1:1, from about 9:1 to 1:1, from about 8:1 to 1:1, from about 7:1 to 1:1, from about 6:1 to 1:1, from about 5:1 to 1:1, from about 4:1 to 1:1, from about 3:1 to 1:1, from about 2:1 to 1:1, from about 20:1 to 1:1.5, from about 10:1 to 1:1.5, from about 9:1 to 1:1.5, from about 8:1 to 1:1.5, from about 7:1 to 1:1.5, from about 6:1 to 1:1.5, from about 5:1 to 1:1.5, from about 4:1 to 1:1.5, from about 3:1 to 1:1.5, from about 2:1 to 1:1.5, from about 1.5:1 to 1:1.5, or from about 1:1 to 1:1.5. In some embodiments, the ratio is in a range of from about 5:1 to 1:1. In some embodiments, the ratio is in a range of from about 5:1 to 1.5:1. In some embodiments, the ratio is in a range of from about 4:1 to 1:1. In some embodiments, the ratio is in a range from about 4:1 to 1.5:1. In some embodiments, the ratio is in a range from about 8:1 to about 1:1. In some embodiments, the ratio is in a range of from about 2.5:1 to 1:1.

In some embodiments, the CD147 antibody undergoes conjugation first and is then further modified to include a CM and MM (resulting in an activatable antibody). In some embodiments, the CD147 activatable antibody is conjugated.

In some embodiments, a method of reducing interchain disulfide bonds in the AB of an activatable CD147 antibody and conjugating an agent, e.g., a thiol-containing agent such as a drug, to the resulting interchain thiols to selectively locate agent(s) on the AB is provided. The method generally includes partially reducing the AB with a reducing agent to form at least two interchain thiols without forming all possible interchain thiols in the activatable antibody; and conjugating the agent to the interchain thiols of the partially reduced AB. For example, the AB of the activatable antibody is partially reduced for about 1 hour at about 37° C. at a desired ratio of reducing agent:activatable antibody. In some embodiments, the ratio of reducing agent to activatable antibody will be in a range from about 20:1 to 1:1, from about 10:1 to 1:1, from about 9:1 to 1:1, from about 8:1 to 1:1, from about 7:1 to 1:1, from about 6:1 to 1:1, from about 5:1 to 1:1, from about 4:1 to 1:1, from about 3:1 to 1:1, from about 2:1 to 1:1, from about 20:1 to 1:1.5, from about 10:1 to 1:1.5, from about 9:1 to 1:1.5, from about 8:1 to 1:1.5, from about 7:1 to 1:1.5, from about 6:1 to 1:1.5, from about 5:1 to 1:1.5, from about 4:1 to 1:1.5, from about 3:1 to 1:1.5, from about 2:1 to 1:1.5, from about 1.5:1 to 1:1.5, or from about 1:1 to 1:1.5. In some embodiments, the ratio is in a range of from about 5:1 to 1:1. In some embodiments, the ratio is in a range of from about 5:1 to 1.5:1. In some embodiments, the ratio is in a range of from about 4:1 to 1:1. In some embodiments, the ratio is in a range from about 4:1 to 1.5:1. In some embodiments, the ratio is in a range from about 8:1 to about 1:1. In some embodiments, the ratio is in a range of from about 2.5:1 to 1:1.

The thiol-containing reagent can be, for example, cysteine or N-acetyl cysteine. The reducing agent can be, for example, TCEP. In some embodiments, the reduced activatable antibody can be purified prior to conjugation, using for example, column chromatography, dialysis, or diafiltration. Alternatively, the reduced antibody is not purified after partial reduction and prior to conjugation.

The invention also provides partially reduced antibodies/activatable antibodies in which at least one interchain disulfide bond in the antibody/activatable antibody has been reduced with a reducing agent without disturbing any intrachain disulfide bonds in the antibody/activatable antibody, wherein the activatable antibody includes an antibody or an antigen binding fragment thereof (AB) that specifically binds to CD147, a masking moiety (MM) that inhibits the binding of the AB of the activatable antibody in an uncleaved state to the CD147 target, and a cleavable moiety (CM) coupled to the AB, wherein the CM is a polypeptide that functions as a substrate for a protease. In some embodiments the MM is coupled to the AB via the CM. In some embodiments, one or more intrachain disulfide bond(s) of the antibody/activatable antibody is not disturbed by the reducing agent. In some embodiments, one or more intrachain disulfide bond(s) of the MM within the antibody/activatable antibody is not disturbed by the reducing agent. In some embodiments, the activatable antibody in the uncleaved state has the structural arrangement from N-terminus to C-terminus as follows: MM-CM-AB or AB-CM-MM. In some embodiments, reducing agent is TCEP.

In yet other embodiments, a method of reducing and conjugating an agent, e.g., a drug, to a CD147 antibody/CD147 activatable antibody resulting in selectivity in the placement of the agent by providing an activatable CD147 antibody with a defined number and positions of lysine and/or cysteine residues. In some embodiments, the defined number of lysine and/or cysteine residues is higher or lower than the number of corresponding residues in the amino acid sequence of the parent antibody or activatable antibody. In some embodiments, the defined number of lysine and/or cysteine residues may result in a defined number of agent equivalents that can be conjugated to the CD147 antibody or activatable CD147 antibody. In some embodiments, the defined number of lysine and/or cysteine residues may result in a defined number of agent equivalents that can be conjugated to the CD147 antibody or activatable CD147 antibody in a site-specific manner. In some embodiments, the modified activatable antibody is modified with one or more non-natural amino acids in a site-specific manner, thus in some embodiments limiting the conjugation of the agents to only the sites of the non-natural amino acids. In some embodiments, the CD147 antibody or activatable CD147 antibody with a defined number and positions of lysine and/or cysteine residues can be partially reduced with a reducing agent as discussed herein such that any conjugation sites in the masking moiety or other non-AB portion of the activatable antibody are not reduced, and conjugating the agent to interchain thiols in the AB.

The disclosure also provides partially reduced activatable antibodies in which at least one interchain disulfide bond in the activatable antibody has been reduced with a reducing agent without disturbing any intrachain disulfide bonds in the activatable antibody, wherein the activatable antibody includes an antibody or an antigen binding fragment thereof (AB) that specifically binds to the target, e.g., CD147, a masking moiety (MM) that inhibits the binding of the AB of the activatable antibody in an uncleaved state to the target, and a cleavable moiety (CM) coupled to the AB, wherein the CM is a polypeptide that functions as a substrate for at least one protease. In some embodiments, the MM is coupled to the AB via the CM. In some embodiments, one or more intrachain disulfide bond(s) of the activatable antibody is not disturbed by the reducing agent. In some embodiments, one or more intrachain disulfide bond(s) of the MM within the activatable antibody is not disturbed by the reducing agent. In some embodiments, the activatable antibody in the uncleaved state has the structural arrangement from N-terminus to C-terminus as follows: MM-CM-AB or AB-CM-MM. In some embodiments, reducing agent is TCEP.

In some embodiments, the agent is linked to the AB using a maleimide caproyl-valine-citrulline linker or a maleimide PEG-valine-citrulline linker. In some embodiments, the agent is linked to the AB using a maleimide caproyl-valine-citrulline linker. In some embodiments, the agent is linked to the AB using a maleimide PEG-valine-citrulline linker In some embodiments, the agent is monomethyl auristatin D (MMAD) linked to the AB using a maleimide PEG-valine-citrulline-para-aminobenzyloxycarbonyl linker, and this linker payload construct is referred to herein as “vc-MMAD.” In some embodiments, the agent is monomethyl auristatin E (MMAE) linked to the AB using a maleimide PEG-valine-citrulline-para-aminobenzyloxycarbonyl linker, and this linker payload construct is referred to herein as “vc-MMAE.”In some embodiments, the agent is linked to the AB using a maleimide PEG-valine-citrulline linker In some embodiments, the agent is monomethyl auristatin D (MMAD) linked to the AB using a maleimide bis-PEG-valine-citrulline-para-aminobenzyloxycarbonyl linker, and this linker payload construct is referred to herein as “PEG2-vc-MMAD.” The structures of vc-MMAD, vc-MMAE, and PEG2-vc-MMAD are shown below:

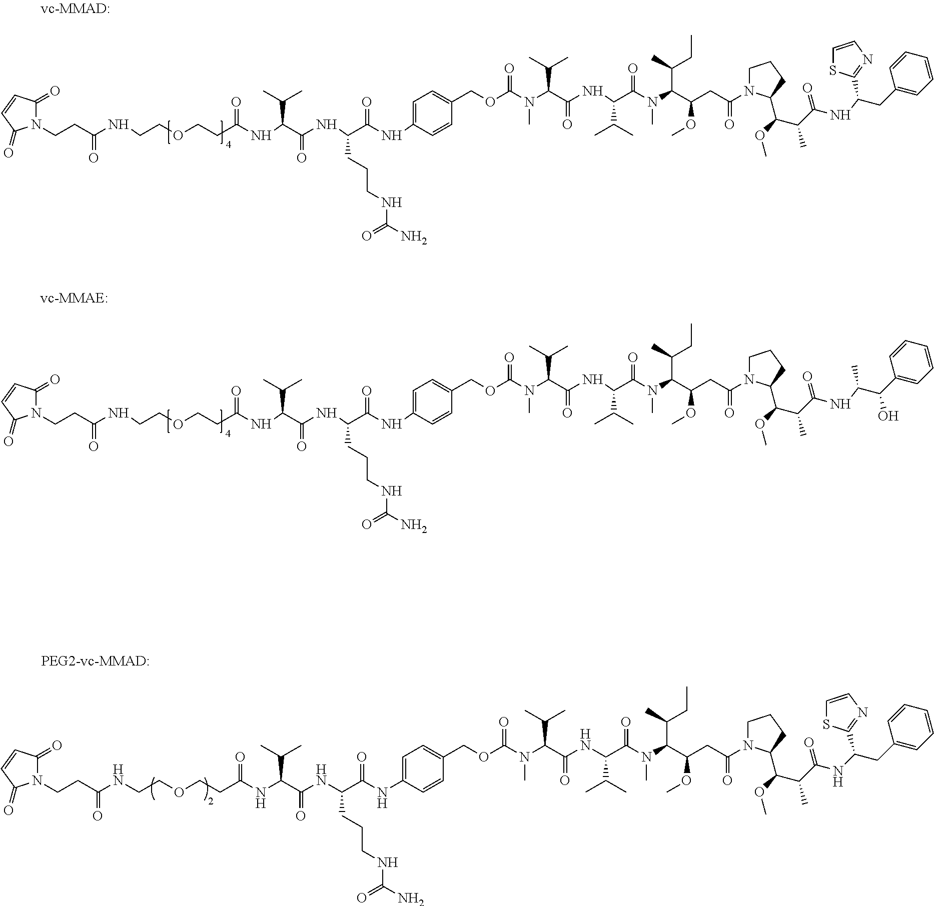

The disclosure also provides conjugated activatable antibodies that include an activatable antibody linked to monomethyl auristatin D (MMAD) payload, wherein the activatable antibody includes an antibody or an antigen binding fragment thereof (AB) that specifically binds to a target, a masking moiety (MM) that inhibits the binding of the AB of the activatable antibody in an uncleaved state to the target, and cleavable moiety (CM) coupled to the AB, and the CM is a polypeptide that functions as a substrate for at least one MMP protease.

In some embodiments, the MMAD-conjugated activatable antibody can be conjugated using any of several methods for attaching agents to ABs: (a) attachment to the carbohydrate moieties of the AB, or (b) attachment to sulfhydryl groups of the AB, or (c) attachment to amino groups of the AB, or (d) attachment to carboxylate groups of the AB.

In some embodiments, the MMAD payload is conjugated to the AB via a linker. In some embodiments, the MMAD payload is conjugated to a cysteine in the AB via a linker. In some embodiments, the MMAD payload is conjugated to a lysine in the AB via a linker. In some embodiments, the MMAD payload is conjugated to another residue of the AB via a linker, such as those residues disclosed herein. In some embodiments, the linker is a thiol-containing linker. In some embodiments, the linker is a cleavable linker. In some embodiments, the linker is a non-cleavable linker. In some embodiments, the linker is selected from the group consisting of the linkers shown in Tables 7 and 8. In some embodiments, the activatable antibody and the MMAD payload are linked via a maleimide caproyl-valine-citrulline linker. In some embodiments, the activatable antibody and the MMAD payload are linked via a maleimide PEG-valine-citrulline linker. In some embodiments, the activatable antibody and the MMAD payload are linked via a maleimide caproyl-valine-citrulline-para-aminobenzyloxycarbonyl linker. In some embodiments, the activatable antibody and the MMAD payload are linked via a maleimide PEG-valine-citrulline-para-aminobenzyloxycarbonyl linker. In some embodiments, the MMAD payload is conjugated to the AB using the partial reduction and conjugation technology disclosed herein.

In some embodiments, the polyethylene glycol (PEG) component of a linker of the present disclosure is formed from 2 ethylene glycol monomers, 3 ethylene glycol monomers, 4 ethylene glycol monomers, 5 ethylene glycol monomers, 6 ethylene glycol monomers, 7 ethylene glycol monomers 8 ethylene glycol monomers, 9 ethylene glycol monomers, or at least 10 ethylene glycol monomers. In some embodiments of the present disclosure, the PEG component is a branched polymer. In some embodiments of the present disclosure, the PEG component is an unbranched polymer. In some embodiments, the PEG polymer component is functionalized with an amino group or derivative thereof, a carboxyl group or derivative thereof, or both an amino group or derivative thereof and a carboxyl group or derivative thereof.

In some embodiments, the PEG component of a linker of the present disclosure is an amino-tetra-ethylene glycol-carboxyl group or derivative thereof. In some embodiments, the PEG component of a linker of the present disclosure is an amino-tri-ethylene glycol-carboxyl group or derivative thereof. In some embodiments, the PEG component of a linker of the present disclosure is an amino-di-ethylene glycol-carboxyl group or derivative thereof. In some embodiments, an amino derivative is the formation of an amide bond between the amino group and a carboxyl group to which it is conjugated. In some embodiments, a carboxyl derivative is the formation of an amide bond between the carboxyl group and an amino group to which it is conjugated. In some embodiments, a carboxyl derivative is the formation of an ester bond between the carboxyl group and an hydroxyl group to which it is conjugated.

Conjugates of the antibody and cytotoxic agent are made using a variety of bifunctional protein-coupling agents such as N-succinimidyl-3-(2-pyridyldithiol) propionate (SPDP), iminothiolane (IT), bifunctional derivatives of imidoesters (such as dimethyl adipimidate HCL), active esters (such as disuccinimidyl suberate), aldehydes (such as glutareldehyde), bis-azido compounds (such as bis (p-azidobenzoyl) hexanediamine), bis-diazonium derivatives (such as bis-(p-diazoniumbenzoyl)-ethylenediamine), diisocyanates (such as tolyene 2,6-diisocyanate), and bis-active fluorine compounds (such as 1,5-difluoro-2,4-dinitrobenzene). For example, a ricin immunotoxin can be prepared as described in Vitetta et al., Science 238: 1098 (1987). Carbon-14-labeled 1-isothiocyanatobenzyl-3-methyldiethylene triaminepentaacetic acid (MX-DTPA) is an exemplary chelating agent for conjugation of radionucleotide to the antibody. (See WO94/11026).

Table 6 lists some of the exemplary pharmaceutical agents that can be employed in the herein described disclosure but in no way is meant to be an exhaustive list.

TABLE 6
Exemplary Pharmaceutical Agents for Conjugation

| | |
|---|---|
| CYTOTOXIC AGENTS | |
| Auristatins | Turbostatin |
| Auristatin E | Phenstatins |
| Monomethyl auristatin D (MMAD) | Hydroxyphenstatin |
| Monomethyl auristatin E (MMAE) | Spongistatin 5 |
| Desmethyl auristatin E (DMAE) | Spongistatin 7 |
| Auristatin F | Halistatin 1 |
| Monomethyl auristatin F (MMAF) | Halistatin 2 |
| Desmethyl auristatin F (DMAF) | Halistatin 3 |
| Auristatin derivatives, e.g., amides thereof | Modified Bryostatins |
| Auristatin tyramine | Halocomstatins |
| Auristatin quinoline | Pyrrolobenzimidazoles (PBI) |
| Dolastatins | Cibrostatin6 |
| Dolastatin derivatives | Doxaliform |
| Dolastatin 16 DmJ | Anthracyclins analogues |
| Dolastatin 16 Dpv | |
| Maytansinoids, e.g. DM-1; DM-4 | |
| Maytansinoid derivatives | Cemadotin analogue (CemCH2-SH) |
| Duocarmycin | Pseudomonas toxin A (PE38) variant |
| Duocarmycin derivatives | Pseudomonas toxin A (ZZ-PE38) variant |
| Alpha-amanitin | ZJ-101 |
| Anthracyclines | OSW-1 |
| Doxorubicin | 4-Nitrobenzyloxycarbonyl Derivatives of |
| | O6-Benzylguanine |
| Daunorubicin | Topoisomerase inhibitors |
| Bryostatins | Hemiasterfin |
| Camptothecin | Cephalotaxine |
| Camptothecin derivatives | Homoharringtonine |
| 7-substituted Camptothecin | Pyrrolobenzodiazepine dimers (PBDs) |
| 10, 11- | Pyrrolobenzodiazepenes |
| Difluoromethylenedioxycamptothecin | |
| Combretastatins | Functionalized pyrrolobenzodiazepenes |
| Debromoaplysiatoxin | Functionalized pyrrolobenzodiazepene |
| | dimers |
| Kahalalide-F | Calicheamicins |
| Discodermolide | Podophyllotoxins |
| Ecteinascidins | Taxanes |
| | Vinca alkaloids |
| ANTIVIRALS | |
| | CONJUGATABLE DETECTION |
| Acyclovir | REAGENTS |
| Vira A | Fluorescein and derivatives thereof |
| Symmetrel | Fluorescein isothiocyanate (MC) |
| ANTIFUNGALS | RADIOPHARMACEUTICALS |
| Nystatin | ¹²⁵I |
| | ¹³¹I |
| ADDITIONAL ANTI-NEOPLASTICS | ⁸⁹Zr |
| Adriamycin | ¹¹¹In |
| Cerubidine | ¹²³I |
| Bleomycin | ¹³¹I |
| Alkeran | ⁹⁹mTc |
| Velban | ²⁰¹Tl |
| Oncovin | ¹³³Xe |
| Fluorouracil | ¹¹C |
| Methotrexate | ⁶²Cu |
| Thiotepa | ¹⁸F |
| Bisantrene | ⁶⁸Ga |
| Novantrone | ¹³N |
| Thioguanine | ¹⁵O |
| Procarabizine | ³⁸K |
| Cytarabine | ⁸²Rb |
| | ⁹⁹mTc (Technetium) |
| ANTI-BACTERIALS | |
| Aminoglycosides | HEAVY METALS |
| Streptomycin | Barium |
| Neomycin | Gold |
| Kanamycin | Platinum |
| Amikacin | |
| Gentamicin | ANTI-MYCOPLASMALS |
| Tobramycin | Tylosine |
| Streptomycin B | Spectinomycin |
| Spectinomycin | |
| Ampicillin | |
| Sulfanilamide | |
| Polymyxin | |
| Chloramphenicol | |

Those of ordinary skill in the art will recognize that a large variety of possible moieties can be coupled to the resultant antibodies of the disclosure. (See, for example, “Conjugate Vaccines”, Contributions to Microbiology and Immunology, J. M. Cruse and R. E. Lewis, Jr (eds), Carger Press, New York, (1989), the entire contents of which are incorporated herein by reference).

Coupling can be accomplished by any chemical reaction that will bind the two molecules so long as the antibody and the other moiety retain their respective activities. This linkage can include many chemical mechanisms, for instance covalent binding, affinity binding, intercalation, coordinate binding and complexation. In some embodiments, the binding is, however, covalent binding. Covalent binding can be achieved either by direct condensation of existing side chains or by the incorporation of external bridging molecules. Many bivalent or polyvalent linking agents are useful in coupling protein molecules, such as the antibodies of the present disclosure, to other molecules. For example, representative coupling agents can include organic compounds such as thioesters, carbodiimides, succinimide esters, diisocyanates, glutaraldehyde, diazobenzenes and hexamethylene diamines. This listing is not intended to be exhaustive of the various classes of coupling agents known in the art but, rather, is exemplary of the more common coupling agents. (See Killen and Lindstrom, Jour. Immun. 133:1335-2549 (1984); Jansen et al., Immunological Reviews 62:185-216 (1982); and Vitetta et al., Science 238:1098 (1987).

In some embodiments, in addition to the compositions and methods provided herein, the activatable antibody can also be modified for site-specific conjugation through modified amino acid sequences inserted or otherwise included in the activatable antibody sequence. These modified amino acid sequences are designed to allow for controlled placement and/or dosage of the conjugated agent within a conjugated activatable antibody. For example, the activatable antibody can be engineered to include cysteine substitutions at positions on light and heavy chains that provide reactive thiol groups and do not negatively impact protein folding and assembly, nor alter antigen binding. In some embodiments, the activatable antibody can be engineered to include or otherwise introduce one or more non-natural amino acid residues within the activatable antibody to provide suitable sites for conjugation. In some embodiments, the activatable antibody can be engineered to include or otherwise introduce enzymatically activatable peptide sequences within the activatable antibody sequence.

Suitable linkers are described in the literature. (See, for example, Ramakrishnan, S. et al., Cancer Res. 44:201-208 (1984) describing use of MBS (M-maleimidobenzoyl-N-hydroxysuccinimide ester). See also, U.S. Pat. No. 5,030,719, describing use of halogenated acetyl hydrazide derivative coupled to an antibody by way of an oligopeptide linker. In some embodiments, suitable linkers include: (i) EDC (1-ethyl-3-(3-dimethylamino-propyl) carbodiimide hydrochloride; (ii) SMPT (4-succinimidyloxycarbonyl-alpha-methyl-alpha-(2-pridyl-dithio)-toluene (Pierce Chem. Co., Cat. (21558G); (iii) SPDP (succinimidyl-6 [3-(2-pyridyldithio) propionamido]hexanoate (Pierce Chem. Co., Cat #21651G); (iv) Sulfo-LC-SPDP (sulfosuccinimidyl 6 [3-(2-pyridyldithio)-propianamide] hexanoate (Pierce Chem. Co. Cat. #2165-G); and (v) sulfo-NHS (N-hydroxysulfo-succinimide: Pierce Chem. Co., Cat. #24510) conjugated to EDC. Additional linkers include, but are not limited to, SMCC ((succinimidyl 4-(N-maleimidomethyl)cyclohexane-1-carboxylate), sulfo-SMCC (sulfosuccinimidyl 4-(N-maleimidomethyl)cyclohexane-1-carboxylate), SPDB (N-succinimidyl-4-(2-pyridyldithio) butanoate), or sulfo-SPDB (N-succinimidyl-4-(2-pyridyldithio)-2-sulfo butanoate).

The linkers described above contain components that have different attributes, thus leading to conjugates with differing physio-chemical properties. For example, sulfo-NHS esters of alkyl carboxylates are more stable than sulfo-NHS esters of aromatic carboxylates. NHS-ester containing linkers are less soluble than sulfo-NHS esters. Further, the linker SMPT contains a sterically hindered disulfide bond, and can form conjugates with increased stability. Disulfide linkages, are in general, less stable than other linkages because the disulfide linkage is cleaved in vitro, resulting in less conjugate available. Sulfo-NHS, in particular, can enhance the stability of carbodimide couplings. Carbodimide couplings (such as EDC) when used in conjunction with sulfo-NHS, forms esters that are more resistant to hydrolysis than the carbodimide coupling reaction alone.

In some embodiments, the linkers are cleavable. In some embodiments, the linkers are non-cleavable. In some embodiments, two or more linkers are present. The two or more linkers are all the same, i.e., cleavable or non-cleavable, or the two or more linkers are different, i.e., at least one cleavable and at least one non-cleavable.

The present disclosure utilizes several methods for attaching agents to ABs: (a) attachment to the carbohydrate moieties of the AB, or (b) attachment to sulfhydryl groups of the AB, or (c) attachment to amino groups of the AB, or (d) attachment to carboxylate groups of the AB. According to the disclosure, ABs can be covalently attached to an agent through an intermediate linker having at least two reactive groups, one to react with AB and one to react with the agent. The linker, which may include any compatible organic compound, can be chosen such that the reaction with AB (or agent) does not adversely affect AB reactivity and selectivity. Furthermore, the attachment of linker to agent might not destroy the activity of the agent. Suitable linkers for reaction with oxidized antibodies or oxidized antibody fragments include those containing an amine selected from the group consisting of primary amine, secondary amine, hydrazine, hydrazide, hydroxylamine, phenylhydrazine, semicarbazide and thiosemicarbazide groups. Such reactive functional groups may exist as part of the structure of the linker, or can be introduced by suitable chemical modification of linkers not containing such groups.

According to the present disclosure, suitable linkers for attachment to reduced ABs include those having certain reactive groups capable of reaction with a sulfhydryl group of a reduced antibody or fragment. Such reactive groups include, but are not limited to: reactive haloalkyl groups (including, for example, haloacetyl groups), p-mercuribenzoate groups and groups capable of Michael-type addition reactions (including, for example, maleimides and groups of the type described by Mitra and Lawton, 1979, J. Amer. Chem. Soc. 101: 3097-3110).

According to the present disclosure, suitable linkers for attachment to neither oxidized nor reduced Abs include those having certain functional groups capable of reaction with the primary amino groups present in unmodified lysine residues in the Ab. Such reactive groups include, but are not limited to, NHS carboxylic or carbonic esters, sulfo-NHS carboxylic or carbonic esters, 4-nitrophenyl carboxylic or carbonic esters, pentafluorophenyl carboxylic or carbonic esters, acyl imidazoles, isocyanates, and isothiocyanates.

According to the present disclosure, suitable linkers for attachment to neither oxidized nor reduced Abs include those having certain functional groups capable of reaction with the carboxylic acid groups present in aspartate or glutamate residues in the Ab, which have been activated with suitable reagents. Suitable activating reagents include EDC, with or without added NHS or sulfo-NHS, and other dehydrating agents utilized for carboxamide formation. In these instances, the functional groups present in the suitable linkers would include primary and secondary amines, hydrazines, hydroxylamines, and hydrazides.

The agent can be attached to the linker before or after the linker is attached to the AB. In certain applications it may be desirable to first produce an AB-linker intermediate in which the linker is free of an associated agent. Depending upon the particular application, a specific agent may then be covalently attached to the linker. In some embodiments, the AB is first attached to the MM, CM and associated linkers and then attached to the linker for conjugation purposes.

Brarched Linkers:

In specific embodiments, branched linkers that have multiple sites for attachment of agents are utilized. For multiple site linkers, a single covalent attachment to an AB would result in an AB-linker intermediate capable of binding an agent at a number of sites. The sites can be aldehyde or sulfhydryl groups or any chemical site to which agents can be attached.

In some embodiments, higher specific activity (or higher ratio of agents to AB) can be achieved by attachment of a single site linker at a plurality of sites on the AB. This plurality of sites can be introduced into the AB by either of two methods. First, one may generate multiple aldehyde groups and/or sulfhydryl groups in the same AB. Second, one may attach to an aldehyde or sulfhydryl of the AB a “branched linker” having multiple functional sites for subsequent attachment to linkers. The functional sites of the branched linker or multiple site linker can be aldehyde or sulfhydryl groups, or can be any chemical site to which linkers can be attached. Still higher specific activities can be obtained by combining these two approaches, that is, attaching multiple site linkers at several sites on the AB.

Cleavable Linkers:

Peptide linkers that are susceptible to cleavage by enzymes of the complement system, such as but not limited to u-plasminogen activator, tissue plasminogen activator, trypsin, plasmin, or another enzyme having proteolytic activity can be used in one embodiment of the present disclosure. According to one method of the present disclosure, an agent is attached via a linker susceptible to cleavage by complement. The antibody is selected from a class that can activate complement. The antibody-agent conjugate, thus, activates the complement cascade and releases the agent at the target site. According to another method of the present disclosure, an agent is attached via a linker susceptible to cleavage by enzymes having a proteolytic activity such as a u-plasminogen activator, a tissue plasminogen activator, plasmin, or trypsin. These cleavable linkers are useful in conjugated activatable antibodies that include an extracellular toxin, e.g., by way of non-limiting example, any of the extracellular toxins shown in Table 6.

Non-limiting examples of cleavable linker sequences are provided in Table 7.

TABLE 7
Exemplary Linker Sequences for Conjugation

| Types of Cleavable Sequences | Amino Acid Sequence |
|---|---|
| Plasmin cleavable sequences | |
| Pro-urokinase | PRFKIIGG (SEQ ID NO: 615) |
| | PRFRIIGG (SEQ ID NO: 616) |
| TGFβ | SSRHRRALD (SEQ ID NO: 617) |
| Plasminogen | RKSSIIIRMRDVNTL (SEQ ID NO: 618) |
| Staphylokinase | SSSFDKGKYKKGDDA (SEQ ID NO: 619) |
| | SSSFDKGKYKRGDDA (SEQ ID NO: 620) |
| Factor Xa cleavable sequences | IEGR (SEQ ID NO: 621) |
| | IDGR (SEQ ID NO: 622) |
| | GGSIDGR (SEQ ID NO: 623) |
| MMP cleavable sequences | |
| Gelatinase A | PLGLWA (SEQ ID NO: 624) |
| Collagenase cleavable sequences | |
| Calf skin collagen (α1(I) chain) | GPQGIAGQ (SEQ ID NO: 625) |
| Calf skin collagen (α2(I) chain) | GPQGLLGA (SEQ ID NO: 626) |
| Bovine cartilage collagen (α1(II) chain) | GIAGQ (SEQ ID NO: 627) |
| Human liver collagen (α1(III) chain) | GPLGIAGI (SEQ ID NO: 628) |
| Human α₂M | GPEGLRVG (SEQ ID NO: 629) |
| Human PZP | YGAGLGVV (SEQ ID NO: 630) |
| | AGLGVVER (SEQ ID NO: 631) |
| | AGLGISSI (SEQ ID NO: 632) |
| Rat α₁M | EPQALAMS (SEQ ID NO: 633) |
| | QALAMSAI (SEQ ID NO: 634) |
| Rat α₂M | AAYHLVSQ (SEQ ID NO: 635) |
| | MDAFLESS (SEQ ID NO: 636) |
| Rat α₁I₃(2J) | ESLPVVAV (SEQ ID NO: 637) |
| Rat α₁I₃(27J) | SAPAVESE (SEQ ID NO: 638) |
| Human fibroblast collagenase | DVAQFVLT (SEQ ID NO: 639) |
| (autolytic cleavages) | VAQFVLTE (SEQ ID NO: 640) |
| | AQFVLTEG (SEQ ID NO: 641) |
| | PVQPIGPQ (SEQ ID NO: 642) |

In addition, agents can be attached via disulfide bonds (for example, the disulfide bonds on a cysteine molecule) to the AB. Since many tumors naturally release high levels of glutathione (a reducing agent) this can reduce the disulfide bonds with subsequent release of the agent at the site of delivery. In some embodiments, the reducing agent that would modify a CM would also modify the linker of the conjugated activatable antibody.

Spacers and Cleavable Elements:

In some embodiments, it may be necessary to construct the linker in such a way as to optimize the spacing between the agent and the AB of the activatable antibody. This can be accomplished by use of a linker of the general structure:

W—(CH₂)n-Q

wherein
W is either —NH—CH₂— or —CH₂—;
Q is an amino acid, peptide; and
n is an integer from 0 to 20.

In some embodiments, the linker may comprise a spacer element and a cleavable element. The spacer element serves to position the cleavable element away from the core of the AB such that the cleavable element is more accessible to the enzyme responsible for cleavage. Certain of the branched linkers described above may serve as spacer elements.

Throughout this discussion, it should be understood that the attachment of linker to agent (or of spacer element to cleavable element, or cleavable element to agent) need not be particular mode of attachment or reaction. Any reaction providing a product of suitable stability and biological compatibility is acceptable.

Serum Complement and Selection of Linkers:

According to one method of the present disclosure, when release of an agent is desired, an AB that is an antibody of a class that can activate complement is used. The resulting conjugate retains both the ability to bind antigen and activate the complement cascade. Thus, according to this embodiment of the present disclosure, an agent is joined to one end of the cleavable linker or cleavable element and the other end of the linker group is attached to a specific site on the AB. For example, if the agent has a hydroxy group or an amino group, it can be attached to the carboxy terminus of a peptide, amino acid or other suitably chosen linker via an ester or amide bond, respectively. For example, such agents can be attached to the linker peptide via a carbodimide reaction. If the agent contains functional groups that would interfere with attachment to the linker, these interfering functional groups can be blocked before attachment and deblocked once the product conjugate or intermediate is made. The opposite or amino terminus of the linker is then used either directly or after further modification for binding to an AB that is capable of activating complement.

Linkers (or spacer elements of linkers) can be of any desired length, one end of which can be covalently attached to specific sites on the AB of the activatable antibody. The other end of the linker or spacer element can be attached to an amino acid or peptide linker.

Thus when these conjugates bind to antigen in the presence of complement the amide or ester bond that attaches the agent to the linker will be cleaved, resulting in release of the agent in its active form. These conjugates, when administered to a subject, will accomplish delivery and release of the agent at the target site, and are particularly effective for the in vivo delivery of pharmaceutical agents, antibiotics, antimetabolites, antiproliferative agents and the like as presented in but not limited to those in Table 6.

Linkers for Release without Complement Activation:

In yet another application of targeted delivery, release of the agent without complement activation is desired since activation of the complement cascade will ultimately lyse the target cell. Hence, this approach is useful when delivery and release of the agent should be accomplished without killing the target cell. Such is the goal when delivery of cell mediators such as hormones, enzymes, corticosteroids, neurotransmitters, genes or enzymes to target cells is desired. These conjugates can be prepared by attaching the agent to an AB that is not capable of activating complement via a linker that is mildly susceptible to cleavage by serum proteases. When this conjugate is administered to an individual, antigen-antibody complexes will form quickly whereas cleavage of the agent will occur slowly, thus resulting in release of the compound at the target site.

Biochemical Cross Linkers:

In some embodiments, the activatable antibody can be conjugated to one or more therapeutic agents using certain biochemical cross-linkers. Cross-linking reagents form molecular bridges that tie together functional groups of two different molecules. To link two different proteins in a step-wise manner, hetero-bifunctional cross-linkers can be used that eliminate unwanted homopolymer formation.

Peptidyl linkers cleavable by lysosomal proteases are also useful, for example, Val-Cit, Val-Ala or other dipeptides. In addition, acid-labile linkers cleavable in the low-pH environment of the lysosome can be used, for example: bis-sialyl ether. Other suitable linkers include cathepsin-labile substrates, particularly those that show optimal function at an acidic pH.

Exemplary hetero-bifunctional cross-linkers are referenced in Table 8.

TABLE 8
Exemplary Hetero-Bifunctional Cross Linkers
HETERO-BIFUNCTIONAL CROSS-LINKERS

| | | | Spacer Arm |
|---|---|---|---|
| | | | Length after |
| | Reactive | Advantages | cross-linking |
| Linker | Toward | and Applications | (Angstroms) |
| SMPT | Primary amines | Greater stability | 11.2 Å |
| | Sulfhydryls | | |
| SPDP | Primary amines | Thiolation | 6.8 Å |
| | Sulfhydryls | Cleavable cross-linking | |
| LC-SPDP | Primary amines | Extended spacer arm | 15.6 Å |
| | Sulfhydryls | | |
| Sulfo-LC- | Primary amines | Extender spacer arm | 15.6 Å |
| SPDP | Sulfhydryls | Water-soluble | |
| SMCC | Primary amines | Stable maleimide | 11.6 Å |
| | | reactive group | |
| | Sulfhydryls | Enzyme-antibody | |
| | | conjugation | |
| | | Hapten-carrier protein | |
| | | conjugation | |
| Sulfo- | Primary amines | Stable maleimide | 11.6 Å |
| SMCC | | reactive group | |
| | Sulfhydryls | Water-soluble | |
| | | Enzyme-antibody | |
| | | conjugation | |
| MBS | Primary amines | Enzyme-antibody | 9.9 Å |
| | | conjugation | |
| | Sulfhydryls | Hapten-carrier protein | |
| | | conjugation | |
| Sulfo-MBS | Primary amines | Water-soluble | 9.9 Å |
| | Sulfhydryls | | |
| SIAB | Primary amines | Enzyme-antibody | 10.6 Å |
| | Sulfhydryls | conjugation | |
| Sulfo- | Primary amines | Water-soluble | 10.6 Å |
| SIAB | Sulfhydryls | | |
| SMPB | Primary amines | Extended spacer arm | 14.5 Å |
| | Sulfhydryls | Enzyme-antibody | |
| | | conjugation | |
| Sulfo- | Primary amines | Extended spacer arm | 14.5 Å |
| SMPB | Sulfhydryls | Water-soluble | |
| EDE/Sulfo- | Primary amines | Hapten-Carrier | 0 |
| NHS | Carboxyl groups | conjugation | |
| ABH | Carbohydrates | Reacts with sugar | 11.9 Å |
| | Nonselective | groups | |

Non-Cleavable Linkers or Direct Attachment:

In some embodiments of the disclosure, the conjugate can be designed so that the agent is delivered to the target but not released. This can be accomplished by attaching an agent to an AB either directly or via a non-cleavable linker.

These non-cleavable linkers may include amino acids, peptides, D-amino acids or other organic compounds that can be modified to include functional groups that can subsequently be utilized in attachment to ABs by the methods described herein. A-general formula for such an organic linker could be

W—(CH₂)n-Q

wherein
W is either —NH—CH₂— or —CH₂—;
Q is an amino acid, peptide; and
n is an integer from 0 to 20.

Non-Cleavable Conjugates:

In some embodiments, a compound can be attached to ABs that do not activate complement. When using ABs that are incapable of complement activation, this attachment can be accomplished using linkers that are susceptible to cleavage by activated complement or using linkers that are not susceptible to cleavage by activated complement.

The antibodies disclosed herein can also be formulated as immunoliposomes. Liposomes containing the antibody are prepared by methods known in the art, such as described in Epstein et al., Proc. Natl. Acad. Sci. USA, 82: 3688 (1985); Hwang et al., Proc. Natl Acad. Sci. USA, 77: 4030 (1980); and U.S. Pat. Nos. 4,485,045 and 4,544,545. Liposomes with enhanced circulation time are disclosed in U.S. Pat. No. 5,013,556.

Particularly useful liposomes can be generated by the reverse-phase evaporation method with a lipid composition comprising phosphatidylcholine, cholesterol, and PEG-derivatized phosphatidylethanolamine (PEG-PE). Liposomes are extruded through filters of defined pore size to yield liposomes with the desired diameter. Fab′ fragments of the antibody of the present disclosure can be conjugated to the liposomes as described in Martin et al., J. Biol. Chem., 257: 286-288 (1982) via a disulfide-interchange reaction.

CD147 Antibody Drug Conjugates and CD147 Activatable Antibody Drug Conjugates

In some embodiments, the antibody drug conjugates (ADCs) and activatable antibody drug conjugates (AADCs) can include one or more polypeptides that include the combination of a light chain sequence or a light chain variable domain sequence, and a heavy chain sequence or a heavy chain variable domain sequence, a linker, and a toxin in a given row of Table 9 or any combination of a light chain sequence or a light chain variable domain sequence, and a heavy chain sequence or a heavy chain variable domain sequence, a linker, and a toxin of Table 9. For example, Combination No. 1 comprises the heavy chain of SEQ ID No. 1, the light chain of SEQ ID No. 5, a vc linker, conjugated to MMAD toxin.

TABLE 9
CD147 ADC and CD147 Activatable ADC Combinations

| | Heavy Chain (HC) | Light Chain (LC) | | |
|---|---|---|---|---|
| | or HC Variable | or LC Variable | | |
| Comb. | Region | Region | | |
| No. | SEQ ID NO. | SEQ ID NO. | Linker | Toxin |
| 1 | 1 | 5 | vc | MMAD |
| 2 | 1 | 5 | PEG2-vc | MMAD |
| 3 | 1 | 5 | vc | MMAE |
| 4 | 1 | 5 | vc | duocarmycin |
| 5 | 1 | 5 | spdb | DM4 |
| 6 | 19 | 23 | vc | MMAD |
| 7 | 19 | 23 | PEG2-vc | MMAD |
| 8 | 19 | 23 | vc | MMAE |
| 9 | 19 | 23 | vc | duocarmycin |
| 10 | 19 | 23 | spdb | DM4 |
| 11 | 1 | 172 | vc | MMAD |
| 12 | 1 | 172 | PEG2-vc | MMAD |
| 13 | 1 | 172 | vc | MMAE |
| 14 | 1 | 172 | vc | duocarmycin |
| 15 | 1 | 172 | spdb | DM4 |
| 16 | 19 | 262 | vc | MMAD |
| 17 | 19 | 262 | PEG2-vc | MMAD |
| 18 | 19 | 262 | vc | MMAE |
| 19 | 19 | 262 | vc | duocarmycin |
| 20 | 19 | 262 | spdb | DM4 |
| 21 | 1 | 173 | vc | MMAD |
| 22 | 1 | 173 | PEG2-vc | MMAD |
| 23 | 1 | 173 | vc | MMAE |
| 24 | 1 | 173 | vc | duocarmycin |
| 25 | 1 | 173 | spdb | DM4 |
| 26 | 19 | 263 | vc | MMAD |
| 27 | 19 | 263 | PEG2-vc | MMAD |
| 28 | 19 | 263 | vc | MMAE |
| 29 | 19 | 263 | vc | duocarmycin |
| 30 | 19 | 263 | spdb | DM4 |
| 31 | 1 | 178 | vc | MMAD |
| 32 | 1 | 178 | PEG2-vc | MMAD |
| 33 | 1 | 178 | vc | MMAE |
| 34 | 1 | 178 | vc | duocarmycin |
| 35 | 1 | 178 | spdb | DM4 |
| 36 | 19 | 268 | vc | MMAD |
| 37 | 19 | 268 | PEG2-vc | MMAD |
| 38 | 19 | 268 | vc | MMAE |
| 39 | 19 | 268 | vc | duocarmycin |
| 40 | 19 | 268 | spdb | DM4 |
| 41 | 1 | 179 | vc | MMAD |
| 42 | 1 | 179 | PEG2-vc | MMAD |
| 43 | 1 | 179 | vc | MMAE |
| 44 | 1 | 179 | vc | duocarmycin |
| 45 | 1 | 179 | spdb | DM4 |
| 46 | 19 | 269 | vc | MMAD |
| 47 | 19 | 269 | PEG2-vc | MMAD |
| 48 | 19 | 269 | vc | MMAE |
| 49 | 19 | 269 | vc | duocarmycin |
| 50 | 19 | 269 | spdb | DM4 |
| 51 | 1 | 180 | vc | MMAD |
| 52 | 1 | 180 | PEG2-vc | MMAD |
| 53 | 1 | 180 | vc | MMAE |
| 54 | 1 | 180 | vc | duocarmycin |
| 55 | 1 | 180 | spdb | DM4 |
| 56 | 19 | 270 | vc | MMAD |
| 57 | 19 | 270 | PEG2-vc | MMAD |
| 58 | 19 | 270 | vc | MMAE |
| 59 | 19 | 270 | vc | duocarmycin |
| 60 | 19 | 270 | spdb | D44 |
| 61 | 1 | 171 | vc | MMAD |
| 62 | 1 | 171 | PEG2-vc | MMAD |
| 63 | 1 | 171 | vc | MMAE |
| 64 | 1 | 171 | vc | duocarmycin |
| 65 | 1 | 171 | spdb | DM4 |
| 66 | 19 | 261 | vc | MMAD |
| 67 | 19 | 261 | PEG2-vc | MMAD |
| 68 | 19 | 261 | vc | MMAE |
| 69 | 19 | 261 | vc | duocarmycin |
| 70 | 19 | 261 | spdb | DM4 |
| 71 | 1 | 163 | vc | MMAD |
| 72 | 1 | 163 | PEG2-vc | MMAD |
| 73 | 1 | 163 | vc | MMAE |
| 74 | 1 | 163 | vc | duocarmycin |
| 75 | 1 | 163 | spdb | DM4 |
| 76 | 19 | 253 | vc | MMAD |
| 77 | 19 | 253 | PEG2-vc | MMAD |
| 78 | 19 | 253 | vc | MMAE |
| 79 | 19 | 253 | vc | duocarmycin |
| 80 | 19 | 253 | spdb | DM4 |
| 81 | 1 | 164 | vc | MMAD |
| 82 | 1 | 164 | PEG2-vc | MMAD |
| 83 | 1 | 164 | vc | MMAE |
| 84 | 1 | 164 | vc | duocarmycin |
| 85 | 1 | 164 | spdb | DM4 |
| 86 | 19 | 254 | vc | MMAD |
| 87 | 19 | 254 | PEG2-vc | MMAD |
| 88 | 19 | 254 | vc | MMAE |
| 89 | 19 | 254 | vc | duocarmycin |
| 90 | 19 | 254 | spdb | DM4 |
| 91 | 1 | 165 | vc | MMAD |
| 92 | 1 | 165 | PEG2-vc | MMAD |
| 93 | 1 | 165 | vc | MMAE |
| 94 | 1 | 165 | vc | duocarmycin |
| 95 | 1 | 165 | spdb | DM4 |
| 96 | 19 | 255 | vc | MMAD |
| 97 | 19 | 255 | PEG2-vc | MMAD |
| 98 | 19 | 255 | vc | MMAE |
| 99 | 19 | 255 | vc | duocarmycin |
| 100 | 19 | 255 | spdb | DM4 |
| 101 | 1 | 166 | vc | MMAD |
| 102 | 1 | 166 | PEG2-vc | MMAD |
| 103 | 1 | 166 | vc | MMAE |
| 104 | 1 | 166 | vc | duocarmycin |
| 105 | 1 | 166 | spdb | DM4 |
| 106 | 19 | 256 | vc | MMAD |
| 107 | 19 | 256 | PEG2-vc | MMAD |
| 108 | 19 | 256 | vc | MMAE |
| 109 | 19 | 256 | vc | duocarmycin |
| 110 | 19 | 256 | spdb | DM4 |
| 111 | 1 | 167 | vc | MMAD |
| 112 | 1 | 167 | PEG2-vc | MMAD |
| 113 | 1 | 167 | vc | MMAE |
| 114 | 1 | 167 | vc | duocarmycin |
| 115 | 1 | 167 | spdb | DM4 |
| 116 | 19 | 257 | vc | MMAD |
| 117 | 19 | 257 | PEG2-vc | MMAD |
| 118 | 19 | 257 | vc | MMAE |
| 119 | 19 | 257 | vc | duocarmycin |
| 120 | 19 | 257 | spdb | DM4 |
| 121 | 1 | 168 | vc | MMAD |
| 122 | 1 | 168 | PEG2-vc | MMAD |
| 123 | 1 | 168 | vc | MMAE |
| 124 | 1 | 168 | vc | duocarmycin |
| 125 | 1 | 168 | spdb | DM4 |
| 126 | 19 | 258 | vc | MMAD |
| 127 | 19 | 258 | PEG2-vc | MMAD |
| 128 | 19 | 258 | vc | MMAE |
| 129 | 19 | 258 | vc | duocarmycin |
| 130 | 19 | 258 | spdb | DM4 |
| 131 | 1 | 169 | vc | MMAD |
| 132 | 1 | 169 | PEG2-vc | MMAD |
| 133 | 1 | 169 | vc | MMAE |
| 134 | 1 | 169 | vc | duocarmycin |
| 135 | 1 | 169 | spdb | DM4 |
| 136 | 19 | 259 | vc | MMAD |
| 137 | 19 | 259 | PEG2-vc | MMAD |
| 138 | 19 | 259 | vc | MMAE |
| 139 | 19 | 259 | vc | duocarmycin |
| 140 | 19 | 259 | spdb | DM4 |
| 141 | 1 | 170 | vc | MMAD |
| 142 | 1 | 170 | PEG2-vc | MMAD |
| 143 | 1 | 170 | vc | MMAE |
| 144 | 1 | 170 | vc | duocarmycin |
| 145 | 1 | 170 | spdb | DM4 |
| 146 | 19 | 160 | vc | MMAD |
| 147 | 19 | 160 | PEG2-vc | MMAD |
| 148 | 19 | 160 | vc | MMAE |
| 149 | 19 | 160 | vc | duocarmycin |
| 150 | 19 | 160 | spdb | DM4 |
| 151 | 1 | 157 | vc | MMAD |
| 152 | 1 | 157 | PEG2-vc | MMAD |
| 153 | 1 | 157 | vc | MMAE |
| 154 | 1 | 157 | vc | duocarmycin |
| 155 | 1 | 157 | spdb | DM4 |
| 156 | 19 | 247 | vc | MMAD |
| 157 | 19 | 247 | PEG2-vc | MMAD |
| 158 | 19 | 247 | vc | MMAE |
| 159 | 19 | 247 | vc | duocarmycin |
| 160 | 19 | 247 | spdb | DM4 |
| 161 | 1 | 158 | vc | MMAD |
| 162 | 1 | 158 | PEG2-vc | MMAD |
| 163 | 1 | 158 | vc | MMAE |
| 164 | 1 | 158 | vc | duocarmycin |
| 165 | 1 | 158 | spdb | DM4 |
| 166 | 19 | 248 | vc | MMAD |
| 167 | 19 | 248 | PEG2-vc | MMAD |
| 168 | 19 | 248 | vc | MMAE |
| 169 | 19 | 248 | vc | duocarmycin |
| 170 | 19 | 248 | spdb | DM4 |
| 171 | 1 | 159 | vc | MMAD |
| 172 | 1 | 159 | PEG2-vc | MMAD |
| 173 | 1 | 159 | vc | MMAE |
| 174 | 1 | 159 | vc | duocarmycin |
| 175 | 1 | 159 | spdb | DM4 |
| 176 | 19 | 249 | vc | MMAD |
| 177 | 19 | 249 | PEG2-vc | MMAD |
| 178 | 19 | 249 | vc | MMAE |
| 179 | 19 | 249 | vc | duocarmycin |
| 180 | 19 | 249 | spdb | DM4 |
| 181 | 1 | 160 | vc | MMAD |
| 182 | 1 | 160 | PEG2-vc | MMAD |
| 183 | 1 | 160 | vc | MMAE |
| 184 | 1 | 160 | vc | duocarmycin |
| 185 | 1 | 160 | spdb | DM4 |
| 186 | 19 | 250 | vc | MMAD |
| 187 | 19 | 250 | PEG2-vc | MMAD |
| 188 | 19 | 250 | vc | MMAE |
| 189 | 19 | 250 | vc | duocarmycin |
| 190 | 19 | 250 | spdb | DM4 |
| 191 | 1 | 161 | vc | MMAD |
| 192 | 1 | 161 | PEG2-vc | MMAD |
| 193 | 1 | 161 | vc | MMAE |
| 194 | 1 | 161 | vc | duocarmycin |
| 195 | 1 | 161 | spdb | DM4 |
| 196 | 19 | 251 | vc | MMAD |
| 197 | 19 | 251 | PEG2-vc | MMAD |
| 198 | 19 | 251 | vc | MMAE |
| 199 | 19 | 251 | vr | duocarmycin |
| 200 | 19 | 251 | spdb | DM4 |
| 201 | 1 | 162 | vc | MMAD |
| 202 | 1 | 162 | PEG2-vc | MMAD |
| 203 | 1 | 162 | vc | MMAE |
| 204 | 1 | 162 | vc | duocarmycin |
| 205 | 1 | 162 | spdb | DM4 |
| 206 | 19 | 252 | vc | MMAD |
| 207 | 19 | 252 | PEG2-vc | MMAD |
| 208 | 19 | 252 | vc | MMAE |
| 209 | 19 | 252 | vc | duocarmycin |
| 210 | 19 | 252 | spdb | DM4 |

An antibody drug conjugate (ADC) of the present disclosure or activatable antibody drug conjugate (AADC) of the present disclosure may include one or more polypeptides that include the combination of amino acid sequences, a linker, and a toxin listed in a given row of Table 9. Therefore, an activatable antibody drug conjugate (ADC) of the present disclosure or activatable antibody drug conjugate (AADC) of the present disclosure that includes the combination of amino acid sequences, a linker, and a toxin listed in a given row or provided as a specific combination is described herein. For example, an activatable antibody drug conjugate of the present disclosure may include the amino acid sequences of combination no. 20, which includes a heavy chain comprising the amino acid sequence of SEQ ID NO: 19, a light chain comprising the amino acid sequence of SEQ ID NO: 262, and a spdb-DM4 linker-toxin. In another example of the AADCs disclosed and described herein, an activatable antibody drug conjugate of the present disclosure may include the amino acid sequences of combination no. 70, which includes a heavy chain comprising the amino acid sequence of SEQ ID NO: 19, a light chain comprising the amino acid sequence of SEQ ID NO: 261, and a spdb-DM4 linker-toxin.

Any of the combinations in Table 9 that list a heavy chain and light chain variable region can be combined with human immunoglobulin constant regions to result in fully human IgGs including IgG1, IgG2, IgG4 or mutated constant regions to result in human IgGs with altered functions such as IgG N297A, IgG1 N297Q, or IgG4 S228P. The combinations described in Table 9 are not limited by the particular combinations shown in any given row, and thus can include any heavy chain sequence or heavy chain variable region sequence from column 2 of Table 9 combined with any light chain sequence or light chain variable region sequence from column 3 of Table 9 combined with any linker from column 4 combined with any toxin from column 5. In addition to the heavy chain sequences or heavy chain variable region sequences listed in column 2, any heavy chain sequence or heavy chain variable region sequence disclosed herein can be used in a combination. In addition to the light chain sequences or light chain variable region sequences listed in column 3, any light chain sequence or light chain variable region sequence disclosed herein can be used in a combination. In addition to the linkers listed in column 4, any linker disclosed herein can be used in a combination. In addition to the toxins listed in column 5, any toxin disclosed herein can be used in a combination.

Multispecific Antibodies and Activatable Antibodies

In some embodiments, the activatable CD147 antibody and/or conjugated activatable CD147 antibody is monospecific. In some embodiments, the activatable CD147 antibody and/or conjugated activatable CD147 antibody is multispecific, e.g., by way of non-limiting example, bispecific or trifunctional.

In some embodiments, the activatable CD147 antibody and/or conjugated activatable CD147 antibody is formulated as part of a pro-Bispecific T Cell Engager (BITE) molecule. In some embodiments, the activatable CD147 antibody and/or conjugated activatable CD147 antibody is formulated as part of a pro-Chimeric Antigen Receptor (CAR) modified T cell or other engineered receptor.

The disclosure accordingly also provides multispecific CD147 antibodies. The multispecific antibodies provided herein are multispecific antibodies that recognize CD147 and at least one or more different antigens or epitopes.

The disclosure also provides multispecific CD147 activatable antibodies. The multispecific activatable antibodies provided herein are multispecific antibodies that recognize CD147 and at least one or more different antigens or epitopes and that include at least one masking moiety (MM) linked to at least one antigen- or epitope-binding domain of the multispecific antibody such that coupling of the MM reduces the ability of the antigen- or epitope-binding domain to bind its target. In some embodiments, the MM is coupled to the antigen- or epitope-binding domain of the multispecific antibody via a cleavable moiety (CM) that functions as a substrate for at least one protease. The activatable multispecific antibodies provided herein are stable in circulation, activated at intended sites of therapy and/or diagnosis but not in normal, i.e., healthy tissue, and, when activated, exhibit binding to a target that is at least comparable to the corresponding, unmodified multispecific antibody.

In some embodiments, the activatable antibody or antigen-binding fragment thereof is incorporated in a multispecific activatable antibody or antigen-binding fragment thereof, where at least one arm of the multispecific activatable antibody specifically binds CD147. In some embodiments, the activatable antibody or antigen-binding fragment thereof is incorporated in a bispecific antibody or antigen-binding fragment thereof, where at least one arm of the bispecific activatable antibody specifically binds CD147.

In some embodiments, the antibody or antigen-binding fragment thereof is incorporated in a multispecific antibody or antigen-binding fragment thereof, where at least one arm of the multispecific antibody or antigen-binding fragment thereof specifically binds CD147. In some embodiments, the antibody or antigen-binding fragment thereof is incorporated in a bispecific antibody or antigen-binding fragment thereof, where at least one arm of the bispecific antibody or antigen-binding fragment thereof specifically binds CD147.

In some embodiments, at least one arm of the multispecific antibody or antigen-binding fragment thereof, e.g., a bispecific antibody or antigen-binding fragment thereof, comprises a heavy chain variable region amino acid sequence selected from the group consisting of SEQ ID NOs: 1-4. In some embodiments, at least one arm of the multispecific antibody or antigen-binding fragment thereof, e.g., a bispecific antibody or antigen-binding fragment thereof, comprises a heavy chain variable region amino acid sequence selected from the group consisting of SEQ ID NOs: 1-3.

In some embodiments, at least one arm of the multispecific antibody or antigen-binding fragment thereof, e.g., a bispecific antibody or antigen-binding fragment thereof, comprises a light chain variable region amino acid sequence selected from the group consisting of SEQ ID NOs: 5-9. In some embodiments, at least one arm of the multispecific antibody or antigen-binding fragment thereof, e.g., a bispecific antibody or antigen-binding fragment thereof, comprises a light chain variable region amino acid sequence selected from the group consisting of SEQ ID NOs: 5-8.

In some embodiments, at least one arm of the multispecific antibody or antigen-binding fragment thereof, e.g., a bispecific antibody or antigen-binding fragment thereof, comprises a heavy chain variable region amino acid sequence selected from the group consisting of SEQ ID NOs: 1-4, and a light chain variable region amino acid sequence selected from the group consisting of SEQ ID NOs: 5-9.

In some embodiments, at least one arm of the multispecific antibody or antigen-binding fragment thereof, e.g., a bispecific antibody or antigen-binding fragment thereof, comprises a heavy chain variable region amino acid sequence c selected from the group consisting of SEQ ID NOs: 1-3, and a light chain variable region amino acid sequence selected from the group consisting of SEQ ID NOs: 5-8.

In some embodiments, at least one arm of the multispecific antibody or antigen-binding fragment thereof, e.g., a bispecific antibody or antigen-binding fragment thereof, comprises a heavy chain variable region amino acid sequence that is at least 90%, 91%, 92%, 93%, 94%, 95%, 96%, 97%, 98% or 99% identical to an amino acid sequence selected from the group consisting of SEQ ID NOs: 1-4. In some embodiments, at least one arm of the multispecific antibody or antigen-binding fragment thereof, e.g., a bispecific antibody or antigen-binding fragment thereof, comprises a heavy chain variable region amino acid sequence that is at least 90%, 91%, 92%, 93%, 94%, 95%, 96%, 97%, 98% or 99% identical to an amino acid sequence selected from the group consisting of SEQ ID NOs: 1-3.

In some embodiments, at least one arm of the multispecific antibody or antigen-binding fragment thereof, e.g., a bispecific antibody or antigen-binding fragment thereof, comprises a light chain variable region amino acid sequence that is at least 90%, 91%, 92%, 93%, 94%, 95%, 96%, 97%, 98% or 99% identical to an amino acid sequence selected from the group consisting of SEQ ID NOs: 5-9. In some embodiments, at least one arm of the multispecific antibody or antigen-binding fragment thereof, e.g., a bispecific antibody or antigen-binding fragment thereof, comprises a light chain variable region amino acid sequence that is at least 90%, 91%, 92%, 93%, 94%, 95%, 96%, 97%, 98% or 99% identical to an amino acid sequence selected from the group consisting of SEQ ID NOs: 5-8.

In some embodiments, at least one arm of the multispecific antibody or antigen-binding fragment thereof, e.g., a bispecific antibody or antigen-binding fragment thereof, comprises a heavy chain variable region amino acid sequence that is at least 90%, 91%, 92%, 93%, 94%, 95%, 96%, 97%, 98% or 99% identical to an amino acid sequence selected from the group consisting of SEQ ID NOs: 1-4, and a light chain variable region amino acid sequence that is at least 90%, 91%, 92%, 93%, 94%, 95%, 96%, 97%, 98% or 99% identical to an amino acid sequence selected from the group consisting of SEQ ID NOs: 5-9.

In some embodiments, at least one arm of the multispecific antibody or antigen-binding fragment thereof, e.g., a bispecific antibody or antigen-binding fragment thereof, comprises a heavy chain variable region amino acid sequence that is at least 90%, 91%, 92%, 93%, 94%, 95%, 96%, 97%, 98% or 99% identical to an amino acid sequence selected from the group consisting of SEQ ID NOs: 1-3, and a light chain variable region amino acid sequence that is at least 90%, 91%, 92/%, 93%, 94%, 95%, 96%, 97%, 98% or 99% identical to an amino acid sequence selected from the group consisting of SEQ ID NOs: 5-8.

In some embodiments, at least one arm of the multispecific antibody or antigen-binding fragment thereof, e.g., a bispecific antibody or antigen-binding fragment thereof, comprises a combination of a variable heavy chain complementarity determining region 1 (VH CDR1, also referred to herein as CDRH1) sequence, a variable heavy chain complementarity determining region 2 (VH CDR2, also referred to herein as CDRH2) sequence, a variable heavy chain complementarity determining region 3 (VH CDR3, also referred to herein as CDRH3) sequence, a variable light chain complementarity determining region 1 (VL CDR1, also referred to herein as CDRL1) sequence, a variable light chain complementarity determining region 2 (VL CDR2, also referred to herein as CDRL2) sequence, and a variable light chain complementarity determining region 3 (VL CDR3, also referred to herein as CDRL3) sequence, wherein at least one complementarity determining region (CDR) sequence is selected from the group consisting of a VH CDR1 sequence comprising the amino acid sequence GFTFSNYWMN (SEQ ID NO: 10) or GFTFSNYWMD (SEQ ID NO: 11); a VH CDR2 sequence comprising the amino acid sequence EIRLKSYNYATH (SEQ ID NO: 12); a VH CDR3 sequence comprising the amino acid sequence AGTDY (SEQ ID NO: 13); a VL CDR1 sequence comprising the amino acid sequence KASQSVRTDVA (SEQ ID NO: 14) or RASQSVRTDVG (SEQ ID NO: 15); a VL CDR2 sequence comprising the amino acid sequence YSSNRYT (SEQ ID NO: 16); and a VL CDR3 sequence comprising the amino acid sequence QQDYSSPFT (SEQ ID NO: 17) or QQDYSSPYT (SEQ ID NO: 18).

In some embodiments, at least one arm of the multispecific antibody or antigen-binding fragment thereof, e.g., a bispecific antibody or antigen-binding fragment thereof, comprises a combination of a VH CDR1 sequence, a VH CDR2 sequence, a VH CDR3 sequence, a VL CDR1 sequence, a VL CDR2 sequence, and a VL CDR3 sequence, wherein at least one CDR sequence is selected from the group consisting of a VH CDR1 sequence that includes a sequence that is at least 90%, 91%, 92%, 93%, 94%, 95%, 96%, 97%, 98%, 99% or more identical to a VH CDR1 sequence comprising the amino acid sequence GFTFSNYWMN (SEQ ID NO: 10) or GFTFSNYWMD (SEQ ID NO: 11); a VH CDR2 sequence that includes a sequence that is at least 90%, 91%, 92%, 93%, 94%, 95%, 96%, 97%, 98%, 99%, or more identical to a VH CDR2 sequence comprising the amino acid sequence EIRLKSYNYATH (SEQ ID NO: 12); a VH CDR3 sequence that includes a sequence that is at least 90%, 91%, 92%, 93%, 94%, 95%, 96%, 97%, 98%, 99% or more identical to a VH CDR3 sequence comprising the amino acid sequence AGTDY (SEQ ID NO: 13); a VL CDR1 sequence that includes a sequence that is at least 90%, 91%, 92%, 93%, 94%, 95%, 96%, 97%, 98%, 99% or more identical to a VL CDR1 sequence comprising the amino acid sequence ASSSVYYMY (SEQ ID NO: 12) or CRASSSVYYMY (SEQ ID NO: 13); a VL CDR2 sequence that includes a sequence that is at least 90%, 91%, 92%, 93%, 94%, 95%, 96%, 97%, 98%, 99% or more identical to a VL CDR2 sequence comprising the amino acid sequence YSSNRYT (SEQ ID NO: 16; and a VL CDR3 sequence that includes a sequence that is at least 90%, 91%, 92%, 93%, 94%, 95%, 96%, 97%, 98%, 99% or more identical to a VL CDR3 sequence comprising the amino acid sequence QQDYSSPFT (SEQ ID NO: 17) or QQDYSSPYT (SEQ ID NO: 18).

In some embodiments, at least one arm of the multispecific antibody or antigen-binding fragment thereof, e.g., a bispecific antibody or antigen-binding fragment thereof, comprises a combination of a VH CDR1 sequence, a VH CDR2 sequence, a VH CDR3 sequence, a VL CDR1 sequence, a VL CDR2 sequence, and a VL CDR3 sequence, wherein the VH CDR1 sequence comprises the amino acid sequence GFTFSNYWMN (SEQ ID NO: 10) or GFTFSNYWMD (SEQ ID NO: 11); the VH CDR2 sequence comprises the amino acid sequence EIRLKSYNYATH (SEQ ID NO: 12); the VH CDR3 sequence comprises the amino acid sequence AGTDY (SEQ ID NO: 13); the VL CDR1 sequence comprises the amino acid sequence KASQSVRTDVA (SEQ ID NO: 14) or RASQSVRTDVG (SEQ ID NO: 15); the VL CDR2 sequence comprises the amino acid sequence YSSNRYT (SEQ ID NO: 16); and the VL CDR3 sequence comprises the amino acid sequence QQDYSSPFT (SEQ ID NO: 17) or QQDYSSPYT (SEQ ID NO: 18).

In some embodiments, at least one arm of the multispecific antibody or antigen-binding fragment thereof, e.g., a bispecific antibody or antigen-binding fragment thereof, comprises a combination of a VH CDR1 sequence, a VH CDR2 sequence, a VH CDR3 sequence, a VL CDR1 sequence, a VL CDR2 sequence, and a VL CDR3 sequence, wherein the VH CDR1 sequence comprises a sequence that is at least 90%, 91%, 92%, 93%, 94%, 95%, 96%, 97%, 98%, 99% or more identical to the amino acid sequence GFTFSNYWMN (SEQ ID NO: 10) or GFTFSNYWMD (SEQ ID NO: 11); the VH CDR2 sequence comprises a sequence that is at least 90%, 91%, 92%, 93%, 94%, 95%, 96%, 97%, 98%, 99% or more identical to the amino acid sequence EIRLKSYNYATH (SEQ ID NO: 12); the VH CDR3 sequence comprises a sequence that is at least 90%, 91%, 92%, 93%, 94%, 95%, 96%, 97%, 98%, 99% or more identical to the amino acid sequence AGTDY (SEQ ID NO: 13); the VL CDR1 sequence comprises a sequence that is at least 90%, 91%, 92%, 93%, 94%, 95%, 96%, 97%, 98%, 99% or more identical to the amino acid sequence KASQSVRTDVA (SEQ ID NO: 14) or RASQSVRTDVG (SEQ ID NO: 15); the VL CDR2 sequence comprises a sequence that is at least 90%, 91%, 92%, 93%, 94%, 95%, 96%, 97%, 98%, 99% or more identical to the amino acid sequence YSSNRYT (SEQ ID NO: 16); and the VL CDR3 sequence comprises a sequence that is at least 90%, 91%, 92%, 93%, 94%, 95%, 96%, 97%, 98%, 99% or more identical to the amino acid sequence QQDYSSPFT (SEQ ID NO: 17) or QQDYSSPYT (SEQ ID NO: 18).

In some embodiments, at least one arm of the multispecific antibody or antigen-binding fragment thereof, e.g., a bispecific antibody or antigen-binding fragment thereof, comprises a heavy chain or a heavy chain variable region that comprises or is derived from a heavy chain amino acid sequence or heavy chain variable region amino acid sequence shown in Table 1. In some embodiments, at least one arm of the multispecific antibody or antigen-binding fragment thereof, e.g., a bispecific antibody or antigen-binding fragment thereof, comprises a light chain or a light chain variable region that comprises or is derived from a light chain amino acid sequence or light chain variable region amino acid sequence shown in Table 1. In some embodiments, at least one arm of the multispecific antibody or antigen-binding fragment thereof, e.g., a bispecific antibody or antigen-binding fragment thereof, comprises a heavy chain or a heavy chain variable region amino acid sequence that comprises or is derived from a heavy chain amino acid sequence or heavy chain variable region amino acid sequence shown in Table 1 and a light chain or a light chain variable region amino acid sequence that comprises or is derived from a light chain amino acid sequence or light chain variable region amino acid sequence shown in Table 1.

In some embodiments, at least one arm of the multispecific antibody or antigen-binding fragment thereof, e.g., a bispecific antibody or antigen-binding fragment thereof, comprises a heavy chain variable region amino acid sequence that is at least 90%, 91%, 92%, 93%, 94%, 95%, 96%, 97%, 98% or 99% identical to an amino acid sequence selected from the group consisting of the heavy chain variable region sequences shown in Table 1. In some embodiments, at least one arm of the multispecific antibody or antigen-binding fragment thereof, e.g., a bispecific antibody or antigen-binding fragment thereof, comprises an amino acid sequence that is at least 90%, 91%, 92%, 93%, 94%, 95%, 96%, 97%, 98% or 99% identical to an amino acid sequence that is selected from the group consisting of the light chain variable region sequences shown in Table 1. In some embodiments, at least one arm of the multispecific antibody or antigen-binding fragment thereof, e.g., a bispecific antibody or antigen-binding fragment thereof, comprises an amino acid sequence that is at least 90%, 91%, 92%, 93%, 94%, 95%, 96%, 97%, 98% or 99%, identical to an amino acid sequence selected from the group consisting of the heavy chain variable region sequences shown in Table 1 and a light chain variable region amino acid sequence that is at least 90%, 91%, 92%, 93%, 94%, 95%, 96%, 97%, 98% or 99% identical to an amino acid sequence selected from the group consisting of the light chain variable region sequences shown in Table 1.

In some embodiments, at least one arm of the multispecific antibody or antigen-binding fragment thereof, e.g., a bispecific antibody or antigen-binding fragment thereof, comprises a combination of a variable heavy chain complementarity determining region 1 (VH CDR1, also referred to herein as CDRH1) sequence, a variable heavy chain complementarity determining region 2 (VH CDR2, also referred to herein as CDRH2) sequence, a variable heavy chain complementarity determining region 3 (VH CDR3, also referred to herein as CDRH3) sequence, a variable light chain complementarity determining region 1 (VL CDR1, also referred to herein as CDRL1) sequence, a variable light chain complementarity determining region 2 (VL CDR2, also referred to herein as CDRL2) sequence, and a variable light chain complementarity determining region 3 (VL CDR3, also referred to herein as CDRL3) sequence, wherein at least one CDR sequence is selected from the group consisting of a VH CDR1 sequence shown in Table 2; a VH CDR2 sequence shown in Table 2; a VH CDR3 sequence shown in Table 2; a VL CDR1 sequence shown in Table 2; a VL CDR2 sequence shown in Table 2; and a VL CDR3 sequence shown in Table 2.

In some embodiments, at least one arm of the multispecific antibody or antigen-binding fragment thereof, e.g., a bispecific antibody or antigen-binding fragment thereof, comprises a combination of a VH CDR1 sequence, a VH CDR2 sequence, a VH CDR3 sequence, a VL CDR1 sequence, a VL CDR2 sequence, and a VL CDR3 sequence, wherein at least one CDR sequence is selected from the group consisting of a VH CDR1 sequence that includes a sequence that is at least 90%, 91%, 92%, 93%, 94%, 95%, 96%, 97%, 98%, 99% or more identical to a VH CDR1 sequence shown in Table 2; a VH CD2 sequence that includes a sequence that is at least 90%, 91%, 92%, 93%, 94%, 95%, 96%, 97%, 98%, 99% or more identical to a VH CDR2 sequence shown in Table 2; a VH CDR3 sequence that includes a sequence that is at least 90%, 91%, 92%, 93%, 940%, 95%, 96%, 97%, 98%, 99% or more identical to a VH CDR3 sequence shown in Table 2; a VL CDR1 sequence that includes a sequence that is at least 90%, 91%, 92%, 93%, 94%, 95%, 96%, 97%, 98%, 99% or more identical to a VL CDR1 sequence shown in Table 2; a VL CDR2 sequence that includes a sequence that is at least 90%, 91%, 920%, 93%, 94%, 95%, 96%, 97%, 98/O, 99% or more identical to a VL CDR2 sequence shown in Table 2; and a VL CDR3 sequence that includes a sequence that is at least 90%, 91%, 92%, 93%, 94%, 95%, 96%, 97%, 98%, 99% or more identical to a VL CDR3 sequence shown in Table 2.

In some embodiments at least one arm of the multispecific antibody or antigen-binding fragment thereof, e.g., a bispecific antibody or antigen-binding fragment thereof, comprises a combination of a VH CDR1 sequence, a VH CDR2 sequence, a VH CDR3 sequence, a VL CDR1 sequence, a VL CDR2 sequence, and a VL CDR3 sequence, wherein the combination is a combination of the six CDR sequences (VH CDR1, VH CDR2, VH CDR3, VL CDR1, VL CDR2, and VL CDR3) shown in a single row in Table 2.

In some embodiments, at least one arm of the multispecific antibody or antigen-binding fragment thereof, e.g., a bispecific antibody or antigen-binding fragment thereof, comprises a light chain variable region that comprise a combination of a VL CDR1 sequence, a VL CDR2 sequence, and a VL CDR3 sequence, wherein the combination is a combination of the three light chain CDR sequences (VL CDR1, VL CDR2, VL CDR3) shown in a single row in Table 2.

In some embodiments, at least one arm of the multispecific antibody or antigen-binding fragment thereof, e.g., a bispecific antibody or antigen-binding fragment thereof, comprises a heavy chain variable region that comprise a combination of a VH CDR1 sequence, a VH CDR2 sequence, and a VH CDR3 sequence, wherein the combination is a combination of the three heavy chain CDR sequences (VH CDR1, VH CDR2, VH CDR3) shown in a single row in Table 2.

In some embodiments, at least one arm of the multispecific antibody or antigen-binding fragment thereof, e.g., a bispecific antibody or antigen-binding fragment thereof, comprises a combination of a VH CDR1 sequence, a VH CDR2 sequence, a VH CDR3 sequence, a VL CDR1 sequence, a VL CDR2 sequence, and a VL CDR3 sequence, wherein each CDR sequence in the combination comprises a sequence that is at least 90%, 91%, 92%, 93%, 94%, 95%, 96%, 97%, 98%, 99% or more identical to the corresponding CDR sequence in a combination of the six CDR sequences (VH CDR1, VH CDR2, VH CDR3, VL CDR1, VL CDR2, and VL CDR3) shown in a single row in Table 2.

In some embodiments, at least one arm of the multispecific antibody or antigen-binding fragment thereof, e.g., a bispecific antibody or antigen-binding fragment thereof, comprises a heavy chain variable region that comprise a combination of a VH CDR1 sequence, a VH CDR2 sequence, and a VH CDR3 sequence, wherein each CDR sequence in the combination comprises a sequence that is at least 90%, 91%, 92%, 93%, 94%, 95%, 96%, 97%, 98%, 99% or more identical to the corresponding CDR sequence in a combination of three heavy chain CDR sequences (VH CDR1, VH CDR2, VH CDR3) shown in a single row in Table 2.

In some embodiments, at least one arm of the multispecific antibody or antigen-binding fragment thereof, e.g., a bispecific antibody or antigen-binding fragment thereof, comprises a light chain variable region that comprise a combination of a VL CDR1 sequence, a VL CDR2 sequence, and a VL CDR3 sequence, wherein each CDR sequence in the combination comprises a sequence that is at least 90%, 91%, 92%, 93%, 94%, 95%, 96%, 97%, 98%, 99% or more identical to the corresponding CDR sequence in a combination of three light chain CDR sequences (VL CDR1, VL CDR2, VL CDR3) shown in a single row in Table 2.

In some embodiments, the multispecific antibodies/activatable antibodies are designed to engage immune effector cells, also referred to herein as immune-effector cell engaging multispecific activatable antibodies. In some embodiments, the multispecific antibodies/activatable antibodies are designed to engage leukocytes, also referred to herein as leukocyte engaging multispecific activatable antibodies. In some embodiments, the multispecific antibodies/activatable antibodies are designed to engage T cells, also referred to herein as T-cell engaging multispecific antibodies/activatable antibodies. In some embodiments, the multispecific antibodies/activatable antibodies engage a surface antigen on a leukocyte, such as on a T cell, on a natural killer (NK) cell, on a myeloid mononuclear cell, on a macrophage, and/or on another immune effector cell. In some embodiments, the immune effector cell is a leukocyte. In some embodiments, the immune effector cell is a T cell. In some embodiments, the immune effector cell is a NK cell. In some embodiments, the immune effector cell is a mononuclear cell, such as a myeloid mononuclear cell. In some embodiments, the multispecific activatable antibodies are designed to bind or otherwise interact with more than one target and/or more than one epitope, also referred to herein as multi-antigen targeting activatable antibodies. As used herein, the terms “target” and “antigen” are used interchangeably.

In some embodiments, immune effector cell engaging multispecific activatable antibodies of the disclosure include a targeting antibody or antigen-binding fragment thereof that binds CD147 and an immune effector cell engaging antibody or antigen-binding portion thereof, where at least one of the targeting antibody or antigen-binding fragment thereof and/or the immune effector cell engaging antibody or antigen-binding portion thereof is masked. In some embodiments, the immune effector cell engaging antibody or antigen binding fragment thereof includes a first antibody or antigen-binding fragment thereof (AB1) that binds a first, immune effector cell engaging target, where the AB1 is attached to a masking moiety (MM1) such that coupling of the MM1 reduces the ability of the AB1 to bind the first target. In some embodiments, the targeting antibody or antigen-binding fragment thereof includes a second antibody or fragment thereof that includes a second antibody or antigen-binding fragment thereof (AB2) that binds CD147, where the AB2 is attached to a masking moiety (MM2) such that coupling of the MM2 reduces the ability of the AB2 to bind CD147. In some embodiments, the immune effector cell engaging antibody or antigen binding fragment thereof includes a first antibody or antigen-binding fragment thereof (AB1) that binds a first, immune effector cell engaging target, where the AB1 is attached to a masking moiety (MM1) such that coupling of the MM1 reduces the ability of the AB1 to bind the first target, and the targeting antibody or antigen-binding fragment thereof includes a second antibody or fragment thereof that includes a second antibody or antigen-binding fragment thereof (AB2) that binds CD147, where the AB2 is attached to a masking moiety (MM2) such that coupling of the MM2 reduces the ability of the AB2 to bind CD147. In some embodiments, the non-immune effector cell engaging antibody is a cancer targeting antibody. In some embodiments the non-immune cell effector antibody is an IgG. In some embodiments the immune effector cell engaging antibody is a scFv. In some embodiments the CD147-targeting antibody (e.g., non-immune cell effector antibody) is an IgG and the immune effector cell engaging antibody is a scFv. In some embodiments, the immune effector cell is a leukocyte. In some embodiments, the immune effector cell is a T cell. In some embodiments, the immune effector cell is a NK cell. In some embodiments, the immune effector cell is a myeloid mononuclear cell.

In some embodiments, T-cell engaging multispecific activatable antibodies of the disclosure include a CD147-targeting antibody or antigen-binding fragment thereof and a T-cell engaging antibody or antigen-binding portion thereof, where at least one of the CD147-targeting antibody or antigen-binding fragment thereof and/or the T-cell engaging antibody or antigen-binding portion thereof is masked. In some embodiments, the T-cell engaging antibody or antigen binding fragment thereof includes a first antibody or antigen-binding fragment thereof (AB1) that binds a first, T-cell engaging target, where the AB1 is attached to a masking moiety (MM1) such that coupling of the MM reduces the ability of the AB1 to bind the first target. In some embodiments, the targeting antibody or antigen-binding fragment thereof includes a second antibody or fragment thereof that includes a second antibody or antigen-binding fragment thereof (AB2) that binds CD147, where the AB2 is attached to a masking moiety (MM2) such that coupling of the MM2 reduces the ability of the AB2 to bind CD147. In some embodiments, the T-cell engaging antibody or antigen binding fragment thereof includes a first antibody or antigen-binding fragment thereof (AB1) that binds a first, T-cell engaging target, where the AB1 is attached to a masking moiety (MM1) such that coupling of the MM1 reduces the ability of the AB1 to bind the first target, and the targeting antibody or antigen-binding fragment thereof includes a second antibody or fragment thereof that includes a second antibody or antigen-binding fragment thereof (AB2) that binds CD147, where the AB2 is attached to a masking moiety (MM2) such that coupling of the MM2 reduces the ability of the AB2 to bind CD147.

In some embodiments of an immune effector cell engaging multispecific antibody/activatable antibody, one antigen is CD147, and another antigen is typically a stimulatory or inhibitory receptor present on the surface of a T-cell, natural killer (NK) cell, myeloid mononuclear cell, macrophage, and/or other immune effector cell, such as, but not limited to, B7-H4, BTLA, CD3, CD4, CD8, CD16a, CD25, CD27, CD28, CD32, CD56, CD137, CTLA-4, GITR, HVEM, ICOS, LAG3, NKG2D, OX40, PD-1, TIGIT, TIM3, or VISTA. In some embodiments, the antigen is a stimulatory receptor present on the surface of a T cell or NK cell; examples of such stimulatory receptors include, but are not limited to, CD3, CD27, CD28, CD137 (also referred to as 4-1BB), GITR, HVEM, ICOS, NKG2D, and OX40. In some embodiments, the antigen is an inhibitory receptor present on the surface of a T-cell; examples of such inhibitory receptors include, but are not limited to, BTLA, CTLA-4, LAG3, PD-1, TIGIT, TIM3, and NK-expressed KIRs. The antibody domain conferring specificity to the T-cell surface antigen may also be substituted by a ligand or ligand domain that binds to a T-cell receptor, a NK-cell receptor, a macrophage receptor, and/or other immune effector cell receptor, such as, but not limited to, B7-1, B7-2, B7H3, PDL1, PDL2, or TNFSF9.

In some embodiments, the T-cell engaging multispecific activatable antibody includes an anti-CD3 epsilon (CD3ε, also referred to herein as CD3e and CD3) scFv and a targeting antibody or antigen-binding fragment thereof, where at least one of the anti-CD3ε scFv and/or the targeting antibody or antigen-binding portion thereof is masked. In some embodiments, the CD3ε scFv includes a first antibody or antigen-binding fragment thereof (AB1) that binds CD3ε, where the AB1 is attached to a masking moiety (MM1) such that coupling of the MM1 reduces the ability of the AB1 to bind CD3ε. In some embodiments, the targeting antibody or antigen-binding fragment thereof includes a second antibody or fragment thereof that includes a second antibody or antigen-binding fragment thereof (AB2) that binds CD147, where the AB2 is attached to a masking moiety (MM2) such that coupling of the MM2 reduces the ability of the AB2 to bind CD147. In some embodiments, the CD3ε scFv includes a first antibody or antigen-binding fragment thereof (AB1) that binds CD3ε, where the AB1 is attached to a masking moiety (MM1) such that coupling of the MM1 reduces the ability of the AB1 to bind CD3ε, and the targeting antibody or antigen-binding fragment thereof includes a second antibody or fragment thereof that includes a second antibody or antigen-binding fragment thereof (AB2) that binds CD147, where the AB2 is attached to a masking moiety (MM2) such that coupling of the MM2 reduces the ability of the AB2 to bind CD147.

In some embodiments, the multi-antigen targeting antibodies and/or multi-antigen targeting activatable antibodies include at least a first antibody or antigen-binding fragment thereof that binds a first target and/or first epitope and a second antibody or antigen-binding fragment thereof that binds a second target and/or a second epitope. In some embodiments, the multi-antigen targeting antibodies and/or multi-antigen targeting activatable antibodies bind two or more different targets. In some embodiments, the multi-antigen targeting antibodies and/or multi-antigen targeting activatable antibodies bind two or more different epitopes on the same target. In some embodiments, the multi-antigen targeting antibodies and/or multi-antigen targeting activatable antibodies bind a combination of two or more different targets and two or more different epitopes on the same target.

In some embodiments, a multispecific activatable antibody comprising an IgG has the IgG variable domains masked. In some embodiments, a multispecific activatable antibody comprising a scFv has the scFv domains masked. In some embodiments, a multispecific activatable antibody has both IgG variable domains and scFv domains, where at least one of the IgG variable domains is coupled to a masking moiety. In some embodiments, a multispecific activatable antibody has both IgG variable domains and scFv domains, where at least one of the scFv domains is coupled to a masking moiety. In some embodiments, a multispecific activatable antibody has both IgG variable domains and scFv domains, where at least one of the IgG variable domains is coupled to a masking moiety and at least one of the scFv domains is coupled to a masking moiety. In some embodiments, a multispecific activatable antibody has both IgG variable domains and scFv domains, where each of the IgG variable domains and the scFv domains is coupled to its own masking moiety. In some embodiments, one antibody domain of a multispecific activatable antibody has specificity for a target antigen and another antibody domain has specificity for a T-cell surface antigen. In some embodiments, one antibody domain of a multispecific activatable antibody has specificity for a target antigen and another antibody domain has specificity for another target antigen. In some embodiments, one antibody domain of a multispecific activatable antibody has specificity for an epitope of a target antigen and another antibody domain has specificity for another epitope of the target antigen.

In a multispecific activatable antibody, a scFv can be fused to the carboxyl terminus of the heavy chain of an IgG activatable antibody, to the carboxyl terminus of the light chain of an IgG activatable antibody, or to the carboxyl termini of both the heavy and light chains of an IgG activatable antibody. In a multispecific activatable antibody, a scFv can be fused to the amino terminus of the heavy chain of an IgG activatable antibody, to the amino terminus of the light chain of an IgG activatable antibody, or to the amino termini of both the heavy and light chains of an IgG activatable antibody. In a multispecific activatable antibody, a scFv can be fused to any combination of one or more carboxyl termini and one or more amino termini of an IgG activatable antibody. In some embodiments, a masking moiety (MM) linked to a cleavable moiety (CM) is attached to and masks an antigen binding domain of the IgG. In some embodiments, a masking moiety (MM) linked to a cleavable moiety (CM) is attached to and masks an antigen binding domain of at least one scFv. In some embodiments, a masking moiety (MM) linked to a cleavable moiety (CM) is attached to and masks an antigen binding domain of an IgG and a masking moiety (MM) linked to a cleavable moiety (CM) is attached to and masks an antigen binding domain of at least one scFv.

The disclosure provides examples of multispecific activatable antibody structures which include, but are not limited to, the following: (VL-CL)₂:(VH-CH1-CH2-CH3-L4-VH*-L3-VL*-L2-CM-L1-MM)₂; (VL-CL)₂:(VH-CH1-CH2-CH3-L4-VL*-L3-VH*-L2-CM-L1-MM)₂; (MM-L1-CM-L2-VL-CL)₂:(VH-CH1-CH2-CH3-L4-VH*-L3-VL*)₂; (MM-L1-CM-L2-VL-CL)₂:(VH-CH1-CH2-CH3-L4-VL*-L3-VH*)₂; (VL-CL)₂:(MM-L1-CM-L2-VL*-L3-VH*-L4-VH-CH1-CH2-CH3)₂; (VL-CL)₂:(MM-L1-CM-L2-VH*-L3-VL*-L4-VH-CH1-CH2-CH3)₂, (MM-L1-CM-L2-VL-CL)₂:(VL*-L3-VH*-L4-VH-CH1-CH2-CH3)₂; (MM-L1-CM-L2-VL-CL)₂:(VH*-L3-VL*-L4-VH-CH1-CH2-CH3)₂; (VL-CL-L4-VH*-L3-VL*-L2-CM-L1-MM)₂:(VH-CH1-CH2-CH3)₂; (VL-CL-L4-VL*-L3-VH*-L2-CM-L1-MM)₂:(VH-CH1-CH2-CH3)₂; (MM-L1-CM-L2-VL*-L3-VH*-L4-VL-CL)₂:(VH-CH1-CH2-CH3)₂; (MM-L1-CM-L2-VH*-L3-VL*-L4-VL-CL)₂:(VH-CH1-CH2-CH3)₂; (VL-CL-L4-VH*-L3-VL*-L2-CM-L1-MM)₂: (MM-L1-CM-L2-VL*-L3-VH*-L4-VH-CH1-CH2-CH3)₂; (VL-CL-L4-VH*-L3-VL*-L2-CM-L1-MM)₂: (MM-L1-CM-L2-VH*-L3-VL*-L4-VH-CH1-CH2-CH3)₂; (VL-CL-L4-VL*-L3-VH*-L2-CM-L1-MM)₂: (MM-L1-CM-L2-VL*-L3-VH*-L4-VH-CH1-CH2-CH3)₂; (VL-CL-L4-VL*-L3-VH*-L2-CM-L1-MM)₂: (MM-L1-CM-L2-VH*-L3-VL*-L4-VH-CH1-CH2-CH3)₂; (VL-CL-L4-VH*-L3-VL*)₂: (MM-L1-CM-L2-VL*-L3-VH*-L4-VH-CH1-CH2-CH3)₂; (VL-CL-L4-VH*-L3-VL*)₂: (MM-L1-CM-L2-VH*-L3-VL*-L4-VH-CH1-CH2-CH3)₂; (VL-CL-L4-VL*-L3-VH*)₂: (MM-L1-CM-L2-VL*-L3-VH*-L4-VH-CH1-CH2-CH3)₂; (VL-CL-L4-VL*-L3-VH*)₂: (MM-L1-CM-L2-VH*-L3-VL*-L4A-VH-CH1-CH2-CH3)₂; (VL-CL-L4-VH*-L3-VL*-L2-CM-L1-MM)₂: (VL*-L3-VH*-L4A-VH-CH1-CH2-CH3)₂; (VL-CL-L4-VH*-L3-VL*-L2-CM-L1-MM)₂: (VH*-L3-VL*-L4-VH-CH1-CH2-CH3)₂; (VL-CL-L4-VL*-L3-VH*-L2-CM-L1-MM)₂: (VL*-L3-VH*-L4-VH-CH1-CH2-CH3)₂; or (VL-CL-L4-VL*-L3-VH*-L2-CM-L1-MM)₂: (VH*-L3-VL*-L4-VH-CH1-CH2-CH3)₂, wherein: VL and VH represent the light and heavy variable domains of the first specificity, contained in the IgG; VL* and VH* represent the variable domains of the second specificity, contained in the scFv; L1 is a linker peptide connecting the masking moiety (MM) and the cleavable moiety (CM); L2 is a linker peptide connecting the cleavable moiety (CM), and the antibody; L3 is a linker peptide connecting the variable domains of the scFv; L4 is a linker peptide connecting the antibody of the first specificity to the antibody of the second specificity; CL is the light-chain constant domain; and CH1, CH2, CH3 are the heavy chain constant domains. The first and second specificities can be toward any antigen or epitope.

In some embodiments of a T-cell engaging multispecific antibody/activatable antibody, one antigen is CD147, and another antigen is typically a stimulatory (also referred to herein as activating) or inhibitory receptor present on the surface of a T-cell, natural killer (NK) cell, myeloid mononuclear cell, macrophage, and/or other immune effector cell, such as, but not limited to, B7-H4, BTLA, CD3, CD4, CD8, CD16a, CD25, CD27, CD28, CD32, CD56, CD137 (also referred to as TNFRSF9), CTLA-4, GITR, HVEM, ICOS, LAG3, NKG2D, OX40, PD-1, TIGIT, TIM3, or VISTA. The antibody domain conferring specificity to the T-cell surface antigen may also be substituted by a ligand or ligand domain that binds to a T-cell receptor, a NK-cell receptor, a macrophage receptor, and/or other immune effector cell receptor.

In some embodiments, the targeting antibody is a CD147 antibody disclosed herein. In some embodiments, the targeting antibody can be in the form an activatable antibody. In some embodiments, the scFv(s) can be in the form of a Pro-scFv (see, e.g., WO 2009/025846, WO 2010/081173).

In some embodiments, the scFv is specific for binding CD3ε, and comprises or is derived from an antibody or fragment thereof that binds CD3ε, e.g., CH2527, FN18, H2C, OKT3, 2C11, UCHT1, or V9. In some embodiments, the scFv is specific for binding CTLA-4 (also referred to herein as CTLA and CTLA4).

In some embodiments, the anti-CTLA-4 scFv includes the amino acid sequence:

(SEQ ID NO: 643)

GGGSGGGGSGSGGGSGGGGSGGGEIVLTQSPGTLSLSPGERATLSCRASQ
SVSSSYLAWYQQKPGQAPRLLIYGASSRATGIPDRFSGSGSGTDFTLTIS
RLEPEDFAVYYCQQYGSSPLTFGGGTKVEIKRSGGSTITSYNVYYTKLSS
SGTQVQLVQTGGGVVQPGRSLRLSCAASGSTFSSYAMSWVRQAPGKGLEW
VSAISGSGGSTYYADSVKGRFTISRDNSKNTLYLQMNSLRAEDTAVYYCA
TNSLYWYFDLWGRGTLVTVSSAS

In some embodiments, the anti-CTLA-4 scFv includes the amino acid sequence that is at least 90%, 91%, 92%, 93%, 94%, 95%, 96%, 97%, 98%, 99% or more identical to the amino acid sequence of SEQ ID NO: 643.

In some embodiments, the anti-CD3s scFv includes the amino acid sequence:

(SEQ ID NO: 644)

GGGSGGGGSGSGGGSGGGGSGGGQVQLQQSGAELARPGASVKMSCKASGY
TFTRYTMEWVKQRPGQGLEWIGYINPSRGYTNYNQKFKDKATLTTDKSSS
TAYMQLSSLTSEDSAVYYCARYYDDHYCLDYWGQGTTLTVSSGGGGSGGG
GSGGGGSQIVLTQSPAIMSASPGEKVTMTCSASSSVSYMNWYQQKSGTSP
KRWIYDTSKLASGVPAHFRGSGSGTSYSLTISGMEAEDAATYYCQQWSSN
PFTFGSGTKLEINR

In some embodiments, the anti-CD3ε scFv includes the amino acid sequence that is at least 90%, 91%, 92%, 93%, 94%, 95%, 96%, 97%, 98%, 99% or more identical to the amino acid sequence of SEQ ID NO: 644.

In some embodiments, the scFv is specific for binding one or more T-cells, one or more NK-cells and/or one or more macrophages. In some embodiments, the scFv is specific for binding a target selected from the group consisting of B7-H4, BTLA, CD3, CD4, CD8, CD16a, CD25, CD27, CD28, CD32, CD56, CD137, CTLA-4, GITR, HVEM, ICOS, LAG3, NKG2D, OX40, PD-1, TIGIT, TIM3, or VISTA.

In some embodiments, the multispecific antibody/activatable antibody also includes an agent conjugated to the AB. In some embodiments, the agent is a therapeutic agent. In some embodiments, the agent is an antineoplastic agent. In some embodiments, the agent is a toxin or fragment thereof. In some embodiments, the agent is conjugated to the multispecific antibody/activatable antibody via a linker. In some embodiments, the agent is conjugated to the AB via a cleavable linker. In some embodiments, the linker is a non-cleavable linker. In some embodiments, the agent is a microtubule inhibitor. In some embodiments, the agent is a nucleic acid damaging agent, such as a DNA alkylator or DNA intercalator, or other DNA damaging agent. In some embodiments, the linker is a cleavable linker. In some embodiments, the agent is an agent selected from the group listed in Table 6. In some embodiments, the agent is a dolastatin. In some embodiments, the agent is an auristatin or derivative thereof. In some embodiments, the agent is auristatin E or a derivative thereof. In some embodiments, the agent is monomethyl auristatin E (MMAE). In some embodiments, the agent is monomethyl auristatin D (MMAD). In some embodiments, the agent is a maytansinoid or maytansinoid derivative. In some embodiments, the agent is DM1 or DM4. In some embodiments, the agent is a duocarmycin or derivative thereof. In some embodiments, the agent is a calicheamicin or derivative thereof. In some embodiments, the agent is a pyrrolobenzodiazepine. In some embodiments, the agent is a pyrrolobenzodiazepine dimer.

In some embodiments, the multispecific antibody/activatable antibody also includes a detectable moiety. In some embodiments, the detectable moiety is a diagnostic agent.

In some embodiments, the multispecific antibody/activatable antibody naturally contains one or more disulfide bonds. In some embodiments, the multispecific activatable antibody can be engineered to include one or more disulfide bonds.

The disclosure also provides an isolated nucleic acid molecule encoding a multispecific antibody/activatable antibody described herein, as well as vectors that include these isolated nucleic acid sequences. The disclosure provides methods of producing a multispecific antibody/activatable antibody by culturing a cell under conditions that lead to expression of the antibody/activatable antibody, wherein the cell comprises such a nucleic acid molecule. In some embodiments, the cell comprises such a vector.

The disclosure also provides a method of manufacturing multispecific CD147 antibodies of the disclosure by (a) culturing a cell comprising a nucleic acid construct that encodes the multispecific antibody under conditions that lead to expression of the multispecific antibody.

The disclosure also provides a method of manufacturing multispecific activatable CD147 antibodies of the disclosure by (a) culturing a cell comprising a nucleic acid construct that encodes the multispecific activatable antibody under conditions that lead to expression of the multispecific activatable antibody, and (b) recovering the multispecific activatable antibody. Suitable AB, MM, and/or CM include any of the AB, MM, and/or CM disclosed herein.

The disclosure also provides multispecific activatable antibodies and/or multispecific activatable antibody compositions that include at least a first antibody or antigen-binding fragment thereof (AB1) that specifically binds a first target or first epitope and a second antibody or antigen-biding fragment thereof (AB2) that binds a second target or a second epitope, where at least AB1 is coupled or otherwise attached to a masking moiety (MM1), such that coupling of the MM1 reduces the ability of AB1 to bind its target. In some embodiments, the MM1 is coupled to AB1 via a first cleavable moiety (CM1) sequence that includes a substrate for a protease, for example, a protease that is co-localized with the target of AB1 at a treatment site or a diagnostic site in a subject. The multispecific activatable antibodies provided herein are stable in circulation, activated at intended sites of therapy and/or diagnosis but not in normal, i.e., healthy tissue, and, when activated, exhibit binding to the target of AB1 that is at least comparable to the corresponding, unmodified multispecific antibody. Suitable AB, MM, and/or CM include any of the AB, MM, and/or CM disclosed herein.

The disclosure also provides compositions and methods that include a multispecific activatable antibody that includes at least a first antibody or antibody fragment (AB1) that specifically binds a target and a second antibody or antibody fragment (AB2), where at least the first AB in the multispecific activatable antibody is coupled to a masking moiety (MM1) that decreases the ability of AB1 to bind its target. In some embodiments, each AB is coupled to a MM that decreases the ability of its corresponding AB to each target. For example, in bispecific activatable antibody embodiments, AB1 is coupled to a first masking moiety (MM1) that decreases the ability of AB1 to bind its target, and AB2 is coupled to a second masking moiety (MM2) that decreases the ability of AB2 to bind its target. In some embodiments, the multispecific activatable antibody comprises more than two AB regions; in such embodiments, AB1 is coupled to a first masking moiety (MM1) that decreases the ability of AB1 to bind its target, AB2 is coupled to a second masking moiety (MM2) that decreases the ability of AB2 to bind its target, AB3 is coupled to a third masking moiety (MM3) that decreases the ability of AB3 to bind its target, and so on for each AB in the multispecific activatable antibody. Suitable AB, MM, and/or CM include any of the AB, MM, and/or CM disclosed herein.

In some embodiments, the multispecific activatable antibody further includes at least one cleavable moiety (CM) that is a substrate for a protease, where the CM links a MM to an AB. For example, in some embodiments, the multispecific activatable antibody includes at least a first antibody or antibody fragment (AB1) that specifically binds a target and a second antibody or antibody fragment (AB2), where at least the first AB in the multispecific activatable antibody is coupled via a first cleavable moiety (CM1) to a masking moiety (MM1) that decreases the ability of AB1 to bind its target. In some bispecific activatable antibody embodiments, AB1 is coupled via CM1 to MM1, and AB2 is coupled via a second cleavable moiety (CM2) to a second masking moiety (MM2) that decreases the ability of AB2 to bind its target. In some embodiments, the multispecific activatable antibody comprises more than two AB regions; in some of these embodiments, AB1 is coupled via CM1 to MM1, AB2 is coupled via CM2 to MM2, and AB3 is coupled via a third cleavable moiety (CM3) to a third masking moiety (MM3) that decreases the ability of AB3 to bind its target, and so on for each AB in the multispecific activatable antibody. Suitable AB, MM, and/or CM include any of the AB, MM, and/or CM disclosed herein.

Activatable Antibodies Having Non-Binding Steric Moieties or Binding Partners for Non-Binding Steric Moieties

The disclosure also provides activatable antibodies that include non-binding steric moieties (NB) or binding partners (BP) for non-binding steric moieties, where the BP recruits or otherwise attracts the NB to the activatable antibody. The activatable antibodies provided herein include, for example, an activatable antibody that includes a non-binding steric moiety (NB), a cleavable linker (CL) and antibody or antibody fragment (AB) that binds a target; an activatable antibody that includes a binding partner for a non-binding steric moiety (BP), a CL and an AB; and an activatable antibody that includes a BP to which an NB has been recruited, a CL and an AB that binds the target. Activatable antibodies in which the NB is covalently linked to the CL and AB of the activatable antibody or is associated by interaction with a BP that is covalently linked to the CL and AB of the activatable antibody are referred to herein as “NB-containing activatable antibodies.” By activatable or switchable is meant that the activatable antibody exhibits a first level of binding to a target when the activatable antibody is in an inhibited, masked or uncleaved state (i.e., a first conformation), and a second level of binding to the target when the activatable antibody is in an uninhibited, unmasked and/or cleaved state (i.e., a second conformation, i.e., activated antibody), where the second level of target binding is greater than the first level of target binding. The activatable antibody compositions can exhibit increased bioavailability and more favorable biodistribution compared to conventional antibody therapeutics.

In some embodiments, activatable antibodies provide for reduced toxicity and/or adverse side effects that could otherwise result from binding of the at non-treatment sites and/or non-diagnostic sites if the AB were not masked or otherwise inhibited from binding to such a site.

CD147 activatable antibodies that include a non-binding steric moiety (NB) can be made using the methods set forth in PCT Publication No. WO 2013/192546, the contents of which are hereby incorporated by reference in their entirety.

Therapeutic Uses of Antibodies, and Activatable Antibodies

The invention provides methods of preventing, delaying the progression of, treating, alleviating a symptom of, or otherwise ameliorating a CD147-mediated disease in a subject by administering a therapeutically effective amount of a CD147 antibody, conjugated CD147 antibody, activatable CD147 antibody and/or conjugated activatable CD147 antibody described herein to a subject in need thereof.

There is provided a CD147 antibody, conjugated CD147 antibody, activatable CD147 antibody and/or conjugated activatable CD147 antibody for use in preventing, delaying the progression of, treating, alleviating a symptom of, or otherwise ameliorating a CD147-mediated disease.

The invention also provides methods of preventing, delaying the progression of, treating, alleviating a symptom of, or otherwise ameliorating cancer in a subject by administering a therapeutically effective amount of a CD147 antibody, conjugated CD147 antibody, activatable CD147 antibody and/or conjugated activatable CD147 antibody described herein to a subject in need thereof.

There is provided a CD147 antibody, conjugated CD147 antibody, activatable CD147 antibody and/or conjugated activatable CD147 antibody for use in preventing, delaying the progression of, treating, alleviating a symptom of, or otherwise ameliorating cancer.

The invention also provides methods of treating, preventing and/or delaying the onset or progression of, or alleviating a symptom associated with aberrant expression and/or activity of CD147 in a subject using antibodies/activatable antibodies that bind CD147, particularly activatable antibodies that bind and neutralize or otherwise inhibit at least one biological activity of CD147 and/or CD147-mediated signaling.

Thus, there is provided antibodies/activatable antibodies that bind CD147 for use in treating, preventing and/or delaying the onset or progression of, ameliorating, or alleviating a symptom associated with aberrant expression and/or activity of CD147.

The invention also provides methods of treating, preventing and/or delaying the onset or progression of, or alleviating a symptom associated with the presence, growth, proliferation, metastasis, and/or activity of cells which are expressing CD147 or aberrantly expressing CD147 in a subject using antibodies/activatable antibodies that bind CD147, particularly activatable antibodies that bind, target, neutralize, kill, or otherwise inhibit at least one biological activity of cells which are expressing or aberrantly expressing CD147.

The invention also provides methods of treating, preventing and/or delaying the onset or progression of, or alleviating a symptom associated with the presence, growth, proliferation, metastasis, and/or activity of cells which are expressing CD147 in a subject using antibodies/activatable antibodies that bind CD147, particularly antibodies/activatable antibodies that bind, target, neutralize, kill, or otherwise inhibit at least one biological activity of cells which are expressing CD147.

The invention also provides methods of treating, preventing and/or delaying the onset or progression of, or alleviating a symptom associated with the presence, growth, proliferation, metastasis, and/or activity of cells which are aberrantly expressing CD147 in a subject using antibodies/activatable antibodies that bind CD147, particularly antibodies/activatable antibodies that bind, target, neutralize, kill, or otherwise inhibit at least one biological activity of cells which are aberrantly expressing CD147.

CD147 is known to be expressed in a variety of cancers, such as, by way of non-limiting example, adenocarcinoma, bile duct (biliary) cancer, bladder cancer, breast cancer, e.g., triple-negative breast cancer and Her2-negative breast cancer; carcinoid cancer; cervical cancer; cholangiocarcinoma; colorectal; endometrial; esophageal cancer; glioma; head and neck cancer, e.g., head and neck squamous cell cancer; leukemia; liver cancer; lung cancer, e.g., NSCLC, SCLC; lymphoma; melanoma; osopharyngeal cancer; ovarian cancer; pancreatic cancer; prostate cancer, e.g., metastatic castration-resistant prostate carcinoma; renal cancer; skin cancer; squamous cell cancer, stomach cancer; testis cancer; thyroid cancer; and urothelial cancer.

In some embodiments, the cancer is associated with a CD147-expressing tumor. In some embodiments, the cancer is due to a CD147-expressing tumor.

A CD147 antibody, conjugated CD147 antibody, activatable CD147 antibody and/or conjugated activatable CD147 antibody used in any of the embodiments of these methods and uses can be administered at any stage of the disease. For example, such a CD147 antibody, conjugated CD147 antibody, activatable CD147 antibody and/or conjugated activatable CD147 antibody can be administered to a patient suffering cancer of any stage, from early to metastatic. The terms subject and patient are used interchangeably herein.

In some embodiments, the subject is a mammal, such as a human, non-human primate, companion animal (e.g., cat, dog, horse), farm animal, work animal, or zoo animal. In some embodiments, the subject is a human. In some embodiments, the subject is a companion animal. In some embodiments, the subject is an animal in the care of a veterinarian.

The CD147 antibody, conjugated CD147 antibody, activatable CD147 antibody and/or conjugated activatable CD147 antibody and therapeutic formulations thereof are administered to a subject suffering from or susceptible to a disease or disorder associated with aberrant CD147 expression and/or activity. A subject suffering from or susceptible to a disease or disorder associated with aberrant CD147 expression and/or activity is identified using any of a variety of methods known in the art. For example, subjects suffering from cancer or other neoplastic condition are identified using any of a variety of clinical and/or laboratory tests such as, physical examination and blood, urine and/or stool analysis to evaluate health status. For example, subjects suffering from inflammation and/or an inflammatory disorder are identified using any of a variety of clinical and/or laboratory tests such as physical examination and/or bodily fluid analysis, e.g., blood, urine and/or stool analysis, to evaluate health status.

Administration of a CD147 antibody, conjugated CD147 antibody, activatable CD147 antibody and/or conjugated activatable CD147 antibody to a patient suffering from a disease or disorder associated with aberrant CD147 expression and/or activity is considered successful if any of a variety of laboratory or clinical objectives is achieved. For example, administration of a CD147 antibody, conjugated CD147 antibody, activatable CD147 antibody and/or conjugated activatable CD147 antibody to a patient suffering from a disease or disorder associated with aberrant CD147 expression and/or activity is considered successful if one or more of the symptoms associated with the disease or disorder is alleviated, reduced, inhibited or does not progress to a further, i.e., worse, state. Administration of a CD147 antibody, conjugated CD147 antibody, activatable CD147 antibody and/or conjugated activatable CD147 antibody to a patient suffering from a disease or disorder associated with aberrant CD147 expression and/or activity is considered successful if the disease or disorder enters remission or does not progress to a further, i.e., worse, state.

In some embodiments, the CD147 antibody, conjugated CD147 antibody, activatable CD147 antibody and/or conjugated activatable CD147 antibody and therapeutic formulations thereof are administered to a subject suffering from or susceptible to a disease or disorder, such as subjects suffering from cancer or other neoplastic condition, wherein the subject's diseased cells are expressing CD147. In some embodiments, the diseased cells are associated with aberrant CD147 expression and/or activity. In some embodiments, the diseased cells are associated with normal CD147 expression and/or activity. A subject suffering from or susceptible to a disease or disorder wherein the subject's diseased cells express CD147 is identified using any of a variety of methods known in the art. For example, subjects suffering from cancer or other neoplastic condition are identified using any of a variety of clinical and/or laboratory tests such as, physical examination and blood, urine and/or stool analysis to evaluate health status. For example, subjects suffering from inflammation and/or an inflammatory disorder are identified using any of a variety of clinical and/or laboratory tests such as physical examination and/or bodily fluid analysis, e.g., blood, urine and/or stool analysis, to evaluate health status.

In some embodiments, the CD147 antibody, conjugated CD147 antibody, activatable CD147 antibody and/or conjugated activatable CD147 antibody and therapeutic formulations thereof are administered to a subject suffering from or susceptible to a disease or disorder associated with cells expressing CD147 or the presence, growth, proliferation, metastasis, and/or activity of such cells, such as subjects suffering from cancer or other neoplastic conditions. In some embodiments, the cells are associated with aberrant CD147 expression and/or activity. In some embodiments, the cells are associated with normal CD147 expression and/or activity. A subject suffering from or susceptible to a disease or disorder associated with cells that express CD147 is identified using any of a variety of methods known in the art. For example, subjects suffering from cancer or other neoplastic condition are identified using any of a variety of clinical and/or laboratory tests such as, physical examination and blood, urine and/or stool analysis to evaluate health status. For example, subjects suffering from inflammation and/or an inflammatory disorder are identified using any of a variety of clinical and/or laboratory tests such as physical examination and/or bodily fluid analysis, e.g., blood, urine and/or stool analysis, to evaluate health status.

Administration of a CD147 antibody, conjugated CD147 antibody, activatable CD147 antibody and/or conjugated activatable CD147 antibody to a patient suffering from a disease or disorder associated with cells expressing CD147 is considered successful if any of a variety of laboratory or clinical objectives is achieved. For example, administration a CD147 antibody, conjugated CD147 antibody, activatable CD147 antibody and/or conjugated activatable CD147 antibody to a patient suffering from a disease or disorder associated with cells expressing CD147 is considered successful if one or more of the symptoms associated with the disease or disorder is alleviated, reduced, inhibited or does not progress to a further, i.e., worse, state. Administration of a CD147 antibody, conjugated CD147 antibody, activatable CD147 antibody and/or conjugated activatable CD147 antibody to a patient suffering from a disease or disorder associated with cells expressing CD147 is considered successful if the disease or disorder enters remission or does not progress to a further, i.e., worse, state.

In some embodiments, the CD147 antibody, conjugated CD147 antibody, activatable CD147 antibody and/or conjugated activatable CD147 antibody is administered during and/or after treatment in combination with one or more additional agents such as, for example, a chemotherapeutic agent, an anti-inflammatory agent, and/or an immunosuppressive agent. In some embodiments, the CD147 antibody, conjugated CD147 antibody, activatable CD147 antibody and/or conjugated activatable CD147 antibody and the additional agent(s) are administered simultaneously. For example, the CD147 antibody, conjugated CD147 antibody, activatable CD147 antibody and/or conjugated activatable CD147 antibody and the additional agent(s) can be formulated in a single composition or administered as two or more separate compositions. In some embodiments, the CD147 antibody, conjugated CD147 antibody, activatable CD147 antibody and/or conjugated activatable CD147 antibody and the additional agent(s) are administered sequentially.

The disclosure also provides methods of treating, alleviating a symptom of, or delaying the progression of a disorder or disease in which diseased cells express CD147 comprising administering a therapeutically effective amount of an antibody/conjugated antibody/activatable antibody/conjugated activatable antibody of the disclosure or a pharmaceutical composition comprising an antibody/conjugated antibody/activatable antibody/conjugated activatable antibody of the disclosure to a subject in need thereof. In some embodiments, the disorder or disease is cancer.

The disclosure also provides methods of treating, alleviating a symptom of, or delaying the progression of a disorder or disease associated with cells expressing CD147 comprising administering a therapeutically effective amount of an antibody/conjugated antibody/activatable antibody/conjugated activatable antibody of the disclosure or a pharmaceutical composition comprising an antibody/conjugated antibody/activatable antibody/conjugated activatable antibody of the disclosure to a subject in need thereof. In some embodiments, the disorder or disease associated with cells expressing CD147 is cancer. In some embodiments, the cancer is an adenocarcinoma, a bile duct (biliary) cancer, a bladder cancer, a bone cancer, a breast cancer, a triple-negative breast cancer, a Her2-negative breast cancer, a carcinoid cancer, a cervical cancer, a cholangiocarcinoma, a colorectal cancer, a colon cancer, an endometrial cancer, an esophageal cancer, a glioma, a head and neck cancer, a head and neck squamous cell cancer, a leukemia, a liver cancer, a lung cancer, a non-small cell lung cancer, a small cell lung cancer, a lymphoma, a melanoma, an oropharyngeal cancer, an ovarian cancer, a pancreatic cancer, a prostate cancer, a metastatic castration-resistant prostate carcinoma, a renal cancer, a sarcoma, a skin cancer, a squamous cell cancer, a stomach cancer, a testis cancer, a thyroid cancer, a urogenital cancer, or a urothelial cancer. In some embodiments, the expression and/or activity of the mammalian CD147 is aberrant. In some embodiments, the method comprises administering an additional agent. In some embodiments, the additional agent is a therapeutic agent.

The disclosure also provides methods of inhibiting or reducing the growth, proliferation, or metastasis of cells expressing mammalian CD147 comprising administering a therapeutically effective amount of an antibody/conjugated antibody/activatable antibody/conjugated activatable antibody of the disclosure or a pharmaceutical composition comprising an antibody/conjugated antibody/activatable antibody/conjugated activatable antibody of the disclosure to a subject in need thereof. In some embodiments, the expression and/or activity of the mammalian CD147 is aberrant. In some embodiments, the method comprises administering an additional agent. In some embodiments, the additional agent is a therapeutic agent.

The disclosure also provides methods of inhibiting, blocking, or preventing the binding of a natural ligand to mammalian CD147, comprising administering a therapeutically effective amount of an antibody/conjugated antibody/activatable antibody/conjugated activatable antibody of the disclosure or a pharmaceutical composition comprising an antibody/conjugated antibody/activatable antibody/conjugated activatable antibody of the disclosure to a subject in need thereof. In some embodiments, the expression and/or activity of the mammalian CD147 is aberrant. In some embodiments, the method comprises administering an additional agent. In some embodiments, the additional agent is a therapeutic agent.

The disclosure also provides methods of treating, alleviating a symptom of, or delaying the progression of a disorder or disease in which diseased cells express CD147 comprising administering a therapeutically effective amount of an antibody/conjugated antibody/activatable antibody/conjugated activatable antibody of the disclosure or a pharmaceutical composition comprising an antibody/conjugated antibody/activatable antibody/conjugated activatable antibody of the disclosure to a subject in need thereof. In some embodiments, the disorder or disease is cancer.

The disclosure also provides methods of treating, alleviating a symptom of, or delaying the progression of a disorder or disease associated with cells expressing CD147 comprising administering a therapeutically effective amount of an antibody/conjugated antibody/activatable antibody/conjugated activatable antibody of the disclosure or a pharmaceutical composition comprising an antibody/conjugated antibody/activatable antibody/conjugated activatable antibody of the disclosure to a subject in need thereof. In some embodiments, the disorder or disease associated with cells expressing CD147 is cancer. In some embodiments, the cancer is an adenocarcinoma, a bile duct (biliary) cancer, a bladder cancer, a bone cancer, a breast cancer, a triple-negative breast cancer, a Her2-negative breast cancer, a carcinoid cancer, a cervical cancer, a cholangiocarcinoma, a colorectal cancer, a colon cancer, an endometrial cancer, an esophageal cancer, a glioma, a head and neck cancer, a head and neck squamous cell cancer, a leukemia, a liver cancer, a lung cancer, a non-small cell lung cancer, a small cell lung cancer, a lymphoma, a melanoma, an oropharyngeal cancer, an ovarian cancer, a pancreatic cancer, a prostate cancer, a metastatic castration-resistant prostate carcinoma, a renal cancer, a sarcoma, a skin cancer, a squamous cell cancer, a stomach cancer, a testis cancer, a thyroid cancer, a urogenital cancer, or a urothelial cancer. In some embodiments, the expression and/or activity of the mammalian CD147 is aberrant. In some embodiments, the method comprises administering an additional agent. In some embodiments, the additional agent is a therapeutic agent.

The disclosure also provides methods of inhibiting or reducing the growth, proliferation, or metastasis of cells expressing mammalian CD147 comprising administering a therapeutically effective amount of an antibody/conjugated antibody/activatable antibody/conjugated activatable antibody of the disclosure or a pharmaceutical composition comprising an antibody/conjugated antibody/activatable antibody/conjugated activatable antibody of the disclosure to a subject in need thereof. In some embodiments, the expression and/or activity of the mammalian CD147 is aberrant. In some embodiments, the method comprises administering an additional agent. In some embodiments, the additional agent is a therapeutic agent.

The disclosure also provides methods of inhibiting, blocking, or preventing the binding of a natural ligand to mammalian CD147, comprising administering a therapeutically effective amount of an antibody/conjugated antibody/activatable antibody/conjugated activatable antibody of the disclosure or a pharmaceutical composition comprising an antibody/conjugated antibody/activatable antibody/conjugated activatable antibody of the disclosure to a subject in need thereof. In some embodiments, the expression and/or activity of the mammalian CD147 is aberrant. In some embodiments, the method comprises administering an additional agent. In some embodiments, the additional agent is a therapeutic agent.

Formulations

It will be appreciated that administration of therapeutic entities in accordance with the disclosure will be administered with suitable carriers, excipients, and other agents that are incorporated into formulations to provide improved transfer, delivery, tolerance, and the like. A multitude of appropriate formulations can be found in the formulary known to all pharmaceutical chemists: Remington's Pharmaceutical Sciences (15th ed, Mack Publishing Company, Easton, Pa. (1975)), particularly Chapter 87 by Blaug, Seymour, therein. These formulations include, for example, powders, pastes, ointments, jellies, waxes, oils, lipids, lipid (cationic or anionic) containing vesicles (such as Lipofectin™), DNA conjugates, anhydrous absorption pastes, oil-in-water and water-in-oil emulsions, emulsions carbowax (polyethylene glycols of various molecular weights), semi-solid gels, and semi-solid mixtures containing carbowax. Any of the foregoing mixtures may be appropriate in treatments and therapies in accordance with the present disclosure, provided that the active ingredient in the formulation is not inactivated by the formulation and the formulation is physiologically compatible and tolerable with the route of administration. See also Baldrick P. “Pharmaceutical excipient development: the need for preclinical guidance.” Regul. Toxicol Pharmacol. 32(2):210-8 (2000), Wang W. “Lyophilization and development of solid protein pharmaceuticals.” Int. J. Pharm. 203(1-2):1-60 (2000), Charman W N “Lipids, lipophilic drugs, and oral drug delivery-some emerging concepts.” J Pharm Sci. 89(8):967-78 (2000), Powell et al. “Compendium of excipients for parenteral formulations” PDA J Pharm Sci Technol. 52:238-311 (1998) and the citations therein for additional information related to formulations, excipients and carriers well known to pharmaceutical chemists.

Therapeutic formulations of the disclosure, which include a CD147 antibody and/or activatable CD147 antibody, such as by way of non-limiting example, an antibody, a conjugated antibody, an activatable antibody and/or a conjugated activatable antibody, are used to prevent, treat or otherwise ameliorate a disease or disorder associated with aberrant target expression and/or activity. For example, therapeutic formulations of the disclosure, which include an antibody, a conjugated antibody, an activatable antibody and/or a conjugated activatable antibody, are used to treat or otherwise ameliorate a cancer or other neoplastic condition, inflammation, an inflammatory disorder, and/or an autoimmune disease. In some embodiments, the cancer is a solid tumor or a hematologic malignancy where the target is expressed. In some embodiments, the cancer is a solid tumor where the target is expressed. In some embodiments, the cancer is a hematologic malignancy where the target is expressed. In some embodiments, the target is expressed on parenchyma (e.g., in cancer, the portion of an organ or tissue that often carries out function(s) of the organ or tissue). In some embodiments, the target is expressed on a cell, tissue, or organ. In some embodiments, the target is expressed on stroma (i.e., the connective supportive framework of a cell, tissue, or organ). In some embodiments, the target is expressed on an osteoblast. In some embodiments, the target is expressed on the endothelium (vasculature). In some embodiments, the target is expressed on a cancer stem cell. In some embodiments, the agent to which the antibody and/or the activatable antibody is conjugated is a microtubule inhibitor. In some embodiments, the agent to which the antibody and/or the activatable antibody is conjugated is a nucleic acid damaging agent.

Efficaciousness of prevention, amelioration or treatment is determined in association with any known method for diagnosing or treating the disease or disorder associated with target expression and/or activity, such as, for example, aberrant target expression and/or activity. Prolonging the survival of a subject or otherwise delaying the progression of the disease or disorder associated with target expression and/or activity, e.g., aberrant target expression and/or activity, in a subject indicates that the antibody, conjugated antibody, activatable antibody and/or conjugated activatable antibody confers a clinical benefit.

An antibody, a conjugated antibody, an activatable antibody and/or a conjugated activatable antibody can be administered in the form of pharmaceutical compositions. Principles and considerations involved in preparing such compositions, as well as guidance in the choice of components are provided, for example, in Remington: The Science And Practice Of Pharmacy 19th ed. (Alfonso R. Gennaro, et al., editors) Mack Pub. Co., Easton, Pa.: 1995; Drug Absorption Enhancement: Concepts, Possibilities, Limitations, And Trends, Harwood Academic Publishers, Langhorne, Pa., 1994; and Peptide And Protein Drug Delivery (Advances In Parenteral Sciences, Vol. 4), 1991, M. Dekker, New York.

In some embodiments where antibody fragments are used, the smallest fragment that specifically binds to the binding domain of the target protein is selected. For example, based upon the variable-region sequences of an antibody, peptide molecules can be designed that retain the ability to bind the target protein sequence. Such peptides can be synthesized chemically and/or produced by recombinant DNA technology. (See, e.g., Marasco et al., Proc. Natl. Acad. Sci. USA, 90: 7889-7893 (1993)). The formulation can also contain more than one active compounds as necessary for the particular indication being treated, for example, in some embodiments, those with complementary activities that do not adversely affect each other. In some embodiments, or in addition, the composition can comprise an agent that enhances its function, such as, for example, a cytotoxic agent, cytokine, chemotherapeutic agent, or growth-inhibitory agent. Such molecules are suitably present in combination in amounts that are effective for the purpose intended.

The active ingredients can also be entrapped in microcapsules prepared, for example, by coacervation techniques or by interfacial polymerization, for example, hydroxymethylcellulose or gelatin-microcapsules and poly-(methylmethacrylate) microcapsules, respectively, in colloidal drug delivery systems (for example, liposomes, albumin microspheres, microemulsions, nano-particles, and nanocapsules) or in macroemulsions.

The formulations to be used for in vivo administration must be sterile. This is readily accomplished by filtration through sterile filtration membranes.

Sustained-release preparations can be prepared. Suitable examples of sustained-release preparations include semipermeable matrices of solid hydrophobic polymers containing the antibody, which matrices are in the form of shaped articles, e.g., films, or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and γ ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(−)-3-hydroxybutyric acid. While polymers such as ethylene-vinyl acetate and lactic acid-glycolic acid enable release of molecules for over 100 days, certain hydrogels release proteins for shorter time periods.

Combination Treatments

In some embodiments, the CD147 antibodies, conjugated CD147 antibodies, activatable CD147 antibodies and/or conjugated activatable CD147 antibodies described herein are used in conjunction with one or more additional agents or a combination of additional agents. Suitable additional agents include current pharmaceutical and/or surgical therapies for an intended application, such as, for example, cancer. For example, the CD147 antibodies, conjugated CD147 antibodies, activatable CD147 antibodies and/or conjugated activatable CD147 antibodies can be used in conjunction with an additional chemotherapeutic agent, anti-neoplastic agent, anti-inflammatory agent, an immunosuppressive agent, an alkylating agent, an anti-metabolite, an anti-microtubule agent, a topoisomerase inhibitor, a cytotoxic antibiotic, and/or any other nucleic acid damaging agent.

In some embodiments, the additional agent(s) is a chemotherapeutic agent, such as a chemotherapeutic agent selected from the group consisting of docetaxel, paclitaxel, abraxane (i.e., albumin-conjugated paclitaxel), doxorubicin, oxaliplatin, carboplatin, cisplatin, irinotecan, and gemcitabine.

In some embodiments, the additional agent(s) is a checkpoint inhibitor, a kinase inhibitor, an agent targeting inhibitors in the tumor microenvironment, and/or a T cell or NK agonist. In some embodiments, the additional agent(s) is radiation therapy, alone or in combination with another additional agent(s) such as a chemotherapeutic or anti-neoplastic agent. In some embodiments, the additional agent(s) is a vaccine, an oncovirus, and/or a DC-activating agent such as, by way of non-limiting example, a toll-like receptor (TLR) agonist and/or α-CD40. In some embodiments, the additional agent(s) is a tumor-targeted antibody designed to kill the tumor via ADCC or via direct conjugation to a toxin (e.g., an antibody drug conjugate (ADC).

In some embodiments, the checkpoint inhibitor is an inhibitor of a target selected from the group consisting of CTLA-4, LAG-3, PD-1, CD147, TIGIT, TIM-3, B7H4, and Vista. In some embodiments, the kinase inhibitor is selected from the group consisting of B-RAFi, MEKi, and Btk inhibitors, such as ibrutinib. In some embodiments, the kinase inhibitor is crizotinib. In some embodiments, the tumor microenvironment inhibitor is selected from the group consisting of an IDO inhibitor, an α-CSF1R inhibitor, an α-CCR4 inhibitor, a TGF-beta, a myeloid-derived suppressor cell, or a T-regulatory cell. In some embodiments, the agonist is selected from the group consisting of Ox40, GITR, CD137, ICOS, CD27, and HVEM.

In some embodiments, the inhibitor is a CTLA-4 inhibitor. In some embodiments, the inhibitor is a LAG-3 inhibitor. In some embodiments, the inhibitor is a PD-1 inhibitor. In some embodiments, the inhibitor is a CD147 inhibitor. In some embodiments, the inhibitor is a TIGIT inhibitor. In some embodiments, the inhibitor is a TIM-3 inhibitor. In some embodiments, the inhibitor is a B7H4 inhibitor. In some embodiments, the inhibitor is a Vista inhibitor. In some embodiments, the inhibitor is a B-RAFi inhibitor. In some embodiments, the inhibitor is a MEKi inhibitor. In some embodiments, the inhibitor is a Btk inhibitor. In some embodiments, the inhibitor is ibrutinib. In some embodiments, the inhibitor is crizotinib. In some embodiments, the inhibitor is an IDO inhibitor. In some embodiments, the inhibitor is an α-CSFIR inhibitor. In some embodiments, the inhibitor is an α-CCR4 inhibitor. In some embodiments, the inhibitor is a TGF-beta. In some embodiments, the inhibitor is a myeloid-derived suppressor cell. In some embodiments, the inhibitor is a T-regulatory cell.

In some embodiments, the agonist is Ox40. In some embodiments, the agonist is GITR. In some embodiments, the agonist is CD137. In some embodiments, the agonist is ICOS. In some embodiments, the agonist is CD27. In some embodiments, the agonist is HVEM.

In some embodiments, the CD147 antibody, conjugated antibody, activatable antibody and/or conjugated activatable antibody is administered during and/or after treatment in combination with one or more additional agents such as, for example, a chemotherapeutic agent, an anti-inflammatory agent, and/or a an immunosuppressive agent. In some embodiments, the CD147 antibody, conjugated CD147 antibody, activatable CD147 antibody and/or conjugated activatable CD147 antibody and the additional agent are formulated into a single therapeutic composition, and the CD147 antibody, conjugated CD147 antibody, activatable CD147 antibody and/or conjugated activatable CD147 antibody and additional agent are administered simultaneously. Alternatively, the CD147 antibody, conjugated CD147 antibody, activatable CD147 antibody and/or conjugated activatable CD147 antibody and additional agent are separate from each other, e.g., each is formulated into a separate therapeutic composition, and the CD147 antibody, conjugated CD147 antibody, activatable CD147 antibody and/or conjugated activatable CD147 antibody and the additional agent are administered simultaneously, or the CD147 antibody, conjugated CD147 antibody, activatable CD147 antibody and/or conjugated activatable CD147 antibody and the additional agent are administered at different times during a treatment regimen. For example, the CD147 antibody, conjugated CD147 antibody, activatable CD147 antibody and/or conjugated activatable CD147 antibody is administered prior to the administration of the additional agent, the CD147 antibody, conjugated CD147 antibody, activatable CD147 antibody and/or conjugated activatable CD147 antibody is administered subsequent to the administration of the additional agent, or the CD147 antibody, conjugated CD147 antibody, activatable CD147 antibody and/or conjugated activatable CD147 antibody and the additional agent are administered in an alternating fashion. As described herein, the CD147 antibody, conjugated CD147 antibody, activatable CD147 antibody and/or conjugated activatable CD147 antibody and additional agent are administered in single doses or in multiple doses.

In some embodiments, the CD147 antibody, conjugated CD147 antibody, activatable CD147 antibody and/or conjugated activatable CD147 antibody and the additional agent(s) are administered simultaneously. For example, the CD147 antibody, conjugated CD147 antibody, activatable CD147 antibody and/or conjugated activatable CD147 antibody and the additional agent(s) can be formulated in a single composition or administered as two or more separate compositions. In some embodiments, the CD147 antibody, conjugated CD147 antibody, activatable CD147 antibody and/or conjugated activatable CD147 antibody and the additional agent(s) are administered sequentially, or the CD147 antibody, conjugated CD147 antibody, activatable CD147 antibody and/or conjugated activatable CD147 antibody and the additional agent are administered at different times during a treatment regimen.

In some embodiments, the CD147 antibody, conjugated CD147 antibody, activatable CD147 antibody and/or conjugated activatable CD147 antibody is administered during and/or after treatment in combination with one or more additional agents such as, by way of non-limiting example, a chemotherapeutic agent, an anti-inflammatory agent, and/or an immunosuppressive agent, such as an alkylating agent, an anti-metabolite, an anti-microtubule agent, a topoisomerase inhibitor, a cytotoxic antibiotic, and/or any other nucleic acid damaging agent. In some embodiments, the additional agent is a taxane, such as paclitaxel (e.g., Abraxane®). In some embodiments, the additional agent is an anti-metabolite, such as gemcitabine. In some embodiments, the additional agent is an alkylating agent, such as platinum-based chemotherapy, such as carboplatin or cisplatin. In some embodiments, the additional agent is a targeted agent, such as a kinase inhibitor, e.g., sorafenib or erlotinib. In some embodiments, the additional agent is a targeted agent, such as another antibody, e.g., a monoclonal antibody (e.g., bevacizumab), a bispecific antibody, or a multispecific antibody. In some embodiments, the additional agent is a proteosome inhibitor, such as bortezomib or carfilzomib. In some embodiments, the additional agent is an immune modulating agent, such as lenolidominde or IL-2. In some embodiments, the additional agent is radiation. In some embodiments, the additional agent is an agent considered standard of care by those skilled in the art. In some embodiments, the additional agent is a chemotherapeutic agent well known to those skilled in the art.

In some embodiments, the additional agent is another antibody or antigen-binding fragment thereof, another conjugated antibody or antigen-binding fragment thereof, another activatable antibody or antigen-binding fragment thereof and/or another conjugated activatable antibody or antigen-binding fragment thereof. In some embodiments the additional agent is another antibody or antigen-binding fragment thereof, another conjugated antibody or antigen-binding fragment thereof, another activatable antibody or antigen-binding fragment thereof and/or another conjugated activatable antibody or antigen-binding fragment thereof against the same target as the first antibody or antigen-binding fragment thereof, the first conjugated antibody or antigen-binding fragment thereof, activatable antibody or antigen-binding fragment thereof and/or a conjugated activatable antibody or antigen-binding fragment thereof, e.g., against CD147. In some embodiments the additional agent is another antibody or antigen-binding fragment thereof, another conjugated antibody or antigen-binding fragment thereof, another activatable antibody or antigen-binding fragment thereof and/or another conjugated activatable antibody or antigen-binding fragment thereof against a target different than the target of the first antibody or antigen-binding fragment thereof, the first conjugated antibody or antigen-binding fragment thereof, activatable antibody or antigen-binding fragment thereof and/or a conjugated activatable antibody or antigen-binding fragment thereof.

As a non-limiting example, the antibody, antigen-binding fragment and/or the AB of an activatable antibody (of the additional agent) is a binding partner for any target listed in Table 10.

TABLE 10
Exemplary Targets

| | | | | | |
|---|---|---|---|---|---|
| 1-92-LFA-3 | CD52 | DL44 | HVEM | LIF-R | STEAP1 |
| Alpha-4 | CD56 | DLK1 | Hyaluronidase | Lewis X | STEAP2 |
| integrin | | | | | |
| Alpha-V | CD64 | DLL4 | ICOS | LIGHT | TAG-72 |
| integrin | | | | | |
| alpha4beta1 | CD70 | DPP-4 | IFNalpha | LRP4 | TAPA1 |
| integrin | | | | | |
| alpha4beta7 | CD147 | DSG1 | IFNbeta | LRRC26 | TGFbeta |
| integrin | | | | | |
| AGR2 | CD74 | EGFR | IFNgamma | MCSP | TIGIT |
| Anti-Lewis-Y | | EGFRviii | IgE | Mesothelin | TIM-3 |
| Apelin J | CD80 | Endothelin B | IgE Receptor | MRP4 | TLR2 |
| receptor | | receptor | (FceRI) | | |
| | | (ETBR) | | | |
| APRIL | CD81 | ENPP3 | IGF | MUC1 | TLR4 |
| B7-H4 | CD86 | EpCAM | IGF1R | Mucin-16 | TLR6 |
| | | | | (MUC16, | |
| | | | | CA-125) | |
| BAFF | CD95 | EPHA2 | IL1B | Na/K ATPase | TLR7 |
| BTLA | CD117 | EPHB2 | IL1R | Neutrophil | TLR8 |
| | | | | elastase | |
| C5 | CD125 | ERBB3 | IL2 | NGF | TLR9 |
| complement | | | | | |
| C-242 | CD132 | F protein of | IL11 | Nicastrin | TMEM31 |
| | (IL-2RG) | RSV | | | |
| CA9 | CD133 | FAP | IL12 | Notch | TNFalpha |
| | | | | Receptors | |
| CA19-9 | CD137 | FGF-2 | IL12p40 | Notch1 | TNFR |
| (Lewis a) | | | | | |
| Carbonic | CD138 | FGF8 | IL-12R, | Notch 2 | TNFRS12 |
| anhydrase 9 | | | IL-12Rbeta1 | | A |
| CD2 | CD166 | FGFR1 | IL13 | Notch 3 | TRAIL-R1 |
| CD3 | CD172A | FGFR2 | IL13R | Notch 4 | TRAIL-R2 |
| CD6 | CD248 | FGFR3 | IL15 | NOV | Transferrin |
| CD9 | CDH6 | FGFR4 | IL17 | OSM-R | Transferrin |
| | | | | | receptor |
| CD11a | CEACAM5 | Folate | IL18 | OX-40 | TRK-A |
| | (CEA) | receptor | | | |
| CD19 | CEACAM6 | GAL3ST1 | IL21 | PAR2 | TRK-B |
| | (NCA-90) | | | | |
| CD20 | CLAUDIN-3 | G-CSF | IL23 | PDGF-AA | uPAR |
| CD22 | CLAUDIN-4 | G-CSFR | IL23R | PDGF-BB | VAP1 |
| CD24 | cMet | GD2 | IL27/IL27R | PDGFRalpha | VCAM-1 |
| | | | (wsx1) | | |
| CD25 | Collagen | GITR | IL29 | PDGFRbeta | VEGF |
| CD27 | Cripto | GLUT1 | IL-31R | PD-1 | VEGF-A |
| CD28 | CSFR | GLUT4 | IL31/IL31R | PD-L1 | VEGF-B |
| CD30 | CSFR-1 | GM-CSF | IL2R | PD-L2 | VEGF-C |
| CD33 | CTLA-4 | GM-CSFR | IL4 | Phosphatidyl- | VEGF-D |
| | | | | serine | |
| CD38 | CTGF | GP IIb/IIIa | IL4R | P1GF | VEGFR1 |
| | | receptors | | | |
| CD40 | CXCL10 | Gp130 | IL6, IL6R | PSCA | VEGFR2 |
| CD40L | CXCL13 | GPIIB/IIIA | Insulin | PSMA | VEGFR3 |
| | | | Receptor | | |
| CD41 | CXCR1 | GPNMB | Jagged | RAAG12 | VISTA |
| | | | Ligands | | |
| CD44 | CXCR2 | GRP78 | Jagged 1 | RAGE | WISP-1 |
| CD44v6 | | HER2/neu | Jagged 2 | SLC44A4 | WISP-2 |
| CD47 | CXCR4 | HGF | LAG-3 | Sphingosine 1 | WISP-3 |
| | | | | Phosphate | |
| CD51 | CYR61 | hGH | | | |

As a non-limiting example, the antibody, antigen-binding fragment and/or the AB of an activatable antibody (of the additional agent) is or is derived from an antibody listed in Table 11.

TABLE 11
Exemplary sources for Abs

| Antibody Trade Name (antibody name) | Target |
|---|---|
| Avastin ™ (bevacizumab) | VEGF |
| Lucentis ™ (ranibizumab) | VEGF |
| Erbitux ™ (cetuximab) | EGFR |
| Vectibix ™ (panitumumab) | EGFR |
| Remicade ™ (infliximab) | TNFα |
| Humira ™ (adalimumab) | TNFα |
| Tysabri ™ (natalizumab) | Integrinα4 |
| Simulect ™ (basiliximab) | IL2R |
| Soliris ™ (eculizumab) | Complement C5 |
| Raptiva ™ (efalizumab) | CD11a |
| Bexxar ™ (tositumomab) | CD20 |
| Zevalin ™ (ibritumomab tiuxetan) | CD20 |
| Rituxan ™ (rituximab) | CD20 |
| Ocrelizumab | CD20 |
| Arzerra ™ (ofatumumab) | CD20 |
| Gazyva ™ (obinutuzumab) | CD20 |
| Zenapax ™ (daclizumab) | CD25 |
| Adcetris ™ (brentuximab vedotin) | CD30 |
| Myelotarg ™ (gemtuzumab) | CD33 |
| Mylotarg ™ (gemtuzumab ozogamicin) | CD33 |
| Campath ™ (alemtuzumab) | CD52 |
| ReoPro ™ (abiciximab) | Glycoprotein receptor IIb/IIIa |
| Xolair ™ (omalizumab) | IgE |
| Herceptin ™ (trastuzumab) | Her2 |
| Kadcyla ™ (trastuzumab emtansine) | Her2 |
| Synagis ™ (palivizumab) | F protein of RSV |
| (ipilimumab) | CTLA-4 |
| (tremelimumab) | CTLA-4 |
| Hu5c8 | CD40L |
| (pertuzumab) | Her2-neu |
| (ertumaxomab) | CD3/Her2-neu |
| Orencia ™ (abatacept) | CTLA-4 |
| (tanezumab) | NGF |
| (bavituximab) | Phosphatidylserine |
| (zalutumumab) | EGFR |
| (mapatumumab) | EGFR |
| (matuzumab) | EGFR |
| (nimotuzumab) | EGFR |
| ICR62 | EGFR |
| mAb 528 | EGFR |
| CH806 | EGFR |
| MDX-447 | EGFR/CD64 |
| (edrecolomab) | EpCAM |
| RAV12 | RAAG12 |
| huJ591 | PSMA |
| Enbrel ™ (etanercept) | TNF-R |
| Amevive ™ (alefacept) | 1-92-LFA-3 |
| Antril ™ , Kineret ™ (ankinra) | IL-1Ra |
| GC1008 | TGFbeta |
| | Notch, e.g., Notch 1 |
| | Jagged 1 or Jagged 2 |
| (adecatumumab) | EpCAM |
| (figitumumab) | IGF1R |
| (tocilizumab) | IL-6 receptor |
| Stelara ™ (ustekinumab) | IL-12/IL-23 |
| Prolia ™ (denosumab) | RANKL |

In some embodiments, the additional antibody or antigen binding fragment thereof, conjugated antibody or antigen binding fragment thereof, activatable antibody or antigen binding fragment thereof, and/or conjugated activatable antibody or antigen binding fragment thereof is a monoclonal antibody, domain antibody, single chain, Fab fragment, a F(ab′)₂ fragment, a scFv, a scAb, a dAb, a single domain heavy chain antibody, or a single domain light chain antibody. In some embodiments, the additional antibody or antigen binding fragment thereof, conjugated antibody or antigen binding fragment thereof, activatable antibody or antigen binding fragment thereof, and/or conjugated activatable antibody or antigen binding fragment thereof is a mouse, other rodent, chimeric, humanized or fully human monoclonal antibody.

Detection, Diagnostics, Imaging, Patient Selection

The invention also provides methods and kits for using the antibodies/conjugated antibodies/activatable antibodies/conjugated activatable antibodies provided herein in a variety of diagnostic and/or prophylactic indications. For example, the invention provides methods and kits for detecting the presence or absence of a cleaving agent and a target of interest in a subject or a sample by (i) contacting a subject or sample with a CD147 activatable antibody, wherein the CD147 activatable antibody comprises a masking moiety (MM), a cleavable moiety (CM) that is cleaved by the cleaving agent, and an antigen binding domain or fragment thereof (AB) that specifically binds the target of interest, wherein the CD147 activatable antibody in an uncleaved, non-activated state comprises a structural arrangement from N-terminus to C-terminus as follows: MM-CM-AB or AB-CM-MM; (a) wherein the MM is a peptide that inhibits binding of the AB to CD147, and wherein the MM does not have an amino acid sequence of a naturally occurring binding partner of the AB and is not a modified form of a natural binding partner of the AB, and (b) wherein, when the AB is in an uncleaved, non-activated state, the MM interferes with specific binding of the AB to CD147, and when the AB is in a cleaved, activated state the MM does not interfere or compete with specific binding of the AB to CD147; and (ii) measuring a level of activated CD147 activatable antibody in the subject or sample, wherein a detectable level of activated CD147 activatable antibody in the subject or sample indicates that the cleaving agent and CD147 are present in the subject or sample and wherein no detectable level of activated CD147 activatable antibody in the subject or sample indicates that the cleaving agent, CD147 or both the cleaving agent and CD147 are absent in the subject or sample. As provided herein, the CD147 activatable antibody can bind both human and cynomolgus CD147.

In some embodiments, the activatable CD147 antibody is an activatable CD147 antibody to which a therapeutic agent is conjugated. In some embodiments, the activatable antibody is not conjugated to an agent. In some embodiments, the activatable CD147 antibody comprises a detectable label. In some embodiments, the detectable label is positioned on the AB. In some embodiments, measuring the level of activatable antibody in the subject or sample is accomplished using a secondary reagent that specifically binds to the activated antibody, wherein the reagent comprises a detectable label. In some embodiments, the secondary reagent is an antibody comprising a detectable label.

In some embodiments of these methods and kits, the activatable CD147 antibody includes a detectable label. In some embodiments of these methods and kits, the detectable label includes an imaging agent, a contrasting agent, an enzyme, a fluorescent label, a chromophore, a dye, one or more metal ions, or a ligand-based label. In some embodiments of these methods and kits, the imaging agent comprises a radioisotope. In some embodiments of these methods and kits, the radioisotope is indium or technetium. In some embodiments of these methods and kits, the contrasting agent comprises iodine, gadolinium or iron oxide. In some embodiments of these methods and kits, the enzyme comprises horseradish peroxidase, alkaline phosphatase, or β-galactosidase. In some embodiments of these methods and kits, the fluorescent label comprises yellow fluorescent protein (YFP), cyan fluorescent protein (CFP), green fluorescent protein (GFP), modified red fluorescent protein (mRFP), red fluorescent protein tdimer2 (RFP tdimer2), HCRED, or a europium derivative. In some embodiments of these methods and kits, the luminescent label comprises an N-methylacrydium derivative. In some embodiments of these methods, the label comprises an Alexa Fluor® label, such as Alex Fluor® 680 or Alexa Fluor® 750. In some embodiments of these methods and kits, the ligand-based label comprises biotin, avidin, streptavidin or one or more haptens.

In some embodiments of these methods and kits, the subject is a mammal. In some embodiments of these methods, the subject is a human. In some embodiments, the subject is a non-human mammal, such as a non-human primate, companion animal (e.g., cat, dog, horse), farm animal, work animal, or zoo animal. In some embodiments, the subject is a rodent.

In some embodiments of these methods and kits, the method is an in vivo method. In some embodiments of these methods, the method is an int situ method. In some embodiments of these methods, the method is an ex vivo method. In some embodiments of these methods, the method is an in vitro method.

In some embodiments of the methods and kits, the method is used to identify or otherwise refine a patient population suitable for treatment with a CD147 activatable antibody of the disclosure, followed by treatment by administering that activatable CD147 antibody and/or conjugated activatable CD147 antibody to a subject in need thereof. For example, patients that test positive for both the target (e.g., CD147) and a protease that cleaves the substrate in the cleavable moiety (CM) of CD147 activatable antibody being tested in these methods are identified as suitable candidates for treatment with such a CD147 activatable antibody comprising such a CM, and the patient is then administered a therapeutically effective amount of the activatable CD147 antibody and/or conjugated activatable CD147 antibody that was tested. Likewise, patients that test negative for either or both of the target (e.g., CD147) and the protease that cleaves the substrate in the CM in the activatable antibody being tested using these methods might be identified as suitable candidates for another form of therapy. In some embodiments, such patients can be tested with other CD147 activatable antibodies until a suitable CD147 activatable antibody for treatment is identified (e.g., a CD147 activatable antibody comprising a CM that is cleaved by the patient at the site of disease). In some embodiments, the patient is then administered a therapeutically effective amount of the activatable CD147 antibody and/or conjugated for which the patient tested positive. Suitable AB, MM, and/or CM include any of the AB, MM, and/or CM disclosed herein.

In some embodiments, the antibody, the conjugated antibody, activatable antibody and/or conjugated activatable antibody contains a detectable label. An intact antibody, or a fragment thereof (e.g., Fab, scFv, or F(ab)₂) is used. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently-labeled secondary antibody and end-labeling of a DNA probe with biotin such that it can be detected with fluorescently-labeled streptavidin. The term “biological sample” is intended to include tissues, cells and biological fluids isolated from a subject, as well as tissues, cells and fluids present within a subject. Included within the usage of the term “biological sample”, therefore, is blood and a fraction or component of blood including blood serum, blood plasma, or lymph. That is, the detection method of the disclosure can be used to detect an analyte mRNA, protein, or genomic DNA in a biological sample in vitro as well as in vivo. For example, in vitro techniques for detection of an analyte mRNA include Northern hybridizations and in situ hybridizations. In vitro techniques for detection of an analyte protein include enzyme linked immunosorbent assays (ELISAs), Western blots, immunoprecipitations, immunochemical staining, and immunofluorescence. In vitro techniques for detection of an analyte genomic DNA include Southern hybridizations. Procedures for conducting immunoassays are described, for example in “ELISA: Theory and Practice: Methods in Molecular Biology”, Vol. 42, J. R. Crowther (Ed.) Human Press, Totowa, N.J., 1995; “Immunoassay”, E. Diamandis and T. Christopoulus, Academic Press, Inc., San Diego, Calif., 1996; and “Practice and Theory of Enzyme Immunoassays”, P. Tijssen, Elsevier Science Publishers, Amsterdam, 1985. Furthermore, in vivo techniques for detection of an analyte protein include introducing into a subject a labeled anti-analyte protein antibody. For example, the antibody can be labeled with a radioactive marker whose presence and location in a subject can be detected by standard imaging techniques.

The antibodies, conjugated antibodies, activatable antibodies and/or conjugated activatable antibodies of the disclosure are also useful in a variety of diagnostic and prophylactic formulations. In one embodiment, an antibody, a conjugated antibody, an activatable antibody and/or a conjugated activatable antibody is administered to patients that are at risk of developing one or more of the aforementioned disorders. A patient's or organ's predisposition to one or more of the aforementioned disorders can be determined using genotypic, serological or biochemical markers.

In some embodiments of the disclosure, an antibody, a conjugated antibody, an activatable antibody and/or a conjugated activatable antibody is administered to human individuals diagnosed with a clinical indication associated with one or more of the aforementioned disorders. Upon diagnosis, an antibody, a conjugated antibody, an activatable antibody and/or a conjugated activatable antibody is administered to mitigate or reverse the effects of the clinical indication.

An antibody, a conjugated antibody, an activatable antibody, and/or a conjugated activatable antibody of the disclosure is also useful in the detection of a target in patient samples and accordingly are useful as diagnostics. For example, the antibodies and/or activatable antibodies, and conjugated versions thereof, of the disclosure are used in in vitro assays, e.g., ELISA, to detect target levels in a patient sample.

In one embodiment, an antibody, a conjugated antibody, an activatable antibody and/or a conjugated activatable antibody of the disclosure is immobilized on a solid support (e.g., the well(s) of a microtiter plate). The immobilized antibody, conjugated antibody, activatable antibody and/or conjugated activatable antibody serves as a capture antibody for any target that may be present in a test sample. Prior to contacting the immobilized antibody and/or activatable antibody, and/or conjugated versions thereof, with a patient sample, the solid support is rinsed and treated with a blocking agent such as milk protein or albumin to prevent nonspecific adsorption of the analyte.

Subsequently the wells are treated with a test sample suspected of containing the antigen, or with a solution containing a standard amount of the antigen. Such a sample is, e.g., a serum sample from a subject suspected of having levels of circulating antigen considered to be diagnostic of a pathology. After rinsing away the test sample or standard, the solid support is treated with a second antibody that is detectably labeled. The labeled second antibody serves as a detecting antibody. The level of detectable label is measured, and the concentration of target antigen in the test sample is determined by comparison with a standard curve developed from the standard samples.

It will be appreciated that based on the results obtained using the antibodies and activatable antibodies of the disclosure, and conjugated versions thereof, in an in vitro diagnostic assay, it is possible to stage a disease in a subject based on expression levels of the target antigen. For a given disease, samples of blood are taken from subjects diagnosed as being at various stages in the progression of the disease, and/or at various points in the therapeutic treatment of the disease. Using a population of samples that provides statistically significant results for each stage of progression or therapy, a range of concentrations of the antigen that may be considered characteristic of each stage is designated.

An antibody, a conjugated antibody, an activatable antibody and/or a conjugated activatable antibody can also be used in diagnostic and/or imaging methods. In some embodiments, such methods are in vitro methods. In some embodiments, such methods are in vivo methods. In some embodiments, such methods are in situ methods. In some embodiments, such methods are ex vivo methods. For example, activatable antibodies having an enzymatically cleavable CM can be used to detect the presence or absence of an enzyme that is capable of cleaving the CM. Such activatable antibodies can be used in diagnostics, which can include in vivo detection (e.g., qualitative or quantitative) of enzyme activity (or, in some embodiments, an environment of increased reduction potential such as that which can provide for reduction of a disulfide bond) through measured accumulation of activated antibodies (i.e., antibodies resulting from cleavage of an activatable antibody) in a given cell or tissue of a given host organism. Such accumulation of activated antibodies indicates not only that the tissue expresses enzymatic activity (or an increased reduction potential depending on the nature of the CM) but also that the tissue expresses target to which the activated antibody binds.

For example, the CM can be selected to be substrate for at least one protease found at the site of a tumor, at the site of a viral or bacterial infection at a biologically confined site (e.g., such as in an abscess, in an organ, and the like), and the like. The AB can be one that binds a target antigen. Using methods as disclosed herein, or when appropriate, methods familiar to one skilled in the art, a detectable label (e.g., a fluorescent label or radioactive label or radiotracer) can be conjugated to an AB or other region of an antibody and/or activatable antibody. Suitable detectable labels are discussed in the context of the above screening methods and additional specific examples are provided below. Using an AB specific to a protein or peptide of the disease state, along with at least one protease whose activity is elevated in the disease tissue of interest, activatable antibodies will exhibit an increased rate of binding to disease tissue relative to tissues where the CM specific enzyme is not present at a detectable level or is present at a lower level than in disease tissue or is inactive (e.g., in zymogen form or in complex with an inhibitor). Since small proteins and peptides are rapidly cleared from the blood by the renal filtration system, and because the enzyme specific for the CM is not present at a detectable level (or is present at lower levels in non-disease tissues or is present in inactive conformation), accumulation of activated antibodies in the disease tissue is enhanced relative to non-disease tissues.

In another example, activatable antibodies can be used to detect the presence or absence of a cleaving agent in a sample. For example, where the activatable antibodies contain a CM susceptible to cleavage by an enzyme, the activatable antibodies can be used to detect (either qualitatively or quantitatively) the presence of an enzyme in the sample. In another example, where the activatable antibodies contain a CM susceptible to cleavage by reducing agent, the activatable antibodies can be used to detect (either qualitatively or quantitatively) the presence of reducing conditions in a sample. To facilitate analysis in these methods, the activatable antibodies can be detectably labeled, and can be bound to a support (e.g., a solid support, such as a slide or bead). The detectable label can be positioned on a portion of the activatable antibody that is not released following cleavage, for example, the detectable label can be a quenched fluorescent label or other label that is not detectable until cleavage has occurred. The assay can be conducted by, for example, contacting the immobilized, detectably labeled activatable antibodies with a sample suspected of containing an enzyme and/or reducing agent for a time sufficient for cleavage to occur, then washing to remove excess sample and contaminants. The presence or absence of the cleaving agent (e.g., enzyme or reducing agent) in the sample is then assessed by a change in detectable signal of the activatable antibodies prior to contacting with the sample e.g., the presence of and/or an increase in detectable signal due to cleavage of the activatable antibody by the cleaving agent in the sample.

Such detection methods can be adapted to also provide for detection of the presence or absence of a target that is capable of binding the AB of the activatable antibodies when cleaved. Thus, the assays can be adapted to assess the presence or absence of a cleaving agent and the presence or absence of a target of interest. The presence or absence of the cleaving agent can be detected by the presence of and/or an increase in detectable label of the activatable antibodies as described above, and the presence or absence of the target can be detected by detection of a target-AB complex e.g., by use of a detectably labeled anti-target antibody.

Activatable antibodies are also useful in in situ imaging for the validation of activatable antibody activation, e.g., by protease cleavage, and binding to a particular target. In situ imaging is a technique that enables localization of proteolytic activity and target in biological samples such as cell cultures or tissue sections. Using this technique, it is possible to confirm both binding to a given target and proteolytic activity based on the presence of a detectable label (e.g., a fluorescent label).

These techniques are useful with any frozen cells or tissue derived from a disease site (e.g. tumor tissue) or healthy tissues. These techniques are also useful with fresh cell or tissue samples.

In these techniques, an activatable antibody is labeled with a detectable label. The detectable label can be a fluorescent dye, (e.g. a fluorophore, Fluorescein Isothiocyanate (FITC), Rhodamine Isothiocyanate (TRITC), an Alexa Fluor@ label), a near infrared (NIR) dye (e.g., Qdot® nanocrystals), a colloidal metal, a hapten, a radioactive marker, biotin and an amplification reagent such as streptavidin, or an enzyme (e.g. horseradish peroxidase or alkaline phosphatase).

Detection of the label in a sample that has been incubated with the labeled, activatable antibody indicates that the sample contains the target and contains a protease that is specific for the CM of the activatable antibody. In some embodiments, the presence of the protease can be confirmed using broad spectrum protease inhibitors such as those described herein, and/or by using an agent that is specific for the protease, for example, an antibody such as A11, which is specific for the protease matriptase and inhibits the proteolytic activity of matriptase; see e.g., International Publication Number WO 2010/129609, published 11 Nov. 2010. The same approach of using broad spectrum protease inhibitors such as those described herein, and/or by using a more selective inhibitory agent can be used to identify a protease that is specific for the CM of the activatable antibody. In some embodiments, the presence of the target can be confirmed using an agent that is specific for the target, e.g., another antibody, or the detectable label can be competed with unlabeled target. In some embodiments, unlabeled activatable antibody could be used, with detection by a labeled secondary antibody or more complex detection system.

Similar techniques are also useful for in vivo imaging where detection of the fluorescent signal in a subject, e.g., a mammal, including a human, indicates that the disease site contains the target and contains a protease that is specific for the CM of the activatable antibody.

These techniques are also useful in kits and/or as reagents for the detection, identification or characterization of protease activity in a variety of cells, tissues, and organisms based on the protease-specific CM in the activatable antibody.

The disclosure provides methods of using the antibodies and/or activatable antibodies in a variety of diagnostic and/or prophylactic indications. For example, the disclosure provides methods of detecting presence or absence of a cleaving agent and a target of interest in a subject or a sample by (i) contacting a subject or sample with an activatable antibody, wherein the activatable antibody comprises a masking moiety (MM), a cleavable moiety (CM) that is cleaved by the cleaving agent, e.g., a protease, and an antigen binding domain or fragment thereof (AB) that specifically binds the target of interest, wherein the activatable antibody in an uncleaved, non-activated state comprises a structural arrangement from N-terminus to C-terminus as follows: MM-CM-AB or AB-CM-MM; (a) wherein the MM is a peptide that inhibits binding of the AB to the target, and wherein the MM does not have an amino acid sequence of a naturally occurring binding partner of the AB and is not a modified form of a natural binding partner of the AB, and (b) wherein, in an uncleaved, non-activated state, the MM interferes with specific binding of the AB to the target, and in a cleaved, activated state the MM does not interfere or compete with specific binding of the AB to the target; and (ii) measuring a level of activated activatable antibody in the subject or sample, wherein a detectable level of activated activatable antibody in the subject or sample indicates that the cleaving agent and the target are present in the subject or sample and wherein no detectable level of activated activatable antibody in the subject or sample indicates that the cleaving agent, the target or both the cleaving agent and the target are absent and/or not sufficiently present in the subject or sample. In some embodiments, the activatable antibody is an activatable antibody to which a therapeutic agent is conjugated. In some embodiments, the activatable antibody is not conjugated to an agent. In some embodiments, the activatable antibody comprises a detectable label. In some embodiments, the detectable label is positioned on the AB. In some embodiments, measuring the level of activatable antibody in the subject or sample is accomplished using a secondary reagent that specifically binds to the activated antibody, wherein the reagent comprises a detectable label. In some embodiments, the secondary reagent is an antibody comprising a detectable label.

The disclosure also provides methods of detecting presence or absence of a cleaving agent in a subject or a sample by (i) contacting a subject or sample with an activatable antibody in the presence of a target of interest, e.g., the target, wherein the activatable antibody comprises a masking moiety (MM), a cleavable moiety (CM) that is cleaved by the cleaving agent, e.g., a protease, and an antigen binding domain or fragment thereof (AB) that specifically binds the target of interest, wherein the activatable antibody in an uncleaved, non-activated state comprises a structural arrangement from N-terminus to C-terminus as follows: MM-CM-AB or AB-CM-MM; (a) wherein the MM is a peptide that inhibits binding of the AB to the target, and wherein the MM does not have an amino acid sequence of a naturally occurring binding partner of the AB and is not a modified form of a natural binding partner of the AB; and (b) wherein, in an uncleaved, non-activated state, the MM interferes with specific binding of the AB to the target, and in a cleaved, activated state the MM does not interfere or compete with specific binding of the AB to the target; and (ii) measuring a level of activated activatable antibody in the subject or sample, wherein a detectable level of activated activatable antibody in the subject or sample indicates that the cleaving agent is present in the subject or sample and wherein no detectable level of activated activatable antibody in the subject or sample indicates that the cleaving agent is absent and/or not sufficiently present in the subject or sample. In some embodiments, the activatable antibody is an activatable antibody to which a therapeutic agent is conjugated. In some embodiments, the activatable antibody is not conjugated to an agent. In some embodiments, the activatable antibody comprises a detectable label. In some embodiments, the detectable label is positioned on the AB. In some embodiments, measuring the level of activatable antibody in the subject or sample is accomplished using a secondary reagent that specifically binds to the activated antibody, wherein the reagent comprises a detectable label. In some embodiments, the secondary reagent is an antibody comprising a detectable label.

The disclosure also provides kits for use in methods of detecting presence or absence of a cleaving agent and the target in a subject or a sample, where the kits include at least an activatable antibody comprises a masking moiety (MM), a cleavable moiety (CM) that is cleaved by the cleaving agent, e.g., a protease, and an antigen binding domain or fragment thereof (AB) that specifically binds the target of interest, wherein the activatable antibody in an uncleaved, non-activated state comprises a structural arrangement from N-terminus to C-terminus as follows: MM-CM-AB or AB-CM-MM; (a) wherein the MM is a peptide that inhibits binding of the AB to the target, and wherein the MM does not have an amino acid sequence of a naturally occurring binding partner of the AB and is not a modified form of a natural binding partner of the AB; and (b) wherein, in an uncleaved, non-activated state, the MM interferes with specific binding of the AB to the target, and in a cleaved, activated state the MM does not interfere or compete with specific binding of the AB to the target; and (ii) measuring a level of activated activatable antibody in the subject or sample, wherein a detectable level of activated activatable antibody in the subject or sample indicates that the cleaving agent is present in the subject or sample and wherein no detectable level of activated activatable antibody in the subject or sample indicates that the cleaving agent is absent and/or not sufficiently present in the subject or sample. In some embodiments, the activatable antibody is an activatable antibody to which a therapeutic agent is conjugated. In some embodiments, the activatable antibody is not conjugated to an agent. In some embodiments, the activatable antibody comprises a detectable label. In some embodiments, the detectable label is positioned on the AB. In some embodiments, measuring the level of activatable antibody in the subject or sample is accomplished using a secondary reagent that specifically binds to the activated antibody, wherein the reagent comprises a detectable label. In some embodiments, the secondary reagent is an antibody comprising a detectable label.

The disclosure also provides methods of detecting presence or absence of a cleaving agent in a subject or a sample by (i) contacting a subject or sample with an activatable antibody, wherein the activatable antibody comprises a masking moiety (MM), a cleavable moiety (CM) that is cleaved by the cleaving agent, e.g., a protease, an antigen binding domain (AB) that specifically binds the target, and a detectable label, wherein the activatable antibody in an uncleaved, non-activated state comprises a structural arrangement from N-terminus to C-terminus as follows: MM-CM-AB or AB-CM-MM; wherein the MM is a peptide that inhibits binding of the AB to the target, and wherein the MM does not have an amino acid sequence of a naturally occurring binding partner of the AB and is not a modified form of a natural binding partner of the AB; wherein, in an uncleaved, non-activated state, the MM interferes with specific binding of the AB to the target, and in a cleaved, activated state the MM does not interfere or compete with specific binding of the AB to the target; and wherein the detectable label is positioned on a portion of the activatable antibody that is released following cleavage of the CM; and (ii) measuring a level of detectable label in the subject or sample, wherein a detectable level of the detectable label in the subject or sample indicates that the cleaving agent is absent and/or not sufficiently present in the subject or sample and wherein no detectable level of the detectable label in the subject or sample indicates that the cleaving agent is present in the subject or sample. In some embodiments, the activatable antibody is an activatable antibody to which a therapeutic agent is conjugated. In some embodiments, the activatable antibody is not conjugated to an agent. In some embodiments, the activatable antibody comprises a detectable label. In some embodiments, the detectable label is positioned on the AB. In some embodiments, measuring the level of activatable antibody in the subject or sample is accomplished using a secondary reagent that specifically binds to the activated antibody, wherein the reagent comprises a detectable label. In some embodiments, the secondary reagent is an antibody comprising a detectable label.

The disclosure also provides kits for use in methods of detecting presence or absence of a cleaving agent and the target in a subject or a sample, where the kits include at least an activatable antibody and/or conjugated activatable antibody (e.g., an activatable antibody to which a therapeutic agent is conjugated) described herein for use in contacting a subject or biological sample and means for detecting the level of activated activatable antibody and/or conjugated activatable antibody in the subject or biological sample, wherein a detectable level of activated activatable antibody in the subject or biological sample indicates that the cleaving agent and the target are present in the subject or biological sample and wherein no detectable level of activated activatable antibody in the subject or biological sample indicates that the cleaving agent, the target or both the cleaving agent and the target are absent and/or not sufficiently present in the subject or biological sample, such that the target binding and/or protease cleavage of the activatable antibody cannot be detected in the subject or biological sample.

The disclosure also provides methods of detecting presence or absence of a cleaving agent in a subject or a sample by (i) contacting a subject or biological sample with an activatable antibody in the presence of the target, and (ii) measuring a level of activated activatable antibody in the subject or biological sample, wherein a detectable level of activated activatable antibody in the subject or biological sample indicates that the cleaving agent is present in the subject or biological sample and wherein no detectable level of activated activatable antibody in the subject or biological sample indicates that the cleaving agent is absent and/or not sufficiently present in the subject or biological sample at a detectable level, such that protease cleavage of the activatable antibody cannot be detected in the subject or biological sample. Such an activatable antibody includes a masking moiety (MM), a cleavable moiety (CM) that is cleaved by the cleaving agent, e.g., a protease, and an antigen binding domain or fragment thereof (AB) that specifically binds the target, wherein the activatable antibody in an uncleaved (i.e., non-activated) state comprises a structural arrangement from N-terminus to C-terminus as follows: MM-CM-AB or AB-CM-MM; (a) wherein the MM is a peptide that inhibits binding of the AB to the target, and wherein the MM does not have an amino acid sequence of a naturally occurring binding partner of the AB; and (b) wherein the MM of the activatable antibody in an uncleaved state interferes with specific binding of the AB to the target, and wherein the MM of an activatable antibody in a cleaved (i.e., activated) state does not interfere or compete with specific binding of the AB to the target. In some embodiments, the activatable antibody is an activatable antibody to which a therapeutic agent is conjugated. In some embodiments, the activatable antibody is not conjugated to an agent. In some embodiments, the detectable label is attached to the masking moiety. In some embodiments, the detectable label is attached to the cleavable moiety N-terminal to the protease cleavage site. In some embodiments, a single antigen binding site of the AB is masked. In some embodiments wherein an antibody of the disclosure has at least two antigen binding sites, at least one antigen binding site is masked and at least one antigen binding site is not masked. In some embodiments all antigen binding sites are masked. In some embodiments, the measuring step includes use of a secondary reagent comprising a detectable label.

The disclosure also provides kits for use in methods of detecting presence or absence of a cleaving agent and the target in a subject or a sample, where the kits include at least an activatable antibody and/or conjugated activatable antibody described herein for use in contacting a subject or biological sample with an activatable antibody in the presence of the target, and measuring a level of activated activatable antibody in the subject or biological sample, wherein a detectable level of activated activatable antibody in the subject or biological sample indicates that the cleaving agent is present in the subject or biological sample and wherein no detectable level of activated activatable antibody in the subject or biological sample indicates that the cleaving agent is absent and/or not sufficiently present in the subject or biological sample at a detectable level, such that protease cleavage of the activatable antibody cannot be detected in the subject or biological sample. Such an activatable antibody includes a masking moiety (MM), a cleavable moiety (CM) that is cleaved by the cleaving agent, e.g., a protease, and an antigen binding domain or fragment thereof (AB) that specifically binds the target, wherein the activatable antibody in an uncleaved (i.e., non-activated) state comprises a structural arrangement from N-terminus to C-terminus as follows: MM-CM-AB or AB-CM-MM; (a) wherein the MM is a peptide that inhibits binding of the AB to the target, and wherein the MM does not have an amino acid sequence of a naturally occurring binding partner of the AB; and (b) wherein the MM of the activatable antibody in an uncleaved state interferes with specific binding of the AB to the target, and wherein the MM of an activatable antibody in a cleaved (i.e., activated) state does not interfere or compete with specific binding of the AB to the target. In some embodiments, the activatable antibody is an activatable antibody to which a therapeutic agent is conjugated. In some embodiments, the activatable antibody is not conjugated to an agent. In some embodiments, the detectable label is attached to the masking moiety. In some embodiments, the detectable label is attached to the cleavable moiety N-terminal to the protease cleavage site. In some embodiments, a single antigen binding site of the AB is masked. In some embodiments wherein an antibody of the disclosure has at least two antigen binding sites, at least one antigen binding site is masked and at least one antigen binding site is not masked. In some embodiments all antigen binding sites are masked. In some embodiments, the measuring step includes use of a secondary reagent comprising a detectable label.

The disclosure also provides kits for use in methods of detecting presence or absence of a cleaving agent in a subject or a sample, where the kits include at least an activatable antibody and/or conjugated activatable antibody described herein for use in contacting a subject or biological sample and means for detecting the level of activated activatable antibody and/or conjugated activatable antibody in the subject or biological sample, wherein the activatable antibody includes a detectable label that is positioned on a portion of the activatable antibody that is released following cleavage of the CM, wherein a detectable level of activated activatable antibody in the subject or biological sample indicates that the cleaving agent is absent and/or not sufficiently present in the subject or biological sample such that the target binding and/or protease cleavage of the activatable antibody cannot be detected in the subject or biological sample, and wherein no detectable level of activated activatable antibody in the subject or biological sample indicates that the cleaving agent is present in the subject or biological sample at a detectable level.

The disclosure provides methods of detecting presence or absence of a cleaving agent and the target in a subject or a sample by (i) contacting a subject or biological sample with an activatable antibody, wherein the activatable antibody includes a detectable label that is positioned on a portion of the activatable antibody that is released following cleavage of the CM and (ii) measuring a level of activated activatable antibody in the subject or biological sample, wherein a detectable level of activated activatable antibody in the subject or biological sample indicates that the cleaving agent, the target or both the cleaving agent and the target are absent and/or not sufficiently present in the subject or biological sample, such that the target binding and/or protease cleavage of the activatable antibody cannot be detected in the subject or biological sample, and wherein a reduced detectable level of activated activatable antibody in the subject or biological sample indicates that the cleaving agent and the target are present in the subject or biological sample. A reduced level of detectable label is, for example, a reduction of about 5%, about 10%, about 15%, about 20%, about 25%, about 30%, about 35%, about 40%, about 45%, about 50%, about 55%, about 60%, about 65%, about 70%, about 75%, about 80%, about 85%, about 900, about 95% and/or about 100%. Such an activatable antibody includes a masking moiety (MM), a cleavable moiety (CM) that is cleaved by the cleaving agent, and an antigen binding domain or fragment thereof (AB) that specifically binds the target, wherein the activatable antibody in an uncleaved (i.e., non-activated) state comprises a structural arrangement from N-terminus to C-terminus as follows: MM-CM-AB or AB-CM-MM; (a) wherein the MM is a peptide that inhibits binding of the AB to the target, and wherein the MM does not have an amino acid sequence of a naturally occurring binding partner of the AB; and (b) wherein the MM of the activatable antibody in an uncleaved state interferes with specific binding of the AB to the target, and wherein the MM of an activatable antibody in a cleaved (i.e., activated) state does not interfere or compete with specific binding of the AB to the target. In some embodiments, the activatable antibody is an activatable antibody to which a therapeutic agent is conjugated. In some embodiments, the activatable antibody is not conjugated to an agent. In some embodiments, the activatable antibody comprises a detectable label. In some embodiments, the detectable label is positioned on the AB. In some embodiments, measuring the level of activatable antibody in the subject or sample is accomplished using a secondary reagent that specifically binds to the activated antibody, wherein the reagent comprises a detectable label. In some embodiments, the secondary reagent is an antibody comprising a detectable label.

The disclosure also provides kits for use in methods of detecting presence or absence of a cleaving agent and the target in a subject or a sample, where the kits include at least an activatable antibody and/or conjugated activatable antibody described herein for use in contacting a subject or biological sample and means for detecting the level of activated activatable antibody and/or conjugated activatable antibody in the subject or biological sample, wherein a detectable level of activated activatable antibody in the subject or biological sample indicates that the cleaving agent, the target or both the cleaving agent and the target are absent and/or not sufficiently present in the subject or biological sample, such that the target binding and/or protease cleavage of the activatable antibody cannot be detected in the subject or biological sample, and wherein a reduced detectable level of activated activatable antibody in the subject or biological sample indicates that the cleaving agent and the target are present in the subject or biological sample. A reduced level of detectable label is, for example, a reduction of about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95% and/or about 100%.

The disclosure also provides methods of detecting presence or absence of a cleaving agent in a subject or a sample by (i) contacting a subject or biological sample with an activatable antibody, wherein the activatable antibody includes a detectable label that is positioned on a portion of the activatable antibody that is released following cleavage of the CM; and (ii) measuring a level of detectable label in the subject or biological sample, wherein a detectable level of the detectable label in the subject or biological sample indicates that the cleaving agent is absent and/or not sufficiently present in the subject or biological sample at a detectable level, such that protease cleavage of the activatable antibody cannot be detected in the subject or biological sample, and wherein a reduced detectable level of the detectable label in the subject or biological sample indicates that the cleaving agent is present in the subject or biological sample. A reduced level of detectable label is, for example, a reduction of about 5%, about 10%, about 15%, about 20%, about 25%, about 30%, about 35%, about 40%, about 45%, about 50%, about 55%, about 60%, about 65%, about 70%, about 75%, about 80%, about 85%, about 900, about 95% and/or about 100%. Such an activatable antibody includes a masking moiety (MM), a cleavable moiety (CM) that is cleaved by the cleaving agent, and an antigen binding domain or fragment thereof (AB) that specifically binds the target, wherein the activatable antibody in an uncleaved (i.e., non-activated) state comprises a structural arrangement from N-terminus to C-terminus as follows: MM-CM-AB or AB-CM-MM; (a) wherein the MM is a peptide that inhibits binding of the AB to the target, and wherein the MM does not have an amino acid sequence of a naturally occurring binding partner of the AB; and (b) wherein the MM of the activatable antibody in an uncleaved state interferes with specific binding of the AB to the target, and wherein the MM of an activatable antibody in a cleaved (i.e., activated) state does not interfere or compete with specific binding of the AB to the target. In some embodiments, the activatable antibody is an activatable antibody to which a therapeutic agent is conjugated. In some embodiments, the activatable antibody is not conjugated to an agent. In some embodiments, the activatable antibody comprises a detectable label. In some embodiments, the detectable label is positioned on the AB. In some embodiments, measuring the level of activatable antibody in the subject or sample is accomplished using a secondary reagent that specifically binds to the activated antibody, wherein the reagent comprises a detectable label. In some embodiments, the secondary reagent is an antibody comprising a detectable label.

The disclosure also provides kits for use in methods of detecting presence or absence of a cleaving agent of interest in a subject or a sample, where the kits include at least an activatable antibody and/or conjugated activatable antibody described herein for use in contacting a subject or biological sample and means for detecting the level of activated activatable antibody and/or conjugated activatable antibody in the subject or biological sample, wherein the activatable antibody includes a detectable label that is positioned on a portion of the activatable antibody that is released following cleavage of the CM, wherein a detectable level of the detectable label in the subject or biological sample indicates that the cleaving agent, the target, or both the cleaving agent and the target are absent and/or not sufficiently present in the subject or biological sample, such that the target binding and/or protease cleavage of the activatable antibody cannot be detected in the subject or biological sample, and wherein a reduced detectable level of the detectable label in the subject or biological sample indicates that the cleaving agent and the target are present in the subject or biological sample. A reduced level of detectable label is, for example, a reduction of about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95% and/or about 100%.

In some embodiments of these methods and kits, the activatable antibody includes a detectable label. In some embodiments of these methods and kits, the detectable label includes an imaging agent, a contrasting agent, an enzyme, a fluorescent label, a chromophore, a dye, one or more metal ions, or a ligand-based label. In some embodiments of these methods and kits, the imaging agent comprises a radioisotope. In some embodiments of these methods and kits, the radioisotope is indium or technetium. In some embodiments of these methods and kits, the contrasting agent comprises iodine, gadolinium or iron oxide. In some embodiments of these methods and kits, the enzyme comprises horseradish peroxidase, alkaline phosphatase, or 3-galactosidase. In some embodiments of these methods and kits, the fluorescent label comprises yellow fluorescent protein (YFP), cyan fluorescent protein (CFP), green fluorescent protein (GFP), modified red fluorescent protein (mRFP), red fluorescent protein tdimer2 (RFP tdimer2), HCRED, or a europium derivative. In some embodiments of these methods and kits, the luminescent label comprises an N-methylacrydium derivative. In some embodiments of these methods, the label comprises an Alexa Fluor® label, such as Alex Fluor® 680 or Alexa Fluor® 750. In some embodiments of these methods and kits, the ligand-based label comprises biotin, avidin, streptavidin or one or more haptens.

In some embodiments of these methods, the method is an in vivo method. In some embodiments of these methods, the method is an in situ method. In some embodiments of these methods, the method is an ex vivo method. In some embodiments of these methods, the method is an in vitro method.

In some embodiments, in situ imaging and/or in vivo imaging are useful in methods to identify which patients to treat. For example, in in situ imaging, the activatable antibodies are used to screen patient samples to identify those patients having the appropriate protease(s) and target(s) at the appropriate location, e.g., at a tumor site.

In some embodiments in situ imaging is used to identify or otherwise refine a patient population suitable for treatment with an activatable antibody of the disclosure. For example, patients that test positive for both the target (e.g., the target) and a protease that cleaves the substrate in the cleavable moiety (CM) of the activatable antibody being tested (e.g., accumulate activated antibodies at the disease site) are identified as suitable candidates for treatment with such an activatable antibody comprising such a CM. Likewise, patients that test negative for either or both of the target (e.g., the target) and the protease that cleaves the substrate in the CM in the activatable antibody being tested using these methods might be identified as suitable candidates for another form of therapy. In some embodiments, such patients that test negative with respect to a first activatable antibody can be tested with other activatable antibodies comprising different CMs until a suitable activatable antibody for treatment is identified (e.g., an activatable antibody comprising a CM that is cleaved by the patient at the site of disease). In some embodiments, the patient is then administered a therapeutically effective amount of the activatable antibody for which the patient tested positive.

In some embodiments in vivo imaging is used to identify or otherwise refine a patient population suitable for treatment with an activatable antibody of the disclosure. For example, patients that test positive for both the target (e.g., the target) and a protease that cleaves the substrate in the cleavable moiety (CM) of the activatable antibody being tested (e.g., accumulate activated antibodies at the disease site) are identified as suitable candidates for treatment with such an activatable antibody comprising such a CM. Likewise, patients that test negative might be identified as suitable candidates for another form of therapy. In some embodiments, such patients that test negative with respect to a first activatable antibody can be tested with other activatable antibodies comprising different CMs until a suitable activatable antibody for treatment is identified (e.g., an activatable antibody comprising a CM that is cleaved by the patient at the site of disease). In some embodiments, the patient is then administered a therapeutically effective amount of the activatable antibody for which the patient tested positive.

In some embodiments of the methods and kits, the method or kit is used to identify or otherwise refine a patient population suitable for treatment with an activatable antibody of the disclosure. For example, patients that test positive for both the target (e.g., the target) and a protease that cleaves the substrate in the cleavable moiety (CM) of the activatable antibody being tested in these methods are identified as suitable candidates for treatment with such an activatable antibody comprising such a CM. Likewise, patients that test negative for both of the targets (e.g., the target) and the protease that cleaves the substrate in the CM in the activatable antibody being tested using these methods might be identified as suitable candidates for another form of therapy. In some embodiments, such patients can be tested with other activatable antibodies until a suitable activatable antibody for treatment is identified (e.g., an activatable antibody comprising a CM that is cleaved by the patient at the site of disease). In some embodiments, patients that test negative for either of the target (e.g., the target) are identified as suitable candidates for treatment with such an activatable antibody comprising such a CM. In some embodiments, patients that test negative for either of the target (e.g., the target) are identified as not being suitable candidates for treatment with such an activatable antibody comprising such a CM. In some embodiments, such patients can be tested with other activatable antibodies until a suitable activatable antibody for treatment is identified (e.g., an activatable antibody comprising a CM that is cleaved by the patient at the site of disease). In some embodiments, the activatable antibody is an activatable antibody to which a therapeutic agent is conjugated. In some embodiments, the activatable antibody is not conjugated to an agent. In some embodiments, the activatable antibody comprises a detectable label. In some embodiments, the detectable label is positioned on the AB. In some embodiments, measuring the level of activatable antibody in the subject or sample is accomplished using a secondary reagent that specifically binds to the activated antibody, wherein the reagent comprises a detectable label. In some embodiments, the secondary reagent is an antibody comprising a detectable label.

In some embodiments, a method or kit is used to identify or otherwise refine a patient population suitable for treatment with an anti-the target activatable antibody and/or conjugated activatable antibody (e.g., activatable antibody to which a therapeutic agent is conjugated) of the disclosure, followed by treatment by administering that activatable antibody and/or conjugated activatable antibody to a subject in need thereof. For example, patients that test positive for both the targets (e.g., the target) and a protease that cleaves the substrate in the cleavable moiety (CM) of the activatable antibody and/or conjugated activatable antibody being tested in these methods are identified as suitable candidates for treatment with such antibody and/or such a conjugated activatable antibody comprising such a CM, and the patient is then administered a therapeutically effective amount of the activatable antibody and/or conjugated activatable antibody that was tested. Likewise, patients that test negative for either or both of the target (e.g., the target) and the protease that cleaves the substrate in the CM in the activatable antibody being tested using these methods might be identified as suitable candidates for another form of therapy. In some embodiments, such patients can be tested with other antibody and/or conjugated activatable antibody until a suitable antibody and/or conjugated activatable antibody for treatment is identified (e.g., an activatable antibody and/or conjugated activatable antibody comprising a CM that is cleaved by the patient at the site of disease). In some embodiments, the patient is then administered a therapeutically effective amount of the activatable antibody and/or conjugated activatable antibody for which the patient tested positive.

In some embodiments of these methods and kits, the MM is a peptide having a length from about 4 to 40 amino acids. In some embodiments of these methods and kits, the activatable antibody comprises a linker peptide, wherein the linker peptide is positioned between the MM and the CM. In some embodiments of these methods and kits, the activatable antibody comprises a linker peptide, where the linker peptide is positioned between the AB and the CM. In some embodiments of these methods and kits, the activatable antibody comprises a first linker peptide (L1) and a second linker peptide (L2), wherein the first linker peptide is positioned between the MM and the CM and the second linker peptide is positioned between the AB and the CM. In some embodiments of these methods and kits, each of L1 and L2 is a peptide of about 1 to 20 amino acids in length, and wherein each of L1 and L2 need not be the same linker. In some embodiments of these methods and kits, one or both of L1 and L2 comprises a glycine-serine polymer. In some embodiments of these methods and kits, at least one of L1 and L2 comprises an amino acid sequence selected from the group consisting of (GS)ₙ, (GSGGS)ₙ (SEQ ID NO: 339) and (GGGS)ₙ (SEQ ID NO: 340), where n is an integer of at least one. In some embodiments of these methods and kits, at least one of L1 and L2 comprises an amino acid sequence having the formula (GGS)ₙ, where n is an integer of at least one. In some embodiments of these methods and kits, at least one of L1 and L2 comprises an amino acid sequence selected from the group consisting of Gly-Gly-Ser-Gly (SEQ ID NO: 341, Gly-Gly-Ser-Gly-Gly (SEQ ID NO: 342), Gly-Ser-Gly-Ser-Gly (SEQ ID NO: 343, Gly-Ser-Gly-Gly-Gly (SEQ ID NO: 344), Gly-Gly-Gly-Ser-Gly (SEQ ID NO: 345), and Gly-Ser-Ser-Ser-Gly (SEQ ID NO: 346).

In some embodiments of these methods and kits, the AB comprises an antibody or antibody fragment sequence selected from the cross-reactive antibody sequences presented herein. In some embodiments of these methods and kits, the AB comprises a Fab fragment, a scFv or a single chain antibody (scAb).

In some embodiments of these methods and kits, the cleaving agent is a protease that is co-localized in the subject or sample with the target and the CM is a polypeptide that functions as a substrate for the protease, wherein the protease cleaves the CM in the activatable antibody when the activatable antibody is exposed to the protease. In some embodiments of these methods and kits, the CM is a polypeptide of up to 15 amino acids in length. In some embodiments of these methods and kits, the CM is coupled to the N-terminus of the AB. In some embodiments of these methods and kits, the CM is coupled to the C-terminus of the AB. In some embodiments of these methods and kits, the CM is coupled to the N-terminus of a VL chain of the AB.

The antibodies, conjugated antibodies, activatable antibodies and/or conjugated activatable antibodies of the disclosure are used in diagnostic and prophylactic formulations. In one embodiment, an activatable antibody is administered to patients that are at risk of developing one or more of the aforementioned inflammation, inflammatory disorders, cancer or other disorders.

A patient's or organ's predisposition to one or more of the aforementioned disorders can be determined using genotypic, serological or biochemical markers.

In some embodiments of the disclosure, an antibody, a conjugated antibody, an activatable antibody and/or a conjugated activatable antibody is administered to human individuals diagnosed with a clinical indication associated with one or more of the aforementioned disorders. Upon diagnosis, an antibody, a conjugated antibody, an activatable antibody and/or a conjugated activatable antibody is administered to mitigate or reverse the effects of the clinical indication.

Antibodies, conjugated antibodies, activatable antibodies and/or conjugated activatable antibodies of the disclosure are also useful in the detection of the target in patient samples and accordingly are useful as diagnostics. For example, the antibodies, conjugated antibodies, the activatable antibodies and/or conjugated activatable antibodies of the disclosure are used in in vitro assays, e.g., ELISA, to detect target levels in a patient sample.

In one embodiment, an antibody and/or activatable antibody of the disclosure is immobilized on a solid support (e.g., the well(s) of a microtiter plate). The immobilized antibody and/or activatable antibody serves as a capture antibody for any target that may be present in a test sample. Prior to contacting the immobilized antibody and/or activatable antibody with a patient sample, the solid support is rinsed and treated with a blocking agent such as milk protein or albumin to prevent nonspecific adsorption of the analyte.

Subsequently the wells are treated with a test sample suspected of containing the antigen, or with a solution containing a standard amount of the antigen. Such a sample is, e.g., a serum sample from a subject suspected of having levels of circulating antigen considered to be diagnostic of a pathology. After rinsing away the test sample or standard, the solid support is treated with a second antibody that is detectably labeled. The labeled second antibody serves as a detecting antibody. The level of detectable label is measured, and the concentration of target antigen in the test sample is determined by comparison with a standard curve developed from the standard samples.

It will be appreciated that based on the results obtained using the antibodies and/or activatable antibodies of the disclosure in an in vitro diagnostic assay, it is possible to stage a disease in a subject based on expression levels of the Target antigen. For a given disease, samples of blood are taken from subjects diagnosed as being at various stages in the progression of the disease, and/or at various points in the therapeutic treatment of the disease. Using a population of samples that provides statistically significant results for each stage of progression or therapy, a range of concentrations of the antigen that may be considered characteristic of each stage is designated.

Antibodies, conjugated antibodies, activatable antibodies and/or conjugated activatable antibodies can also be used in diagnostic and/or imaging methods. In some embodiments, such methods are in vitro methods. In some embodiments, such methods are in vivo methods. In some embodiments, such methods are in situ methods. In some embodiments, such methods are ex vivo methods. For example, activatable antibodies having an enzymatically cleavable CM can be used to detect the presence or absence of an enzyme that is capable of cleaving the CM. Such activatable antibodies can be used in diagnostics, which can include in vivo detection (e.g., qualitative or quantitative) of enzyme activity (or, in some embodiments, an environment of increased reduction potential such as that which can provide for reduction of a disulfide bond) through measured accumulation of activated antibodies (i.e., antibodies resulting from cleavage of an activatable antibody) in a given cell or tissue of a given host organism. Such accumulation of activated antibodies indicates not only that the tissue expresses enzymatic activity (or an increased reduction potential depending on the nature of the CM) but also that the tissue expresses target to which the activated antibody binds.

For example, the CM can be selected to be a protease substrate for a protease found at the site of a tumor, at the site of a viral or bacterial infection at a biologically confined site (e.g., such as in an abscess, in an organ, and the like), and the like. The AB can be one that binds a target antigen. Using methods familiar to one skilled in the art, a detectable label (e.g., a fluorescent label or radioactive label or radiotracer) can be conjugated to an AB or other region of an activatable antibody. Suitable detectable labels are discussed in the context of the above screening methods and additional specific examples are provided below. Using an AB specific to a protein or peptide of the disease state, along with a protease whose activity is elevated in the disease tissue of interest, activatable antibodies will exhibit an increased rate of binding to disease tissue relative to tissues where the CM specific enzyme is not present at a detectable level or is present at a lower level than in disease tissue or is inactive (e.g., in zymogen form or in complex with an inhibitor). Since small proteins and peptides are rapidly cleared from the blood by the renal filtration system, and because the enzyme specific for the CM is not present at a detectable level (or is present at lower levels in non-disease tissues or is present in inactive conformation), accumulation of activated antibodies in the disease tissue is enhanced relative to non-disease tissues.

In another example, activatable antibodies can be used to detect the presence or absence of a cleaving agent in a sample. For example, where the activatable antibodies contain a CM susceptible to cleavage by an enzyme, the activatable antibodies can be used to detect (either qualitatively or quantitatively) the presence of an enzyme in the sample. In another example, where the activatable antibodies contain a CM susceptible to cleavage by reducing agent, the activatable antibodies can be used to detect (either qualitatively or quantitatively) the presence of reducing conditions in a sample. To facilitate analysis in these methods, the activatable antibodies can be detectably labeled, and can be bound to a support (e.g., a solid support, such as a slide or bead). The detectable label can be positioned on a portion of the activatable antibody that is not released following cleavage, for example, the detectable label can be a quenched fluorescent label or other label that is not detectable until cleavage has occurred. The assay can be conducted by, for example, contacting the immobilized, detectably labeled activatable antibodies with a sample suspected of containing an enzyme and/or reducing agent for a time sufficient for cleavage to occur, then washing to remove excess sample and contaminants. The presence or absence of the cleaving agent (e.g., enzyme or reducing agent) in the sample is then assessed by a change in detectable signal of the activatable antibodies prior to contacting with the sample e.g., the presence of and/or an increase in detectable signal due to cleavage of the activatable antibody by the cleaving agent in the sample.

Such detection methods can be adapted to also provide for detection of the presence or absence of a target that is capable of binding the AB of the activatable antibodies when cleaved. Thus, the assays can be adapted to assess the presence or absence of a cleaving agent and the presence or absence of a target of interest. The presence or absence of the cleaving agent can be detected by the presence of and/or an increase in detectable label of the activatable antibodies as described above, and the presence or absence of the target can be detected by detection of a target-AB complex e.g., by use of a detectably labeled anti-target antibody.

Activatable antibodies are also useful in in situ imaging for the validation of activatable antibody activation, e.g., by protease cleavage, and binding to a particular target. In situ imaging is a technique that enables localization of proteolytic activity and target in biological samples such as cell cultures or tissue sections. Using this technique, it is possible to confirm both binding to a given target and proteolytic activity based on the presence of a detectable label (e.g., a fluorescent label).

These techniques are useful with any frozen cells or tissue derived from a disease site (e.g. tumor tissue) or healthy tissues. These techniques are also useful with fresh cell or tissue samples.

In these techniques, an activatable antibody is labeled with a detectable label. The detectable label can be a fluorescent dye, (e.g. Fluorescein Isothiocyanate (FITC), Rhodamine Isothiocyanate (TRITC), a near infrared (NIR) dye (e.g., Qdot® nanocrystals), a colloidal metal, a hapten, a radioactive marker, biotin and an amplification reagent such as streptavidin, or an enzyme (e.g. horseradish peroxidase or alkaline phosphatase).

Detection of the label in a sample that has been incubated with the labeled, activatable antibody indicates that the sample contains the target and contains a protease that is specific for the CM of the activatable antibody. In some embodiments, the presence of the protease can be confirmed using broad spectrum protease inhibitors such as those described herein, and/or by using an agent that is specific for the protease, for example, an antibody such as A11, which is specific for the protease matriptase and inhibits the proteolytic activity of matriptase; see e.g., International Publication Number WO 2010/129609, published 11 Nov. 2010. The same approach of using broad spectrum protease inhibitors such as those described herein, and/or by using a more selective inhibitory agent can be used to identify a protease or class of proteases specific for the CM of the activatable antibody. In some embodiments, the presence of the target can be confirmed using an agent that is specific for the target, e.g., another antibody, or the detectable label can be competed with unlabeled target. In some embodiments, unlabeled activatable antibody could be used, with detection by a labeled secondary antibody or more complex detection system.

Similar techniques are also useful for in vivo imaging where detection of the fluorescent signal in a subject, e.g., a mammal, including a human, indicates that the disease site contains the target and contains a protease that is specific for the CM of the activatable antibody.

These techniques are also useful in kits and/or as reagents for the detection, identification or characterization of protease activity in a variety of cells, tissues, and organisms based on the protease-specific CM in the activatable antibody.

In some embodiments, in situ imaging and/or in vivo imaging are useful in methods to identify which patients to treat. For example, in in situ imaging, the activatable antibodies are used to screen patient samples to identify those patients having the appropriate protease(s) and target(s) at the appropriate location, e.g., at a tumor site.

In some embodiments in situ imaging is used to identify or otherwise refine a patient population suitable for treatment with an activatable antibody of the disclosure. For example, patients that test positive for both the target and a protease that cleaves the substrate in the cleavable moiety (CM) of the activatable antibody being tested (e.g., accumulate activated antibodies at the disease site) are identified as suitable candidates for treatment with such an activatable antibody comprising such a CM. Likewise, patients that test negative for either or both of the target and the protease that cleaves the substrate in the CM in the activatable antibody being tested using these methods are identified as suitable candidates for another form of therapy (i.e., not suitable for treatment with the activatable antibody being tested). In some embodiments, such patients that test negative with respect to a first activatable antibody can be tested with other activatable antibodies comprising different CMs until a suitable activatable antibody for treatment is identified (e.g., an activatable antibody comprising a CM that is cleaved by the patient at the site of disease).

In some embodiments in vivo imaging is used to identify or otherwise refine a patient population suitable for treatment with an activatable antibody of the disclosure. For example, patients that test positive for both the target and a protease that cleaves the substrate in the cleavable moiety (CM) of the activatable antibody being tested (e.g., accumulate activated antibodies at the disease site) are identified as suitable candidates for treatment with such an activatable antibody comprising such a CM. Likewise, patients that test negative are identified as suitable candidates for another form of therapy (i.e., not suitable for treatment with the activatable antibody being tested). In some embodiments, such patients that test negative with respect to a first activatable antibody can be tested with other activatable antibodies comprising different CMs until a suitable activatable antibody for treatment is identified (e.g., an activatable antibody comprising a CM that is cleaved by the patient at the site of disease).

Pharmaceutical Compositions

The antibodies, conjugated antibodies, activatable antibodies and/or conjugated activatable antibodies of the disclosure (also referred to herein as “active compounds”), and derivatives, fragments, analogs and homologs thereof, can be incorporated into pharmaceutical compositions suitable for administration. Such compositions typically comprise the antibody, the conjugated antibody, activatable antibody and/or conjugated activatable antibody and a pharmaceutically acceptable carrier. As used herein, the term “pharmaceutically acceptable carrier” is intended to include any and all solvents, dispersion media, coatings, antibacterial and antifungal agents, isotonic and absorption delaying agents, and the like, compatible with pharmaceutical administration. Suitable carriers are described in the most recent edition of Remington's Pharmaceutical Sciences, a standard reference text in the field, which is incorporated herein by reference. Suitable examples of such carriers or diluents include, but are not limited to, water, saline, ringer's solutions, dextrose solution, and 5% human serum albumin. Liposomes and non-aqueous vehicles such as fixed oils may also be used. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

Pharmaceutical compositions according to the invention can include an antibody/activatable antibody of the invention and a carrier. These pharmaceutical compositions can be included in kits, such as, for example, diagnostic kits.

In some embodiments, the pharmaceutical composition comprises an antibody of the disclosure, an activatable antibody of the disclosure, a conjugated antibody or the disclosure, and/or a conjugated activatable antibody of the disclosure, and a carrier. In some embodiments, the pharmaceutical composition comprises an additional agent. In some embodiments, the additional agent is a therapeutic agent.

A pharmaceutical composition of the disclosure is formulated to be compatible with its intended route of administration. Examples of routes of administration include parenteral, e.g., intravenous, intradermal, subcutaneous, oral (e.g., inhalation), transdermal (i.e., topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid (EDTA); buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In some embodiments, it will be desirable to include isotonic agents, for example, sugars, polyalcohols such as manitol, sorbitol, sodium chloride in the composition. Prolonged absorption of the injectable compositions can be brought about by including in the composition an agent that delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser that contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the disclosure are dictated by and directly dependent on the unique characteristics of the active compound and the particular therapeutic effect to be achieved, and the limitations inherent in the art of compounding such an active compound for the treatment of individuals.

The pharmaceutical compositions can be included in a container, pack, or dispenser together with instructions for administration.

The invention will be further described in the following examples, which do not limit the scope of the invention described in the claims.

### EXAMPLES

### Example 1. Characterization of Anti-Human CD147 Antibodies

The studies provided herein were designed to evaluate binding of anti-human CD147 antibodies of the disclosure.

An anti-human CD147 monoclonal antibody of the present disclosure was obtained using mouse hybridoma technology in accordance with techniques known in the art. Mice were immunized with human CD147 extracellular domain (ECD) and subsequent hybridomas were screened for binding to human CD147 ECD by ELISA and subsequently confirmed to be cytotoxic in a piggyback assay and confirmed to bind cell surfaces by FACS. The mouse CD147 3A11 monoclonal antibody of the present disclosure includes a heavy chain variable region (VH) of SEQ ID NO: 4, and a light chain variable region (VL) of SEQ ID NO: 9, and was used where described herein as a positive control.

The mouse anti-human CD147 monoclonal antibody 3A11 was humanized to form humanized anti-human CD147 heavy chains hu 3A11 Hc1 (VH of SEQ ID NO: 1), hu 3A11 Hc2 (VH of SEQ ID NO: 2), and hu 3A11 Hc3 (VH of SEQ ID NO: 3), as well as humanized anti-3A11 light chains hu 3A11 Lc1 (VL of SEQ ID NO: 5), hu 3A11 Lc2 (VL of SEQ ID NO: 6), hu 3A11 Lc3 (VL of SEQ ID NO: 7), and hu 3A11 Lc4 (VL of SEQ ID NO: 8).

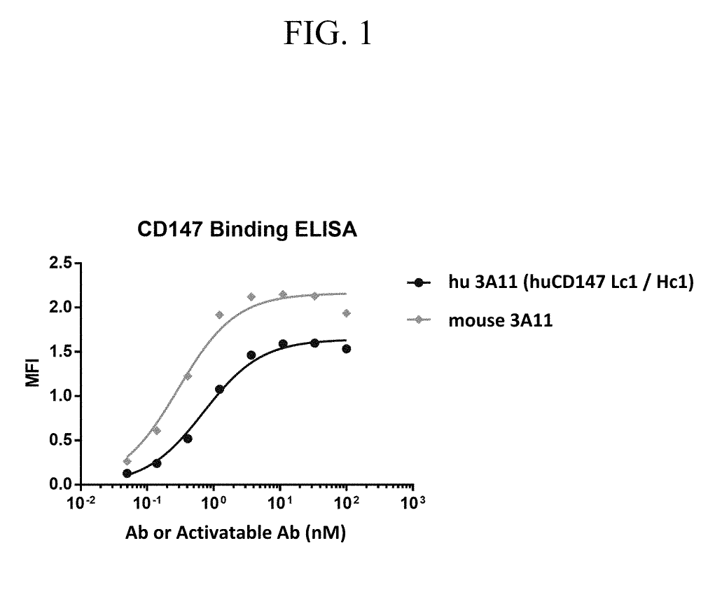

As shown in FIG. 1, the binding affinity of humanized anti-human CD147 monoclonal antibodies of the present disclosure to human CD147 polypeptide was assayed using an ELISA. The antibody of the present disclosure that was assayed in this exemplary study was hu 3A11 Lc1/Hc1 (VL of SEQ ID NO: 5 and VH of SEQ ID NO: 1). Mouse anti-human CD147 monoclonal antibody 3A11 (VL of SEQ ID NO: 8 and VH of SEQ ID NO: 4) was assayed as a reference. Using a standard ELISA protocol, human CD147 protein was absorbed to ELISA plates and subsequently incubated with the indicated concentration of antibody of the present disclosure. Bound humanized antibody of the present disclosure was detected with anti-human Fab-peroxidase secondary antibody and the mouse 3A11 monoclonal antibody was detected with anti-mouse Fc-peroxidase secondary antibody, and Ultra TMB detection (Thermo Fisher Scientific). The apparent equilibrium dissociation constants (K_{D}) of this exemplary binding study are shown in Table 12.

TABLE 12
Equilibrium Dissociation Constants of Mouse Anti-human
CD147 Antibodies to Human CD147

| Cell Species | K_{D} (nM) |
|---|---|
| hu 3A11 (hu Lc1/hu Hc1) | 0.7099 |
| Mouse 3A11 | 0.2930 |

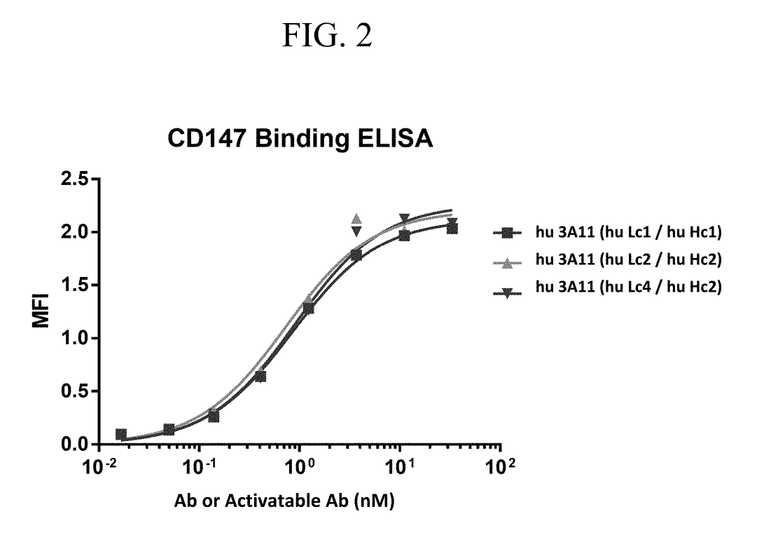

As shown in FIG. 2, the binding affinity of humanized anti-human CD147 monoclonal antibodies of the present disclosure to human CD147 polypeptide were assayed using an ELISA. The antibodies of the present disclosure that were assayed in this exemplary study were hu 3A11 Lc1/hu 3A11 Hc1 (VL of SEQ ID NO: 5 and VH of SEQ ID NO: 1), hu 3A11 Lc2/hu 3A11 Hc2 (VL of SEQ ID NO: 6 and VH of SEQ ID NO: 2), hu 3A11 Lc4/hu 3A11 Hc2 (VL of SEQ ID NO: 8 and VH of SEQ ID NO: 2). Using a standard ELISA protocol, human CD147 protein was absorbed to ELISA plates and subsequently incubated with the indicated concentration of antibody of the present disclosure. Bound humanized antibody of the present disclosure was detected with anti-human Fab-peroxidase secondary antibody, and Ultra TMB detection (Thermo Fisher Scientific). The apparent equilibrium dissociation constants (K_{D}) of this exemplary binding study are shown in Table 13.

TABLE 13
Equilibrium Dissociation Constants of Humanized Anti-human
CD147 Antibodies to Human CD147

| Cell Species | K_{D} (nM) |
|---|---|
| hu 3A11 (hu Lc1/hu Hc1) | 0.8518 |
| hu 3A11 (hu Lc2/hu Hc2) | 0.7261 |
| hu 3A11 (hu Lc4/hu Hc2) | 0.9017 |

### Example 2. Anti-Human CD147 Mediated Cell Line Cytotoxicity

This Example shows that exemplary human cell lines demonstrated sensitivity to anti-human CD147-targeted cytotoxicity.

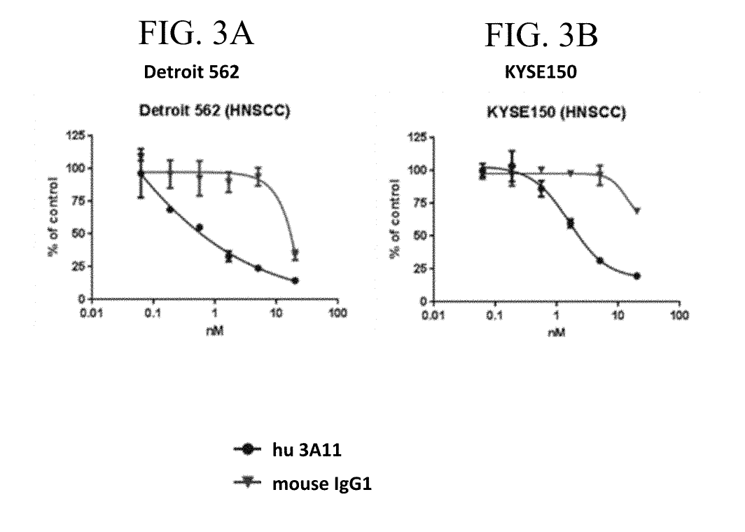

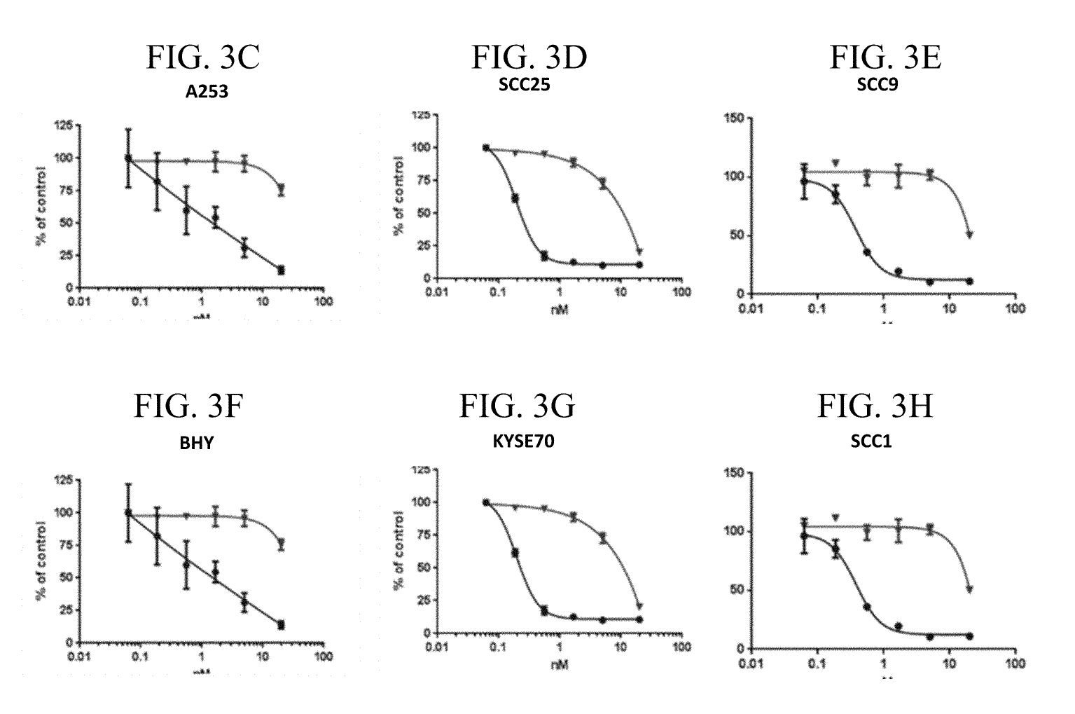

FIGS. 3A-3H show the ability of a humanized anti-human CD147 hu 3A11 Lc1/Hc1 antibody of the present disclosure to induce cytotoxicity in certain cell lines. The cell lines tested in this exemplary assay were derived from various carcinomas (Detroit 562, KYSE150, A253, SCC25, SCC9, BHY, KYSE70, and SCC1). In this exemplary assay, the indicated cell lines were cultured using standard cell culture techniques and plated at densities between 500-1000 cells per well. Humanized anti-human CD147 3A11 Lc1/Hc1 monoclonal antibody of the present disclosure (VH of SEQ ID NO: 1; VL of SEQ ID NO: 5) and anti-human mouse IgG1-MMAE control were applied to the cell lines at the indicated concentrations and vc-MMAE conjugated secondary antibody (mouse anti-human secondary antibody conjugated to vc-MMAE) was added at equal concentration to the anti-human CD147 antibody. Plates were visually monitored for cell death and evaluated for cytotoxicity after 3 to 7 days using Celltiter Glo (Promega). The secondary antibody conjugated to vc-MMAE alone was used a control. As shown in FIGS. 3A-3H, exemplary graphs showing cell viability treated by the anti-human CD147 antibody and vc-MMAE conjugated secondary antibody relative to untreated cells are depicted. The EC50 for multiple exemplary cell lines treated by anti-human CD147 antibodies of the present disclosure are shown in Table 14.

TABLE 14
Anti-human CD147 Mediated Cytotoxicity

| Cell line | Origin | EC50 (nM) |
|---|---|---|
| RT11-2/84 | Bladder cancer | 1.94 |
| HCC 1806 | Triple-negative breast cancer | 0.68 |
| HCC 70 | Triple-negative breast cancer | 0.94 |
| HCC1143 | Triple-negative breast cancer | <1.0 |
| HCC1937 | Triple-negative breast cancer | <1.0 |
| HCC1954 | Triple-negative breast cancer | <1.0 |
| HCT 116 | Colorectal cancer | 1.8 |
| LOVO | Colorectal cancer | <1.0 |
| LS174T | Colorectal cancer | ~1 |
| LS411N | Colorectal cancer | <1.0 |
| SW1417 | Colorectal cancer | 0.8 |
| sw48 | Colorectal cancer | <1.0 |
| sw480 | Colorectal cancer | <1.0 |
| BHY | Head-neck squamous cell | <1.0 |
| | carcinoma | |
| Detroit562 | Head-neck squamous cell | 0.008 |
| | carcinoma | |
| KYSE150 | Head-neck squamous cell | 1.6 |
| | carcinoma | |
| KYSE70 | Head-neck squamous cell | 0.13 |
| | carcinoma | |
| SCC1 | Head-neck squamous cell | 0.14 |
| | carcinoma | |
| SCC25 | Head-neck squamous cell | 0.2 |
| | carcinoma | |
| SCC9 | Head-neck squamous cell | 0.37 |
| | carcinoma | |
| H1975 | Lung cancer | 0.09 |
| H292 | Lung cancer | 0.07 |
| H358 | Lung cancer | 0.09 |
| H520 | Lung cancer | 11.19 |
| H1581 | Non-small cell lung cancer | 0.039 |
| H2444 | Non-small cell lung cancer | 1.8 |
| H2405 | Small cell lung cancer | 0.3 |
| H69 | Small cell lung cancer | ~1.0 |
| EFO-21 | Ovarian cancer | 1.4 |
| SKOV3 | Ovarian cancer | 0.95 |
| aspc1 | Pancreatic cancer | 20.5 |
| HPAC | Pancreatic cancer | 0.21 |
| HPAF2 | Pancreatic cancer | <1.0 |
| miapaca1 | Pancreatic cancer | 0.65 |
| PC3 | Prostate cancer | <1.0 |
| A253 | Salivary gland cancer | <1.0 |

### Example 3. Mask Discovery

The studies provided herein were designed to identify and characterize masking moieties for use in activatable anti-human CD147 antibodies of the disclosure.

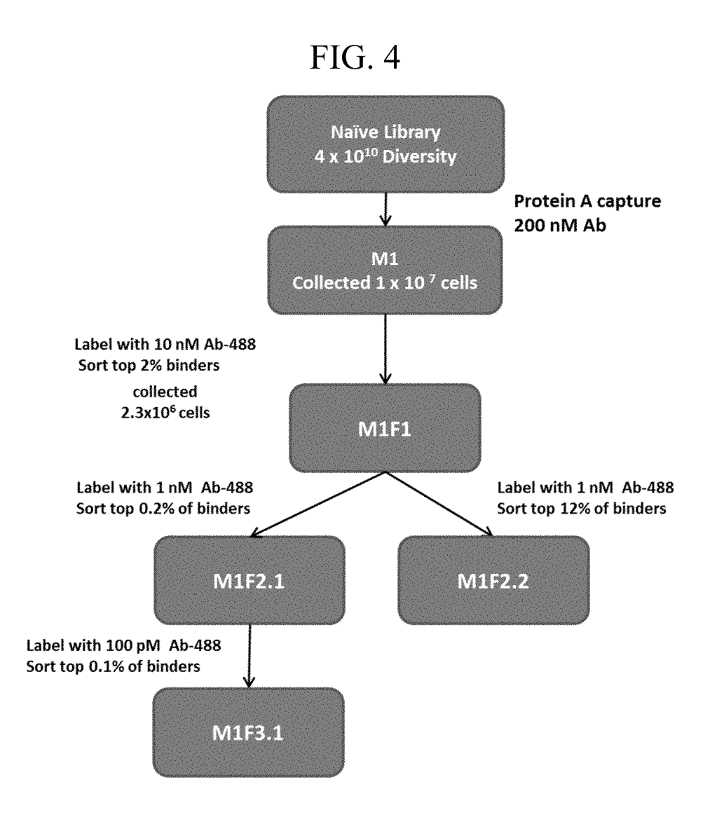

Humanized anti-human CD147 3A11 mouse monoclonal antibody huHc1/huLc1 (VH of SEQ ID NO: 1 and VL of SEQ ID NO: 5) of the present disclosure, was used to screen a random X₁₅ peptide library with a total diversity of 4×10¹⁰, where X is any amino acid, using a method similar to that described in PCT International Publication Number WO 2010/081173, published 15 Jul. 2010. The screening included one round of magnetic-activated cell sorting (MACS) and three rounds of fluorescence-activated cell sorting (FACS). The MACS and FACS sorting scheme is depicted schematically on FIG. 4 The MACS sorting was done with protein-A Dynabeads (Invitrogen) and the anti-human CD147 3A11 antibody were used at a concentration of 200 nM. For the MACS round, approximately 1×10¹² cells were screened for binding and 1×10⁷ cells were collected.

Anti-human CD147 3A11 of the present disclosure was conjugated with AlexaFluor-488 (Invitrogen) using standard protocols. CD147 binding activity was confirmed and anti-human CD147 3A11 conjugated with AlexaFluor-488 (Ab-488) was used as a fluorescent probe for all FACS rounds. Bacterial cells collected from the FACS round were subsequently stained and positive clones were labeled and sorted as follows (and as described in further detail in U.S. Patent Application Publication No. US 2009/0062142): 10 nM 3A11-488 in FACS round 1 collecting the brightest 2% of cells (M1F1), 1 nM 3A11-488 in FACS round 2.1 collecting the top brightest 0.2% of cell (M1F2.1) or brightest 12% of cells (M1F2.2), 100 μM 3A11-488 in FACS round 3.1 with an 8 minute off-rate screening in PBS at room temperature collecting the top 0.1% of cells. Individual clones from the M1F2.1, M1F2.2, and M1F3.1 populations were sequenced and shown in Table 15.

TABLE 15
Anti-human CD147 masking moieties (MM)

| | |
|---|---|
| Clone# | Sequence |

MACS Round M1F2.1

| | | |
|---|---|---|
| JS10215 | RYQSCHSYWCTEGNH | (SEQ ID NO: 30) |
| JS10217 | QSLFCSGFRCDQYAS | (SEQ ID NO: 31) |
| JS10219 | KHGPCHFRLCPQNTS | (SEQ ID NO: 32) |
| JS10221 | DHGPCHYVSCTTINH | (SEQ ID NO: 33) |
| JS10228 | KHGPCHFRLCPQNTS | (SEQ ID NO: 34) |
| JS10247 | MHSYCHYRMCDGHGT | (SEQ ID NO: 35) |
| JS10218 | WQRECSQKNICQYYI | (SEQ ID NO: 36) |
| JS10222 | THGPCHFKPNCSYPT | (SEQ ID NO: 37) |
| JS10229 | NMRWCTPEINCTHHT | (SEQ ID NO: 38) |
| JS10233 | LHGPCHYDLKCKNNT | (SEQ ID NO: 39) |
| JS10235 | LHGPCHFLQFCDKTL | (SEQ ID NO: 40) |
| JS10239 | AHGPCHYNTECNSNK | (SEQ ID NO: 41) |
| JS10241 | WGTFCSAKNICHLYN | (SEQ ID NO: 42) |
| JS10252 | VHSACHYNLNCINNN | (SEQ ID NO: 43) |
| JS10253 | VHGPCHWSVECLSNV | (SEQ ID NO: 44) |
| JS10224 | SMLCVPDTWMCRLAN | (SEQ ID NO: 45) |
| JS10216 | HGPCHYNFNSGCAQF | (SEQ ID NO: 46) |
| JS10243 | LYGCGEFVAERCPRH | (SEQ ID NO: 47) |
| JS10220 | VLCGGMGLKFGTCRM | (SEQ ID NO: 48) |
| JS10251 | PCGDHYYFIKYGCNE | (SEQ ID NO: 49) |
| JS10214 | TDCVCRLWHCCVGVL | (SEQ ID NO: 50) |
| JS10225 | AYPCPCRQQHCCHDQ | (SEQ ID NO: 51) |
| JS10245 | SHTACRYNTCCPHHS | (SEQ ID NO: 52) |

MACS Round M1F2.2

| | | |
|---|---|---|
| JS10276 | THGPCHYKECDWMTI | (SEQ ID NO: 53) |
| JS10283 | DNNVCWKHYCQSQYY | (SEQ ID NO: 54) |
| JS10285 | RYQSCHSYWCTEGNH | (SEQ ID NO: 55) |
| JS10254 | LPQNCHSYYACNFNT | (SEQ ID NO: 56) |
| JS10256 | WQRECSQKNICQYYI | (SEQ ID NO: 57) |
| JS10260 | LDRRCTVEFGCLSSS | (SEQ ID NO: 58) |
| JS10273 | TLQTCHSYFQCTQSQ | (SEQ ID NO: 59) |
| JS10275 | LHGPCHFLQFCDKTL | (SEQ ID NO: 60) |
| JS10261 | PQTCHSYLVTNCNLN | (SEQ ID NO: 61) |
| JS10267 | LQMCHSYFQHSCENR | (SEQ ID NO: 62) |
| JS10274 | VQSCHSYYVAWCHRG | (SEQ ID NO: 63) |
| JS10282 | QQTCHSYYTNYCSQT | (SEQ ID NO: 64) |
| JS10287 | QADCFNQSWMSCLSY | (SEQ ID NO: 65) |
| JS10259 | VWCSSGIPNRACLHH | (SEQ ID NO: 66) |
| JS10284 | HPCKSTPSARQCKYN | (SEQ ID NO: 67) |
| JS10265 | MQCSHSYFIQHYCNK | (SEQ ID NO: 68) |
| JS10271 | VLCGGMGLKFGTCRM | (SEQ ID NO: 69) |
| JS10280 | VLCGWGELRWGECVT | (SEQ ID NO: 70) |
| JS10255 | ECHLRGPHPQHHCNK | (SEQ ID NO: 71) |
| JS10264 | TCLHLTRFNYLSCNK | (SEQ ID NO: 72) |
| JS10268 | DCELFPQNSYHGCIN | (SEQ ID NO: 73) |
| JS10290 | TCERITMHNYIHCPN | (SEQ ID NO: 74) |

MACS Round M1F3.1

| | | |
|---|---|---|
| JS10414 | THGPCHFKPNCSYPT | (SEQ ID NO: 75) |
| JS10419 | LHGPCHYDLKCKNNT | (SEQ ID NO: 76) |
| JS10445 | SHGPCHFDYQCINNT | (SEQ ID NO: 77) |
| JS10443 | AHGPCHYNTECNSNK | (SEQ ID NO: 78) |
| JS10422 | LHGPCHYMNTCHNVK | (SEQ ID NO: 79) |
| JS10415 | LHGPCHFNNCNTLKL | (SEQ ID NO: 80) |
| JS10431 | IHGPCHYNECIMSKN | (SEQ ID NO: 81) |
| JS10428 | WHGPCHYTKCDDHTM | (SEQ ID NO: 82) |
| JS10444 | THGPCHYKECDWMTI | (SEQ ID NO: 83) |
| JS10417 | DHGPCHYVSCTTINH | (SEQ ID NO: 84) |
| JS10446 | LHGLCHYRNCDLPQR | (SEQ ID NO: 85) |
| JS10427 | KHGPCHFRLCPQNTS | (SEQ ID NO: 86) |
| JS10416 | WQRECSQKNICQYYI | (SEQ ID NO: 87) |
| JS10440 | TCLHLTRFNYLSCNK | (SEQ ID NO: 88) |
| JS10423 | FSCGFRGGYMRLCGG | (SEQ ID NO: 89) |

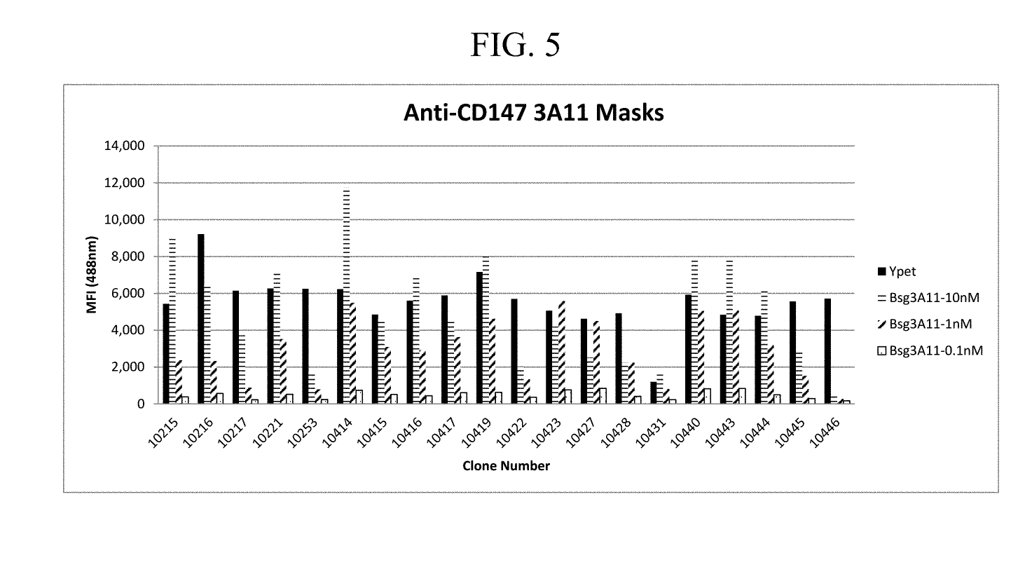

As shown in FIG. 5, the binding affinity of the humanized anti-human CD147 3A11 monoclonal antibody of the present disclosure was tested against individual bacterial clones displaying certain anti-human CD147 masking moiety clones. In this exemplary assay, each bacterial clone was bound with 10 nM, 1 nM, or 0.1 nM of AlexaFluor-488 labeled humanized 3A11 antibody of the present disclosure (VH of SEQ ID NO: 1 and VL of SEQ ID NO: 5). The cells were labeled with yPet (100 nM) to measure surface expression of each clone. The masking moieties that were tested are indicated with the clone number as shown above.

These masking peptides were used to generate anti-human CD147 activatable antibodies of the disclosure. The sequences for certain of these anti-human CD147 activatable antibodies are shown below in Table 17. In some embodiments, these anti-human CD147 activatable antibodies include cleavable moiety 2001 (ISSGLLSGRSDNH; SEQ ID NO: 406), cleavable moiety 3001 (AVGLLAPPGGLSGRSDNH; SEQ ID NO: 412), cleavable moiety 2007 (ISSGLLSGRSDIH; SEQ ID NO: 684), cleavable moiety 2008 (ISSGLLSGRSDQH; SEQ ID NO: 685), cleavable moiety 2011 (ISSGLLSGRSDNP; SEQ ID NO: 688), cleavable moiety 2012 (ISSGLLSGRSANP; SEQ ID NO: 689), cleavable moiety 2013 (ISSGLLSGRSANI; SEQ ID NO: 690), cleavable moiety 3007 (AVGLLAPPGGLSGRSDIH; SEQ ID NO: 692), cleavable moiety 3008 (AVGLLAPPGGLSGRSDQH; SEQ ID NO: 693), cleavable moiety 3011 (AVGLLAPPGGLSGRSDNP; SEQ ID NO: 696), cleavable moiety 3012 (AVGLLAPPGGLSGRSANP; SEQ ID NO: 697), or cleavable moiety 3013 (AVGLLAPPGGLSGRSANI; SEQ ID NO: 698), as indicated.

While certain sequences shown below include the spacer sequence of SEQ ID NO: 645, those of ordinary skill in the art appreciate that the activatable anti-human CD147 antibodies of the disclosure can include any suitable spacer sequence, such as, for example, a spacer sequence selected from the group consisting of QGQSGQG (SEQ ID NO: 645), QGQSGQ (SEQ ID NO: 424), QGQSG (SEQ ID NO: 646), QGQS (SEQ ID NO: 647), QGQ (SEQ ID NO: 648), QG (SEQ ID NO: 649), GQSGQG (SEQ ID NO: 666), QSGQG (SEQ ID NO: 667), SGQG (SEQ ID NO: 668), GQG (SEQ ID NO: 669), G, or Q. In some embodiments, the activatable anti-human CD147 antibodies of the disclosure can have no spacer sequence joined to its N-terminus.

TABLE 16
Anti-human CD147 Antibody Sequences

1. huCD147 3A11 Hc1 Heavy Chain:
Amino Acid sequence
EVQLVESGGGLVQPGGSLRLSCAASGFTFSNYWMNWVRQAPGKGLEWVGEIRLKSYNYATHYAASVKGRF
TISRDDSKNSVYLQMNSLKTEDTAVYYCTAAGTDYWGQGTLVTVSSASTKGPSVFPLAPSSKSTSGGTAA
LGCLVKDYFPEPVTVSWNSGALTSGVHTFPAVLQSSGLYSLSSVVTVPSSSLGTQTYICNVNHKPSNTKV
DKKVEPKSCDKTHTCPPCPAPELLGGPSVFLFPPKPKDTLMISRTPEVTCVVVDVSHEDPEVKFNWYVDG
VEVHNAKTKPREEQYNSTYRVVSVLTVLHQDWLNGKEYKCKVSNKALPAPIEKTISKAKGQPREPQVYTL
PPSREEMTKNQVSLTCLVKGFYPSDIAVEWESNGQPENNYKTTPPVLDSDGSFFLYSKLTVDKSRWQQGN
VFSCSVMHEALHNHYTQKSLSLSPGK (SEQ ID NO: 19)
2. huCD147 3A11 Hc2 Heavy Chain:
Amino Acid sequence
EVQLVESGGGLVQPGGSLRLSCAASGFTFSNYWMNWVRQAPGKGLEWVGEIRLKSYNYATHYAASVKGRF
TISRDDSKNSLYLQMNSLKTEDTAVYYCARAGTDYWGQGTLVTVSSASTKGPSVFPLAPSSKSTSGGTAA
LGCLVKDYFPEPVTVSWNSGALTSGVHTFPAVLQSSGLYSLSSVVTVPSSSLGTQTYICNVNHKPSNTKV
DKKVEPKSCDKTHTCPPCPAPELLGGPSVFLFPPKPKDTLMISRTPEVTCVVVDVSHEDPEVKFNWYVDG
VEVHNAKTKPREEQYNSTYRVVSVLTVLHQDWLNGKEYKCKVSNKALPAPIEKTISKAKGQPREPQVYTL
PPSREEMTKNQVSLTCLVKGFYPSDIAVEWESNGQPENNYKTTPPVLDSDGSFFLYSKLTVDKSRWQQGN
VFSCSVMHEALHNHYTQKSLSLSPGK (SEQ ID NO: 20)
3. huCD147 3A11 Hc3 Light Chain:
Amino Acid sequence
EVQLVESGGGLVQPGGSLRLSCAASGFTFSNYWMNWVRQAPGKGLEWVGEIRLKSYNYATHYVASVKGRF
TISRDDSKNSVYLQMNSLKTEDTAVYYCTAAGTDYWGQGTLLTVSSASTKGPSVFPLAPSSKSTSGGTAA
LGCLVKDYFPEPVTVSWNSGALTSGVHTFPAVLQSSGLYSLSSVVTVPSSSLGTQTYICNVNHKPSNTKV
DKKVEPKSCDKTHTCPPCPAPELLGGPSVFLFPPKPKDTLMISRTPEVTCVVVDVSHEDPEVKFNWYVDG
VEVHNAKTKPREEQYNSTYRVVSVLTVLHQDWLNGKEYKCKVSNKALPAPIEKTISKAKGQPREPQVYTL
PPSREEMTKNQVSLTCLVKGFYPSDIAVEWESNGQPENNYKTTPPVLDSDGSFFLYSKLTVDKSRWQQGN
VFSCSVMHEALHNHYTQKSLSLSPGK (SEQ ID NO: 21)
4. huCD147 3A11 Lc1 Light Chain:
Amino Acid sequence
DIQMTQSPSSLSASVGDRVTITCRASQSVRTDVGWYQQKPGKAPKLLIYYSSNRYTGVPSRFSGSGSGTD
FTLTISSLQPEDFATYYCQQDYSSPYTFGQGTKLEIKRTVAAPSVFIFPPSDEQLKSGTASVVCLLNNFY
PREAKVQWKVDNALQSGNSQESVTEQDSKDSTYSLSSTLTLSKADYEKHKVYACEVTHQGLSSPVTKSFN
RGEC (SEQ ID NO: 23)
5. huCD147 3A11 Lc2 Light Chain:
Amino Acid sequence
DIQMTQSPSSLSASVGDRVTITCKASQSVRTDVAWYQQKPGKAPKLLIYYSSNRYTGVPSRFSGSGSGTD
FTLTISSLQPEDFATYYCQQDYSSPFTFGQGTKLEIKRTVAAPSVFIFPPSDEQLKSGTASVVCLLNNFY
PREAKVQWKVDNALQSGNSQESVTEQDSKDSTYSLSSTLTLSKADYEKHKVYACEVTHQGLSSPVTKSFN
RGEC (SEQ ID NO: 24)
6. huCD147 3A11 Lc3 Light Chain:
Amino Acid sequence
DIQMTQSPSSLSVSVGDRVTITCKASQSVRTDVAWYQQKPGKAPKLLIYYSSNRYTGVPSRFSGSGSGTD
FTFTISSVQPEDFATYYCQQDYSSPFTFGQGTKLEIKRTVAAPSVFIFPPSDEQLKSGTASVVCLLNNFY
PREAKVQWKVDNALQSGNSQESVTEQDSKDSTYSLSSTLTLSKADYEKHKVYACEVTHQGLSSPVTKSFN
RGEC (SEQ ID NO: 25)
7. huCD147 3A11 Lc4 Light Chain:
Amino Acid sequence
DIQMTQSPSSLSVSVGDRVTITCKASQSVRTDVAWYQQKPGKAPKLLIYYSSNRYTGVPSRFSGSGYGTD
FTFTISSVQPEDFATYYCQQDYSSPFTFGQGTKLEIKRTVAAPSVFIFPPSDEQLKSGTASVVCLLNNFY
PREAKVQWKVDNALQSGNSQESVTEQDSKDSTYSLSSTLTLSKADYEKHKVYACEVTHQGLSSPVTKSFN
RGEC (SEQ ID NO: 26)
10. huCD147 3A11 Hc1 VH domain:
Amino Acid sequence
EVQLVESGGGLVQPGGSLRLSCAASGFTFSNYWMDWVRQAPGKGLEWVGEIRLKSYNYATHYAASVKGRF
TISRDDSKNSVYLQMNSLKTEDTAVYYCTAAGTDYWGQGTLVTVSS (SEQ ID NO: 1)
11. huCD147 3A11 Hc2 VH domain:
Amino Acid sequence
EVQLVESGGGLVQPGGSLRLSCAASGFTFSNYWMNWVRQAPGKGLEWVGEIRLKSYNYATHYAASVKGRF
TISRDDSKNSLYLQMNSLKTEDTAVYYCARAGTDYWGQGTLVTVSS (SEQ ID NO: 2)
12. huCD147 3A11 Hc3 VH domain:
Amino Acid sequence
EVQLVESGGGLVQPGGSLRLSCAASGFTFSNYWMNWVRQAPGKGLEWVGEIRLKSYNYATHYVASVKGRF
TISRDDSKNSVYLQMNSLKTEDTAVYYCTAAGTDYWGQGTLLTVSS (SEQ ID NO: 3)
13. huCD147 3A11 Lc1 VL domain:
Amino Acid sequence
DIQMTQSPSSLSASVGDRVTITCRASQSVRTDVGWYQQKPGKAPKLLIYYSSNRYTGVPSRFSGSGSGTD
FTLTISSLQPEDFATYYCQQDYSSPYTFGQGTKLEIK (SEQ ID NO: 5)
14. huCD147 3A11 Lc2 VL domain:
Amino Acid sequence
DIQMTQSPSSLSASVGDRVTITCKASQSVRTDVAWYQQKPGKAPKLLIYYSSNRYTGVPSRFSGSGSGTD
FTLTISSLQPEDFATYYCQQDYSSPFTFGQGTKLEIK (SEQ ID NO: 6)
15. huCD147 3A11 Lc3 VL domain:
Amino Acid sequence
DIQMTQSPSSLSVSVGDRVTITCKASQSVRTDVAWYQQKPGKAPKLLIYYSSNRYTGVPSRFSGSGSGTD
FTFTISSVQPEDFATYYCQQDYSSPFTFGQGTKLEIK (SEQ ID NO: 7)
16. huCD147 3A11 Lc4 VL domain:
Amino Acid sequence
DIQMTQSPSSLSVSVGDRVTITCKASQSVRTDVAWYQQKPGKAPKLLIYYSSNRYTGVPSRFSGSGYGTD
FTFTISSVQPEDFATYYCQODYSSPFTFGQGTKLEIK (SEQ ID NO: 8)
17. mouse 3A11 VH domain:
Amino Acid sequence
EVKLEESGGGLVQPGGSMKLSCVASGFTFSNYWMNWVRQSPEKGLEWVGEIRLKSYNYATHYVESVEGRF
TISRDDSKSSVYLQMNNLRAEDTGIYYCTAAGTDYWGQGTTLTVSS (SEQ ID NO: 4)
18. mouse 3A11 VL domain:
Amino Acid sequence
SIVMTQIPKILLVSAGDRVTITCKASQSVRTDVAWYQQKPGQSPKLLIYYSSNRYTGVPDRFTGSGYGTD
FTFTISTVQAEDLAVYFCQQDYSSPFTFGSGTKLEIK (SEQ ID NO: 9)

TABLE 17
Anti-human CD147 Activatable Antibody Sequences

19. [Spacer (SEQ ID NO: 645)][huCD147 3A11 Lc1-215-3001 VL domain (SEQ
ID NO: 140)]
Amino acid sequence
[QGQSGQG][RYQSCHSYWCTEGNHGGGSSGGSAVGLLAPPGGLSGRSDNHGGSDIQMTQSPSS
LSASVGDRVTITCRASQSVRTDVGWYQQKPGKAPKLLIYYSSNRYTGVPSRFSGSGSGTDFTLT
ISSLQPEDFATYYCQQDYSSPYTFGQGTKLEIK] (SEQ ID NO: 185)
21. [Spacer (SEQ ID NO: 645)][huCD147 3A11 Lc1-216-3001 VL domain (SEQ
ID NO: 141)]
Amino acid sequence
[QGQSGQG][HGPCHYNFNSGCAQFGGGSSGGSAVGLLAPPGGLSGRSDNHGGSDIQMTQSPSS
LSASVGDRVTITCRASQSVRTDVGWYQQKPGKAPKLLIYYSSNRYTGVPSRFSGSGSGTDFTLT
ISSLQPEDFATYYCQQDYSSPYTFGQGTKLEIK] (SEQ ID NO: 186)
22. [Spacer (SEQ ID NO: 645)][huCD147 3A11 Lc1-217-3001 VL domain (SEQ
ID NO: 142)]
Amino acid sequence
[QGQSGQG][QSLFCSGFRCDQYASGGGSSGGSAVGLLAPPGGLSGRSDNHGGSDIQMTQSPSS
LSASVGDRVTITCRASQSVRTDVGWYQQKPGKAPKLLIYYSSNRYTGVPSRFSGSGSGTDFTLT
ISSLQPEDFATYYCQQDYSSPYTFGQGTKLEIK] (SEQ ID NO: 187)
23. [Spacer (SEQ ID NO: 645)][huCD147 3A11 Lc1-221-3001 VL domain (SEQ
ID NO: 143)]
Amino acid sequence
[QGQSGQG][DHGPCHYVSCTTINHGGGSSGGSAVGLLAPPGGLSGRSDNHGGSDIQMTQSPSS
LSASVGDRVTITCRASQSVRTDVGWYQQKPGKAPKLLIYYSSNRYTGVPSRFSGSGSGTDFTLT
ISSLQPEDFATYYCQQDYSSPYTFGQGTKLEIK] (SEQ ID NO: 188)
24. [Spacer (SEQ ID NO: 645)][huCD147 3A11 Lc1-253-3001 VL domain (SEQ
ID NO: 144)]
Amino acid sequence
[QGQSGQG][VHGPCHWSVECLSNVGGGSSGGSAVGLLAPPGGLSGRSDNHGGSDIQMTQSPSS
LSASVGDRVTITCRASQSVRTDVGWYQQKPGKAPKLLIYYSSNRYTGVPSRFSGSGSGTDFTLT
ISSLQPEDFATYYCQQDYSSPYTFGQGTKLEIK] (SEQ ID NO: 189)
25. [Spacer (SEQ ID NO: 645)][huCD147 3A11 Lc1-414-3001 VL domain (SEQ
ID NO: 145)]
Amino acid sequence
[QGQSGQG][THGPCHFKPNCSYPTGGGSSGGSAVGLLAPPGGLSGRSDNHGGSDIQMTQSPSS
LSASVGDRVTITCRASQSVRTDVGWYQQKPGKAPKLLIYYSSNRYTGVPSRFSGSGSGTDFTLT
ISSLQPEDFATYYCQQDYSSPYTFGQGTKLEIK] (SEQ ID NO: 190)
26. [Spacer (SEQ ID NO: 645)][huCD147 3A11 Lc1-415-3001 VL domain (SEQ
ID NO: 146)]
Amino acid sequence
[QGQSGQG][LHGPCHFNNCNTLKLGGGSSGGSAVGLLAPPGGLSGRSDNHGGSDIQMTQSPSS
LSASVGDRVTITCRASQSVRTDVGWYQQKPGKAPKLLIYYSSNRYTGVPSRFSGSGSGTDFTLT
ISSLQPEDFATYYCQQDYSSPYTFGQGTKLEIK] (SEQ ID NO: 191)
27. [Spacer (SEQ ID NO: 645)][huCD147 3A11 Lc1-416-3001 VL domain (SEQ
ID NO: 147)]
Amino acid sequence
[QGQSGQG][WQRECSQKNICOYYIGGGSSGGSAVGLLAPPGGLSGRSDNHGGSDIQMTQSPSS
LSASVGDRVTITCRASQSVRTDVGWYQQKPGKAPKLLIYYSSNRYTGVPSRFSGSGSGTDFTLT
ISSLQPEDFATYYCQQDYSSPYTFGQGTKLEIK] (SEQ ID NO: 192)
28. [Spacer (SEQ ID NO: 645)][huCD147 3A11 Lc1-419-3001 VL domain (SEQ
ID NO: 148)]
Amino acid sequence
[QGQSGQG][LHGPCHYDLKCKNNTGGGSSGGSAVGLLAPPGGLSGRSDNHGGSDIQMTQSPSS
LSASVGDRVTITCRASQSVRTDVGWYQQKPGKAPKLLIYYSSNRYTGVPSRFSGSGSGTDFTLT
ISSLQPEDFATYYCQQDYSSPYTFGQGTKLEIK] (SEQ ID NO: 193)
29. [Spacer (SEQ ID NO: 645)][huCD147 3A11 Lc1-422-3001 VL domain (SEQ
ID NO: 149)]
Amino acid sequence
[QGQSGQG][LHGPCHYMNTCHNVKGGGSSGGSAVGLLAPPGGLSGRSDNHGGSDIQMTQSPSS
LSASVGDRVTITCRASQSVRTDVGWYQQKPGKAPKLLIYYSSNRYTGVPSRFSGSGSGTDFTLT
ISSLQPEDFATYYCQQDYSSPYTFGQGTKLEIK] (SEQ ID NO: 194)
30. [Spacer (SEQ ID NO: 645)][huCD147 3A11 Lc1-423-3001 VL domain (SEQ
ID NO: 150)]
Amino acid sequence
[QGQSGQG][FSCGFRGGYMRLCGGGGGSSGGSAVGLLAPPGGLSGRSDNHGGSDIQMTQSPSS
LSASVGDRVTITCRASQSVRTDVGWYQQKPGKAPKLLIYYSSNRYTGVPSRFSGSGSGTDFTLT
ISSLQPEDFATYYCQQDYSSPYTFGQGTKLEIK] (SEQ ID NO: 195)
31. [Spacer (SEQ ID NO: 645)][huCD147 3A11 Lc1-427-3001 VL domain (SEQ
ID NO: 151)]
Amino acid sequence
[QGQSGQG][KHGPCHFRLCPQNTSGGGSSGGSAVGLLAPPGGLSGRSDNHGGSDIQMTQSPSS
LSASVGDRVTITCRASQSVRTDVGWYQQKPGKAPKLLIYYSSNRYTGVPSRFSGSGSGTDFTLT
ISSLQPEDFATYYCQQDYSSPYTFGQGTKLEIK] (SEQ ID NO: 196)
32. [Spacer (SEQ ID NO: 645)][huCD147 3A11 Lc1-428-3001 VL domain (SEQ
ID NO: 152)]
Amino acid sequence
[QGQSGQG][WHGPCHYTKCDDHTMGGGSSGGSAVGLLAPPGGLSGRSDNHGGSDIQMTQSPSS
LSASVGDRVTITCRASQSVRTDVGWYQQKPGKAPKLLIYYSSNRYTGVPSRFSGSGSGTDFTLT
ISSLQPEDFATYYCQQDYSSPYTFGQGTKLEIK] (SEQ ID NO: 197)
33. [Spacer (SEQ ID NO: 645)][huCD147 3A11 Lc1-440-3001 VL domain (SEQ
ID NO: 153)]
Amino acid sequence
[QGQSGQG][TCLHLTRFNYLSCNKGGGSSGGSAVGLLAPPGGLSGRSDNHGGSDIQMTQSPSS
LSASVGDRVTITCRASQSVRTDVGWYQQKPGKAPKLLIYYSSNRYTGVPSRFSGSGSGTDFTLT
ISSLQPEDFATYYCQQDYSSPYTFGQGTKLEIK] (SEQ ID NO: 198)
34. [Spacer (SEQ ID NO: 645)][huCD147 3A11 Lc1-443-3001 VL domain (SEQ
ID NO: 154)]
Amino acid sequence
[QGQSGQG][AHGPCHYNTECNSNKGGGSSGGSAVGLLAPPGGLSGRSDNHGGSDIQMTQSPSS
LSASVGDRVTITCRASQSVRTDVGWYQQKPGKAPKLLIYYSSNRYTGVPSRFSGSGSGTDFTLT
ISSLQPEDFATYYCQQDYSSPYTFGQGTKLEIK] (SEQ ID NO: 199)
35. [Spacer (SEQ ID NO: 645)][huCD147 3A11 Lc1-444-3001 VL domain (SEQ
ID NO: 155)]
Amino acid sequence
[QGQSGQG][THGPCHYKECDWMTIGGGSSGGSAVGLLAPPGGLSGRSDNHGGSDIQMTQSPSS
LSASVGDRVTITCRASQSVRTDVGWYQQKPGKAPKLLIYYSSNRYTGVPSRFSGSGSGTDFTLT
ISSLQPEDFATYYCQQDYSSPYTFGQGTKLEIK] (SEQ ID NO: 200)
36. [Spacer (SEQ ID NO: 645)][huCD147 3A11 Lc1-445-3001 VL domain (SEQ
ID NO: 156)]
Amino acid sequence
[QGQSGQG][SHGPCHFDYQCINNTGGGSSGGSAVGLLAPPGGLSGRSDNHGGSDIQMTQSPSS
LSASVGDRVTITCRASQSVRTDVGWYQQKPGKAPKLLIYYSSNRYTGVPSRFSGSGSGTDFTLT
ISSLQPEDFATYYCQQDYSSPYTFGQGTKLEIK] (SEQ ID NO: 201)

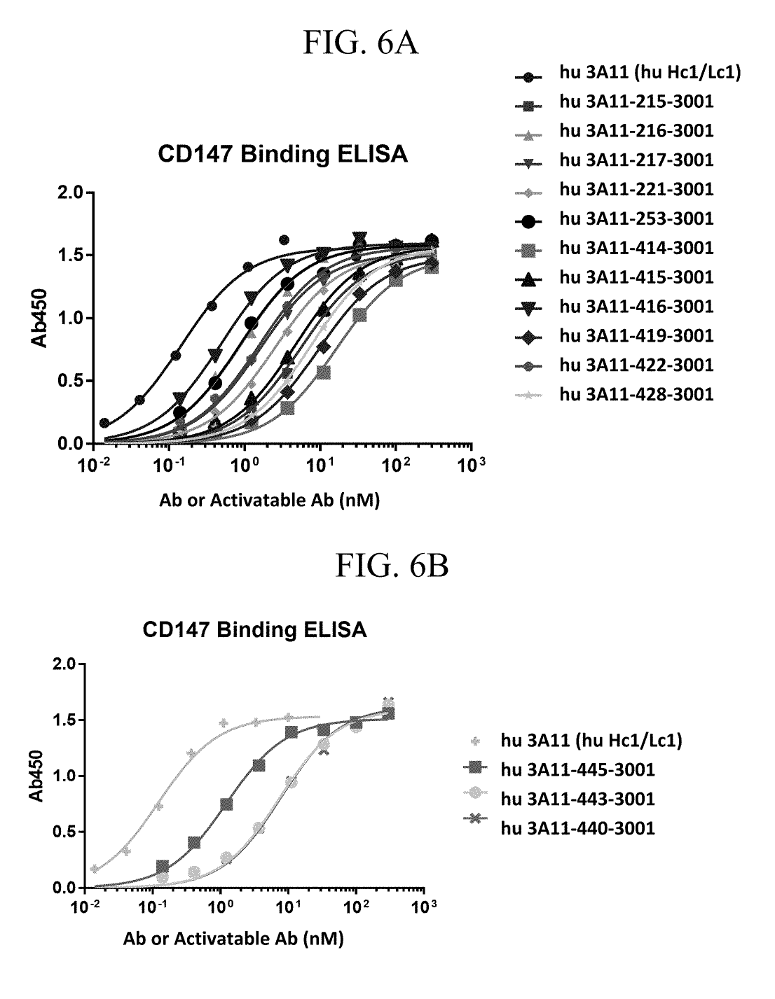

As shown in FIGS. 6A and 6B, the binding affinity of the anti-human CD147 activatable antibodies of the present disclosure to human CD147 polypeptide were assayed using an ELISA. The activatable antibodies of the present disclosure that were assayed in this exemplary study were (a) hu 3A11 Hc1/Lc1-215-3001 (VL of SEQ ID NO: 140 and VH of SEQ ID NO: 1), (b) hu 3A11 Hc1/Lc1-216-3001 (VL of SEQ ID NO: 141 and VH of SEQ ID NO: 1), (c) hu 3A11 Hc1/Lc1-217-3001 (VL of SEQ ID NO: 142 and VH of SEQ ID NO: 1), (d) hu 3A11 Hc1/Lc1-221-3001 (VL of SEQ ID NO: 143 and VH of SEQ ID NO: 1), (e) hu 3A11 Hc1/Lc1-253-3001 (VL of SEQ ID NO: 144 and VH of SEQ ID NO: 1), (f) hu 3A11 Hc1/Lc1-414-3001 (VL of SEQ ID NO: 145 and VH of SEQ ID NO: 1), (g) hu 3A11 Hc1/Lc1-415-3001 (VL of SEQ ID NO: 146 and VH of SEQ ID NO: 1), (h) hu 3A11 Hc1/Lc1-416-3001 (VL of SEQ ID NO: 147 and VH of SEQ ID NO: 1), (i) hu 3A11 Hc1/Lc1-419-3001 (VL of SEQ ID NO: 148 and VH of SEQ ID NO: 1), (j) hu 3A11 He 1/Lc1-422-3001 (VL of SEQ ID NO: 149 and VH of SEQ ID NO: 1), (k) hu 3A11 Hc1/Lc1-428-3001 (VL of SEQ ID NO: 152 and VH of SEQ ID NO: 1), (l) hu 3A11 Hc1/Lc1-445-3001 (VL of SEQ ID NO: 156 and VH of SEQ ID NO: 1), (m) hu 3A11 He 1/Lc1-443-3001 (VL of SEQ ID NO: 154 and VH of SEQ ID NO: 1), and (n) hu 3A11 Hc1/Lc1-440-3001 (VL of SEQ ID NO: 153 and VH of SEQ ID NO: 1). Humanized anti-human CD147 antibodies of the present disclosure hu 3A11 Hc1/Lc1 (VL of SEQ ID NO: 5 and VH of SEQ ID NO: 1) were used as a control.

Using a standard ELISA protocol, human CD147 protein was absorbed to ELISA plates and subsequently incubated with the indicated concentration of antibody of the present disclosure. Bound humanized activatable antibody of the present disclosure was detected with anti-human Fab-peroxidase secondary antibody and Ultra TMB detection (Thermo Fisher Scientific). The apparent dissociation constants of this exemplary binding study are shown in Table 18

TABLE 18
Equilibrium Dissociation Constants of Anti-human CD147
Activatable Antibodies To Human CD147

| Ab | K_{D} (nM) |
|---|---|
| hu 3A11 (hu Hc1/Lc1) | 0.14 |
| hu 3A11-215-3001 | 5.53 |
| hu 3A11-216-3001 | 0.89 |
| hu 3A11-217-3001 | 1.61 |
| hu 3A11-221-3001 | 2.50 |
| hu 3A11-253-3001 | 0.87 |
| hu 3A11-414-3001 | 15.97 |
| hu 3A11-415-3001 | 4.72 |
| hu 3A11-416-3001 | 0.49 |
| hu 3A11-419-3001 | 9.43 |
| hu 3A11-422-3001 | 1.55 |
| hu 3A11-428-3001 | 7.63 |
| hu 3A11-445-3001 | 7.86 |
| hu 3A11-443-3001 | 7.40 |
| hu 3A11-440-3001 | 1.23 |

### Example 4. Mask Glycosylation Mutants and Truncation Evaluation

The studies provided herein were designed to identify and characterize masking moieties with glycosylation mutants and truncated masking moieties for use in activatable anti-human CD147 antibodies of the disclosure.

As shown in Table 19, variants of the JS10414 and JS10419 masking mutants were constructed that included either amino acid substitutions to remove potential glycosylation sites, or variants that were truncations of the isolated masking moieties.

TABLE 19
Anti-human CD147 truncated masking moieties (MM)

| Clone# | Sequence | |
|---|---|---|
| JS10414 | THGPCHFKPNCSYPT | (SEQ ID NO: 75) |
| JS10414.1 | THGPCHFKPNC | (SEQ ID NO: 90) |
| JS10414.2 | THGPCHFKPQCSYPT | (SEQ ID NO: 91) |
| JS10414.3 | THGPCHFKPNCAYPT | (SEQ ID NO: 92) |
| JS10414.4 | THGPCHFRPNCAYPT | (SEQ ID NO: 93) |
| JS10419 | LHGPCHYDLKCKNNT | (SEQ ID NO: 76) |
| JS10419.1 | LHGPCHYDLKCKQNT | (SEQ ID NO: 94) |
| JS10419.2 | LHGPCHYDLKCKNN | (SEQ ID NO: 95) |
| JS10419.3 | LHGPCHYDLKCK | (SEQ ID NO: 96) |
| JS10419.4 | LHGPCHYDLKC | (SEQ ID NO: 97) |
| JS10415.5 | LHGPCHYDLRC | (SEQ ID NO: 98) |
| JS10415 | LHGPCHFNNCNTLKL | (SEQ ID NO: 80) |
| JS10415.1 | LHGPCHFNNCNTL | (SEQ ID NO: 99) |
| JS10440 | TCLHLTRFNYLSCNK | (SEQ ID NO: 88) |
| JS10440.1 | TCLHLTRFNYLSC | (SEQ ID NO: 100) |

As shown in Table 20, activatable antibodies of the present invention were constructed to include certain of the masking moiety variants of the present invention.

TABLE 20
Anti-human CD147 Activatable Antibody Sequences

37. [Spacer (SEQ ID NO: 645)][huCD147 3A11 Lc1-414.1-3001 VL
domain (SEQ ID NO: 157)]
Amino acid sequence
[QGQSGQG][THGPCHFKPNCGGGSSGGSAVGLLAPPGGLSGRSDNHGGSDIQMTQSPSSLSAS
VGDRVTITCRASQSVRTDVGWYQQKPGKAPKLLIYYSSNRYTGVPSRFSGSGSGTDFTLTISSL
QPEDFATYYCQQDYSSPYTFGQGTKLEIK] (SEQ ID NO: 202)
38. [Spacer (SEQ ID NO: 645)][huCD147 3A11 Lc1-414.2-3001 VL
domain (SEQ ID NO: 158)]
Amino acid sequence
[QGQSGQG][THGPCHFKPQCSYPTGGGSSGGSAVGLLAPPGGLSGRSDNHGGSDIQMTQSPSS
LSASVGDRVTITCRASQSVRTDVGWYQQKPGKAPKLLIYYSSNRYTGVPSRFSGSGSGTDFTLT
ISSLQPEDFATYYCQQDYSSPYTFGQGTKLEIK] (SEQ ID NO: 203)
39. [Spacer (SEQ ID NO: 645)][huCD147 3A11 Lc1-414.3-3001 VL
domain (SEQ ID NO: 159)]
Amino acid sequence
[QGQSGQG][THGPCHFKPNCAYPTGGGSSGGSAVGLLAPPGGLSGRSDNHGGSDIQMTQSPSS
LSASVGDRVTITCRASQSVRTDVGWYQQKPGKAPKLLIYYSSNRYTGVPSRFSGSGSGTDFTLT
ISSLQPEDFATYYCQQDYSSPYTFGQGTKLEIK] (SEQ ID NO: 204)
40. [Spacer (SEQ ID NO: 645)][huCD147 3A11 Lc1-419.1-3001 VL
domain (SEQ ID NO: 160)]
Amino acid sequence
[QGQSGQG][LHGPCHYDLKCKQNTGGGSSGGSAVGLLAPPGGLSGRSDNHGGSDIQMTQSPSS
LSASVGDRVTITCRASQSVRTDVGWYQQKPGKAPKLLIYYSSNRYTGVPSRFSGSGSGTDFTLT
ISSLQPEDFATYYCQQDYSSPYTFGQGTKLEIK] (SEQ ID NO: 205)
41. [Spacer (SEQ ID NO: 645)][huCD147 3A11 Lc1-419.2-3001 VL
domain (SEQ ID NO: 161)]
Amino acid sequence
[QGQSGQG][LHGPCHYDLKCKNNGGGSSGGSAVGLLAPPGGLSGRSDNHGGSDIQMTQSPSSL
SASVGDRVTITCRASQSVRTDVGWYQQKPGKAPKLLIYYSSNRYTGVPSRFSGSGSGTDFTLTI
SSLQPEDFATYYCQQDYSSPYTFGQGTKLEIK] (SEQ ID NO: 206)
42. [Spacer (SEQ ID NO: 645)][huCD147 3A11 Lc1-419.3-3001 VL
domain (SEQ ID NO: 162)]
Amino acid sequence
[QGQSGQG][LHGPCHYDLKCKGGGSSGGSAVGLLAPPGGLSGRSDNHGGSDIQMTQSPSSLSA
SVGDRVTITCRASQSVRTDVGWYQQKPGKAPKLLIYYSSNRYTGVPSRFSGSGSGTDFTLTISS
LQPEDFATYYCQQDYSSPYTFGQGTKLEIK] (SEQ ID NO: 207)
43. [Spacer (SEQ ID NO: 645)][huCD147 3A11 Lc1-414.3-2012 VL
domain (SEQ ID NO: 163)]
Amino acid sequence
[QGQSGQG][THGPCHFKPNCAYPTGGGSSGGSISSGLLSGRSANPGGGSDIQMTQSPSSLSAS
VGDRVTITCRASQSVRTDVGWYQQKPGKAPKLLIYYSSNRYTGVPSRFSGSGSGTDFTLTISSL
QPEDFATYYCQQDYSSPYTFGQGTKLEIK] (SEQ ID NO: 208)
44. [Spacer (SEQ ID NO: 645)][huCD147 3A11 Lc1-414.4-2012 VL
domain (SEQ ID NO: 164)]
Amino acid sequence
[QGQSGQG][THGPCHFRPNCAYPTGGGSSGGSISSGLLSGRSANPGGGSDIQMTQSPSSLSAS
VGDRVTITCRASQSVRTDVGWYQQKPGKAPKLLIYYSSNRYTGVPSRFSGSGSGTDFTLTISSL
QPEDFATYYCQQDYSSPYTFGQGTKLEIK] (SEQ ID NO: 209)
45. [Spacer (SEQ ID NO: 645)][huCD147 3A11 Lc1-415-2012 VL
domain (SEQ ID NO: 165)]
Amino acid sequence
[QGQSGQG][LHGPCHFNNCNTLKLGGGSSGGSISSGLLSGRSANPGGGSDIQMTQSPSSLSAS
VGDRVTITCRASQSVRTDVGWYQQKPGKAPKLLIYYSSNRYTGVPSRFSGSGSGTDFTLTISSL
QPEDFATYYCQQDYSSPYTFGQGTKLEIK] (SEQ ID NO: 210)
46. [Spacer (SEQ ID NO: 645)][huCD147 3A11 Lc1-415.1-2012 VL
domain (SEQ ID NO: 166)]
Amino acid sequence
[QGQSGQG][LHGPCHFNNCNTLGGGSSGGSISSGLLSGRSANPGGGSDIQMTQSPSSLSASVG
DRVTITCRASQSVRTDVGWYQQKPGKAPKLLIYYSSNRYTGVPSRFSGSGSGTDFTLTISSLQP
EDFATYYCQQDYSSPYTFGQGTKLEIK] (SEQ ID NO: 211)
46. [Spacer (SEQ ID NO: 645)][huCD147 3A11 Lc1-419.3-2012 VL
domain (SEQ ID NO: 167)]
Amino acid sequence
[QGQSGQG][LHGPCHYDLKCKGGGSSGGSISSGLLSGRSANPGGGSDIQMTQSPSSLSASVGD
RVTITCRASQSVRTDVGWYQQKPGKAPKLLIYYSSNRYTGVPSRFSGSGSGTDFTLTISSLQPE
DFATYYCQQDYSSPYTFGQGTKLEIK] (SEQ ID NO: 212)
47. [Spacer (SEQ ID NO: 645)][huCD147 3A11 Lc1-419.4-2012 VL
domain (SEQ ID NO: 168)]
Amino acid sequence
[QGQSGQG][LHGPCHYDLKCGGGSSGGSISSGLLSGRSANPGGGSDIQMTQSPSSLSASVGDR
VTITCRASQSVRTDVGWYQQKPGKAPKLLIYYSSNRYTGVPSRFSGSGSGTDFTLTISSLQPED
FATYYCQQDYSSPYTFGQGTKLEIK] (SEQ ID NO: 213)
48. [Spacer (SEQ ID NO: 645)][huCD147 3A11 Lc1-419.5-2012 VL
domain (SEQ ID NO: 169)]
Amino acid sequence
[QGQSGQG][LHGPCHYDLRCGGGSSGGSISSGLLSGRSANPGGGSDIQMTQSPSSLSASVGDR
VTITCRASQSVRTDVGWYQQKPGKAPKLLIYYSSNRYTGVPSRFSGSGSGTDFTLTISSLQPED
FATYYCQQDYSSPYTFGQGTKLEIK] (SEQ ID NO: 214)
49. [Spacer (SEQ ID NO: 645)][huCD147 3A11 Lc1-440-2012 VL
domain (SEQ ID NO: 170)]
Amino acid sequence
[QGQSGQG][TCLHLTRFNYLSCNKGGGSSGGSISSGLLSGRSANPGGGSDIQMTQSPSSLSAS
VGDRVTITCRASQSVRTDVGWYQQKPGKAPKLLIYYSSNRYTGVPSRFSGSGSGTDFTLTISSL
QPEDFATYYCQQDYSSPYTFGQGTKLEIK] (SEQ ID NO: 215)
50. [Spacer (SEQ ID NO: 645)][huCD147 3A11 Lc1-440.1-2012 VL
domain (SEQ ID NO: 171)]
Amino acid sequence
[QGQSGQG][TCLHLTRFNYLSCGGGSSGGSISSGLLSGRSANPGGGSDIQMTQSPSSLSASVG
DRVTITCRASQSVRTDVGWYQQKPGKAPKLLIYYSSNRYTGVPSRFSGSGSGTDFTLTISSLQP
EDFATYYCQQDYSSPYTFGQGTKLEIK] (SEQ ID NO: 216)
51. [Spacer (SEQ ID NO: 645)][huCD147 3A11 Lc1-215-3001 (SEQ ID
NO: 230)]
Amino acid sequence
[QGQSGQG][RYQSCHSYWCTEGNHGGGSSGGSAVGLLAPPGGLSGRSDNHGGSDIQMTQSPSS
LSASVGDRVTITCRASQSVRTDVGWYQQKPGKAPKLLIYYSSNRYTGVPSRFSGSGSGTDFTLT
ISSLQPEDFATYYCQQDYSSPYTFGQGTKLEIKRTVAAPSVFIFPPSDEQLKSGTASVVCLLNN
FYPREAKVQWKVDNALQSGNSQESVTEQDSKDSTYSLSSTLTLSKADYEKHKVYACEVTHQGLS
SPVTKSFNRGEC] (SEQ ID NO: 285)
52. [Spacer (SEQ ID NO: 645)][huCD147 3A11 Lc1-216-3001 (SEQ ID
NO: 231)]
Amino acid sequence
[QGQSGQG][HGPCHYNFNSGCAQFGGGSSGGSAVGLLAPPGGLSGRSDNHGGSDIQMTQSPSS
LSASVGDRVTITCRASQSVRTDVGWYQQKPGKAPKLLIYYSSNRYTGVPSRFSGSGSGTDFTLT
ISSLQPEDFATYYCQQDYSSPYTFGQGTKLEIKRTVAAPSVFIFPPSDEQLKSGTASVVCLLNN
FYPREAKVQWKVDNALQSGNSQESVTEQDSKDSTYSLSSTLTLSKADYEKHKVYACEVTHQGLS
SPVTKSFNRGEC] (SEQ ID NO: 286)
53. [Spacer (SEQ ID NO: 645)][huCD147 3A11 Lc1-217-3001 (SEQ ID
NO: 232)]
Amino acid sequence
[QGQSGQG][QSLFCSGFRCDQYASGGGSSGGSAVGLLAPPGGLSGRSDNHGGSDIQMTQSPSS
LSASVGDRVTITCRASQSVRTDVGWYQQKPGKAPKLLIYYSSNRYTGVPSRFSGSGSGTDFTLT
ISSLQPEDFATYYCQQDYSSPYTFGQGTKLEIKRTVAAPSVFIFPPSDEQLKSGTASVVCLLNN
FYPREAKVQWKVDNALQSGNSQESVTEQDSKDSTYSLSSTLTLSKADYEKHKVYACEVTHQGLS
SPVTKSFNRGEC] (SEQ ID NO: 287)
54. [Spacer (SEQ ID NO: 645)][huCD147 3A11 Lc1-221-3001 (SEQ ID
NO: 233)]
Amino acid sequence
[QGQSGQG][DHGPCHYVSCTTINHGGGSSGGSAVGLLAPPGGLSGRSDNHGGSDIQMTQSPSS
LSASVGDRVTITCRASQSVRTDVGWYQQKPGKAPKLLIYYSSNRYTGVPSRFSGSGSGTDFTLT
ISSLQPEDFATYYCQQDYSSPYTFGQGTKLEIKRTVAAPSVFIFPPSDEQLKSGTASVVCLLNN
FYPREAKVQWKVDNALQSGNSQESVTEQDSKDSTYSLSSTLTLSKADYEKHKVYACEVTHQGLS
SPVTKSFNRGEC] (SEQ ID NO: 288)
55. [Spacer (SEQ ID NO: 645)][huCD147 3A11 Lc1-253-3001 (SEQ ID
NO: 234)]
Amino acid sequence
[QGQSGQG][VHGPCHWSVECLSNVGGGSSGGSAVGLLAPPGGLSGRSDNHGGSDIQMTQSPSS
LSASVGDRVTITCRASQSVRTDVGWYQQKPGKAPKLLIYYSSNRYTGVPSRFSGSGSGTDFTLT
ISSLQPEDFATYYCQQDYSSPYTFGQGTKLEIKRTVAAPSVFIFPPSDEQLKSGTASVVCLLNN
FYPREAKVQWKVDNALQSGNSOESVTEQDSKDSTYSLSSTLTLSKADYEKHKVYACEVTHQGLS
SPVTKSFNRGEC] (SEQ ID NO: 289)
56. [Spacer (SEQ ID NO: 645)][huCD147 3A11 Lc1-414-3001 (SEQ ID
NO: 235)]
Amino acid sequence
[QGQSGQG][THGPCHFKPNCSYPTGGGSSGGSAVGLLAPPGGLSGRSDNHGGSDIQMIQSPSS
LSASVGDRVTITCRASQSVRTDVGWYQQKPGKAPKLLIYYSSNRYTGVPSRFSGSGSGTDFTLT
ISSLQPEDFATYYCQQDYSSPYTFGQGTKLEIKRTVAAPSVFIFPPSDEQLKSGTASVVCLLNN
FYPREAKVQWKVDNALQSGNSQESVTEQDSKDSTYSLSSTLTLSKADYEKHKVYACEVTHQGLS
SPVTKSFNRGEC] (SEQ ID NO: 280)
57. [Spacer (SEQ ID NO: 645)][huCD147 3A11 Lc1-415-3001 (SEQ ID
NO: 236)]
Amino acid sequence
[QGQSGQG][LHGPCHFNNCNTLKLGGGSSGGSAVGLLAPPGGLSGRSDNHGGSDIQMTQSPSS
LSASVGDRVTITCRASQSVRTDVGWYQQKPGKAPKLLIYYSSNRYTGVPSRFSGSGSGTDFTLT
ISSLQPEDFATYYCQQDYSSPYTFGQGTKLEIKRTVAAPSVFIFPPSDEQLKSGTASVVCLLNN
FYPREAKVQWKVDNALQSGNSQESVTEQDSKDSTYSLSSTLTLSKADYEKHKVYACEVTHQGLS
SPVTKSFNRGEC] (SEQ ID NO: 281)
58. [Spacer (SEQ ID NO: 645)][huCD147 3A11 Lc1-416-3001 (SEQ ID
NO: 237)]
Amino acid sequence
[QGQSGQG][WQRECSQKNICQYYIGGGSSGGSAVGLLAPPGGLSGRSDNHGGSDIQMTQSPSS
LSASVGDRVTITCRASQSVRTDVGWYQQKPGKAPKLLIYYSSNRYTGVPSRFSGSGSGTDFTLT
ISSLQPEDFATYYCQQDYSSPYTFGQGTKLEIKRTVAAPSVFIFPPSDEQLKSGTASVVCLLNN
FYPREAKVQWKVDNALQSGNSQESVTEQDSKDSTYSLSSTLTLSKADYEKHKVYACEVTHQGLS
SPVTKSFNRGEC] (SEQ ID NO: 282)
59. [Spacer (SEQ ID NO: 645)][huCD147 3A11 Lc1-419-3001 (SEQ ID
NO: 238)]
Amino acid sequence
[QGQSGQG][LHGPCHYDLKCKNNTGGGSSGGSAVGLLAPPGGLSGRSDNHGGSDIQMTQSPSS
LSASVGDRVTITCRASQSVRTDVGWYQQKPGKAPKLLIYYSSNRYTGVPSRFSGSGSGTDFTLT
ISSLQPEDFATYYCQQDYSSPYTFGQGTKLEIKRTVAAPSVFIFPPSDEQLKSGTASVVCLLNN
FYPREAKVQWKVDNALQSGNSQESVTEQDSKDSTYSLSSTLTLSKADYEKHKVYACEVTHQGLS
SPVTKSFNRGEC] (SEQ ID NO: 283)
60. [Spacer (SEQ ID NO: 645)][huCD147 3A11 Lc1-422-3001 (SEQ ID
NO: 239)]
Amino acid sequence
[QGQSGQG][LHGPCHYMNTCHNVKGGGSSGGSAVGLLAPPGGLSGRSDNHGGSDIQMTQSPSS
LSASVGDRVTITCRASQSVRTDVGWYQQKPGKAPKLLIYYSSNRYTGVPSRFSGSGSGTDFTLT
ISSLQPEDFATYYCQQDYSSPYTFGQGTKLEIKRTVAAPSVFIFPPSDEQLKSGTASVVCLLNN
FYPREAKVQWKVDNALQSGNSQESVTEQDSKDSTYSLSSTLTLSKADYEKHKVYACEVTHQGLS
SPVTKSFNRGEC] (SEQ ID NO: 284)
61. [Spacer (SEQ ID NO: 645)][huCD147 3A11 Lc1-423-3001 (SEQ ID
NO: 240)]
Amino acid sequence
[QGQSGQG][FSCGFRGGYMRLCGGGGGSSGGSAVGLLAPPGGLSGRSDNHGGSDIQMTQSPSS
LSASVGDRVTITCRASQSVRTDVGWYQQKPGKAPKLLIYYSSNRYTGVPSRFSGSGSGTDFTLT
ISSLQPEDFATYYCQQDYSSPYTFGQGTKLEIKRTVAAPSVFIFPPSDEQLKSGTASVVCLLNN
FYPREAKVQWKVDNALQSGNSQESVTEQDSKDSTYSLSSTLTLSKADYEKHKVYACEVTHQGLS
SPVTKSFNRGEC] (SEQ ID NO: 285)
62. [Spacer (SEQ ID NO: 645)][huCD147 3A11 Lc1-427-3001 (SEQ ID
NO: 241)]
Amino acid sequence
[QGQSGQG][KHGPCHFRLCPQNTSGGGSSGGSAVGLLAPPGGLSGRSDNHGGSDIQMTQSPSS
LSASVGDRVTITCRASQSVRTDVGWYQQKPGKAPKLLIYYSSNRYTGVPSRFSGSGSGTDFTLT
ISSLQPEDFATYYCQQDYSSPYTFGQGTKLEIKRTVAAPSVFIFPPSDEQLKSGTASVVCLLNN
FYPREAKVQWKVDNALQSGNSQESVTEQDSKDSTYSLSSTLTLSKADYEKHKVYACEVTHQGLS
SPVTKSFNRGEC] (SEQ ID NO: 286)
63. [Spacer (SEQ ID NO: 645)][huCD147 3A11 Lc1-428-3001 (SEQ ID
NO: 242)]
Amino acid sequence
[QGQSGQG][WHGPCHYTKCDDHTMGGGSSGGSAVGLLAPPGGLSGRSDNHGGSDIQMTQSPSS
LSASVGDRVTITCRASQSVRTDVGWYQQKPGKAPKLLIYYSSNRYTGVPSRFSGSGSGTDFTLT
ISSLQPEDFATYYCQQDYSSPYTFGQGTKLEIKRTVAAPSVFIFPPSDEQLKSGTASVVCLLNN
FYPREAKVQWKVDNALQSGNSQESVTEQDSKDSTYSLSSTLTLSKADYEKHKVYACEVTHQGLS
SPVTKSFNRGEC] (SEQ ID NO: 287)
64. [Spacer (SEQ ID NO: 645)][huCD147 3A11 Lc1-440-3001 (SEQ ID
NO: 243)]
Amino acid sequence
[QGQSGQG][TCLHLTRFNYLSCNKGGGSSGGSAVGLLAPPGGLSGRSDNHGGSDIQMTQSPSS
LSASVGDRVTITCRASQSVRTDVGWYQQKPGKAPKLLIYYSSNRYTGVPSRFSGSGSGTDFTLT
ISSLQPEDFATYYCQQDYSSPYTFGQGTKLEIKRTVAAPSVFIFPPSDEQLKSGTASVVCLLNN
FYPREAKVQWKVDNALQSGNSQESVTEQDSKDSTYSLSSTLTLSKADYEKHKVYACEVTHQGLS
SPVTKSFNRGEC] (SEQ ID NO: 288)
65. [Spacer (SEQ ID NO: 645)][huCD147 3A11 Lc1-443-3001 (SEQ ID
NO: 244)]
Amino acid sequence
[QGQSGQG][AHGPCHYNTECNSNKGGGSSGGSAVGLLAPPGGLSGRSDNHGGSDIQMTQSPSS
LSASVGDRVTITCRASQSVRTDVGWYQQKPGKAPKLLIYYSSNRYTGVPSRFSGSGSGTDFTLT
ISSLQPEDFATYYCQQDYSSPYTFGQGTKLEIKRTVAAPSVFIFPPSDEQLKSGTASVVCLLNN
FYPREAKVQWKVDNALQSGNSQESVTEQDSKDSTYSLSSTLTLSKADYEKHKVYACEVTHQGLS
SPVTKSFNRGEC] (SEQ ID NO: 289)
66. [Spacer (SEQ ID NO: 645)][huCD147 3A11 Lc1-444-3001 (SEQ ID
NO: 245)]
Amino acid sequence
[QGQSGQG][THGPCHYKECDWMTIGGGSSGGSAVGLLAPPGGLSGRSDNHGGSDIQMTQSPSS
LSASVGDRVTITCRASQSVRTDVGWYQQKPGKAPKLLIYYSSNRYTGVPSRFSGSGSGTDFTLT
ISSLQPEDFATYYCQQDYSSPYTFGQGTKLEIKRTVAAPSVFIFPPSDEQLKSGTASVVCLLNN
FYPREAKVQWKVDNALQSGNSQESVTEQDSKDSTYSLSSTLTLSKADYEKHKVYACEVTHQGLS
SPVTKSFNRGEC] (SEQ ID NO: 290)
67. [Spacer (SEQ ID NO: 645)][huCD147 3A11 Lc1-445-3001 (SEQ ID
NO: 246)]
Amino acid sequence
[QGQSGQG][SHGPCHFDYQCINNTGGGSSGGSAVGLLAPPGGLSGRSDNHGGSDIQMTQSPSS
LSASVGDRVTITCRASQSVRTDVGWYQQKPGKAPKLLIYYSSNRYTGVPSRFSGSGSGTDFTLT
ISSLQPEDFATYYCQQDYSSPYTFGQGTKLEIKRTVAAPSVFIFPPSDEQLKSGTASVVCLLNN
FYPREAKVQWKVDNALQSGNSQESVTEQDSKDSTYSLSSTLTLSKADYEKHKVYACEVTHQGLS
SPVTKSFNRGEC] (SEQ ID NO: 291)
68. [Spacer (SEQ ID NO: 645)][huCD147 3A11 Lc1-414.1-3001 (SEQ
ID NO: 247)]
Amino acid sequence
[QGQSGQG][THGPCHFKPNCGGGSSGGSAVGLLAPPGGLSGRSDNHGGSDIQMTOSPSSLSAS
VGDRVTITCRASQSVRTDVGWYQQKPGKAPKLLIYYSSNRYTGVPSRFSGSGSGTDFTLTISSL
QPEDFATYYCQQDYSSPYTFGQGTKLEIKRTVAAPSVFIFPPSDEQLKSGTASVVCLLNNFYPR
EAKVQWKVDNALQSGNSQESVTEQDSKDSTYSLSSTLTLSKADYEKHKVYACEVTHQGLSSPVT
KSFNRGEC] (SEQ ID NO: 292)
69. [Spacer (SEQ ID NO: 645)][huCD147 3A11 Lc1-414.2-3001 (SEQ
ID NO: 248)]
Amino acid sequence
[QGQSGQG][THGPCHFKPQCSYPTGGGSSGGSAVGLLAPPGGLSGRSDNHGGSDIQMTQSPSS
LSASVGDRVTITCRASQSVRTDVGWYQQKPGKAPKLLIYYSSNRYTGVPSRFSGSGSGTDFTLT
ISSLQPEDFATYYCQQDYSSPYTFGQGTKLEIKRTVAAPSVFIFPPSDEQLKSGTASVVCLLNN
FYPREAKVQWKVDNALQSGNSQESVTEQDSKDSTYSLSSTLTLSKADYEKHKVYACEVTHQGLS
SPVTKSFNRGEC] (SEQ ID NO: 293)
70. [Spacer (SEQ ID NO: 645)][huCD147 3A11 Lc1-414.3-3001 (SEQ
ID NO: 249)]
Amino acid sequence
[QGQSGQG][THGPCHFKPNCAYPTGGGSSGGSAVGLLAPPGGLSGRSDNHGGSDIQMTQSPSS
LSASVGDRVTITCRASQSVRTDVGWYQQKPGKAPKLLIYYSSNRYTGVPSRFSGSGSGTDFTLT
ISSLQPEDFATYYCQQDYSSPYTFGQGTKLEIKRTVAAPSVFIFPPSDEQLKSGTASVVCLLNN
FYPREAKVQWKVDNALQSGNSQESVTEQDSKDSTYSLSSTLTLSKADYEKHKVYACEVTHQGLS
SPVTKSFNRGEC] (SEQ ID NO: 294)
71. [Spacer (SEQ ID NO: 645)][huCD147 3A11 Lc1-419.1-3001 (SEQ
ID NO: 250)]
Amino acid sequence
[QGQSGQG][LHGPCHYDLKCKQNTGGGSSGGSAVGLLAPPGGLSGRSDNHGGSDIQMTQSPSS
LSASVGDRVTITCRASQSVRTDVGWYQQKPGKAPKLLIYYSSNRYTGVPSRFSGSGSGTDFTLT
ISSLQPEDFATYYCQQDYSSPYTFGQGTKLEIKRTVAAPSVFIFPPSDEQLKSGTASVVCLLNN
FYPREAKVQWKVDNALQSGNSQESVTEQDSKDSTYSLSSTLTLSKADYEKHKVYACEVTHQGLS
SPVTKSFNRGEC] (SEQ ID NO: 295)
72. [Spacer (SEQ ID NO: 645)][huCD147 3A11 Lc1-419.2-3001 (SEQ
ID NO: 251)]
Amino acid sequence
[QGQSGQG][LHGPCHYDLKCKNNGGGSSGGSAVGLLAPPGGLSGRSDNHGGSDIQMTQSPSSL
SASVGDRVTITCRASQSVRTDVGWYQQKPGKAPKLLIYYSSNRYTGVPSRFSGSGSGTDFTLTI
SSLQPEDFATYYCQQDYSSPYTFGQGTKLEIKRTVAAPSVFIFPPSDEQLKSGTASVVCLLNNF
YPREAKVQWKVDNALQSGNSQESVTEQDSKDSTYSLSSTLTLSKADYEKHKVYACEVTHQGLSS
PVTKSFNRGEC] (SEQ ID NO: 296)
73. [Spacer (SEQ ID NO: 645)][huCD147 3A11 Lc1-419.3-3001 (SEQ
ID NO: 252)]
Amino acid sequence
[QGQSGQG][LHGPCHYDLKCKGGGSSGGSAVGLLAPPGGLSGRSDNHGGSDIQMTQSPSSLSA
SVGDRVTITCRASQSVRTDVGWYQQKPGKAPKLLIYYSSNRYTGVPSRFSGSGSGTDFTLTISS
LQPEDFATYYCQQDYSSPYTFGQGTKLEIKRTVAAPSVFIFPPSDEQLKSGTASVVCLLNNFYP
REAKVQWKVDNALQSGNSQESVTEQDSKDSTYSLSSTLTLSKADYEKHKVYACEVTHQGLSSPV
TKSFNRGEC] (SEQ ID NO: 297)
74. [Spacer (SEQ ID NO: 645)][huCD147 3A11 Lc1-414.3-2012 (SEQ
ID NO: 253)]
Amino acid sequence
[QGQSGQG][THGPCHFKPNCAYPTGGGSSGGSISSGLLSGRSANPGGGSDIQMTQSPSSLSAS
VGDRVTITCRASQSVRTDVGWYQQKPGKAPKLLIYYSSNRYTGVPSRFSGSGSGTDFTLTISSL
QPEDFATYYCQQDYSSPYTFGQGTKLEIKRTVAAPSVFIFPPSDEQLKSGTASVVCLLNNFYPR
EAKVQWKVDNALQSGNSQESVTEQDSKDSTYSLSSTLTLSKADYEKHKVYACEVTHQGLSSPVT
KSFNRGEC] (SEQ ID NO: 298)
75. [Spacer (SEQ ID NO: 645)][huCD147 3A11 Lc1-414.4-2012 (SEQ
ID NO: 254)]
Amino acid sequence
[QGQSGQG][THGPCHFRPNCAYPTGGGSSGGSISSGLLSGRSANPGGGSDIQMTQSPSSLSAS
VGDRVTITCRASQSVRTDVGWYQQKPGKAPKLLIYYSSNRYTGVPSRFSGSGSGTDFTLTISSL
QPEDFATYYCQQDYSSPYTFGQGTKLEIKRTVAAPSVFIFPPSDEQLKSGTASVVCLLNNFYPR
EAKVQWKVDNALQSGNSQESVTEQDSKDSTYSLSSTLTLSKADYEKHKVYACEVTHQGLSSPVT
KSFNRGEC] (SEQ ID NO: 299)
76. [Spacer (SEQ ID NO: 645)][huCD147 3A11 Lc1-415-2012 (SEQ ID
NO: 255)]
Amino acid sequence
[QGQSGQG][LHGPCHFNNCNTLKLGGGSSGGSISSGLLSGRSANPGGGSDIQMTQSPSSLSAS
VGDRVTITCRASQSVRTDVGWYQQKPGKAPKLLIYYSSNRYTGVPSRFSGSGSGTDFTLTISSL
QPEDFATYYCQQDYSSPYTFGQGTKLEIKRTVAAPSVFIFPPSDEQLKSGTASVVCLLNNFYPR
EAKVQWKVDNALQSGNSQESVTEQDSKDSTYSLSSTLTLSKADYEKHKVYACEVTHQGLSSPVT
KSFNRGEC] (SEQ ID NO: 300)
77. [Spacer (SEQ ID NO: 645)][huCD147 3A11 Lc1-415.1-2012 (SEQ
ID NO: 256)]
Amino acid sequence
[QGQSGQG][LHGPCHFNNCNTLGGGSSGGSISSGLLSGRSANPGGGSDIQMTQSPSSLSASVG
DRVTITCRASQSVRTDVGWYQQKPGKAPKLLIYYSSNRYTGVPSRFSGSGSGTDFTLTISSLQP
EDFATYYCQQDYSSPYTFGQGTKLEIKRTVAAPSVFIFPPSDEQLKSGTASVVCLLNNFYPREA
KVQWKVDNALQSGNSQESYTEQDSKDSTYSLSSTLTLSKADYEKHKVYACEVTHQGLSSPVTKS
FNRGEC] (SEQ ID NO: 301)
78. [Spacer (SEQ ID NO: 645)][huCD147 3A11 Lc1-419.3-2012 (SEQ
ID NO: 257)]
Amino acid sequence
[QGQSGQG][LHGPCHYDLKCKGGGSSGGSISSGLLSGRSANPGGGSDIQMTQSPSSLSASVGD
RVTITCRASQSVRTDVGWYQQKPGKAPKLLIYYSSNRYTGVPSRFSGSGSGTDFTLTISSLQPE
DFATYYCQQDYSSPYTFGQGTKLEIKRTVAAPSVFIFPPSDEQLKSGTASVVCLLNNFYPREAK
VQWKVDNALQSGNSQESVTEQDSKDSTYSLSSTLTLSKADYEKHKVYACEVTHQGLSSPVTKSF
NRGEC] (SEQ ID NO: 302)
79. [Spacer (SEQ ID NO: 645)][huCD147 3A11 Lc1-419.4-2012 (SEQ
ID NO: 258)]
Amino acid sequence
[QGQSGQG][LHGPCHYDLKCGGGSSGGSISSGLLSGRSANPGGGSDIQMTQSPSSLSASVGDR
VTITCRASQSVRTDVGWYQQKPGKAPKLLIYYSSNRYTGVPSRFSGSGSGTDFTLTISSLQPED
FATYYCQQDYSSPYTFGQGTKLEIKRTVAAPSVFIFPPSDEQLKSGTASVVCLLNNFYPREAKV
QWKVDNALQSGNSQESVTEQDSKDSTYSLSSTLTLSKADYEKHKVYACEVTHQGLSSPVTKSFN
RGEC] (SEQ ID NO: 303)
80. [Spacer (SEQ ID NO: 645)][huCD147 3A11 Lc1-419.5-2012 (SEQ
ID NO: 259)]
Amino acid sequence
[QGQSGQG][LHGPCHYDLRCGGGSSGGSISSGLLSGRSANPGGGSDIQMTQSPSSLSASVGDR
VTITCRASQSVRTDVGWYQQKPGKAPKLLIYYSSNRYTGVPSRFSGSGSGTDFTLTISSLQPED
FATYYCQQDYSSPYTFGQGTKLEIKRTVAAPSVFIFPPSDEQLKSGTASVVCLLNNFYPREAKV
QWKVDNALQSGNSQESVTEQDSKDSTYSLSSTLTLSKADYEKHKVYACEVTHQGLSSPVTKSFN
RGEC] (SEQ ID NO: 304)
81. [Spacer (SEQ ID NO: 645)][huCD147 3A11 Lc1-440-2012 (SEQ ID
NO: 260)]
Amino acid sequence
[QGQSGQG][TCLHLTRFNYLSCNKGGGSSGGSISSGLLSGRSANPGGGSDIQMTQSPSSLSAS
VGDRVTITCRASQSVRTDVGWYQQKPGKAPKLLIYYSSNRYTGVPSRFSGSGSGTDFTLTISSL
QPEDFATYYCQQDYSSPYTFGQGTKLEIKRTVAAPSVFIFPPSDEQLKSGTASVVCLLNNFYPR
EAKVQWKVDNALQSGNSQESVTEQDSKDSTYSLSSTLTLSKADYEKHKVYACEVTHQGLSSPVT
KSFNRGEC] (SEQ ID NO: 305)
82. [Spacer (SEQ ID NO: 645)][huCD147 3A11 Lc1-440.1-2012 (SEQ
ID NO: 261)]
Amino acid sequence
[QGQSGQG][TCLHLTRFNYLSCGGGSSGGSISSGLLSGRSANPGGGSDIQMTQSPSSLSASVG
DRVTITCRASQSVRTDVGWYQQKPGKAPKLLIYYSSNRYTGVPSRFSGSGSGTDFTLTISSLQP
EDFATYYCQQDYSSPYTFGQGTKLEIKRTVAAPSVFIFPPSDEQLKSGTASVVCLLNNFYPREA
KVQWKVDNALQSGNSQESVTEQDSKDSTYSLSSTLTLSKADYEKHKVYACEVTHQGLSSPVTKS
FNRGEC] (SEQ ID NO: 306)
83. [Spacer (SEQ ID NO: 645)][huCD147 3A11 Lc1-440.1-2001 VL
domain (SEQ ID NO: 172)]
Amino acid sequence
[QGQSGQG][TCLHLTRFNYLSCGGGSSGGSISSGLLSGRSDNHGGGSDIQMTQSPSSLSASVG
DRVTITCRASQSVRTDVGWYQQKPGKAPKLLIYYSSNRYTGVPSRFSGSGSGTDFTLTISSLQP
EDFATYYCQQDYSSPYTFGQGTKLEIK] (SEQ ID NO: 217)
84. [Spacer (SEQ ID NO: 645)][huCD147 3A11 Lc1-440.1-3001 VL
domain (SEQ ID NO: 173)]
Amino acid sequence
[QGQSGQG][TCLHLTRFNYLSCGGGSSGGSAVGLLAPPGGLSGRSDNHGGSDIQMTQSPSSLS
ASVGDRVTITCRASQSVRTDVGWYQQKPGKAPKLLIYYSSNRYTGVPSRFSGSGSGTDFTLTIS
SLQPEDFATYYCQQDYSSPYTFGQGTKLEIK] (SEQ ID NO: 218)
85. [Spacer (SEQ ID NO: 645)][huCD147 3A11 Lc1-440.1-2007 VL
domain (SEQ ID NO: 174)]
Amino acid sequence
[QGQSGQG][TCLHLTRFNYLSCGGGSSGGSISSGLLSGRSDIHGGGSDIQMTQSPSSLSASVG
DRVTITCRASQSVRTDVGWYQQKPGKAPKLLIYYSSNRYTGVPSRFSGSGSGTDFTLTISSLQP
EDFATYYCQQDYSSPYTFGQGTKLEIK] (SEQ ID NO: 219)
86. [Spacer (SEQ ID NO: 645)][huCD147 3A11 Lc1-440.1-3007 VL
domain (SEQ ID NO: 175)]
Amino acid sequence
[QGQSGQG][TCLHLTRFNYLSCGGGSSGGSAVGLLAPPGGLSGRSDIHGGSDIQMTQSPSSLS
ASVGDRVTITCRASQSVRTDVGWYQQKPGKAPKLLIYYSSNRYTGVPSRFSGSGSGTDFTLTIS
SLQPEDFATYYCQQDYSSPYTFGQGTKLEIK] (SEQ ID NO: 220)
87. [Spacer (SEQ ID NO: 645)][huCD147 3A11 Lc1-440.1-2008 VL
domain (SEQ ID NO: 176)]
Amino acid sequence
[QGQSGQG][TCLHLTRFNYLSCGGGSSGGSISSGLLSGRSDQHGGGSDIQMTQSPSSLSASVG
DRVTITCRASQSVRTDVGWYQQKPGKAPKLLIYYSSNRYTGVPSRFSGSGSGTDFTLTISSLQP
EDFATYYCQQDYSSPYTFGQGTKLEIK] (SEQ ID NO: 221)
88. [Spacer (SEQ ID NO: 645)][huCD147 3A11 Lc1-440.1-3008 VL
domain (SEQ ID NO: 177)]
Amino acid sequence
[QGQSGQG][TCLHLTRFNYLSCGGGSSGGSAVGLLAPPGGLSGRSDQHGGSDIQMTQSPSSLS
ASVGDRVTITCRASQSVRTDVGWYQQKPGKAPKLLIYYSSNRYTGVPSRFSGSGSGTDFTLTIS
SLQPEDFATYYCQQDYSSPYTFGQGTKLEIK] (SEQ ID NO: 222)
89. [Spacer (SEQ ID NO: 645)][huCD147 3A11 Lc1-440.1-2011 VL
domain (SEQ ID NO: 178)]
Amino acid sequence
[QGQSGQG][TCLHLTRFNYLSCGGGSSGGSISSGLLSGRSDNPGGGSDIQMTQSPSSLSASVG
DRVTITCRASQSVRTDVGWYQQKPGKAPKLLIYYSSNRYTGVPSRFSGSGSGTDFTLTISSLQP
EDFATYYCQQDYSSPYTFGQGTKLEIK] (SEQ ID NO: 223)
90. [Spacer (SEQ ID NO: 645)][huCD147 3A11 Lc1-440.1-3011 VL
domain (SEQ ID NO: 179)]
Amino acid sequence
[QGQSGQG][TCLHLTRFNYLSCGGGSSGGSAVGLLAPPGGLSGRSDNPGGSDIQMTQSPSSLS
ASVGDRVTITCRASQSVRTDVGWYQQKPGKAPKLLIYYSSNRYTGVPSRFSGSGSGTDFTLTIS
SLQPEDFATYYCQQDYSSPYTFGQGTKLEIK] (SEQ ID NO: 224)
91. [Spacer (SEQ ID NO: 645)][huCD147 3A11 Lc1-440.1-3012 VL
domain (SEQ ID NO: 180)]
Amino acid sequence
[QGQSGQG][TCLHLTRFNYLSCGGGSSGGSAVGLLAPPGGLSGRSANPGGSDIQMTQSPSSLS
ASVGDRVTITCRASQSVRTDVGWYQQKPGKAPKLLIYYSSNRYTGVPSRFSGSGSGTDFTLTIS
SLQPEDFATYYCQQDYSSPYTFGQGTKLEIK] (SEQ ID NO: 225)
92. [Spacer (SEQ ID NO: 645)][huCD147 3A11 Lc1-440.1-2013 VL
domain (SEQ ID NO: 181)]
Amino acid sequence
[QGQSGQG][TCLHLTRFNYLSCGGGSSGGSISSGLLSGRSANIGGGSDIQMTQSPSSLSASVG
DRVTITCRASQSVRTDVGWYQQKPGKAPKLLIYYSSNRYTGVPSRFSGSGSGTDFTLTISSLQP
EDFATYYCQQDYSSPYTFGQGTKLEIK] (SEQ ID NO: 226)
93. [Spacer (SEQ ID NO: 645)][huCD147 3A11 Lc1-440.1-3013 VL
domain (SEQ ID NO: 182)]
Amino acid sequence
[QGQSGQG][TCLHLTRFNYLSCGGGSSGGSAVGLLAPPGGLSGRSANIGGSDIQMTQSPSSLS
ASVGDRVTITCRASQSVRTDVGWYQQKPGKAPKLLIYYSSNRYTGVPSRFSGSGSGTDFTLTIS
SLQPEDFATYYCQQDYSSPYTFGQGTKLEIK] (SEQ ID NO: 227)
94. [Spacer (SEQ ID NO: 645)][huCD147 3A11 Lc1-440.1-2001 (SEQ
ID NO: 262)]
Amino acid sequence
[QGQSGQG][TCLHLTRFNYLSCGGGSSGGSISSGLLSGRSDNHGGGSDIQMTQSPSSLSASVG
DRVTITCRASQSVRTDVGWYQQKPGKAPKLLIYYSSNRYTGVPSRFSGSGSGTDFTLTISSLQP
EDFATYYCQQDYSSPYTFGQGTKLEIKRTVAAPSVFIFPPSDEQLKSGTASVVCLLNNFYPREA
KVQWKVDNALQSGNSQESVTEQDSKDSTYSLSSTLTLSKADYEKHKVYACEVTHQGLSSPVTKS
FNRGEC] (SEQ ID NO: 307)
95. [Spacer (SEQ ID NO: 645)][huCD147 3A11 Lc1-440.1-3001 (SEQ
ID NO: 263)]
Amino acid sequence
[QGQSGQG][TCLHLTRFNYLSCGGGSSGGSAVGLLAPPGGLSGRSDNHGGSDIQMTQSPSSLS
ASVGDRVTITCRASQSVRTDVGWYQQKPGKAPKLLIYYSSNRYTGVPSRFSGSGSGTDFTLTIS
SLQPEDFATYYCQQDYSSPYTFGQGTKLEIKRTVAAPSVFIFPPSDEQLKSGTASVVCLLNNFY
PREAKVQWKVDNALQSGNSQESVTEQDSKDSTYSLSSTLTLSKADYEKHKVYACEVTHQGLSSP
VTKSFNRGEC] (SEQ ID NO: 308)
96. [Spacer (SEQ ID NO: 645)][huCD147 3A11 Lc1-440.1-2007 (SEQ
ID NO: 264)]
Amino acid sequence
[QGQSGQG][TCLHLTRFNYLSCGGGSSGGSISSGLLSGRSDIHGGGSDIQMTQSPSSLSASVG
DRVTITCRASQSVRTDVGWYQQKPGKAPKLLIYYSSNRYTGVPSRFSGSGSGTDFTLTISSLQP
EDFATYYCQQDYSSPYTFGQGTKLEIKRTVAAPSVFIFPPSDEQLKSGTASVVCLLNNFYPREA
KVQWKVDNALQSGNSQESVTEQDSKDSTYSLSSTLTLSKADYEKHKVYACEVTHQGLSSPVTKS
FNRGEC] (SEQ ID NO: 309)
97. [Spacer (SEQ ID NO: 645)][huCD147 3A11 Lc1-440.1-3007 (SEQ
ID NO: 265)]
Amino acid sequence
[QGQSGQG][TCLHLTRFNYLSCGGGSSGGSAVGLLAPPGGLSGRSDIHGGSDIQMTQSPSSLS
ASVGDRVTITCRASQSVRTDVGWYQQKPGKAPKLLIYYSSNRYTGVPSRFSGSGSGTDFTLTIS
SLQPEDFATYYCQQDYSSPYTFGQGTKLEIKRTVAAPSVFIFPPSDEQLKSGTASVVCLLNNFY
PREAKVQWKVDNALQSGNSQESVTEQDSKDSTYSLSSTLTLSKADYEKHKVYACEVTHQGLSSP
VTKSFNRGEC] (SEQ ID NO: 310)
98. [Spacer (SEQ ID NO: 645)][huCD147 3A11 Lc1-440.1-2008 (SEQ
ID NO: 266)]
Amino acid sequence
[QGQSGQG][TCLHLTRFNYLSCGGGSSGGSISSGLLSGRSDQHGGGSDIQMTQSPSSLSASVG
DRVTITCRASQSVRTDVGWYQQKPGKAPKLLIYYSSNRYTGVPSRFSGSGSGTDFTLTISSLQP
EDFATYYCQQDYSSPYTFGQGTKLEIKRTVAAPSVFIFPPSDEQLKSGTASVVCLLNNFYPREA
KVQWKVDNALQSGNSQESVTEQDSKDSTYSLSSTLTLSKADYEKHKVYACEVTHQGLSSPVTKS
FNRGEC] (SEQ ID NO: 311)
99. [Spacer (SEQ ID NO: 645)][huCD147 3A11 Lc1-440.1-3008 (SEQ
ID NO: 267)]
Amino acid sequence
[QGQSGQG][TCLHLTRFNYLSCGGGSSGGSAVGLLAPPGGLSGRSDQHGGSDIQMTQSPSSLS
ASVGDRVTITCRASQSVRTDVGWYQQKPGKAPKLLIYYSSNRYTGVPSRFSGSGSGTDFTLTIS
SLQPEDFATYYCQQDYSSPYTFGQGTKLEIKRTVAAPSVFIFPPSDEQLKSGTASVVCLLNNFY
PREAKVQWKVDNALQSGNSQESVTEQDSKDSTYSLSSTLTLSKADYEKHKVYACEVTHQGLSSP
VTKSFNRGEC] (SEQ ID NO: 312)
100. [Spacer (SEQ ID NO: 645)][huCD147 3A11 Lc1-440.1-2011 (SEQ
ID NO: 268)]
Amino acid sequence
[QGQSGQG][TCLHLTRFNYLSCGGGSSGGSISSGLLSGRSDNPGGGSDIQMTQSPSSLSASVG
DRVTITCRASQSVRTDVGWYQQKPGKAPKLLIYYSSNRYTGVPSRFSGSGSGTDFTLTISSLQP
EDFATYYCQQDYSSPYTFGQGTKLEIKRTVAAPSVFIFPPSDEQLKSGTASVVCLLNNFYPREA
KVQWKVDNALQSGNSQESVTEQDSKDSTYSLSSTLTLSKADYEKHKVYACEVTHQGLSSPVTKS
FNRGEC] (SEQ ID NO: 313)
101. [Spacer (SEQ ID NO: 645)][huCD147 3A11 Lc1-440.1-3011 (SEQ
ID NO: 269)]
Amino acid sequence
[QGQSGQG][TCLHLTRFNYLSCGGGSSGGSAVGLLAPPGGLSGRSDNPGGSDIQMTQSPSSLS
ASVGDRVTITCRASQSVRTDVGWYQQKPGKAPKLLIYYSSNRYTGVPSRFSGSGSGTDFTLTIS
SLQPEDFATYYCQQDYSSPYTFGQGTKLEIKRTVAAPSVFIFPPSDEQLKSGTASVVCLLNNFY
PREAKVQWKVDNALQSGNSQESVTEQDSKDSTYSLSSTLTLSKADYEKHKVYACEVTHQGLSSP
VTKSFNRGEC] (SEQ ID NO: 314)
102. [Spacer (SEQ ID NO: 645)][huCD147 3A11 Lc1-440.1-3012 (SEQ
ID NO: 270)]
Amino acid sequence
[QGQSGQG][TCLHLTRFNYLSCGGGSSGGSAVGLLAPPGGLSGRSANPGGSDIQMTQSPSSLS
ASVGDRVTITCRASQSVRTDVGWYQQKPGKAPKLLIYYSSNRYTGVPSRFSGSGSGTDFTLTIS
SLQPEDFATYYCQQDYSSPYTFGQGTKLEIKRTVAAPSVFIFPPSDEQLKSGTASVVCLLNNFY
PREAKVQWKVDNALQSGNSQESVTEODSKDSTYSLSSTLTLSKADYEKHKVYACEVTHQGLSSP
VTKSFNRGEC] (SEQ ID NO: 315)
103. [Spacer (SEQ ID NO: 645)][huCD147 3A11 Lc1-440.1-2013 (SEQ
ID NO: 271)]
Amino acid sequence
[QGQSGQG][TCLHLTRFNYLSCGGGSSGGSISSGLLSGRSANIGGGSDIQMTQSPSSLSASVG
DRVTITCRASQSVRTDVGWYQQKPGKAPKLLIYYSSNRYTGVPSRFSGSGSGTDFTLTISSLQP
EDFATYYCQQDYSSPYTFGQGTKLEIKRTVAAPSVFIFPPSDEQLKSGTASVVCLLNNFYPREA
KVQWKVDNALQSGNSQESVTEQDSKDSTYSLSSTLTLSKADYEKHKVYACEVTHQGLSSPVTKS
FNRGEC] (SEQ ID NO: 316)
104. [Spacer (SEQ ID NO: 645)][huCD147 3A11 Lc1-440.1-3013 (SEQ
ID NO: 272)]
Amino acid sequence
[QGQSGQG][TCLHLTRFNYLSCGGGSSGGSAVGLLAPPGGLSGRSANIGGSDIQMTQSPSSLS
ASYGDRVTITCRASQSVRTDVGWYQQKPGKAPKLLIYYSSNRYTGVPSRFSGSGSGTDFTLTIS
SLQPEDFATYYCQQDYSSPYTFGQGTKLEIKRTVAAPSVFIFPPSDEQLKSGTASVVCLLNNFY
PREAKVQWKVDNALQSGNSQESVTEODSKDSTYSLSSTLTLSKADYEKHKVYACEVTHQGLSSP
VTKSFNRGEC] (SEQ ID NO: 317)

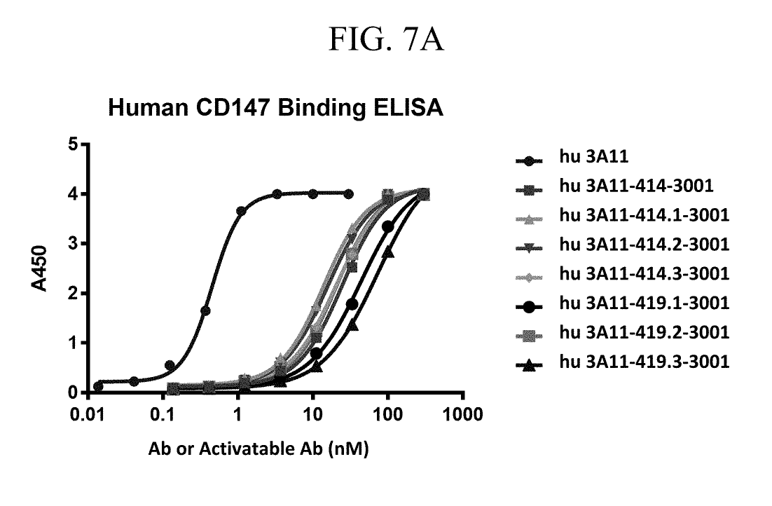

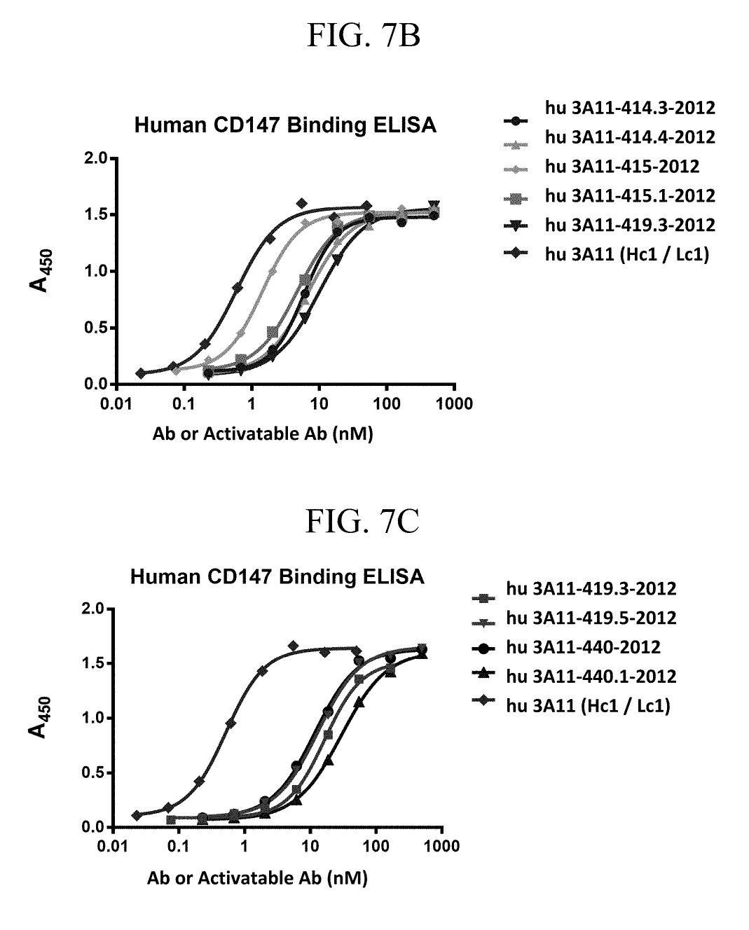

As shown in FIGS. 7A, 7B, and 7C, the binding affinity of anti-human CD147 activatable antibodies of the present disclosure to human CD147 polypeptide were assayed using an ELISA. The activatable antibodies of the present disclosure that were assayed by ELISA for binding to human CD147 polypeptide in the exemplary study depicted in FIG. 7A were (a) hu 3A11 Hc1/Lc1-414-3001 (VL of SEQ ID NO: 145 and VH of SEQ ID NO: 1), (b) hu 3A11 Hc1/Lc1-414.1-3001 (VL of SEQ ID NO: 157 and VH of SEQ ID NO: 1), (c) hu 3A11 Hc1/Lc1-414.2-3001 (VL of SEQ ID NO: 158 and VH of SEQ ID NO: 1), (d) hu 3A11 Hc1/Lc1-414.3-3001 (VL of SEQ ID NO: 159 and VH of SEQ ID NO: 1), (e) hu 3A11 He 1/Lc1-419.1-3001 (VL of SEQ ID NO: 160 and VH of SEQ ID NO: 1), (f) hu 3A11 Hc1/Lc1-419.2-3001 (VL of SEQ ID NO: 161 and VH of SEQ ID NO: 1), (g) hu 3A11 Hc1/Lc1-419.3-3001 (VL of SEQ ID NO: 162 and VH of SEQ ID NO: 1), with humanized anti-human CD147 3A11 antibody of the present disclosure (VL of SEQ ID NO: 5 and VH of SEQ ID NO: 1) also assayed.

The activatable antibodies of the present disclosure that were assayed by ELISA for binding to human CD147 polypeptide in the exemplary study depicted in FIG. 7B were (a) hu 3A11 Hc1/Lc1-414.3-2012 (VL of SEQ ID NO: 163 and VH of SEQ ID NO: 1), (b) hu 3A11 Hc1/Lc1-414.4-2012 (VL of SEQ ID NO: 164 and VH of SEQ ID NO: 1), (c) hu 3A11 He 1/Lc1-415-2012 (VL of SEQ ID NO: 165 and VH of SEQ ID NO: 1), (d) hu 3A11 Hc1/Lc1-415.1-2012 (VL of SEQ ID NO: 166 and VH of SEQ ID NO: 1), and (e) hu 3A11 Hc1/Lc1-419.3-2012 (VL of SEQ ID NO: 167 and VH of SEQ ID NO: 1), with humanized anti-human CD147 3A11 antibody of the present disclosure (VL of SEQ ID NO: 5 and VH of SEQ ID NO: 1) also assayed.

The activatable antibodies of the present disclosure that were assayed by ELISA for binding to human CD147 polypeptide in the exemplary study depicted in FIG. 7C were (a) hu 3A11 Hc1/Lc1-419.4-2012 (VL of SEQ ID NO: 168 and VH of SEQ ID NO: 1), (b) hu 3A11 Hc1/Lc1-419.5-2012 (VL of SEQ ID NO: 169 and VH of SEQ ID NO: 1), (c) hu 3A11 Hc1/Lc1-440-2012 (VL of SEQ ID NO: 170 and VH of SEQ ID NO: 1), and (d) hu 3A11 Hc1/Lc1-440.1-2012 (VL of SEQ ID NO: 171 and VH of SEQ ID NO: 1), with humanized anti-human CD147 3A11 antibody of the present disclosure (VL of SEQ ID NO: 5 and VH of SEQ ID NO: 1) also assayed.

For the exemplary assay depicted in FIGS. 7A-7C, standard ELISA protocol was used, in which human CD147 protein was absorbed to ELISA plates and subsequently incubated with the indicated concentration of antibody of the present disclosure. Bound humanized activatable antibody of the present disclosure was detected with anti-human Fab-peroxidase secondary antibody and Ultra TMB detection (Thermo Fisher Scientific). The apparent dissociation constants of this exemplary binding study are shown in Table 21.

TABLE 21
Equilibrium Dissociation Constants of Anti-human CD147
Activatable Antibodies To Human CD147

| Ab | K_{D} (nM) |
|---|---|
| hu 3A11 (Lc1/Hc1) | 0.45 |
| hu 3A11-414-3001 | 24.69 |
| hu 3A11-414.1-3001 | 13.76 |
| hu 3A11-414.2-3001 | 15.65 |
| hu 3A11-414.3-3001 | 20.23 |
| hu 3A11-419.1-3001 | 43.97 |
| hu 3A11-419.2-3001 | 20.76 |
| hu 3A11-419.3-3001 | 73.5 |
| hu 3A11-414.3-2012 | 5.92 |
| hu 3A11-414.4-2012 | 6.73 |
| hu 3A11-415-2012 | 1.43 |
| hu 3A11-415.1-2012 | 4.68 |
| hu 3A11-419.3-2012 | 10.0 |
| hu 3A11 (Hc1/Lc1) | 0.59 |

### Example 5. CD147 Expression in Multiple Primary and Metastatic Tumors

This Example shows that CD147 is expressed in a large variety of primary and metastatic tumor types by immunohistochemical (IHC) staining using an anti-human CD147 antibody.

Table 22 shows that CD147 is highly expressed in a large number of primary and metastatic tumor samples, using IHC staining with an anti-human CD147 antibody on multiple primary tumors and metastatic tissue microarrays (TMA). In this exemplary study, human CD147 was detected using mouse monoclonal anti-human CD147 antibody (clone MEM-M6/1, Abcam code ab78106) at 10 ug/ml after standard heat-induced epitope retrieval method in sodium citrate buffer (10 mM sodium citrate, 0.05% Tween-20, pH 6.0).

TABLE 22
CD147 Expression Level By Immunohistochemistry (IHC)

| | | | % of cores with moderate |
|---|---|---|---|
| | | # of cores | or strong CD147 IHC |
| | | analyzed | staining |
| Breast | Overall | 144 | 61% |
| | Her2+ | 26 | 65% |
| | ER+ | 12 | 42% |
| | TNBC | 62 | 66% |
| | Metastatic | 44 | 79% |
| Prostate | Overall | 71 | 68% |
| Lung | Overall | 70 | 84% |
| | Adeno NSCLC | 21 | 81% |
| | Squamous | 31 | 90% |
| | NSCLC | | |
| Esophageal | Overall | 97 | 91% |
| Head & | Overall | 69 | 94% |
| Neck | | | |
| Colon | Overall | 57 | 69% |

### Example 6. CD147 Expression in Multiple Primary and Metastatic Tumors

This Example shows that CD147 is expressed in a variety of patient-derived primary tumor types by immunohistochemical (IHC) staining using an anti-human CD147 antibody.

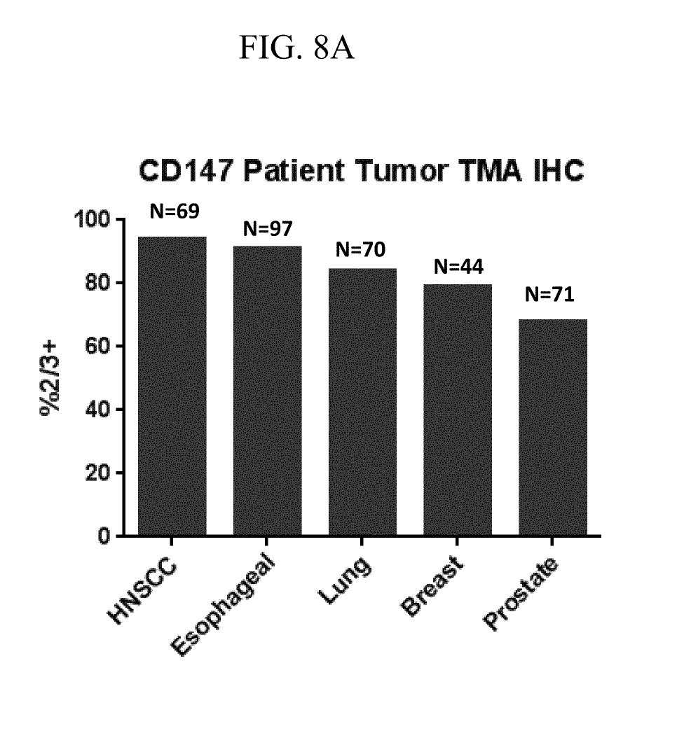

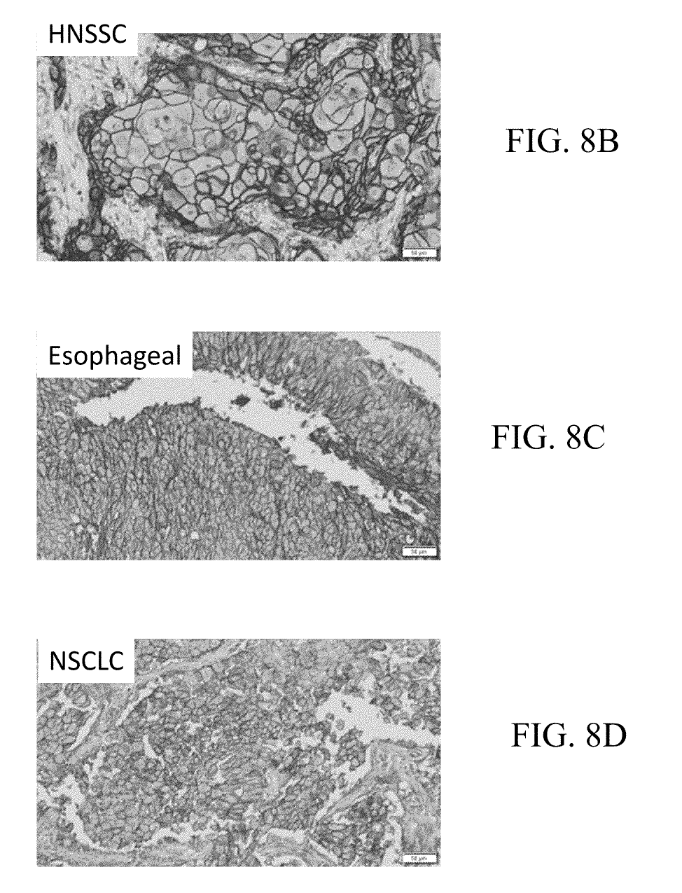

FIGS. 8A, 8B, 8C, and 8D show that CD147 is highly expressed in a large number of primary and metastatic tumor samples, using IHC staining with a commercially-purchased anti-human CD147 antibody on multiple primary tumors and metastatic tissue microarrays (TMA). FIG. 8A shows that CD147 is moderately or highly expressed in a large number and variety of patient-derived metastatic tumor samples, using IHC staining with a commercially-purchased anti-human CD147 mouse monoclonal antibody (clone MEM-M6/1, Abcam code ab78106) on multiple patient-derived tumor tissue microarrays (TMA). FIG. 8A shows a summary of the level of IHC staining of CD147 of the TMAs shows that a large number of cores derived from multiple patient-derived metastatic samples showed a strong CD147 signal.

FIGS. 8B, 8C, and 8D show an IHC staining with a commercially-purchased anti-human CD147 mouse monoclonal antibody (clone MEM-M6/1, Abcam code ab78106) in head-neck squamous cell carcinoma cancer (FIG. 8B), esophageal cancer (FIG. 8C), and non-small cell lung cancer (FIG. 8D). FIGS. 8B, 8C, and 8D show that these exemplary cancer types demonstrate a high level of CD147 signal.

### Example 7: CD147 Expression and Sensitivity to Anti-Human CD147-Mediated Cytotoxicity In Multiple Cell Lines

This Example shows that CD147 is expressed in high levels in many tumor-derived cell lines, and that many of these cell lines demonstrated sensitivity to anti-human CD147-targeted cytotoxicity.

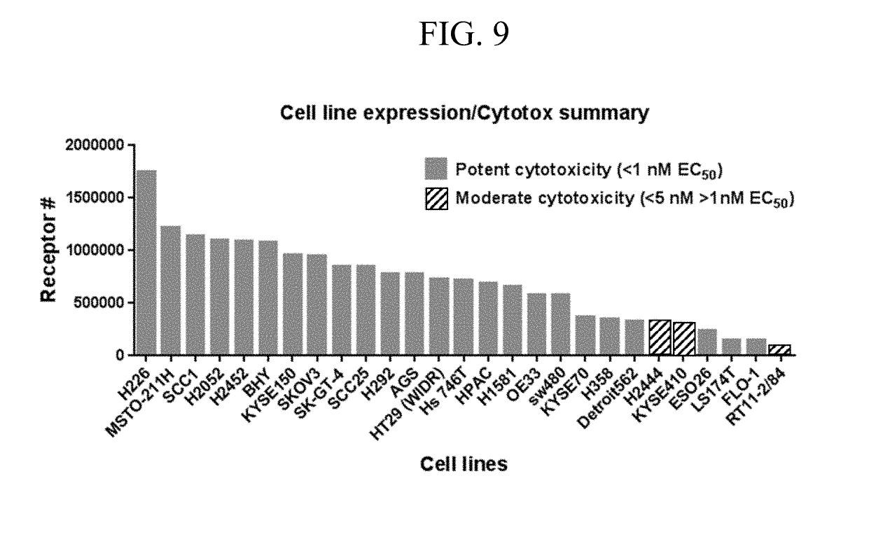

FIG. 9 shows the maximum binding or relative expression of CD147 in the indicated cell lines by FACS analysis. FACS staining was carried out using the 3A11 anti-human CD147 antibody of the present disclosure (VH of SEQ ID NO: 1, VL of SEQ ID NO: 5), followed by an Alexa Fluor 647 conjugated goat anti mouse secondary antibody, with the height of the bar for a given cell line corresponding to the relative amount of CD147-derived signal and number of cell-surface receptors for anti-human CD147 for that cell line. The color of the bar for a given cell line shows the relative sensitivity of the corresponding cell line in an in vitro cytotoxicity assay where the cell line was treated with anti-human CD147 antibody of the present disclosure (VH of SEQ ID NO: 1, VL of SEQ ID NO: 5) in the presence of an anti-human secondary antibody conjugated to vc-MMAE toxin. A cell line was categorized as showing “potent” cytotoxicity when treated with the combined anti-human CD147 antibody and secondary antibody drug conjugate if the EC50 of less than 1 nM was observed upon treatment with the combined antibody drug conjugate. A cell line was categorized as showing “moderate” cytotoxicity when treated with combined anti-human CD147 antibody and secondary antibody drug conjugate if the EC50 of between 5 nM and 1 nM was observed upon treatment with the combined antibody drug conjugate. FIG. 9 shows that an anti-human CD147 drug conjugate of the present invention demonstrated potent or moderate cytotoxicity against multiple cancer-derived cell lines. In certain embodiments, the anti-human CD147 antibody drug conjugate of the present invention demonstrated potent or moderate cytotoxicity against forty-four (44) of fifty-nine (59) cancer-derived cell lines tested. In certain embodiments, the anti-human CD147 antibody drug conjugate of the present invention demonstrated potent or moderate cytotoxicity against cancer cell lines derived from breast cancer, colorectal cancer, esophageal cancer, gastric cancer, head and neck squamous cell carcinoma, non-small cell lung cancer, mesothelioma, esophageal cancer, ovarian cancer, pancreatic cancer, and prostate cancer. A list of the cell lines tested, their tissue(s) of origin, and the observed EC50 is shown below in Table 23.

TABLE 23
In Vitro Cytotoxieity of Anti-human CD147 Antibody Drug Conjugate
To Various Cell Lines

| | | EC50 (nM) |
|---|---|---|
| | Tissue/Cancer Type | [NC = no kill |
| Cell Line | of Origin | observed] |
| RT11-2/84 | Bladder cancer | 1.94 |
| SKBR3 | Breast (Her2+) | 0.38 |
| HCC 1806 | Breast (TNBC) | 0.68 |
| HCC 70 | Breast (TNBC) | 0.94 |
| HCC1143 | Breast (TNBC) | <1.0 |
| HCC1937 | Breast (TNBC) | <1.0 |
| HCC1954 | Breast (TNBC) | <1.0 |
| HT29 (WIDR) | CRC | 0.87 |
| DLD-1 | CRC | NC |
| HCT 116 | CRC | 1.8 |
| LOVO | CRC | <1.0 |
| LS174T | CRC | ~1 |
| LS411N | CRC | <1.0 |
| SW1417 | CRC | 0.8 |
| sw48 | CRC | <1.0 |
| sw480 | CRC | <1.0 |
| WIDR (derived from | CRC | |
| HT29) | | |
| AGS | gastric adenocarcinoma | 0.09 |
| SK-GT-4 | gastric adenocarcinoma | <0.1 |
| | fundus, well | |
| | differentiated | |
| SNU-1 | gastric carcinoma | 0.21 |
| SNU-16 | gastric carcinoma | 1.14 |
| SNU-5 | gastric carcinoma | 0.11 |
| ESO26 | gastroeoesophageal | 0.06 |
| | junction | |
| | adenocarcinoma | |
| BHY | HNSCC | <1.0 |
| Detroit562 | HNSCC | 0.008 |
| KYSE150 | HNSCC | 0.88 |
| KYSE70 | HNSCC | 0.13 |
| SCC1 | HNSCC | 0.14 |
| SCC25 | HNSCC | 0.2 |
| SCC9 | HNSCC | 0.37 |
| H1975 | Lung cancer | 0.09 |
| H292 | Lung cancer | 0.07 |
| H358 | Lung cancer | 0.09 |
| H520 | Lung cancer | 11.19 |
| H727 | Lung cancer | NC |
| H1581 | Lung (NSCLC) | 0.039 |
| H2444 | Lung (NSCLC) | 1.8 |
| H2141 | Lung (SCLC) | Ambiguous |
| H2405 | Lung (SCLC) | 0.3 |
| H526 | Lung (SCLC) | NC |
| H69 | Lung (SCLC) | ~1.0 |
| H889 | Lung (SCLC) | Ambiguous |
| H2052 | Mesothelioma | 0.03 |
| H226 | Mesothelioma | 0.45 |
| H2452 | Mesothelioma | 0.94 |
| MSTO-211H | Mesothelioma | 0.37 |
| FLO-1 | oeoesophageal distal | 0.51 |
| | adenocarcinoma | |
| KYSE410 | oeoesophageal | 1.35 |
| | squamous cell | |
| | carcinoma | |
| OE33 | oesophageal carcinoma | 0.29 |
| OACM5.1 C | oesophagus distal | 1.1 |
| | adenocarcinoma C | |
| | Barretts | |
| EFO-21 | Ovarian cancer | 1.4 |
| SKOV3 | Ovarian cancer | 0.95 |
| aspc1 | Pancreatic cancer | 20.5 |
| HPAC | Pancreatic cancer | 0.21 |
| HPAF2 | Pancreatic cancer | <1.0 |
| H5766T | Pancreatic cancer | Ambiguous |
| miapaca1 | Pancreatic cancer | 0.65 |
| PC3 | Prostate cancer | <1.0 |
| A253 | Salivary gland cancer | <1.0 |
| Hs 746T | stomach carcinoma | 0.06 |

TNBC = triple-negative breast cancer;
NSCLC = non-small cell lung cancer;
SCLC = small cell lung cancer;
HNSCC = head & neck squamous cell carcinoma;
CRC = colorectal cancer

### Example 8: Anti-Human CD147 Binding to Human and Cynomolgus CD147

This Example shows that anti-human CD147 antibodies of the present disclosure bind CD147 on human and cynomolgus cell lines with similar relative affinities.

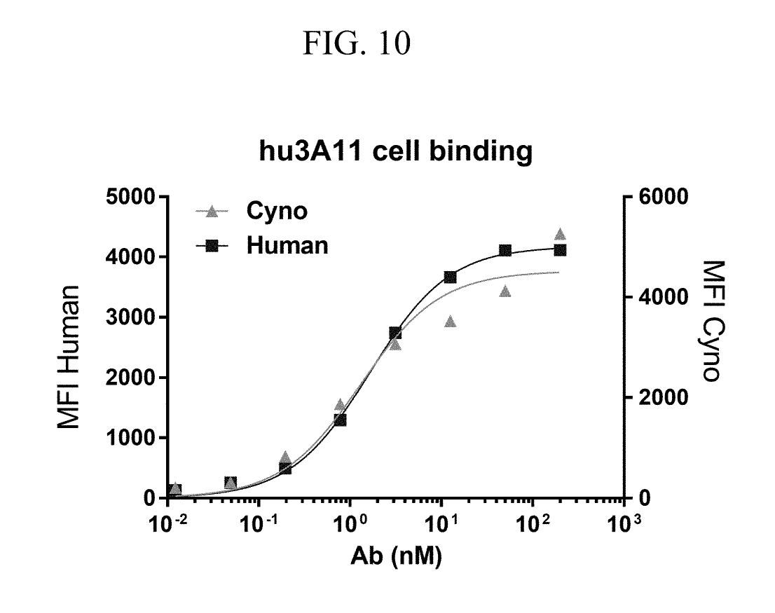

FIG. 10 shows the binding affinity of hu3A11 anti-human CD147 antibody of the present disclosure (VH of SEQ ID NO: 1, VL of SEQ ID NO: 5) to the human KYSE-70 esophageal epithelial cell line and the cynomolgus primary kidney epithelial cell line. In this study, the binding of the humanized anti-human CD147 antibody of the present disclosure to the indicated cell lines were performed using a standard FACS labelling method. Briefly, cells were labeled with the indicated antibodies of the present disclosure: anti-human CD147 antibody (humanized anti-human CD147 hu3A11 antibody; VH of SEQ ID NO: 1, VL of SEQ ID NO: 5) at the indicated concentrations and subsequently detected with an Alexa Fluor 647 labeled goat anti-human IgG secondary antibody. These results show that anti-human CD147 hu3A11 antibody binds the human cell line with a K_{D} of 1.654 nM, which is similar to the affinity of the antibody to the cynomolgus cell line (K_{D} of 1.297 nM).

### Example 9: Anti-Human CD147 Activatable Antibody Drug Conjugates Binding Activity and Cytotoxicity

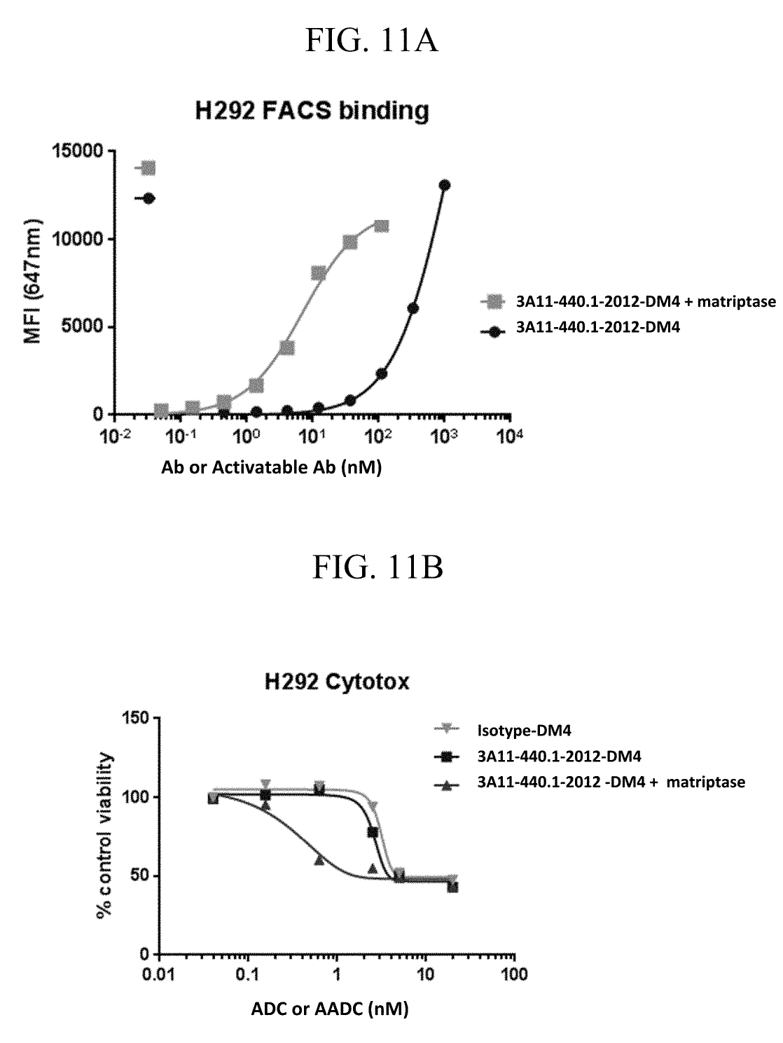

Binding of anti-human CD147 activatable antibody drug conjugates of the present disclosure to the NCI H292 (also referred to herein as H292) cell line was evaluated using FACS. Briefly, cells were incubated with huCD147 activatable antibody drug conjugates (or activatable antibody at the indicated concentrations and subsequently detected with an Alexa Fluor 647 labeled goat anti-human IgG secondary antibody. As shown in FIG. 11A, intact anti-human CD147 activatable antibody drug conjugates (anti-human CD147 hu3A11-440.1-2012-spdb-DM4, VH of SEQ ID NO: 1, VL of SEQ ID NO: 171) of the present disclosure show an affinity of K_{d}>100 nM to H292 cells (FIG. 11A). When the anti-human CD147 AADC of the present disclosure is treated with a protease (matriptase), the binding affinity of the proteolytically-activated anti-human CD147 AADC significantly increases.

FIG. 11B shows the ability of activated anti-human CD147 activatable antibody conjugated to DM4 (anti-human CD147 hu3A11-440.1-2012-spdb-DM4, VH of SEQ ID NO: 1, VL of SEQ ID NO: 171) to kill cells in vitro with an EC50 higher than that of intact anti-human CD147 activatable antibody conjugated to DM4. The cytotoxicity of the intact anti-human CD147 activatable antibody conjugated to DM4 was similar to that of an isotype antibody conjugated to DM4. The EC50 are summarized in Table 24 below.

TABLE 24
In Vitro Cytotoxicity of Anti-human CD147 Activatable Antibodies
To Human H292 Cells

| Ab | K_{D} (nM) |
|---|---|
| Isotype-spdb-DM4 | 1286 |
| hu 3A11-440.1-2012-spdb-DM4 | 408 |
| hu 3A11-440.1-2012-spdb-DM4 + | 0.18 |
| matriptase | |

### Example 10: Activatable Anti-Human CD147-AADC In Vivo Efficacy in HT29 and H292 Xenograft Models

This Example shows that anti-human CD147 activatable antibodies with conjugated toxins (AADCs) of the present disclosure are efficacious in a mouse xenograft model compared to a vehicle control.

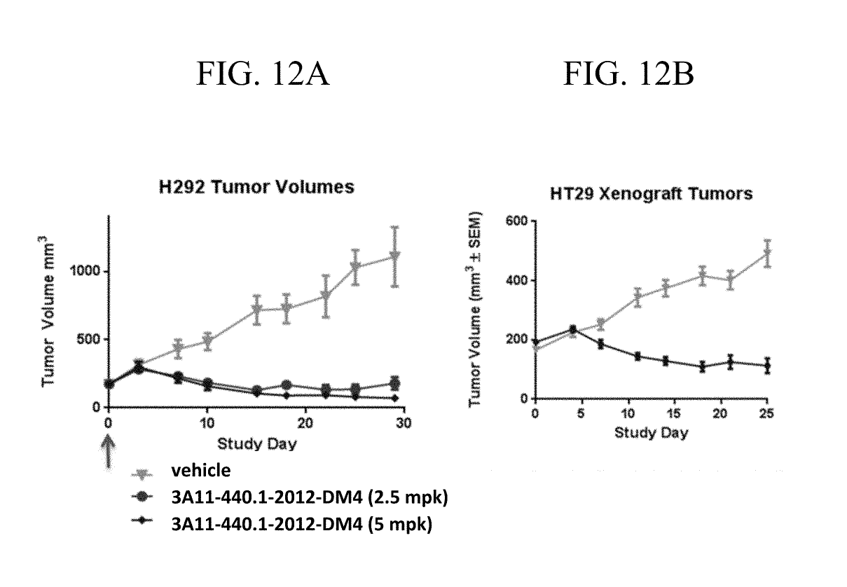

FIGS. 12A and 12B shows the efficacy of an anti-human CD147 activatable antibody drug conjugates (anti-human CD147 hu3A11-440.1-2012-DM4, VH of SEQ ID NO: 1, VL of SEQ ID NO: 171) in a mouse H292 (NSCLC) xenograft model (FIG. 12A) and a mouse HT29 (CRC) xenograft model (FIG. 12B). Tumors were grown to an average of 150 mm³; then the mice were randomized into different groups and dosed on day 0 with the indicated test articles. Mean tumor volume±SEM for each group is plotted. The activatable antibody drug conjugates of the present disclosure (anti-human CD147 hu3A11-440.1-2012-DM4, VH of SEQ ID NO: 1, VL of SEQ ID NO: 171) administered at 2.5 mg/kg or 5 mg/kg showed significantly higher efficacy than a vehicle control. Mean tumor volume±SEM for each group is plotted. Both the ADC and AADC induce tumor regressions.

### Example 11: In Vivo Toxicity of Anti-Human CD147-AADC in Cynomolgus Monkeys

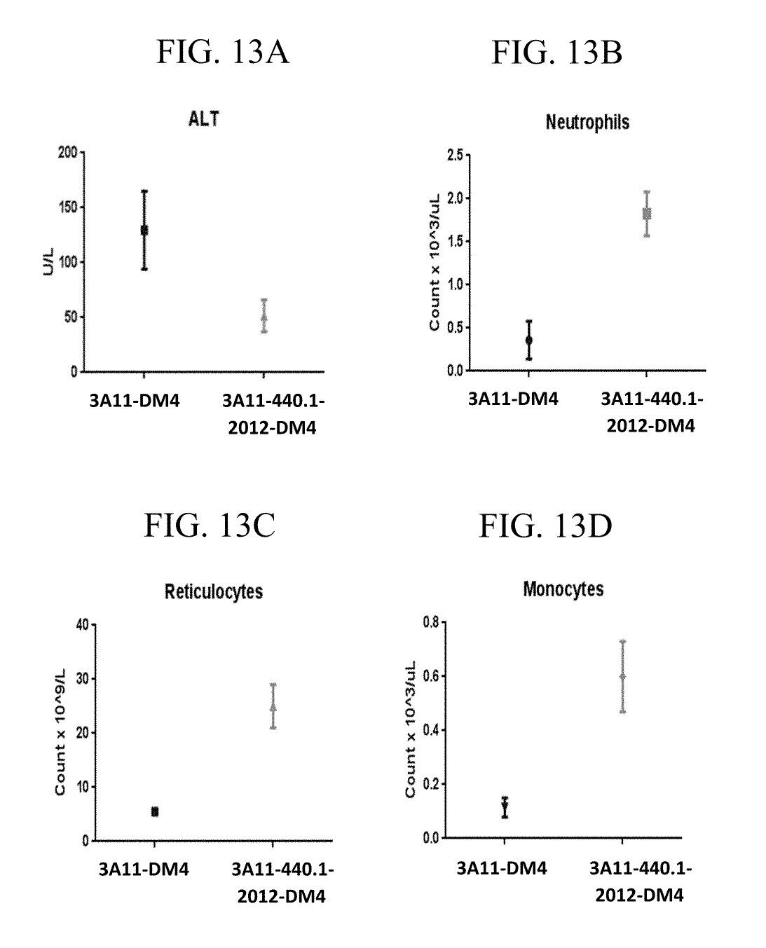

The tolerability of intact anti-human CD147 activatable antibody conjugated to DM4 (AADC) and anti-human CD147 antibody conjugated to DM4 (ADC) in cynomolgus monkeys was evaluated after a single 5 mg/kg dose (FIGS. 13A to 13D).

The studies depicted in FIGS. 13A to 13D were conducted as part of a 3 week tolerability study (non-terminal); single dose n=2. In this study, a single dose of intact activatable anti-human CD147 hu3A11-440.1-2012-spdb-DM4 (“AADC”) (anti-human CD147 hu3A11-440.1-2012-spdb-DM4, VH of SEQ ID NO: 1, VL of SEQ ID NO: 171) of the present disclosure having a drug-antibody ratio (DAR) of about 4.1 was dosed in cynomolgus monkeys at 4.4 mg/kg and anti-human CD147 hu3A11-spdb-DM4 (“ADC”) (anti-human CD147 hu3A11-spdb-DM4, VH of SEQ ID NO: 1, VL of SEQ ID NO: 5) of the present disclosure having a DAR of about 3.6 was dosed at 5 mg/kg. The conjugated activatable antibody was well tolerated at 5 mg/mg. No evidence of on or off-target toxicity was observed. There were no clinical signs, weight loss, clinical chemistry or hematologic findings.

FIGS. 13A-13D show that anti-human CD147 activatable antibody drug conjugates (AADCs) of the present disclosure (anti-human CD147 hu3A11-440.1-2012-spdb-DM4, VH of SEQ ID NO: 1, VL of SEQ ID NO: 171) demonstrate a higher tolerance in cynomolgus monkeys based on an absence of marked changes in each hematology readout and liver toxicity, as compared to monkeys treated with the corresponding parental anti-human CD147 ADC (anti-human CD147 hu3A11-spdb-DM4, VH of SEQ ID NO: 1, VL of SEQ ID NO: 5). In particular, monkeys treated with 5 mg/kg of the parental anti-human CD147 ADC showed higher levels liver toxicity based on levels of alanine transaminase (ALT) (FIG. 13A), severe neutropenia based on neutrophil counts (FIG. 13B), and decreased reticulocytes and monocytes (FIGS. 13C and 13D) as compared to monkeys treated with activatable anti-human CD147 AADCs of the present disclosure, which showed no marked changes in all of these hematological readouts (FIGS. 13A-13D).

### Example 12: In Vivo Pharmacokinetics and Tolerability of Anti-Human CD147-AADC and Anti-Human CD147 ADC in Cynomolgus Monkeys

The pharmacokinetics and tolerability in cynomolgus monkeys of an intact anti-human CD147 activatable antibody conjugated to DM4 (AADC) of the present disclosure and an anti-human CD147 antibody conjugated to DM4 (ADC) of the present disclosure were evaluated after a single 5 mg/kg dose of each was administered to male and female monkeys. The total serum level of human IgG was measured using an anti-human IgG sandwich ELISA using standard protocols.

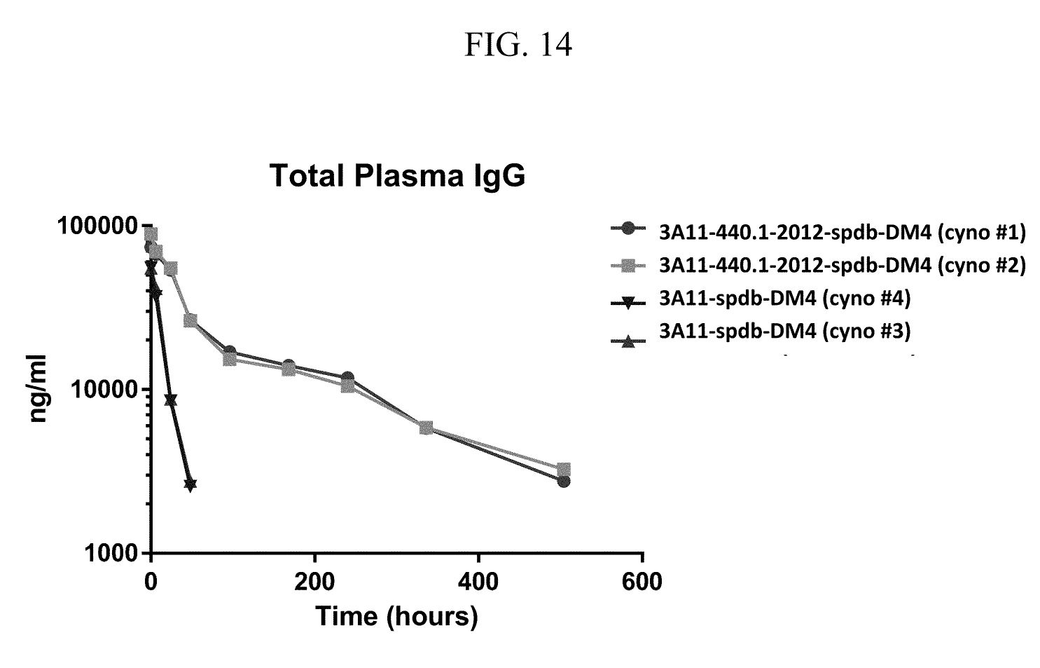

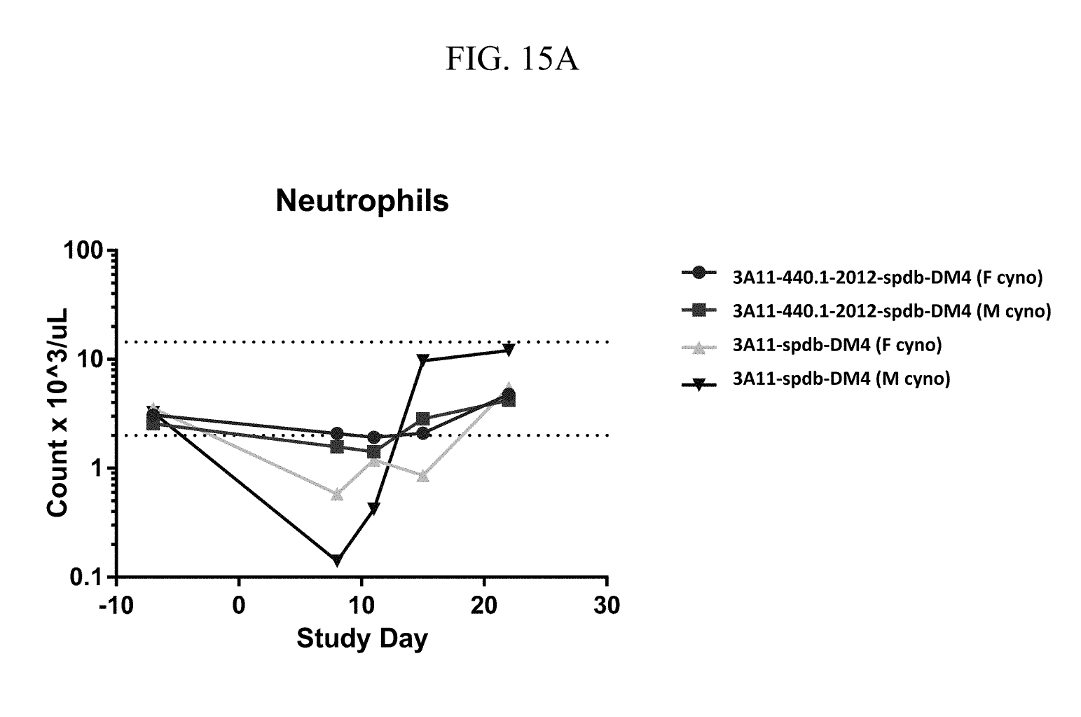

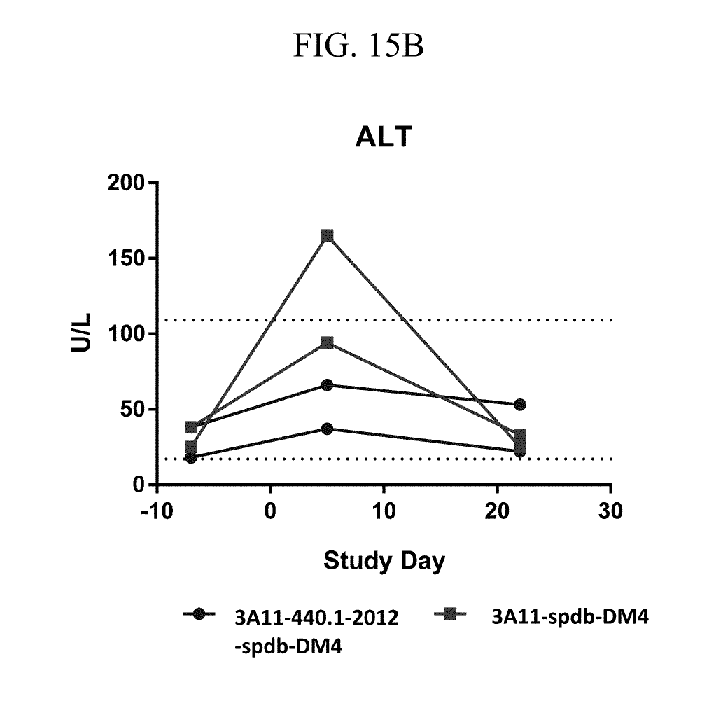

FIG. 14 show the pharmacokinetics of the hu3A11-440.1-2012-DM4 (VH of SEQ ID NO: 1, VL of SEQ ID NO: 171) activatable antibody drug conjugate (AADC) and the hu3A11-DM4 (VH of SEQ ID NO: 1, VL of SEQ ID NO: 5) antibody drug conjugate (ADC) of the present disclosure in cynomolgus monkeys, demonstrating that the AADC had a significantly longer half-life in both the male and female animals that were studied relative to the ADC. The total serum level of human IgG was measured using an anti-human IgG sandwich ELISA using standard protocols. FIGS. 15A and 15B show that the conjugated activatable anti-human CD147 antibodies (AADC) of the present disclosure tested was well tolerated in that it did not trigger an increase in markers of liver function (based on levels of alanine transaminase (ALT)) or neutropenia (based on neutrophil counts) as compared to the conjugated anti-human CD147 antibody (ADC) tested.

### Example 13: In Vitro Binding Affinity of Anti-CD147 Antibody to Glycosylated and Deglycosylated CD147

An exemplary study of the in vitro binding affinity of anti-human CD147 antibodies of the present disclosure to CD147 antigen is presented herein. Native human CD147 includes three arginine (N—) glycosylation sites. Glycosylation of these sites can result in a high-glycosylation CD147 isoform (˜40-60 kDa with complex carbohydrates) and a low-glycosylation form (˜32 kDa with mannose).

In this example, recombinant human CD147-Fc chimeric protein (R&D Systems, cat. no. 927-EMN-050) was deglycosylated of N- and simple O-linked carbohydrates using the Protein Deglycosylation Mix II (˜16 hrs incubation at 37° C.; New England Biolabs, cat. no. P6044S) or the Enzymatic Protein Deglycosylation Kit (˜1.5 days+incubation at 37° C.; Sigma-Aldrich, cat. no. EDEGLY) in accordance with their respective protocols. The glycosylated CD147-Fc fusion protein was observed to migrate at ˜62 kDa, while the CD147-Fc fusion protein treated with either deglycosylation kit was observed to migrate at 52 and 48 kDa. Bovine fetuin, which includes both N- and O-linked carbohydrates, was used a control for the extent of deglycosylation.

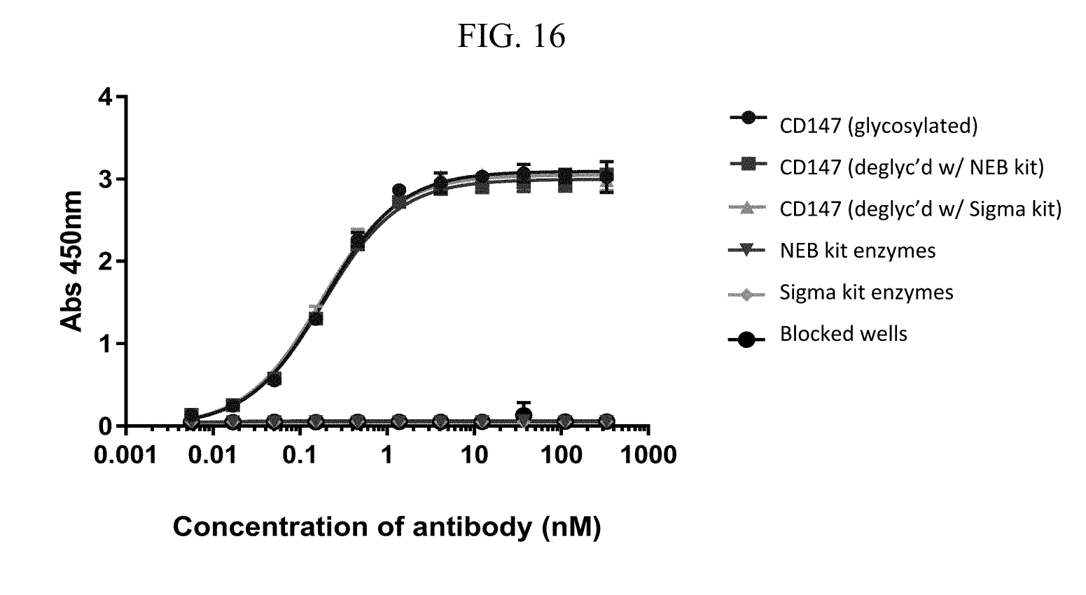

After buffer exchange with PBS, the deglycosylated or glycosylated proteins were adsorbed onto ELISA plates at 1 μg/mL. As shown in FIG. 16, in vitro binding affinity of humanized anti-CD147 monoclonal antibody of the present disclosure (3A11 Lc1/Hc1 with VL of SEQ ID NO: 5 and VH of SEQ ID NO: 1) was determined using a standard ELISA protocol by incubating the adsorbed protein with the indicated concentration of the anti-CD147 humanized antibody of the present disclosure. Bound humanized antibody of the present disclosure was detected with anti-human Fab-peroxidase secondary antibody and the mouse 3A11 monoclonal antibody was detected with anti-mouse Fc-peroxidase secondary antibody, and Ultra TMB detection (Thermo Fisher Scientific). The apparent equilibrium dissociation constants (K_{D}) of this exemplary binding study are shown in Table 25.

TABLE 25
In Vitro Binding Affinity of Anti-human CD147 Antibodies
To Glycosylated and Deglycosylated Human CD147

| Antigen | Bₘₐₓ | K_{D} (nM) |
|---|---|---|
| CD147-Fc (glycosylated) | 3.095 | 0.1914 |
| CD147-Fc (deglyc-NEB) | 2.997 | 0.1851 |
| CD147-Fc (deglyc-Sigma) | 3.050 | 0.1753 |

These exemplary data demonstrate that the anti-CD147 humanized antibody of the present disclosure bound to glycosylated and deglycosylated CD147 antigen with comparable affinity.

While the invention has been described in conjunction with the detailed description thereof, the foregoing description is intended to illustrate and not limit the scope of the invention, which is defined by the scope of the appended claims. Other aspects, advantages, and modifications are within the scope of the following.

1. An isolated antibody or an antigen binding fragment thereof (AB) that specifically binds human CD147 and cynomolgus monkey CD147.

2. The isolated antibody of claim 1, wherein the AB comprises an amino acid sequence comprising amino acid sequences selected from the group consisting of:
(a) the VH CDR1 sequence GFTFSNYWMN (SEQ ID NO: 10) or GFTFSNYWMD (SEQ ID NO: 11); the VH CDR2 sequence EIRLKSYNYATH (SEQ ID NO: 12); the VH CDR3 sequence AGTDY (SEQ ID NO: 13); the VL CDR1 sequence KASQSVRTDVA (SEQ ID NO: 14) or RASQSVRTDVG (SEQ ID NO: 15); the VL CDR2 sequence YSSNRYT (SEQ ID NO: 16); and the VL CDR3 sequence QQDYSSPFT (SEQ ID NO: 17) or QQDYSSPYT (SEQ ID NO: 18),
(b) the VH CDR1 sequence GFTFSNYWMD (SEQ ID NO: 11); the VH CDR2 sequence EIRLKSYNYATH (SEQ ID NO: 12); the VH CDR3 sequence AGTDY (SEQ ID NO: 13); the VL CDR1 sequence RASQSVRTDVG (SEQ ID NO: 15); the VL CDR2 sequence YSSNRYT (SEQ ID NO: 16); and the VL CDR3 sequence QQDYSSPYT (SEQ ID NO: 18), and
(c) the VH CDR1 sequence GFTFSNYWMN (SEQ ID NO: 10); the VH CDR2 sequence EIRLKSYNYATH (SEQ ID NO: 12); the VH CDR3 sequence AGTDY (SEQ ID NO: 13); the VL CDR1 sequence KASQSVRTDVA (SEQ ID NO: 14); the VL CDR2 sequence YSSNRYT (SEQ ID NO: 16); and the VL CDR3 sequence QQDYSSPFT (SEQ ID NO: 17).

3. The isolated antibody of claim 1, wherein the AB comprises a heavy chain variable region comprising an amino acid sequence selected from the group consisting of SEQ ID NOs: 1-4, and a light chain variable region comprising an amino acid sequence selected from the group consisting of SEQ ID NOs: 5-9.

4. An isolated antibody or antigen binding fragment thereof that binds to the same epitope on human CD147 and/or cynomolgus monkey CD147 as the AB of claim 2.

5. An isolated antibody or antigen binding fragment thereof that inhibits the binding of the AB of claim 2 to human CD147 and/or cynomolgus monkey CD147.

6. An activatable antibody that, in an activated state, specifically binds to human CD147 and/or cynomolgus monkey CD147 comprising:
an antibody or an antigen binding fragment thereof (AB) that specifically binds to human CD147 and cynomolgus monkey CD147;
a masking moiety (MM) coupled to the AB, wherein the MM inhibits the binding of the AB to CD147 when the activatable antibody is in an uncleaved state; and
a cleavable moiety (CM) coupled to the AB, wherein the CM is a polypeptide that functions as a substrate for a protease.

7. The activatable antibody of claim 6, wherein the MM has one or more of the characteristics selected from the group consisting of:
(i) the MM has a dissociation constant for binding to the AB that is greater than the dissociation constant of the AB to CD147;
(ii) the MM does not interfere or compete with the AB for binding to CD147 when the activatable antibody is in a cleaved state;
(iii) the MM is a polypeptide of no more than 40 amino acids in length;
(iv) the MM polypeptide sequence is different from that of human CD147;
(v) the MM polypeptide sequence is no more than 50% identical to any natural binding partner of the AB; and
(vi) the MM comprises an amino acid sequence selected from the group consisting of SEQ ID NOs: 30-100.

8-13. (canceled)

14. The activatable antibody of claim 6, wherein the CM has one or more of the characteristics selected from the group consisting of:
(i) the CM is a substrate for a protease that is active in diseased tissue; and
(ii) the CM comprises an amino acid sequence selected from the group consisting of SEQ ID NOs: 356-423, 680-698, 713, 714, and 789-808.

15-16. (canceled)

17. The activatable antibody of claim 6, wherein the antigen binding fragment thereof is selected from the group consisting of a Fab fragment, a F(ab′)₂ fragment, a scFv, a scAb, a dAb, a single domain heavy chain antibody, and a single domain light chain antibody.

18. (canceled)

19. The activatable antibody of claim 6, wherein AB has one or more of the characteristics selected from the group consisting of:
(i) the AB comprises the VH CDR1 sequence GFTFSNYWMN (SEQ ID NO: 10) or GFTFSNYWMD (SEQ ID NO: 11); the VH CDR2 sequence EIRLKSYNYATH (SEQ ID NO: 12); the VH CDR3 sequence AGTDY (SEQ ID NO: 13); the VL CDR1 sequence KASQSVRTDVA (SEQ ID NO: 14) or RASQSVRTDVG (SEQ ID NO: 15); the VL CDR2 sequence YSSNRYT (SEQ ID NO: 16); and the VL CDR3 sequence QQDYSSPFT (SEQ ID NO: 17) or QQDYSSPYT (SEQ ID NO: 18); and
(ii) the AB comprises a heavy chain variable region comprising an amino acid sequence selected from the group consisting of SEQ ID NOs: 1-3, and a light chain variable region comprising an amino acid sequence selected from the group consisting of SEQ ID NOs: 5-8.

20. (canceled)

21. The activatable antibody of claim 6, wherein the AB is linked to the CM.

22. The activatable antibody of claim 6, wherein the AB is linked directly to the CM.

23. The activatable antibody of claim 6, wherein the AB is linked to the CM via a linking peptide.

24. The activatable antibody of claim 6, wherein the MM is linked to the CM such that the activatable antibody in an uncleaved state comprises the structural arrangement from N-terminus to C-terminus as follows: MM-CM-AB or AB-CM-MM.

25. The activatable antibody of claim 6, wherein the activatable antibody comprises a linking peptide between the MM and the CM, a linking peptide between the CM and the AB, or both a linking peptide between the MM and the CM and a linking peptide between the CM and the AB.

26. (canceled)

27. The activatable antibody of claim 6, wherein the activatable antibody comprises a first linking peptide (LP1) and a second linking peptide (LP2), and wherein the activatable antibody in the uncleaved state has the structural arrangement from N-terminus to C-terminus as follows: MM-LP1-CM-LP2-AB or AB-LP2-CM-LP1-MM.

28. The activatable antibody of claim 27, wherein the two linking peptides are not identical to each other.

29. The activatable antibody of claim 27, wherein each of LP1 and LP2 is a peptide of about 1 to 20 amino acids in length.

30. The activatable antibody of claim 6, wherein the activatable antibody has one or more of the characteristics selected from the group consisting of:
(a) the activatable antibody comprises the heavy chain sequence selected from the group consisting of SEQ ID NOs: 19-22 and a light chain comprising an amino acid sequence selected from the group consisting of SEQ ID NOs: 23-27, 230-272, and 275-317;
(b) the activatable antibody comprises a combination of amino acid sequences, wherein the combination of amino acid sequences is selected from a single row in Table 4, wherein for a given combination,
(i) the heavy chain of the AB comprises the amino acid sequences of the VH CDR sequences corresponding to the given combination in the single row listed in Table 4,
(ii) the light chain of the AB comprises the amino acid sequences of the VL CDR sequences corresponding to the given combination in the single row listed in Table 4,
(ii) the MM comprises the amino acid sequence of the mask sequence (MM) corresponding to the given combination in the single row listed in Table 4, and
(iv) the CM comprises the amino acid sequence of the substrate sequence (CM) corresponding to the given combination in the single row listed in Table 4; and
(c) the activatable antibody comprises a combination of amino acid sequences, wherein for a given combination of amino acid sequences,
(i) the heavy chain of the AB comprises the amino acid sequences of the VH sequence or VH CDR sequences selected from the group consisting of: the VH sequence or VH CDR sequences listed in the corresponding column of Table 5,
(ii) the light chain of the AB comprises the amino acid sequences of the VL sequence or VL CDR sequences selected from the group consisting of: the VL sequence or VL CDR sequences listed in the corresponding column of Table 5,
(iii) the MM comprises the amino acid sequence of the mask sequence (MM) selected from the group consisting of: the MM sequences listed in the corresponding column of Table 5, and
(iv) the CM comprises the amino acid sequence of the substrate sequence (CM) selected from the group consisting of: the CM sequences listed in the corresponding column of Table 5.

31-32. (canceled)

33. An activatable antibody comprising an antibody or an antigen binding fragment thereof (AB) that specifically binds to mammalian CD147, a MM, and a CM, wherein the activatable antibody has one or more of the following characteristics selected from the group consisting of:
(i) the activatable antibody comprises a heavy chain sequence comprising an amino acid sequence selected from the group consisting of SEQ ID NOs: 19-22; and
a light chain comprising an amino acid sequence selected from the group consisting of SEQ ID NOs: 23-27, 230-272, and 275-317;
(ii) the MM comprises an amino acid sequence selected from the group consisting of SEQ ID NOs: 30-100, and the CM comprises an amino acid sequence selected from the group consisting of SEQ ID NOs: 356-423, 680-698, 713, 714, and 789-808;
(iii) the AB comprises the VH CDR1 sequence GFTFSNYWMN (SEQ ID NO: 10) or GFTFSNYWMD (SEQ ID NO: 11); the VH CDR2 sequence EIRLKSYNYATH (SEQ ID NO: 12); the VH CDR3 sequence AGTDY (SEQ ID NO: 13); the VL CDR1 sequence KASQSVRTDVA (SEQ ID NO: 14) or RASQSVRTDVG (SEQ ID NO: 15); the VL CDR2 sequence YSSNRYT (SEQ ID NO: 16); and the VL CDR3 sequence QQDYSSPFT (SEQ ID NO: 17) or QQDYSSPYT (SEQ ID NO: 18);
(iv) the activatable antibody comprises a heavy chain variable region comprising an amino acid sequence selected from the group consisting of SEQ ID NOs: 1-3, and a light chain variable region comprising an amino acid sequence selected from the group consisting of SEQ ID NOs: 5-8, 140-182, and 185-227; and
(v) the AB comprises the VH CDR1 sequence GFTFSNYWMN (SEQ ID NO: 10) or GFTFSNYWMD (SEQ ID NO: 11); the VH CDR2 sequence EIRLKSYNYATH (SEQ ID NO: 12); the VH CDR3 sequence AGTDY (SEQ ID NO: 13); the VL CDR1 sequence KASQSVRTDVA (SEQ ID NO: 14) or RASQSVRTDVG (SEQ ID NO: 15); the VL CDR2 sequence YSSNRYT (SEQ ID NO: 16); and the VL CDR3 sequence QQDYSSPFT (SEQ ID NO: 17) or QQDYSSPYT (SEQ ID NO: 18), the MM comprises an amino acid sequence selected from the group consisting of SEQ ID NOs: 30-100, and the CM comprises an amino acid sequence selected from the group consisting of SEQ ID NOs: 356-423, 680-698, 713, 714, and 789-808.

34-37. (canceled)

38. An activatable anti-human CD147 antibody comprising
an antibody or an antigen binding fragment thereof (AB) that specifically binds to mammalian CD147, wherein the AB specifically binds to the same epitope on human CD147 and/or cynomolgus monkey CD147 as the isolated antibody of claim 2 or wherein the AB specifically cross-competes with the isolated antibody of claim 2 for binding to human CD147 and/or cynomolgus monkey CD147;
a masking moiety (MM) coupled to the AB that inhibits the binding of the AB to CD147 when the activatable antibody is in an uncleaved state; and
a cleavable moiety (CM) coupled to the AB, wherein the CM is a polypeptide that functions as a substrate for a protease.

39. (canceled)

40. A conjugated activatable antibody comprising the activatable antibody of claim 6 conjugated to an agent.

41. The conjugated activatable antibody of claim 40, wherein the agent has one or more of the characteristics selected from the group consisting of:
(a) the agent is a toxin or fragment thereof;
(b) the agent is a microtubule inhibitor;
(c) the agent is a nucleic acid damaging agent;
(d) the agent is a dolastatin or a derivative thereof;
(e) the agent is an auristatin or a derivative thereof;
(f) the agent is a maytansinoid or a derivative thereof;
(g) the agent is a duocarmycin or a derivative thereof;
(h) the agent is a calicheamicin or a derivative thereof;
(i) the agent is a pyrrolobenzodiazepine or a derivative thereof;
(i) the agent is auristatin E or a derivative thereof;
(k) the agent is monomethyl auristatin E (MMAE);
(l) the agent is monomethyl auristatin D (MMAD);
(m) the agent is the maytansinoid DM1;
(n) the agent is the maytansinoid DM4;
(o) the agent is a detectable moiety; and
(p) the agent is a diagnostic agent.

42-50. (canceled)

51. The conjugated activatable antibody of claim 40, wherein the agent is conjugated to the AB via a linker.

52. The conjugated activatable antibody of claim 40, wherein the linker with which the agent is conjugated to the AB comprises an SPDB moiety, a vc moiety, or a PEG2-vc moiety.

53. The conjugated activatable antibody of claim 40, wherein the agent is a toxin conjugated to the AB via a linker, and wherein the linker and the toxin conjugated to the AB comprise a moiety selected from the group consisting of: an SPDB-DM4 moiety, a vc-MMAD moiety, a vc-MMAE moiety, a vc-duocarmycin moiety, or a PEG2-vc-MMAD moiety.

54. The conjugated activatable antibody of claim 51, wherein the linker is a cleavable linker.

55. The conjugated activatable antibody of claim 51, wherein the linker is a non-cleavable linker.

56-57. (canceled)

58. A conjugated activatable antibody that, in an activated state, binds CD147 comprising:
(a) an activatable antibody comprising:
(i) an antibody or an antigen binding fragment thereof (AB) that specifically binds to human CD147 and/or cynomolgus monkey CD147;
(ii) a masking moiety (MM) coupled to the AB that inhibits the binding of the AB to CD147 when the activatable antibody is in an uncleaved state;
(iii) a cleavable moiety (CM) coupled to the AB, wherein the CM is a polypeptide that functions as a substrate for a protease; and
(b) an agent conjugated to the AB.

59. The conjugated activatable antibody of claim 58, wherein the agent is selected from the group consisting of a dolastatin or a derivative thereof, an auristatin or a derivative thereof, a maytansinoid or a derivative thereof, a duocarmycin or a derivative thereof, a calicheamicin or a derivative thereof, a pyrrolobenzodiazepine or a derivative thereof, auristatin E, monomethyl auristatin F (MMAF), monomethyl auristatin E (MMAE), monomethyl auristatin D (MMAD), maytansinoid DM4, maytansinoid DM1, a calicheamicin, a duocarmycin, a pyrrolobenzodiazepine, and a pyrrolobenzodiazepine dimer.

60. (canceled)

61. The conjugated activatable antibody of claim 58, wherein the agent is conjugated to the AB via a linker.

62. The conjugated activatable antibody of claim 58, wherein the linker with which the agent is conjugated to the AB comprises an SPDB moiety, a vc moiety, or a PEG2-vc moiety.

63. The conjugated activatable antibody of claim 58, wherein the agent is a toxin conjugated to the AB via a linker, and wherein the linker and the toxin conjugated to the AB comprise a moiety selected from the group consisting of: an SPDB-DM4 moiety, a vc-MMAD moiety, a vc-MMAE moiety, a vc-duocarmycin moiety, or a PEG2-vc-MMAD moiety.

64. The conjugated activatable antibody of claim 58, wherein the activatable antibody has one or more of the characteristics selected from the group consisting of:
(i) the activatable antibody comprises the VH CDR1 sequence GFTFSNYWMN (SEQ ID NO: 10) or GFTFSNYWMD (SEQ ID NO: 11); the VH CDR2 sequence EIRLKSYNYATH (SEQ ID NO: 12); the VH CDR3 sequence AGTDY (SEQ ID NO: 13); the VL CDR1 sequence KASQSVRTDVA (SEQ ID NO: 14) or RASQSVRTDVG (SEQ ID NO: 15); the VL CDR2 sequence YSSNRYT (SEQ ID NO: 16); and the VL CDR3 sequence QQDYSSPFT (SEQ ID NO: 17) or QQDYSSPYT (SEQ ID NO: 18);
(ii) the activatable antibody comprises a heavy chain variable region comprising an amino acid sequence selected from the group consisting of SEQ ID NOs: 1-3, and a light chain variable region comprising an amino acid sequence selected from the group consisting of SEQ ID NOs: 5-8, 140-182, and 185-227; and
(iii) the activatable antibody comprises the heavy chain comprising an amino acid sequence of SEQ ID NOs: 19-22 and a light chain comprising an amino acid sequence selected from the group consisting of SEQ ID NOs: 23-27, 230-272, and 275-317.

65. (canceled)

66. The conjugated activatable antibody of claim 58, wherein the MM comprises an amino acid sequence selected from the group consisting of SEQ ID NOs: 30-100.

67. (canceled)

68. The conjugated activatable antibody of claim 58, wherein the CM comprises an amino acid sequence selected from the group consisting of SEQ ID NOs: 356-423, 680-698, 713, 714, and 789-808.

69. (canceled)

70. The conjugated activatable antibody of claim 58, wherein the activatable antibody has one or more of the following characteristics selected from the group consisting of:
(i) the activatable antibody comprises a combination of amino acid sequences, wherein the combination of amino acid sequences is selected from a single row in Table 4,
wherein for a given combination,
(a) the heavy chain of the AB comprises the amino acid sequences of the VH CDR sequences corresponding to the given combination in the single row listed in Table 4,
(b) the light chain of the AB comprises the amino acid sequences of the VL CDR sequences corresponding to the given combination in the single row listed in Table 4,
(c) the MM comprises the amino acid sequence of the mask sequence (MM) corresponding to the given combination in the single row listed in Table 4, and
(d) the CM comprises the amino acid sequence of the substrate sequence (CM) corresponding to the given combination in the single row listed in Table 4; and
(ii) the activatable antibody comprises a combination of amino acid sequences, wherein for a given combination of amino acid sequences,
(a) the heavy chain of the AB comprises the amino acid sequences of the VH sequence or VH CDR sequences selected from the group consisting of: the VH sequence or VH CDR sequences listed in the corresponding column of Table 5,
(b) the light chain of the AB comprises the amino acid sequences of the VL sequence or VL CDR sequences selected from the group consisting of: the VL sequence or VL CDR sequences listed in the corresponding column of Table 5,
(c) the MM comprises the amino acid sequence of the mask sequence (MM) selected from the group consisting of: the MM sequences listed in the corresponding column of Table 5, and
(d) the CM comprises the amino acid sequence of the substrate sequence (CM) selected from the group consisting of: the CM sequences listed in the corresponding column of Table 5.

71-72. (canceled)

73. A conjugated antibody comprising:
(a) an antibody or an antigen binding fragment thereof (AB) that specifically binds to mammalian CD147, wherein the AB comprises:
(i) the VH CDR1 sequence GFTFSNYWMN (SEQ ID NO: 10) or GFTFSNYWMD (SEQ ID NO: 11); the VH CDR2 sequence EIRLKSYNYATH (SEQ ID NO: 12); the VH CDR3 sequence AGTDY (SEQ ID NO: 13); the VL CDR1 sequence KASQSVRTDVA (SEQ ID NO: 14) or RASQSVRTDVG (SEQ ID NO: 15); the VL CDR2 sequence YSSNRYT (SEQ ID NO: 16); and the VL CDR3 sequence QQDYSSPFT (SEQ ID NO: 17) or QQDYSSPYT (SEQ ID NO: 18), or
(ii) a heavy chain variable region comprising an amino acid sequence selected from the group consisting of SEQ ID NOs: 1-4, and a light chain variable region comprising an amino acid sequence selected from the group consisting of SEQ ID NOs: 5-9, 140-182, and 185-227 or
(iii) a heavy chain comprising an amino acid sequence selected from the group consisting of SEQ ID NOs: 19-22, and a light chain comprising an amino acid sequence selected from the group consisting of SEQ ID NOs: 23-27, 230-272, and 275-317;
(b) an agent conjugated to the AB, wherein the agent is selected from the group consisting of auristatin E, monomethyl auristatin F (MMAF), monomethyl auristatin E (MMAE), monomethyl auristatin D (MMAD), maytansinoid DM4, maytansinoid DM1, a pyrrolobenzodiazepine, a pyrrolobenzodiazepine dimer, a duocarmycin, and a calicheamicin.

74. A conjugated activatable antibody that, in an activated state, binds to CD147, comprising:
(a) an activatable antibody comprising:
(i) an antibody or an antigen binding fragment thereof (AB) that specifically binds to human CD147 and cynomolgus monkey CD147;
(ii) a masking moiety (MM) coupled to the AB that inhibits the binding of the AB to CD147 when the activatable antibody is in an uncleaved state;
(iii) a cleavable moiety (CM) coupled to the AB, wherein the CM is a polypeptide that functions as a substrate for a protease; and
(b) an agent conjugated to the AB,
wherein the activatable antibody comprises:
(i) the VH CDR1 sequence GFTFSNYWMN (SEQ ID NO: 10) or GFTFSNYWMD (SEQ ID NO: 11); the VH CDR2 sequence EIRLKSYNYATH (SEQ ID NO: 12); the VH CDR3 sequence AGTDY (SEQ ID NO: 13); the VL CDR1 sequence KASQSVRTDVA (SEQ ID NO: 14) or RASQSVRTDVG (SEQ ID NO: 15); the VL CDR2 sequence YSSNRYT (SEQ ID NO: 16); and the VL CDR3 sequence QQDYSSPFT (SEQ ID NO: 17) or QQDYSSPYT (SEQ ID NO: 18), or
(ii) a heavy chain variable region comprising an amino acid sequence selected from the group consisting of SEQ ID NOs: 1-4, and a light chain variable region comprising an amino acid sequence selected from the group consisting of SEQ ID NOs: 5-9, 140-182, and 185-227, or
(iii) a heavy chain comprising an amino acid sequence selected from the group consisting of SEQ ID NOs: 19-22, and a light chain comprising an amino acid sequence selected from the group consisting of SEQ ID NOs: 23-27, 230-272, and 275-317; and
wherein the agent is selected from the group consisting of auristatin E, monomethyl auristatin F (MMAF), monomethyl auristatin E (MMAE), monomethyl auristatin D (MMAD), maytansinoid DM4, maytansinoid DM1, a pyrrolobenzodiazepine, a pyrrolobenzodiazepine dimer, a duocarmycin, and a calicheamicin.

75. The conjugated activatable antibody of claim 74, wherein the MM comprises an amino acid sequence selected from the group consisting of SEQ ID NOs: 30-100.

76. (canceled)

77. The conjugated activatable antibody of claim 74, wherein the CM comprises an amino acid sequence selected from the group consisting of SEQ ID NOs: 356-423, 680-698, 713, 714, and 789-808.

78. (canceled)

79. The conjugated activatable antibody of claim 74, wherein the agent is conjugated to the AB via a linker, and wherein the linker to which the agent is conjugated to the AB comprises an SPDB moiety, a vc moiety, or a PEG2-vc moiety.

80. The conjugated activatable antibody of claim 74, wherein the agent is a toxin conjugated to the AB via a linker, and wherein the linker and the toxin conjugated to the AB comprise a moiety selected from the group consisting of: an SPDB-DM4 moiety, a vc-MMAD moiety, a vc-MMAE moiety, a vc-duocarmycin moiety, or a PEG2-vc-MMAD moiety.

81. A conjugated activatable antibody or conjugated antibody comprising:
an antibody or antigen binding fragment thereof (AB) that, in an activated state, binds CD147; and
a toxin conjugated to the AB via a linker,
wherein the conjugated activatable antibody or the conjugated antibody comprises amino acid sequences, a linker, and a toxin selected from a single row in Table 9, wherein for the given combination:
(a) the AB comprises a heavy chain comprising the amino acid sequence of the heavy chain sequence or heavy chain variable domain sequence corresponding to the given combination in the single row listed in Table 9,
(b) the AB comprises a light chain comprising the amino acid sequence of the light chain sequence or light chain variable domain sequence corresponding to the given combination in the single row listed in Table 9, and
(c) the linker and the toxin comprise the linker and the toxin corresponding to the given combination in the single row listed in Table 9.

82. A pharmaceutical composition comprising the activatable antibody of claim 6; and a carrier.

83. The pharmaceutical composition of claim 82 comprising an additional agent.

84. The pharmaceutical composition of claim 83, wherein the additional agent is a therapeutic agent.

85. An isolated nucleic acid molecule encoding the activatable antibody of claim 6.

86. A vector comprising the isolated nucleic acid molecule of claim 85.

87. A method of producing an antibody or an activatable antibody by culturing a cell under conditions that lead to expression of the antibody or the activatable antibody, wherein the cell comprises the nucleic acid molecule of claim 85.

88. A method of manufacturing an activatable antibody that, in an activated state, specifically binds CD147, the method comprising:
(a) culturing a cell comprising a nucleic acid construct that encodes the activatable antibody under conditions that lead to expression of the activatable antibody, wherein the activatable antibody comprises an activatable antibody of claim 6; and
(b) recovering the activatable antibody.

89. A method of treating, alleviating a symptom of, or delaying the progression of a disorder or disease in which diseased cells express CD147 comprising administering a therapeutically effective amount of the conjugated activatable antibody of claim 40 to a subject in need thereof.

90. The method of claim 89, wherein the disorder or disease is cancer.

91. A method of treating, alleviating a symptom of, or delaying the progression of a disorder or disease associated with cells expressing CD147 comprising administering a therapeutically effective amount of the conjugated activatable antibody of claim 40 to a subject in need thereof.

92. The method of claim 91, wherein the disorder or disease associated with cells expressing CD147 is cancer.

93. The method of claim 92, wherein the cancer is an adenocarcinoma, a bile duct (biliary) cancer, a bladder cancer, a bone cancer, a breast cancer, a triple-negative breast cancer, a Her2-negative breast cancer, a carcinoid cancer, a cervical cancer, a cholangiocarcinoma, a colorectal cancer, a colon cancer, an endometrial cancer, an esophageal cancer, a glioma, a head and neck cancer, a head and neck squamous cell cancer, a leukemia, a liver cancer, a lung cancer, a non-small cell lung cancer, a small cell lung cancer, a lymphoma, a melanoma, an oropharyngeal cancer, an ovarian cancer, a pancreatic cancer, a prostate cancer, a metastatic castration-resistant prostate carcinoma, a renal cancer, a sarcoma, a skin cancer, a squamous cell cancer, a stomach cancer, a testis cancer, a thyroid cancer, a urogenital cancer, or a urothelial cancer.

94. A method of inhibiting or reducing the growth, proliferation, or metastasis of cells expressing mammalian CD147 comprising administering a therapeutically effective amount of the conjugated activatable antibody of claim 40 to a subject in need thereof.

95. A method of inhibiting, blocking, or preventing the binding of a natural ligand to mammalian CD147, comprising administering a therapeutically effective amount of the conjugated activatable antibody of claim 40 to a subject in need thereof.

96. The method of any one of claim 95, wherein the expression and/or activity of the mammalian CD147 is aberrant.

97. The method of claim 89, wherein the method comprises administering an additional agent.

98. The method of claim 97, wherein the additional agent is a therapeutic agent.

99. The isolated antibody of claim 1, wherein the isolated antibody or antigen binding fragment thereof specifically binds to deglycosylated CD147 and glycosylated CD147.

100. The isolated antibody of claim 99, wherein the CD147 is human CD147.

101. The activatable antibody of claim 6, wherein the AB specifically binds to deglycosylated CD147 and glycosylated CD147.

102. The activatable antibody of claim 101, wherein the CD147 is human CD147.

103. A pharmaceutical composition comprising the conjugated activatable antibody of claim 40; and a carrier.

104. The pharmaceutical composition of claim 103 comprising an additional agent.

105. The pharmaceutical composition of claim 104, wherein the additional agent is a therapeutic agent.
